SEQUENCE LISTING (207 sequences)

SEQ ID NO: 1 - protein, 274 aa, Homo sapiens
AESHLSLLYHLTAVSSPAPGTPAFWVSGWLGPQQYLSYNSLRGEAEPCGAWVWENQVSWYWEKETTDLRIKEKLFLEAFKALGGKGPYTLQGLLGCELGPDNTSVPTAKFALNGEEFMNFDLKQGTWGGDWPEALAISQRWQQQDKAANKELTFLLFSCPHRLREHLERGRGNLEWKEPPSMRLKARPSSPGFSVLTCSAFSFYPPELQLRFLRNGLAAGTGQGDFGPNSDGSFHASSSLTVKSGDEHHYCCIVQHAGLAQPLRVELESPAKSS

SEQ ID NO: 2 - skipped

SEQ ID NO: 3 - protein, 17 aa, synthetic construct
  VARIANT 3: note = A, D, E, G, H, P, Q, R, S, T, or V
  VARIANT 4: note = A, D, E, G, K, M, N, P, Q, R, S, T, V, W, or Y
  VARIANT 8: note = A, D, G, H, K, L, M, Q, S, T, or V
  VARIANT 9: note = A, D, E, F, G, H, I, K, L, M, N, P, Q, R, S, T, V, W, or Y
  VARIANT 10: note = A, D, E, H, S, T, V, or Y
AIXXGGGXXXYADSVKG

SEQ ID NO: 4 - protein, 12 aa, synthetic construct
  VARIANT 2: note = A, D, E, F, G, I, L, M, Q, S, T, V, W, or Y
  VARIANT 3: note = L or N
  VARIANT 4: note = F, L, W, or Y
  VARIANT 6: note = A, D, E, K, L, M, Q, S, or W
  VARIANT 7: note = L, M, or V
  VARIANT 9: note = A, D, E, F, G, H, K, L, M, Q, R, S, T, V, W or Y
  VARIANT 11: note = A, E, L, M, Q, S, T, V, or W
  VARIANT 12: note = T or Y
DXXXTXXTXYXX

SEQ ID NO: 5 - skipped

SEQ ID NO: 6 - protein, 10 aa, synthetic construct
  VARIANT 3: note = A, D, E, G, H, P, Q, R, S, T, or V
  VARIANT 4: note = A, D, E, G, K, M, N, P, Q, R, S, T, V, W, or Y
  VARIANT 8: note = A, D, G, H, K, L, M, Q, S, T, or V
  VARIANT 9: note = A, D, E, F, G, H, I, K, L, M, N, P, Q, R, S, T, V, W, or Y
  VARIANT 10: note = A, D, E, H, S, T, V, or Y
AIXXGGGXXX

SEQ ID NO: 7 - protein, 12 aa, synthetic construct
  VARIANT 2: note = A, D, E, F, G, I, L, M, Q, S, T, V, W, or Y
  VARIANT 3: note = L or N
  VARIANT 4: note = F, L, W, or Y
  VARIANT 6: note = A, D, E, K, L, M, Q, S, or W
  VARIANT 7: note = L, M, or V
  VARIANT 9: note = A, D, E, F, G, H, K, L, M, Q, R, S, T, V, W or Y
  VARIANT 11: note = A, E, L, M, Q, S, T, V, or W
  VARIANT 12: note = T or Y
DXXXTXXTXYXX

SEQ ID NO: 8 - protein, 5 aa, synthetic construct
SYAMY

SEQ ID NO: 9 - protein, 17 aa, synthetic construct
AISSGGGSTDYADSVKG

SEQ ID NO: 10 - protein, 12 aa, synthetic construct
DTLYTSLTSYSY

SEQ ID NO: 11 - protein, 10 aa, synthetic construct
GFTFSSYAMY

SEQ ID NO: 12 - protein, 10 aa, synthetic construct
AISSGGGSTD

SEQ ID NO: 13 - protein, 12 aa, synthetic construct
DTLYTSLTSYSY

SEQ ID NO: 14 - protein, 121 aa, synthetic construct
  REGION 1..121: note = T0263018B11-parent
EVQLVESGGGVVQPGGSLRLSCAASGFTFSSYAMYWVRQAPGKGLEWVSAISSGGGSTDYADSVKGRFTISRDNSKNTLYLQMNSLRPEDTALYYCAADTLYTSLTWYSYWGQGTLVTVSS

SEQ ID NO: 15 - protein, 121 aa, synthetic construct
  REGION 1..121: note = T0263018B11-W100cS
EVQLVESGGGVVQPGGSLRLSCAASGFTFSSYAMYWVRQAPGKGLEWVSAISSGGGSTDYADSVKGRFTISRDNSKNTLYLQMNSLRPEDTALYYCAADTLYTSLTSYSYWGQGTLVTVSS

SEQ ID NO: 16 - protein, 5 aa, synthetic construct
SFGMS

SEQ ID NO: 17 - protein, 17 aa, synthetic construct
SISGSGSDTLYADSVKG

SEQ ID NO: 18 - protein, 6 aa, synthetic construct
GGSLSR

SEQ ID NO: 19 - protein, 10 aa, synthetic construct
GFTFRSFGMS

SEQ ID NO: 20 - protein, 10 aa, synthetic construct
SISGSGSDTL

SEQ ID NO: 21 - protein, 6 aa, synthetic construct
GGSLSR

SEQ ID NO: 22 - protein, 221 aa, synthetic construct
  REGION 1..221: note = Amino acid sequences of variant Fc regions (WO2015100299)
CPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKFHYTQKSLSLSPG

SEQ ID NO: 23 - protein, 227 aa, synthetic construct
  REGION 1..227: note = Amino acid sequences of variant Fc regions
DKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKFHYTQKSLSLSPGK

SEQ ID NO: 24 - protein, 226 aa, synthetic construct
  REGION 1..226: note = Amino acid sequences of variant Fc regions
DKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKFHYTQKSLSLSPG

SEQ ID NO: 25 - protein, 217 aa, synthetic construct
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 26 - protein, 217 aa, synthetic construct
APEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 27 - protein, 216 aa, synthetic construct
APPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 28 - skipped

SEQ ID NO: 29 - protein, 5 aa, synthetic construct
GGGGS

SEQ ID NO: 30 - protein, 7 aa, synthetic construct
SGGSGGS

SEQ ID NO: 31 - protein, 8 aa, synthetic construct
GGGGSGGS

SEQ ID NO: 32 - protein, 9 aa, synthetic construct
GGGGSGGGS

SEQ ID NO: 33 - protein, 10 aa, synthetic construct
GGGGSGGGGS

SEQ ID NO: 34 - protein, 15 aa, synthetic construct
GGGGSGGGGSGGGGS

SEQ ID NO: 35 - protein, 18 aa, synthetic construct
GGGGSGGGGSGGGGSGGS

SEQ ID NO: 36 - protein, 20 aa, synthetic construct
GGGGSGGGGSGGGGSGGGGS

SEQ ID NO: 37 - protein, 25 aa, synthetic construct
GGGGSGGGGSGGGGSGGGGSGGGGS

SEQ ID NO: 38 - protein, 30 aa, synthetic construct
GGGGSGGGGSGGGGSGGGGSGGGGSGGGGS

SEQ ID NO: 39 - protein, 35 aa, synthetic construct
GGGGSGGGGSGGGGSGGGGSGGGGSGGGGSGGGGS

SEQ ID NO: 40 - protein, 40 aa, synthetic construct
GGGGSGGGGSGGGGSGGGGSGGGGSGGGGSGGGGSGGGGS

SEQ ID NO: 41 - protein, 15 aa, synthetic construct
EPKSCDKTHTCPPCP

SEQ ID NO: 42 - protein, 24 aa, synthetic construct
GGGGSGGGSEPKSCDKTHTCPPCP

SEQ ID NO: 43 - protein, 12 aa, synthetic construct
EPKTPKPQPAAA

SEQ ID NO: 44 - protein, 62 aa, synthetic construct
ELKTPLGDTTHTCPRCPEPKSCDTPPPCPRCPEPKSCDTPPPCPRCPEPKSCDTPPPCPRCP

SEQ ID NO: 45 - protein, 115 aa, synthetic construct [sequence omitted]

SEQ ID NO: 46 - protein, 115 aa, synthetic construct [sequence omitted]

SEQ ID NO: 47 - protein, 116 aa, synthetic construct
EVQLVESGGGVVQPGNSLRLSCAASGFTFSSFGMSWVRQAPGKGLEWVSSISGSGSDTLYADSVKGRFTISRDNAKTTLYLQMNSLRPEDTATYYCTIGGSLSRSSQGTLVTVSSA

SEQ ID NO: 48 - protein, 116 aa, synthetic construct
EVQLVESGGGVVQPGGSLRLSCAASGFTFRSFGMSWVRQAPGKGPEWVSSISGSGSDTLYADSVKGRFTISRDNSKNTLYLQMNSLRPEDTATYYCTIGGSLSRSSQGTLVTVSSA

SEQ ID NO: 49 - protein, 115 aa, synthetic construct [sequence omitted]

SEQ ID NO: 50 - protein, 116 aa, synthetic construct
EVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVKVSSA

SEQ ID NO: 51 - protein, 115 aa, synthetic construct
EVQLVESGGGVVQPGNSLRLSCAASGFTFSSFGMSWVRQAPGKGLEWVSSISGSGSDTLYADSVKGRFTISRDNAKTTLYLQMNSLRPEDTALYYCTIGGSLSRSSQGTLVTVSS

SEQ ID NO: 52 - protein, 116 aa, synthetic construct
EVQLVESGGGVVQPGNSLRLSCAASGFTFSSFGMSWVRQAPGKGLEWVSSISGSGSDTLYADSVKGRFTISRDNAKTTLYLQMNSLRPEDTALYYCTIGGSLSRSSQGTLVTVSSA

SEQ ID NO: 53 - protein, 117 aa, synthetic construct
EVQLVESGGGVVQPGNSLRLSCAASGFTFSSFGMSWVRQAPGKGLEWVSSISGSGSDTLYADSVKGRFTISRDNAKTTLYLQMNSLRPEDTALYYCTIGGSLSRSSQGTLVTVSSAA

SEQ ID NO: 54 - protein, 118 aa, synthetic construct
EVQLVESGGGVVQPGNSLRLSCAASGFTFSSFGMSWVRQAPGKGLEWVSSISGSGSDTLYADSVKGRFTISRDNAKTTLYLQMNSLRPEDTALYYCTIGGSLSRSSQGTLVTVSSAAA

SEQ ID NO: 55 - protein, 116 aa, synthetic construct
EVQLVESGGGVVQPGNSLRLSCAASGFTFSSFGMSWVRQAPGKGLEWVSSISGSGSDTLYADSVKGRFTISRDNAKTTLYLQMNSLRPEDTALYYCTIGGSLSRSSQGTLVTVSSG

SEQ ID NO: 56 - protein, 117 aa, synthetic construct
EVQLVESGGGVVQPGNSLRLSCAASGFTFSSFGMSWVRQAPGKGLEWVSSISGSGSDTLYADSVKGRFTISRDNAKTTLYLQMNSLRPEDTALYYCTIGGSLSRSSQGTLVTVSSGG

SEQ ID NO: 57 - protein, 118 aa, synthetic construct
EVQLVESGGGVVQPGNSLRLSCAASGFTFSSFGMSWVRQAPGKGLEWVSSISGSGSDTLYADSVKGRFTISRDNAKTTLYLQMNSLRPEDTALYYCTIGGSLSRSSQGTLVTVSSGGG

SEQ ID NO: 58 - protein, 115 aa, synthetic construct
EVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSS

SEQ ID NO: 59 - protein, 116 aa, synthetic construct
  REGION 1..116: note = Alb223
EVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSSA

SEQ ID NO: 60 - protein, 115 aa, synthetic construct
  REGION 1..115: note = Alb23002(E1D)
DVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSS

SEQ ID NO: 61 - protein, 217 aa, synthetic construct
APELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 62 - protein, 382 aa, synthetic construct
  REGION 1..381: note = C-terminal fusion construct (TP069)
DKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKFHYTQKSLSLSPGGGGGSGGGGSGGGGSGGGGSGGGGSGGGGSGGGGSEVQLVESGGGVVQPGGSLRLSCAASGFTFSSYAMYWVRQAPGKGLEWVSAISSGGGSTDYADSVKGRFTISRDNSKNTLYLQMNSLRPEDTALYYCAADTLYTSLTSYSYWGQGTLVTVSS

SEQ ID NO: 63 - protein, 347 aa, synthetic construct
  REGION 1..347: note = N-terminal fusion construct (TP067)
EVQLVESGGGVVQPGGSLRLSCAASGFTFSSYAMYWVRQAPGKGLEWVSAISSGGGSTDYADSVKGRFTISRDNSKNTLYLQMNSLRPEDTALYYCAADTLYTSLTSYSYWGQGTLVTVSSDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKFHYTQKSLSLSPG

SEQ ID NO: 64-65 - skipped (2 entries)

SEQ ID NO: 66 - protein, 10 aa, synthetic construct
  REGION 1..10: note = Short G1 hinge
DKTHTCPPCP

SEQ ID NO: 67 - protein, 12 aa, synthetic construct
  REGION 1..12: note = T0263018B11 parent (CDR3 Kabat)
DTLYTSLTWYSY

SEQ ID NO: 68 - protein, 12 aa, synthetic construct
  REGION 1..12: note = T0263018B11 parent (CDR3 AbM)
DTLYTSLTWYSY

SEQ ID NO: 69 - protein, 4 aa, synthetic construct
KERE

SEQ ID NO: 70 - protein, 4 aa, synthetic construct
KQRE

SEQ ID NO: 71 - protein, 4 aa, synthetic construct
GLEW

SEQ ID NO: 72 - protein, 4 aa, synthetic construct
KQRE

SEQ ID NO: 73 - protein, 5 aa, synthetic construct
KEREL

SEQ ID NO: 74 - protein, 5 aa, synthetic construct
KEREF

SEQ ID NO: 75 - protein, 5 aa, synthetic construct
KQREL

SEQ ID NO: 76 - protein, 5 aa, synthetic construct
KQREF

SEQ ID NO: 77 - protein, 5 aa, synthetic construct
KEREG

SEQ ID NO: 78 - protein, 5 aa, synthetic construct
KQREW

SEQ ID NO: 79 - protein, 5 aa, synthetic construct
KQREG

SEQ ID NO: 80 - protein, 4 aa, synthetic construct
TERE

SEQ ID NO: 81 - protein, 5 aa, synthetic construct
TEREL

SEQ ID NO: 82 - protein, 4 aa, synthetic construct
TQRE

SEQ ID NO: 83 - protein, 5 aa, synthetic construct
TQREL

SEQ ID NO: 84 - protein, 4 aa, synthetic construct
KECE

SEQ ID NO: 85 - protein, 5 aa, synthetic construct
KECEL

SEQ ID NO: 86 - protein, 5 aa, synthetic construct
KECER

SEQ ID NO: 87 - protein, 4 aa, synthetic construct
KQCE

SEQ ID NO: 88 - protein, 5 aa, synthetic construct
KQCEL

SEQ ID NO: 89 - protein, 4 aa, synthetic construct
RERE

SEQ ID NO: 90 - protein, 5 aa, synthetic construct
REREG

SEQ ID NO: 91 - protein, 4 aa, synthetic construct
RQRE

SEQ ID NO: 92 - protein, 5 aa, synthetic construct
RQREL

SEQ ID NO: 93 - protein, 5 aa, synthetic construct
RQREF

SEQ ID NO: 94 - protein, 5 aa, synthetic construct
RQREW

SEQ ID NO: 95 - protein, 4 aa, synthetic construct
QERE

SEQ ID NO: 96 - protein, 5 aa, synthetic construct
QEREG

SEQ ID NO: 97 - protein, 4 aa, synthetic construct
QQRE

SEQ ID NO: 98 - protein, 5 aa, synthetic construct
QQREW

SEQ ID NO: 99 - protein, 5 aa, synthetic construct
QQREL

SEQ ID NO: 100 - protein, 5 aa, synthetic construct
QQREF

SEQ ID NO: 101 - protein, 4 aa, synthetic construct
KGRE

SEQ ID NO: 102 - protein, 4 aa, synthetic construct
KGRE

SEQ ID NO: 103 - protein, 5 aa, synthetic construct
KGREG

SEQ ID NO: 104 - protein, 4 aa, synthetic construct
KDRE

SEQ ID NO: 105 - protein, 5 aa, synthetic construct
KDREV

SEQ ID NO: 106 - protein, 5 aa, synthetic construct
DECKL

SEQ ID NO: 107 - protein, 5 aa, synthetic construct
NVCEL

SEQ ID NO: 108 - protein, 4 aa, synthetic construct
GVEW

SEQ ID NO: 109 - protein, 4 aa, synthetic construct
GLER

SEQ ID NO: 110 - protein, 4 aa, synthetic construct
DQEW

SEQ ID NO: 111 - protein, 4 aa, synthetic construct
DLEW

SEQ ID NO: 112 - protein, 4 aa, synthetic construct
GIEW

SEQ ID NO: 113 - protein, 4 aa, synthetic construct
ELEW

SEQ ID NO: 114 - protein, 4 aa, synthetic construct
GPEW

SEQ ID NO: 115 - protein, 4 aa, synthetic construct
EWLP

SEQ ID NO: 116 - protein, 4 aa, synthetic construct
GPER

SEQ ID NO: 117 - protein, 4 aa, synthetic construct
GLER

SEQ ID NO: 118 - protein, 4 aa, synthetic construct
ELEW

SEQ ID NO: 119 - protein, 226 aa, synthetic construct
DKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALKFHYTQKSLSLSPG

SEQ ID NO: 120 - protein, 226 aa, synthetic construct
DKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALKFHYTQKSLSLSPG

SEQ ID NO: 121 - protein, 226 aa, synthetic construct
DKTHTCPPCPAPELLGGPSVFLFPPKPKDTLYISRDPEVTCVVVDVSHEDPEVKFNWYVDGVEVHNAKTKPREEQYNSTYRVVSVLQVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHYHYTQKSLSLSPG

SEQ ID NO: 122 - protein, 226 aa, synthetic construct
DKTHTCPPCPAPELLGGPSVFLFPPKPKDTLYISRDPEVTCVVVDVSHEDPEVKFNWYVDGVEVHNAKTKPREEQYNSTYRVVSVLQVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHYHYTQKSLSLSPG

SEQ ID NO: 123 - protein, 226 aa, synthetic construct
DKTHTCPPCPAPELLGGPSVFLFPPKPKDTLYISRDPEVTCVVVDVSHEDPEVKFNWYVDGVEVHNAKTKPREEQYNSTYRVVSVLQVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHYHYTQKSLSLSPG

SEQ ID NO: 124 - protein, 382 aa, synthetic construct
DKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALKFHYTQKSLSLSPGGGGGSGGGGSGGGGSGGGGSGGGGSGGGGSGGGGSEVQLVESGGGVVQPGGSLRLSCAASGFTFSSYAMYWVRQAPGKGLEWVSAISSGGGSTDYADSVKGRFTISRDNSKNTLYLQMNSLRPEDTALYYCAADTLYTSLTSYSYWGQGTLVTVSS

SEQ ID NO: 125 - protein, 226 aa, synthetic construct; identical to SEQ ID NO: 120

SEQ ID NO: 126 - protein, 347 aa, synthetic construct
DVQLVESGGGVVQPGGSLRLSCAASGFTFSSYAMYWVRQAPGKGLEWVSAISSGGGSTDYADSVKGRFTISRDNSKNTLYLQMNSLRPEDTALYYCAADTLYTSLTSYSYWGQGTLVTVSSDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALKFHYTQKSLSLSPG

SEQ ID NO: 127 - protein, 226 aa, synthetic construct; identical to SEQ ID NO: 120

SEQ ID NO: 128 - protein, 10 aa, synthetic construct
GFSFSDYYMY

SEQ ID NO: 129 - protein, 10 aa, synthetic construct
AISSGGSSTY

SEQ ID NO: 130 - protein, 12 aa, synthetic construct
DYLSVPDPSYEY

SEQ ID NO: 131 - protein, 121 aa, synthetic construct
EVQLVESGGGVVQPGGSLRLSCAASGFSFSDYYMYWVRQAPGKGLEWVSAISSGGSSTYYADSVKGRFTISRDNSKNTVYLQMNSLRPEDTALYYCAADYLSVPDPSYEYWGQGTLVTVSS

SEQ ID NO: 132 - protein, 25 aa, synthetic construct
EVQLVESGGGVVQPGGSLRLSCAAS

SEQ ID NO: 133 - protein, 14 aa, synthetic construct
WVRQAPGKGLEWVS

SEQ ID NO: 134 - protein, 39 aa, synthetic construct
YADSVKGRFTISRDNSKNTVYLQMNSLRPEDTALYYCAA

SEQ ID NO: 135 - protein, 11 aa, synthetic construct
WGQGTLVTVSS

SEQ ID NO: 136 - protein, 5 aa, synthetic construct
DYYMY

SEQ ID NO: 137 - protein, 17 aa, synthetic construct
AISSGGSSTYYADSVKG

SEQ ID NO: 138 - protein, 30 aa, synthetic construct
EVQLVESGGGVVQPGGSLRLSCAASGFSFS

SEQ ID NO: 139 - skipped

SEQ ID NO: 140 - protein, 32 aa, synthetic construct
RFTISRDNSKNTVYLQMNSLRPEDTALYYCAA

SEQ ID NO: 141 - protein, 10 aa, synthetic construct
GFTLDYYAIG

SEQ ID NO: 142 - protein, 10 aa, synthetic construct
CISSSGDSTY

SEQ ID NO: 143 - protein, 17 aa, synthetic construct
DPPSYWTGTGCLYGYRY

SEQ ID NO: 144 - protein, 126 aa, synthetic construct
EVQLVESGGGLVQPGGSLRLSCAASGFTLDYYAIGWFRQAPGKEREGVLCISSSGDSTYYADSVKGRFTISRDNAKNTVYLQMDSLNPEDTAVYYCAVDPPSYWTGTGCLYGYRYWGQGTLVTVSS

SEQ ID NO: 145 - protein, 5 aa, synthetic construct
YYAIG

SEQ ID NO: 146 - protein, 17 aa, synthetic construct
CISSSGDSTYYADSVKG

SEQ ID NO: 147 - protein, 25 aa, synthetic construct [sequence omitted]

SEQ ID NO: 148 - protein, 14 aa, synthetic construct
WFRQAPGKEREGVL

SEQ ID NO: 149 - skipped

SEQ ID NO: 150 - protein, 39 aa, synthetic construct
YADSVKGRFTISRDNAKNTVYLQMDSLNPEDTAVYYCAV

SEQ ID NO: 151 - protein, 30 aa, synthetic construct
EVQLVESGGGLVQPGGSLRLSCAASGFTLD

SEQ ID NO: 152 - skipped

SEQ ID NO: 153 - protein, 32 aa, synthetic construct
RFTISRDNAKNTVYLQMDSLNPEDTAVYYCAV

SEQ ID NO: 154 - skipped

SEQ ID NO: 155 - protein, 10 aa, synthetic construct
GFTFSDYGMG

SEQ ID NO: 156 - protein, 10 aa, synthetic construct
YISSDGGETS

SEQ ID NO: 157 - skipped

SEQ ID NO: 158 - protein, 112 aa, synthetic construct
DVQLVESGGGLVQPGGSLRLSCAASGFTFSDYGMGWVRQAPGKGLEVVSYISSDGGETSYADSVKGRFTISRDNAKNMLYLQMSSLKLEDTALYYCAKGGVRGQGTLVTVSS

SEQ ID NO: 159 - protein, 5 aa, synthetic construct
DYGMG

SEQ ID NO: 160 - protein, 17 aa, synthetic construct
YISSDGGETSYADSVKG

SEQ ID NO: 161 - protein, 25 aa, synthetic construct
DVQLVESGGGLVQPGGSLRLSCAAS

SEQ ID NO: 162 - protein, 14 aa, synthetic construct
WVRQAPGKGLEVVS

SEQ ID NO: 163 - protein, 39 aa, synthetic construct
YADSVKGRFTISRDNAKNMLYLQMSSLKLEDTALYYCAK

SEQ ID NO: 164 - protein, 11 aa, synthetic construct
RGQGTLVTVSS

SEQ ID NO: 165 - protein, 30 aa, synthetic construct
DVQLVESGGGLVQPGGSLRLSCAASGFTFS

SEQ ID NO: 166 - protein, 30 aa, synthetic construct
EVQLVESGGGLVQPGGSLRLSCAASGFTFS

SEQ ID NO: 167 - protein, 32 aa, synthetic construct
RFTISRDNAKNMLYLQMSSLKLEDTALYYCAK

SEQ ID NO: 168 - protein, 112 aa, synthetic construct
EVQLVESGGGLVQPGGSLRLSCAASGFTFSDYGMGWVRQAPGKGLEVVSYISSDGGETSYADSVKGRFTISRDNAKNMLYLQMSSLKLEDTALYYCAKGGVRGQGTLVTVSS

SEQ ID NO: 169 - protein, 25 aa, synthetic construct [sequence omitted]

SEQ ID NO: 170 - protein, 25 aa, synthetic construct
EVQLVESGGGVVQPGGSLRLSCAAS

SEQ ID NO: 171 - protein, 14 aa, synthetic construct
WVRQAPGKGPEWVS

SEQ ID NO: 172 - protein, 39 aa, synthetic construct
YADSVKGRFTISRDNSKNTLYLQMNSLRPEDTALYYCTI

SEQ ID NO: 173 - protein, 11 aa, synthetic construct
SSQGTLVTVSS

SEQ ID NO: 174 - protein, 30 aa, synthetic construct
EVQLVESGGGVVQPGGSLRLSCAASGFTFR

SEQ ID NO: 175 - protein, 32 aa, synthetic construct
RFTISRDNSKNTLYLQMNSLRPEDTALYYCTI

SEQ ID NO: 176-184 - skipped (9 entries)

SEQ ID NO: 185 - protein, 121 aa, synthetic construct
DVQLVESGGGVVQPGGSLRLSCAASGFTFSSYAMYWVRQAPGKGLEWVSAISSGGGSTDYADSVKGRFTISRDNSKNTLYLQMNSLRPEDTALYYCAADTLYTSLTSYSYWGQGTLVTVSS

SEQ ID NO: 186 - protein, 121 aa, synthetic construct
EVQLVESGGGVVQPGGSLRLSCAASGFTFSSYAMYWVRQAPGKGLEWVSAISSGGGSTDYADSVKGRFTISRDNSKNTLSLQMNSLRPEDTALYYCAADTLYTSLESYSYWGQGTLVTVSS

SEQ ID NO: 187 - protein, 121 aa, synthetic construct
DVQLVESGGGVVQPGGSLRLSCAASGFTFSSYAMYWVRQAPGKGLEWVSAISSGGGSTDYADSVKGRFTISRDNSKNTLSLQMNSLRPEDTALYYCAADTLYTSLESYSYWGQGTLVTVSS

SEQ ID NO: 188 - skipped

SEQ ID NO: 189 - protein, 12 aa, synthetic construct
DTLYTSLESYSY

SEQ ID NO: 190 - protein, 25 aa, synthetic construct
EVQLVESGGGVVQPGGSLRLSCAAS

SEQ ID NO: 191-199 - skipped (9 entries)

SEQ ID NO: 200 - protein, 126 aa, synthetic construct
EVQLVESGGGVVQAGGSLSISCAASGGSLSNYVLGWFRQAPGKEREFVAAINWRGDITIGPPNVEGRFTISRDNAKNTGYLQMNSLAPDDTALYYCGAGTPLNPGAYIYDWSYDYWGRGTLVTVSS

SEQ ID NO: 201 - protein, 245 aa, synthetic construct
DIQLTQSPSSLSASVGDRVTLTCQATQDIDNNLNWYQQKPGKAPKLLIYDASNLETGVPSRFSGSGSGTDFTFTISDLQPEDVATYYCQQYYNLPLTFGGGTKVDIKRSRGGGGSGGGGSGGGGSLEMAEVQLVQSGAEVKKPGASVKVSCKASGYTFTSYDINWVRQATGQGLEWMGWMNPNSGNTGYAQKFQGRVTMTRNTSISTAYMELSSLRSEDTAVYYCARGVDLGDGWGQGTLVTVSS

SEQ ID NO: 202 - protein, 30 aa, synthetic construct
EVQLVESGGGVVQPGGSLRLSCAASGFTFS

SEQ ID NO: 203 - protein, 14 aa, synthetic construct
WVRQAPGKGLEWVS

SEQ ID NO: 204 - protein, 39 aa, synthetic construct
YADSVKGRFTISRDNSKNTLSLQMNSLRPEDTALYYCAA

SEQ ID NO: 205 - protein, 32 aa, synthetic construct
RFTISRDNSKNTLSLQMNSLRPEDTALYYCAA

SEQ ID NO: 206 - protein, 25 aa, synthetic construct
DVQLVESGGGVVQPGGSLRLSCAAS

SEQ ID NO: 207 - protein, 30 aa, synthetic construct
DVQLVESGGGVVQPGGSLRLSCAASGFTFS

(19) United States
(12) Patent Application Publication
(10) Pub. No.: US 2025/0034260 A1
(43) Pub. Date: Jan. 30, 2025

(54) FCRN ANTAGONISTS FOR TREATMENT OF IGG-RELATED DISEASES AND DISORDERS

(71) Applicants: Ablynx N.V., Ghent-Zwijnaarde (BE); Sanofi, Paris (FR)

(72) Inventors: Tom Van Bogaert, Gentbrugge (BE); Judith Verhelst, Heusden (BE)

(73) Assignee: Sanofi, Paris (FR)

(21) Appl. No.: 18/764,573

(22) Filed: Jul. 5, 2024

(30) Foreign Application Priority Data
Jul. 5, 2023 (EP) 23 306 136.5

(51) Int. Cl. C07K 16/28; A61K 39/00; A61P 37/06

(52) U.S. Cl. CPC C07K 16/283; A61P 37/06; A61K 2039/505; C07K 2317/52; C07K 2317/62; C07K 2317/92; C07K 2317/94

(57) ABSTRACT

The present invention relates to FcRn antagonists which bind to the neonatal Fc receptor (FcRn) and interfere with binding of FcRn's natural ligands, the Fc region of IgG. In particular, the invention relates to FcRn antagonists comprising at least one first polypeptide specifically binding to an epitope on Fc receptor (FcRn) and a second polypeptide which competes with wild-type IgG1 Fc region for binding to FcRn. The invention also relates to fusion proteins comprising the FcRn antagonists, compositions comprising the FcRn antagonists and/or fusion proteins and the use of the FcRn antagonists, compositions comprising the FcRn antagonists and/or fusion proteins in medicine, in particular in the treatment of IgG-mediated disorders.

### REFERENCE TO AN ELECTRONIC SEQUENCE LISTING

The contents of the electronic sequence listing (A084870236US00-SEQ-JRV.xml; Size: 183,900 bytes; and Date of Creation: Jul. 5, 2024) is herein incorporated by reference in its entirety.

### FIELD OF THE INVENTION

The present invention relates to FcRn antagonists which bind to the neonatal Fc receptor (FcRn) and interfere with binding of FcRn's natural ligands, the Fc region of IgG.

In particular, the present invention relates to novel and improved FcRn antagonists comprising at least two polypeptides, a first polypeptide specifically binding to an epitope on FcRn and a second polypeptide which competes with wild-type IgG1 Fc region for binding to FcRn.

The invention further relates to nucleic acids encoding such molecules or part of such molecules; to host cells comprising such nucleic acids and/or expressing or capable of expressing such FcRn antagonists; to compositions, and in particular to pharmaceutical compositions that comprise such FcRn antagonists, nucleic acids and/or host cells; and to uses of such molecules, nucleic acids, host cells and/or compositions, in particular for therapeutic purposes.

### TECHNOLOGICAL BACKGROUND

The neonatal Fc receptor (FcRn) is a beta (β)-2-microglobulin (β2m) associated protein that is widely expressed with predominant expression on parenchymal, endothelial and hematopoietic cells. FcRn is predominantly localised intracellularly within vesicular networks, particularly endosomes.

FcRn has two known ligands, IgG and albumin, which together account for almost 70% of total serum proteins. IgG and albumin bind to FcRn at distinct sites. Immunoglobulin gamma (IgG) antibodies are fundamental to the immune response. IgG has a prolonged serum half-life due to its interaction with FcRn, which protects it from intracellular degradation by a cellular recycling mechanism. FcRn binds to endocytosed IgG and protects the IgG from being transported to degradative lysosomes by recycling it back to the extracellular compartment. This recycling mechanism is strictly pH-dependent and binding to FcRn is favoured at low pH (e.g., acidic endosomal pH, which is typically below 6.5) following acidification of the endosomal compartment. When IgG binds to FcRn, it escapes degradation in the lysosome. On return to the cell surface, at extracellular physiological pH (which is typically around pH 7.4), the binding is weakened, resulting in the release of IgG into the bloodstream.

Modulation of the FcRn/IgG interaction may be beneficial in therapy. It has been found that FcRn-mediated IgG recycling is the dominant process for maintaining IgG plasma concentrations in humans (Xiao, J Biomed Biotechnol. (2012) 2012:282989). For example, FcRn antagonists, which block the binding of IgG to FcRn, are being used to increase the clearance of IgG from the circulation. This may be desirable in diseases where IgG antibodies play a key role in the pathology, such as autoimmune diseases where circulating autoreactive antibodies cause the pathology, inflammatory diseases and diseases where the pathology is characterised by overexpression of IgG antibodies. Increased clearance of an undesired antibody can be achieved by using an FcRn antagonist, such as an antibody with an engineered Fc, that binds to FcRn with high affinity and does not dissociate rapidly at near neutral pH. Such engineered antibodies are called Abdegs, for antibodies that enhance IgG degradation (Swiercz et al, J Nucl Med. (2014) 55(7): 1204-7). Such FcRn antagonists would not be released from cells but would instead be predicted to remain bound to FcRn and block binding of other, lower affinity IgGs. As a result, FcRn function would be blocked and endogenous or undesired IgGs would be directed into the lysosomal pathway for degradation.

Examples of antibodies to human FcRn which block or reduce the binding of IgG to FcRn have been described in, e.g., WO 2014/019727. WO 2019/118791 relates to antibodies that bind FcRn with high affinity. WO 2015/167293 relates to antibodies that competitively inhibit the binding of IgG to FcRn.

Engineered Fc fragments that compete with and inhibit the binding of IgG have also been described, see, e.g., WO 2019/110823, WO 2015/100299 and WO 2021/016571. These Fc fragments have been shown to increase the rate of serum IgG clearance in the treated subject. These molecules have increased affinity and reduced pH dependence compared to IgG and therefore block FcRn. As a result, endogenous or unwanted IgG is directed to the lysosomal pathway for degradation.

However, the duration of the efficacy of known FcRn antagonists is thought to be reduced because either the affinity for FcRn is low, resulting in incomplete and/or poor blocking, or—if the affinity is high enough to block FcRn sufficiently—the half-life of the FcRn antagonist is automatically reduced as a direct consequence of extensively blocking the recycling mechanism modulated by FcRn.

For instance, FcRn antagonists that bind pH-independently, such as some anti-FcRn antibodies, efficiently block FcRn at neutral pH by ensuring that FcRn is already occupied at the cell surface and remains bound in the endosome (i.e., at acidic endosomal pH). For instance, Nipocalimab binds with picomolar affinity to FcRn at both acidic endosomal and extracellular physiological pH, occupying FcRn throughout the recycling pathway, see, e.g., Wolfe et al., “IgG regulation through FcRn blocking: A novel mechanism for the treatment of myasthenia gravis”, Journal of the Neurological Sciences, 2021, 430:118074. However, because of this tight binding, these FcRn antagonists are more likely to be targeted to the degradation pathway and therefore show very low half-lives in circulation.

FcRn antagonists that bind pH-dependently (i.e., with a lower affinity at extracellular physiological pH), such as some engineered Fc fragments, e.g., Efgartigimod, lead to less efficient blockade but are degraded to a lesser extent. For instance, the pH-dependent binding displayed by Efgartigimod allows it to dissociate from FcRn during the recycling process, leading to a serum half-life of 4.89 days when dosed at 10 mg/kg (Howard et al., “Randomized phase 2 study of FcRn antagonist efgartigimod in generalized myasthenia gravis”, Neurology, 2019, 92:e2661-e2673). This is a relatively long half-life compared with that of full-length monoclonal antibodies targeting FcRn, which have half-lives 1 day, but higher half-lives without compromising FcRn blocking would be desirable (e.g., Blumberg et al., “Blocking FcRn in humans reduces circulating IgG levels and inhibits IgG immune complex-mediated immune responses”, Sci. Adv. 2019, 5). See, e.g., Wolfe et al., “IgG regulation through FcRn blocking: A novel mechanism for the treatment of myasthenia gravis”, Journal of the Neurological Sciences, 2021, 430:118074.

Hence, it is desirable to identify a “sweet spot”, where there is sufficient blocking of FcRn to obtain an antagonistic effect (i.e., enough binding at extracellular physiological pH) but, at the same time, the binding at extracellular physiological pH should not be too tight, so that sufficient half-life of the antagonist is guaranteed. In addition, there should be an optimum in extracellular physiological pH affinity, to balance the rapid clearance and achieving sufficiently long FcRn antagonism.

As described above, known FcRn antagonists have reduced half-lives and/or block FcRn insufficiently. This leads to suboptimal dosing regimens for patients (i.e., treatment at high doses and/or administration at high frequency).

There is therefore a need for novel FcRn antagonists, e.g., with improved FcRn binding properties and/or longer half-lives in the circulation, which may be administered either at lower doses and/or at lower frequencies.

### SUMMARY OF THE INVENTION

The present invention solves the above problem and provides neonatal Fc receptor (FcRn, also called FcRP, FcRB or Brambell receptor) antagonists comprising (a) at least one first polypeptide which binds to a specific epitope on FcRn and (b) a second polypeptide which competes with wild-type IgG1 Fc region for binding to FcRn.

The at least two FcRn-binding polypeptides ((a) and (b)) comprised in the FcRn antagonists of the present invention show a synergistic or cooperative effect, which allows for an improved binding to FcRn and/or improved (or at least not worsened) half-life of the FcRn antagonists.

In some embodiments, the presence of the first polypeptide (a) and the second polypeptide (b) in the FcRn antagonists of the present invention provides for an increased avidity of the FcRn antagonists for FcRn.

In some embodiments, the first polypeptide competes with wild-type IgG1 Fc region for binding to FcRn. Hence, in this embodiment, both the first and second polypeptide comprised in the FcRn antagonists of the present invention compete with wild-type IgG1 Fc region for binding to FcRn. In certain embodiments, the first polypeptide (a) that competes with wild-type IgG1 Fc region for binding to FcRn comprises or consists of an immunoglobulin single variable domain (ISVD).

In a preferred embodiment, the first polypeptide (a) specifically binding to an epitope on FcRn receptor does not compete with wild-type IgG1 Fc region for binding to FcRn, and preferably comprises or consists of an immunoglobulin single variable domain (ISVD).

In some embodiments, the second polypeptide (b) which competes with wild-type IgG1 Fc region for binding to FcRn comprises or consists of an antibody or a fragment thereof, preferably a Fc domain or a fragment thereof.

In a preferred embodiment, the first polypeptide (a) is an ISVD which specifically binds to an epitope on FcRn, which epitope comprises at least one of the following amino acid residues 1A, 2E, 3S, 4H, 5L, 32P, 97E, 98L, 99G, 100P, 101D, 102N, 103T, 164R, 167L, 168E, 171R, 174L, 175E, 177K, 204Y, 205P, 206P, 207E, 208L, 209Q, 255Q, 256H, 257A, 259L, 260A, 261Q, and/or 262P, amino acid residues being numbered according to SEQ ID NO: 1.

In another preferred embodiment, the first polypeptide (a) is an ISVD which specifically binds to an epitope in FcRn, which epitope comprises at least one of the following combinations of amino acid residues:
i) 4H and 5L, and/or
ii) 98L, 99G, 100P, 101D and 102N, and/or
iii) 167L, 171R, 174L, 175E and 177K, and/or
iv) 255Q, 256H, 257A, 259L, 260A and 262P,
   wherein the amino acid residues are numbered according to SEQ ID NO: 1,
   preferably which epitope comprises at least one of the following combinations of amino acid residues:
i) 2E, 3S, 4H and 5L, and/or
ii) 97E, 98L, 99G, 100P, 101D and 102N, and/or
iii) 98L, 99G, 100P, 101D, 102N and 103T, and/or
iv) 167L, 168E, 171R, 174L, 175E and 177K, and/or
v) 205P, 206P and 207E, and/or
vi) 255Q, 256H, 257A, 259L, 260A, 261Q, and 262P,
   wherein the amino acid residues are numbered according to SEQ ID NO: 1,
   or at least one of the following combinations of amino acid residues:
i) 1A, 2E, 3S, 4H and 5L, and/or
ii) 164R, 167L, 168E, 171R, 174L, 175E and 177K, and/or
iii) 204Y, 205P, 206P and 230E, and/or
iv) 205P, 206P, 207E and 208L,
   wherein the amino acid residues are numbered according to SEQ ID NO: 1.

In other preferred embodiments, the first polypeptide (a) is an ISVD which specifically binds to an epitope in FcRn, which epitope comprises at least the following amino acid residues 4H, 5L, 98L, 99G, 100P, 101D, 102N, 167L, 171R, 174L, 175E, 177K, 207E, 255Q, 256H, 257A, 259L, 260A, and 262P, wherein the amino acid residues are numbered according to SEQ ID NO: 1, or at least the following amino acid residues 2E, 3S, 4H, 5L, 32P, 97E, 98L, 99G, 100P, 101D, 102N, 103T, 167L, 168E, 171R, 174L, 175E, 177K, 205P, 206P, 207E, 255Q, 256H, 257A, 259L, 260A, 261Q, and 262P, wherein the amino acid residues are numbered according to SEQ ID NO: 1,

or at least the following amino acid residues 1A, 2E, 3S, 4H, 5L, 32P, 97E, 98L, 99G, 100P, 101D, 102N, 103T, 164R, 167L, 168E, 171R, 174L, 175E, 177K, 204Y, 205P, 206P, 207E, 208L, 209Q 255Q, 256H, 257A, 259L, 260A, 261Q, and 262P, wherein the amino acid residues are numbered according to SEQ ID NO: 1.

For instance, in a preferred embodiment, the first polypeptide (a) is an ISVD which specifically binds to an epitope in FcRn, wherein the ISVD comprises or consists of SEQ ID NO: 14, SEQ ID NO: 15 or SEQ ID NO: 185.

For instance, in a preferred embodiment, the first polypeptide (a) is an ISVD which specifically binds to an epitope in FcRn, wherein the ISVD comprises or consists of SEQ ID NO: 14 (T0263018B11-parent, EVQLVESGGGVVQPGGSLRLSCAASGFTFSSYAMYWVRQAPGKGLEWVSAISSGGGSTDYADSVKGRF TISRDNSKNTLYLQMNSLRPEDTALYYCAADTLYTSLTWYSYWGQGTLVTVSS), or SEQ ID NO: 15 (T0263018B11-W100cS, EVQLVESGGGVVQPGGSLRLSCAASGFTFSSYAMYWVRQAPGKGLEWVSAISSGGGSTDYADSVKGRF TISRDNSKNTLYLQMNSLRPEDTALYYCAADTLYTSLTSYSYWGQGTLVTVSS), or SEQ ID NO.: 185 (T0263018B11(E1D,W100cS), DVQLVESGGGVVQPGGSLRLSCAASGFTFSSYAMYWVRQAPGKGLEWVSAISSGGGSTDYADSVKGRF TISRDNSKNTLYLQMNSLRPEDTALYYCAADTLYTSLTSYSYWGQGTLVTVSS), or SEQ ID NO.: 186 (T0263018B11(Y79S,T100bE,W100cS)),EVQLVESGGGVVQPGGSLRLSCAASGFTFSSYAMYWVRQ APGKGLEWVSAISSGGGSTDYADSVKGRFTISRDNSKNTLSLQMNSLRPEDTALYYCAADTLYTSLESYSY WGQGTLVTVSS), or SEQ ID NO.: 187 (T0263018B11(D1E, Y79S,T100bE,W100cS), DVQLVESGGGVVQPGGSLRLSCAASGFTFSSYAMYWVRQAPGKGLEWVSAISSGGGSTDYADSVKGRF TISRDNSKNTLSLQMNSLRPEDTALYYCAADTLYTSLESYSYWGQGTLVTVSS), or SEQ ID NO.: 131 (T0263091D07, EVQLVESGGGVVQPGGSLRLSCAASGFSFSDYYMYWVRQAPGKGLEWVSAISSGGSSTYYADSVKGRFT ISRDNSKNTVYLQMNSLRPEDTALYYCAADYLSVPDPSYEYWGQGTLVTVSS) or SEQ ID NO: 144 (T0263204B12, EVQLVESGGGLVQPGGSLRLSCAASGFTLDYYAIGWFRQAPGKEREGVLCISSSGDSTYYADSVKGRFTIS RDNAKNTVYLQMDSLNPEDTAVYYCAVDPPSYWTGTGCLYGYRYWGQGTLVTVSS), or SEQ ID NO.: 158 (DVQLVESGGGLVQPGGSLRLSCAASGFTFSDYGMGWVRQAPGKGLEVVSYISSDGGETSYADSVKGRF TISRDNAKNMLYLQMSSLKLEDTALYYCAKGGVRGQGTLVTVSS), or SEQ ID NO.: 168 (EVQLVESGGGLVQPGGSLRLSCAASGFTFSDYGMGWVRQAPGKGLEVVSYISSDGGETSYADSVKGRFT ISRDNAKNMLYLQMSSLKLEDTALYYCAKGGVRGQGTLVTVSS).

For instance, in another preferred embodiment, the first polypeptide (a) is an ISVD which specifically binds to an epitope in FcRn, wherein the ISVD comprises or consists of SEQ ID NO: 186 or SEQ ID NO: 187.

In other preferred embodiments, the second polypeptide (b) comprises or consists of a Fc domain or fragment thereof which comprises at least one, preferably all, of the following amino acids at the following positions:
a) a tyrosine (Y) at amino acid position 252,
b) a threonine (T) at amino acid position 254,
c) a glutamic acid (E) at amino acid position 256,
d) a lysine (K) at amino acid position 433,
e) a phenylalanine (F) at amino acid position 434, and/or
f) a tyrosine (Y) at amino acid position 436;
   according to Eu numbering.

In other preferred embodiments, the second polypeptide (b) comprises or consists of a Fc domain or fragment thereof which comprises a combination of the following four amino acid residues:
a) a tyrosine (Y) at amino acid position 252,
b) an aspartic acid (D) or a glutamic acid (E) at amino acid position 256,
c) a tryptophan (W) or a glutamine (Q) at amino acid position 307, and
d) a phenylalanine (F) or a tyrosine (Y) at amino acid position 434;
   according to Eu numbering.

In other preferred embodiments, the second polypeptide (b) comprises or consists of a Fc domain or fragment thereof which comprises a combination of the following four amino acid residues: a tyrosine (Y) at amino acid position 252, an aspartic acid (D) at amino acid position 256, a glutamine (Q) at amino acid position 307, and a tyrosine (Y) at amino acid position 434, according to Eu numbering.

In other embodiments, the invention relates to a fusion protein which comprises the FcRn antagonists of the present invention and at least one further moiety, such as an ISVD. In some embodiments, the further ISVD is an ISVD specifically binding to (human) serum albumin, and/or an ISVD specifically binding to a therapeutic target. For instance, the at least one further ISVD comprised in the fusion protein of the present invention consists of 4 framework regions (FR1 to FR4 respectively) and 3 complementarity determining regions (CDR1 to CDR3 respectively), wherein:
CDR1 is SFGMS (SEQ ID NO: 16),
CDR2 is SISGSGSDTLYADSVKG (SEQ ID NO: 17) and
CDR3 is GGSLSR (SEQ ID NO: 18),
   wherein the CDR sequences are determined according to Kabat; and/or
   wherein
CDR1 is GFTFRSFGMS (SEQ ID NO: 19),
CDR2 is SISGSGSDTL (SEQ ID NO: 20) and
CDR3 is GGSLSR (SEQ ID NO: 21),
   wherein the CDR sequences are determined according to AbM numbering.

Further provided is a nucleic acid molecule encoding the FcRn antagonists and/or the fusion protein of the present invention, vectors comprising the nucleic acid molecule of the present invention, host cells comprising the FcRn antagonists and/or fusion protein of the invention, or the nucleic acid molecule or vector encoding the FcRn antagonists and/or fusion protein of the invention.

The present invention further provides methods for producing the FcRn antagonists and/or fusion protein of the present invention.

The foregoing and other features and advantages of the present invention will be more fully understood from the following detailed description of illustrative embodiments taken in conjunction with the accompanying drawings.

### BRIEF DESCRIPTION OF THE FIGURES

FIG. 1. Schematic drawing of the structural format of FcRn antagonists TP067, TP069 according to specific embodiments of the invention. The control FcRn antagonist TP061 (comprising SEQ ID NO: 23 or 24) comprises an IgG1 Fc YTE-KF Fc domain. TP067 (comprising SEQ ID NOs: 126 and 127) and TP069 (comprising SEQ ID NOs: 124 and 125) are FcRn antagonists according to the invention, comprising the same IgG1 Fc YTE-KF Fc domain as TP061 linked to a Nanobody® VHH (ISVD) specifically binding to FcRn (ellipse shape hatched in grey, FcRn VHH in FIG. 1, SEQ ID NO: 15). The Fc domain and Nanobody® VHH sequences in these polypeptide constructs were fused to the N- and/or C-terminus of one of the Fc chains, via an IgG1 hinge (DKTHT, SEQ ID NO: 66) and/or a GS linker (35GS linker, SEQ ID NO: 39), respectively (TP067, TP069), as described in the present application. In the IgG1 Fc YTE-KF Fc domain of the FcRn antagonists, both CH3 domains comprised therein comprise knob in hole mutations. These mutations serve to engineer an interface between a first and second polypeptide (CH3 domain) for hetero oligomerization using the “knobs and holes” technology as described in, for example, in U.S. Pat. No. 8,216,805, WO 1996/27011, Ridgway, J B et al., “‘Knobs-into-holes’ engineering of antibody CH3 domains for heavy chain heterodimerization”, Protein engineering, 1996, 9, 7:617-21 or in Merchant et al., “An efficient route to human bispecific IgG”, Nature Biotechnology, 1998, 16: 677-681. The amino acids HY in the CH3 domain of the Fc domain were mutated into RF, such as the mutations H435R and Y436F in CH3 domain, as described by Jendeberg, L. et al. (“Engineering of Fc1 and Fc3 from human immunoglobulin G to analyze subclass specificity for staphylococcal protein A”, J. Immunological Meth., 1997, 201:25-34).

FIGS. 2A and 2B. (FIG. 2A) Mean (+/−SD, n=3) serum concentration-time profiles of hIgG clearance after administration of FcRn antagonists TP067 or TP069, compared to a control FcRn antagonist (TP061) via quantitative LC-MS/MS analysis. To mimic relevant competition with hIgG, Tg32 mice were preloaded intravenously with 500 mg/kg Privigen® (arrow) followed by administration of PBS (placebo group) or FcRn antagonists TP061, TP067 or TP069, the latter marked as time zero in the graph. Blood samples were collected at the indicated time points. (FIG. 2B) Clearance of IgG plotted as the percentage of the hIgG concentration, with the data of (FIG. 2A).

FIG. 3. Mean (+/−SD, n=3) serum concentration-time profiles of the FcRn antagonists TP067 or TP069 compared to a control FcRn antagonist (TP061) via quantitative LC-MS/MS analysis. To mimic relevant competition with hIgG, Tg32 mice were preloaded intravenously with 500 mg/kg Privigen® (arrow), followed by administration of FcRn antagonists TP061, TP067 or TP069. Blood samples were collected at the indicated time points.

FIGS. 4A and 4B. Schematic drawing of the structural format of FcRn antagonists TPP-122907, TPP-122908, TPP-122909, TPP-122903, TPP-122904, TPP-122905, TPP-122912, TPP-122910, TPP-122946 and TPP-122947 according to specific embodiments of the invention. Control TPP-122902 and benchmark antibody TPP-122914 are also represented.

FIG. 5. Compiled mean (SD) Privigen® concentration vs time profiles for all constructs based on an antagonistic Fc domain.

FIG. 6. Mean (SD) Privigen® concentration vs time profiles of TPP-122905 and TPP-122907 vs placebo, TPP-122902 and TPP-122914.

FIG. 7. Mean (SD) Privigen® concentration vs time profiles of TPP-122910 and TPP-122907 vs placebo and TPP-122902.

FIG. 8. Mean (SD) Privigen® concentration vs time profiles of symmetrical constructs TPP-122903 and TPP-122904 vs placebo, TPP-122902 and TPP-122907.

FIG. 9. Mean (SD) Privigen® concentration vs time profiles of TPP-122908, TPP-122909 and TPP-122912 vs placebo and TPP-122902.

FIG. 10. Mean (SD) Privigen® concentration vs time profiles of TPP-122946 and TPP-122947 vs placebo.

### DETAILED DESCRIPTION OF THE INVENTION

### Definitions

Unless indicated or defined otherwise, all terms used have their usual meaning in the art, which will be clear to the skilled person. Reference is, for example, made to the standard handbooks, such as Sambrook et al., 1989 (Molecular Cloning: A Laboratory Manual, 2^{nd} Ed., Vols. 1-3, Cold Spring Harbor Laboratory Press), Ausubel et al., 1987 (Current protocols in molecular biology, Green Publishing and Wiley Interscience, New York), Lewin 1985 (Genes II, John Wiley & Sons, New York, N.Y.), Old et al., 1981 (Principles of Gene Manipulation: An Introduction to Genetic Engineering, 2^{nd} Ed., University of California Press, Berkeley, CA), Roitt et al., 2001 (Immunology, 6^{th} Ed., Mosby/Elsevier, Edinburgh), Roitt et al., 2001 (Roitt's Essential Immunology, 10^{th} Ed., Blackwell Publishing, UK), and Janeway et al., 2005 (Immunobiology, 6^{th} Ed., Garland Science Publishing/Churchill Livingstone, New York), as well as to the general background art cited herein.

Unless indicated otherwise, all methods, steps, techniques and manipulations that are not specifically described in detail herein can be performed and have been performed in a manner known per se, as will be clear to the skilled person. Reference is, for example, again made to the standard handbooks and the general background art mentioned herein and to the further references cited therein; as well as to for example the following reviews: Presta 2006 (Adv. Drug Deliv. Rev., 58: 640), Levin and Weiss 2006 (Mol. Biosyst., 2: 49), Irving et al., 2001 (J. Immunol. Methods, 248: 31), Schmitz et al., 2000 (Placenta 21 Suppl. A: S106), Gonzales et al., 2005 (Tumour Biol., 26: 31), which describe techniques for protein engineering, such as affinity maturation and other techniques for improving the specificity and other desired properties of proteins such as immunoglobulins.

It must be noted that as used herein, the singular forms “a”, “an”, and “the”, include plural references unless the context clearly indicates otherwise. Thus, for example, reference to “a reagent” includes one or more of such different reagents and reference to “the method” includes reference to equivalent steps and methods known to those of ordinary skill in the art that could be modified or substituted for the methods described herein.

Unless otherwise indicated, the term “at least” preceding a series of elements is to be understood to refer to every element in the series. Those skilled in the art will recognize or be able to ascertain using no more than routine experimentation, many equivalents to the specific embodiments of the invention described herein. Such equivalents are intended to be encompassed by the present invention.

The term “and/or” wherever used herein includes the meaning of “and”, “or” and “all or any other combination of the elements connected by said term.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integer or step. When used herein the term “comprising” can be substituted with the term “containing” or “including” or sometimes when used herein with the term “having”.

“Similar”, as used herein, is interchangeable for alike, analogous, comparable, corresponding, and -like, and is meant to have the same or common characteristics, and/or in a quantifiable manner to show comparable results i.e., with a variation of maximum 20%, 10%, more preferably 5%, or even more preferably 1%, or less.

The term “sequence” as used herein (for example in terms like “immunoglobulin sequence”, “antibody sequence”, “variable domain sequence”, “V_{HH} sequence” or “protein sequence”), should generally be understood to include both the relevant amino acid sequence as well as nucleic acids or nucleotide sequences encoding the same, unless the context requires a more limited interpretation. Amino acid sequences are interpreted to mean a single amino acid or an unbranched sequence of two or more amino acids, depending on the context. Nucleotide sequences are interpreted to mean an unbranched sequence of 3 or more nucleotides.

When a nucleotide sequence or amino acid sequence is said to “comprise” another nucleotide sequence or amino acid sequence, respectively, or to “essentially consist of” another nucleotide sequence or amino acid sequence, this may mean that the latter nucleotide sequence or amino acid sequence has been incorporated into the first mentioned nucleotide sequence or amino acid sequence, respectively, but more usually this generally means that the first mentioned nucleotide sequence or amino acid sequence comprises within its sequence a stretch of nucleotides or amino acid residues, respectively, that has the same nucleotide sequence or amino acid sequence, respectively, as the latter sequence, irrespective of how the first mentioned sequence has actually been generated or obtained (which may for example be by any suitable method described herein). By means of a non-limiting example, when an ISVD is said to comprise a CDR sequence, this may mean that said CDR sequence has been incorporated into the ISVD, but more usually this generally means that the ISVD contains within its sequence a stretch of amino acid residues with the same amino acid sequence as said CDR sequence, irrespective of how said ISVD has been generated or obtained. It should also be noted that when the latter amino acid sequence has a specific biological or structural function, it preferably has essentially the same, a similar or an equivalent biological or structural function in the first-mentioned amino acid sequence (in other words, the first-mentioned amino acid sequence is preferably such that the latter sequence is capable of performing essentially the same, a similar or an equivalent biological or structural function).

For example, when an ISVD is said to comprise a CDR sequence or framework sequence, respectively, the CDR sequence and framework are preferably capable, in said ISVD, of functioning as a CDR sequence or framework sequence, respectively. Also, when a nucleotide sequence is said to comprise another nucleotide sequence, the first-mentioned nucleotide sequence is preferably such that, when it is expressed into an expression product (e.g., a polypeptide), the amino acid sequence encoded by the latter nucleotide sequence forms part of said expression product (in other words, that the latter nucleotide sequence is in the same reading frame as the first-mentioned, larger nucleotide sequence).

Amino acids are organic compounds that contain amino[a](—NH⁺₃) and carboxylate (—CO⁻₂) functional groups, along with a side chain (R group) specific to each amino acid—Amino acid residues will be indicated interchangeably herein according to the standard three-letter or one-letter amino acid code, as mentioned in Table 1 below.

TABLE 1
Common amino acids

| 1-Letter Code | 3-Letter Code | Amino Acid Name |
|---|---|---|
| A | Ala | Alanine |
| C | Cys | Cysteine |
| D | Asp | Aspartic acid |
| E | Glu | Glutamic acid |
| F | Phe | Phenylalanine |
| G | Gly | Glycine |
| H | His | Histidine |
| I | Ile | Isoleucine |
| K | Lys | Lysine |
| L | Leu | Leucine |
| M | Met | Methionine |
| N | Asn | Asparagine |
| P | Pro | Proline |
| Q | Gln | Glutamine |
| R | Arg | Arginine |
| S | Ser | Serine |
| T | Thr | Threonine |
| V | Val | Valine |
| W | Trp | Tryptophan |
| X | Xaa | Unspecified |
| Y | Tyr | Tyrosine |

When an amino acid residue is indicated as “X” or “Xaa”, it means that the amino acid residue is unspecified, unless the context requires a more limited interpretation. For example, if the description provides an amino acid sequence of a CDR wherein one (or more) of the amino acid residue(s) is (are) indicated with “X”, the description may further specify which amino acid residue(s) is (can be) present at that specific position of the CDR.

Amino acids are those amino acids commonly found in naturally occurring proteins and are listed in Table 1. Any amino acid sequence that contains post-translationally modified amino acids may be described as the amino acid sequence that is initially translated using the symbols shown in the Table 1 with the modified positions, e.g., hydroxylations or glycosylations, but these modifications shall not be shown explicitly in the amino acid sequence. Any peptide or protein that can be expressed as a sequence modified linkages, cross links and end caps, non-peptidyl bonds, etc., is embraced by this definition. The terms “protein”, “peptide”, “protein/peptide”, and “polypeptide” are used interchangeably throughout the disclosure, and each has the same meaning for purposes of this disclosure. Each term refers to an organic compound made of a linear chain of two or more amino acids. The compound may have ten or more amino acids; twenty-five or more amino acids; fifty or more amino acids; one hundred or more amino acids, two hundred or more amino acids, and even three hundred or more amino acids. The skilled artisan will appreciate that polypeptides generally comprise fewer amino acids than proteins, although there is no art-recognized cut-off point of the number of amino acids that distinguish a polypeptide from a protein; those polypeptides may be made by chemical synthesis or recombinant methods; and those proteins are generally made in vitro or in vivo by recombinant methods as known in the art.

By convention, the amide bond in the primary structure of polypeptides is in the order that the amino acids are written, in which the amine end (N-terminus) of a polypeptide is always on the left, while the acid end (C-terminus) is on the right.

Any amino acid sequence that contains post-translationally modified amino acids may be described as the amino acid sequence that is initially translated using the symbols shown in Table 1 with the modified positions; e.g., hydroxylations or glycosylations, but these modifications shall not be shown explicitly in the amino acid sequence. Any peptide or protein that can be expressed as a sequence modified linkages, cross links and end caps, non-peptidyl bonds, etc., is embraced by this definition.

The term “domain” as used herein generally refers to a globular region of an antibody chain, and in particular to a globular region of a heavy chain antibody, or to a polypeptide that essentially consists of such a globular region. Usually, such a domain will comprise peptide loops (for example 3 or 4 peptide loops) stabilized, for example, as a sheet or by disulfide bonds.

The term “about” used in the context of the parameters or parameter ranges of the provided herein shall have the following meanings. Unless indicated otherwise, where the term “about” is applied to a particular value or to a range, the value or range is interpreted as being as accurate as the method used to measure it. If no error margins are specified in the application, the last decimal place of a numerical value indicates its degree of accuracy. Where no other error margins are given, the maximum margin is ascertained by applying the rounding-off convention to the last decimal place, e.g., for a pH value of about pH 2.7, the error margin is 2.65-2.74. In the present application, any parameter indicated with the term “about” is also contemplated as being disclosed without the term “about”. In other words, embodiments referring to a parameter value using the term “about” shall also describe an embodiment directed to the numerical value of said parameter as such. For example, an embodiment specifying a pH of “about pH 2.7” shall also disclose an embodiment specifying a pH of “pH 2.7” as such; an embodiment specifying a pH range of “between about pH 2.7 and about pH 2.1” shall also describe an embodiment specifying a pH range of “between pH 2.7 and pH 2.1”, etc.

For the purposes of comparing two or more nucleotide sequences, the percentage of “sequence identity” between a first nucleotide sequence and a second nucleotide sequence may be calculated by dividing [the number of nucleotides in the first nucleotide sequence that are identical to the nucleotides at the corresponding positions in the second nucleotide sequence] by [the total number of nucleotides in the first nucleotide sequence] and multiplying by [100%], in which each deletion, insertion, substitution or addition of a nucleotide in the second nucleotide sequence—compared to the first nucleotide sequence—is considered as a difference at a single nucleotide (position). Alternatively, the degree of sequence identity between two or more nucleotide sequences may be calculated using a known computer algorithm for sequence alignment such as NCBI Blast v2.0, using standard settings. Some other techniques, computer algorithms and settings for determining the degree of sequence identity are for example described in WO 04/037999, EP 0967284, EP 1085089, WO 00/55318, WO 00/78972, WO 98/49185 and GB 2357768. Usually, for the purpose of determining the percentage of “sequence identity” between two nucleotide sequences in accordance with the calculation method outlined hereinabove, the nucleotide sequence with the greatest number of nucleotides will be taken as the “first” nucleotide sequence, and the other nucleotide sequence will be taken as the “second” nucleotide sequence.

For the purposes of comparing two or more amino acid sequences, the percentage of “sequence identity” between a first amino acid sequence and a second amino acid sequence (also referred to herein as “amino acid identity”) may be calculated by dividing [the number of amino acid residues in the first amino acid sequence that are identical to the amino acid residues at the corresponding positions in the second amino acid sequence] by [the total number of amino acid residues in the first amino acid sequence] and multiplying by [100%], in which each deletion, insertion, substitution or addition of an amino acid residue in the second amino acid sequence—compared to the first amino acid sequence—is considered as a difference at a single amino acid residue (position), i.e., as an “amino acid difference” as defined herein. Alternatively, the degree of sequence identity between two amino acid sequences may be calculated using a known computer algorithm, such as those mentioned above for determining the degree of sequence identity for nucleotide sequences, again using standard settings. Usually, for the purpose of determining the percentage of “sequence identity” between two amino acid sequences in accordance with the calculation method outlined hereinabove, the amino acid sequence with the greatest number of amino acid residues will be taken as the “first” amino acid sequence, and the other amino acid sequence will be taken as the “second” amino acid sequence.

Also, in determining the degree of sequence identity between two amino acid sequences, the skilled person may take into account so-called “conservative” amino acid substitutions, which can generally be described as amino acid substitutions in which an amino acid residue is replaced with another amino acid residue of similar chemical structure, and which has little or essentially no influence on the 3D structure, function, activity, or other biological properties of the polypeptide. Such conservative amino acid substitutions are well known in the art, for example from WO 04/037999, GB 335768, WO 98/49185, WO 00/46383, and WO 01/09300; and (preferred) types and/or combinations of such substitutions may be selected on the basis of the pertinent teachings from WO 04/037999 as well as WO 98/49185 and from the further references cited therein.

Such conservative substitutions preferably are substitutions in which one amino acid within the following groups (a)-(e) is substituted by another amino acid residue within the same group: (a) small aliphatic, nonpolar or slightly polar residues: Ala, Ser, Thr, Pro and Gly; (b) polar, negatively charged residues and their (uncharged) amides: Asp, Asn, Glu and Gln; (c) polar, positively charged residues: His, Arg and Lys; (d) large aliphatic, nonpolar residues: Met, Leu, lie, Val and Cys; and (e) aromatic residues: Phe, Tyr and Trp. Particularly preferred conservative substitutions are as follows: Ala into Gly or into Ser; Arg into Lys; Asn into Gln or into His; Asp into Glu; Cys into Ser; Gln into Asn; Glu into Asp; Gly into Ala or into Pro; His into Asn or into Gln; Ile into Leu or into Val; Leu into lie or into Val; Lys into Arg, into Gln or into Glu; Met into Leu, into Tyr or into lie; Phe into Met, into Leu or into Tyr; Ser into Thr; Thr into Ser; Trp into Tyr; Tyr into Trp; and/or Phe into Val, into lie or into Leu.

When comparing two immunoglobulin single variable domains, the term “amino acid difference” refers to an insertion, deletion or substitution of a single amino acid residue on a position of the first sequence, compared to the second sequence; it being understood that two immunoglobulin single variable domains can contain one, two or more such amino acid differences.

For the purposes of comparing two or more immunoglobulin single variable domains or other amino acid sequences such as, e.g., the polypeptides of the invention etc., the percentage of “sequence identity” between a first amino acid sequence and a second amino acid sequence (also referred to herein as “amino acid identity”) may be calculated or determined as described in paragraph f) on pages 49 and 50 of WO 08/020079 (incorporated herein by reference), such as by dividing [the number of amino acid residues in the first amino acid sequence that are identical to the amino acid residues at the corresponding positions in the second amino acid sequence] by [the total number of amino acid residues in the first amino acid sequence] and multiplying by [100%], in which each deletion, insertion, substitution or addition of an amino acid residue in the second amino acid sequence—compared to the first amino acid sequence—is considered as a difference at a single amino acid residue (position), i.e., as an “amino acid difference” as defined herein; alternatively, the degree of sequence identity between two amino acid sequences may be calculated using a known computer algorithm for sequence alignment, such as NCBI Blast v2.0, using standard settings. Some other techniques, computer algorithms and settings for determining the degree of sequence identity are for example described in WO 04/037999, EP 0 967 284, EP 1 085 089, WO 00/55318, WO 00/78972, WO 98/49185 and GB 2 357 768-A.

Usually, for the purpose of determining the percentage of “sequence identity” between two amino acid sequences in accordance with the calculation method outlined hereinabove, the amino acid sequence with the greatest number of amino acid residues will be taken as the “first” amino acid sequence, and the other amino acid sequence will be taken as the “second” amino acid sequence.

Also, in determining the degree of sequence identity between two immunoglobulin single variable domains, the skilled person may take into account so-called “conservative” amino acid substitutions, which can generally be described as amino acid substitutions in which an amino acid residue is replaced with another amino acid residue of similar chemical structure and which has little or essentially no influence on the function, activity or other biological properties of the polypeptide. Such conservative amino acid substitutions are well known in the art, for example from WO 04/037999, GB-A-3 357 768, WO 98/49185, WO 00/46383 and WO 01/09300; and (preferred) types and/or combinations of such substitutions may be selected on the basis of the pertinent teachings from WO 04/037999 as well as WO 98/49185 and from the further references cited therein. Examples of conservative substitutions are described herein further below.

Any amino acid substitutions applied to the polypeptides described herein may also be based on the analysis of the frequencies of amino acid variations between homologous proteins of different species developed by Schulz et al. 1978 (Principles of Protein Structure, Springer-Verlag), on the analyses of structure forming potentials developed by Chou and Fasman 1975 (Biochemistry 13: 211) and 1978 (Adv. Enzymol. 47: 45-149), and on the analysis of hydrophobicity patterns in proteins developed by Eisenberg et al. 1984 (Proc. Natl. Acad. Sci. USA 81: 140-144), Kyte & Doolittle 1981 (J Molec. Biol. 157: 105-132), and Goldman et al. 1986 (Ann. Rev. Biophys. Chem. 15: 321-353), all incorporated herein in their entirety by reference. Information on the primary, secondary and tertiary structure of ISVDs is given in the description herein and in the general background art cited above. Also, for this purpose, the crystal structure of a V-m domain from a llama is for example given by Desmyter et al. 1996 (Nature Structural Biology, 3: 803), Spinelli et al. 1996 (Natural Structural Biology 3: 752-757), and Decanniere et al. 1999 (Structure, 7: 361). Further information about some of the amino acid residues that in conventional V_{H} domains form the V_{H}/V_{L} interface and potential camelizing substitutions on these positions can be found in the prior art cited above.

Immunoglobulin single variable domains and nucleic acid sequences are said to be “exactly the same” if they have 100% sequence identity (as defined herein) over their entire length.

The term “wild-type” refers to a gene or gene product isolated from a naturally occurring source. A wild-type gene is that which is most frequently observed in a population and is thus arbitrarily designed the “normal” or “wild-type” form of the gene. In contrast, the term “modified”, “mutant” or “variant” refers to a gene or gene product that displays modifications in sequence, post-translational modifications and/or functional properties (i.e., altered characteristics) when compared to the wild-type gene or gene product. It is noted that naturally occurring mutants can be isolated; these are identified by the fact that they have altered characteristics when compared to the wild-type gene or gene product. Alternatively, a variant may also include synthetic molecules, e.g., a chemokine ligand variant may be similar in structure and/or function to the natural chemokine, but may concern a small molecule, or a synthetic peptide or protein, which is man-made. Said variants with different functional properties may concerns super-agonists, super antagonists, among other functional differences, as known to the skilled person.

In the context of the present invention, the terms “specificity”, “binding specifically” or “specific binding” refer to the number of different target molecules, such as antigens, to which a particular binding unit can bind with sufficiently high affinity (see below). “Specificity”, “binding specifically” or “specific binding” are used interchangeably herein with “selectivity”, “binding selectively” or “selective binding”. Generally, binding units, such as binding ISVDs, specifically bind to their designated targets.

The specificity/selectivity of a binding unit can be determined based on affinity. The affinity denotes the strength or stability of a molecular interaction. The affinity is commonly given by the K_{D}, or dissociation constant, which has units of mol/litre (or M). The affinity can also be expressed as an association constant, K_{A}, which equals 1/K_{D} and has units of (mol/litre)⁻¹ (or M⁻¹).

The affinity is a measure for the binding strength between a moiety and a binding site on a target molecule: the lower the value of the K_{D}, the stronger the binding strength between a target molecule and a targeting moiety.

The K_{D}-value characterizes the strength of a molecular interaction also in a thermodynamic sense as it is related to the change of free energy (DG) of binding by the well-known relation DG=RT·ln(K_{D}) (equivalently DG=−RT·ln(K_{A})), where R equals the gas constant, T equals the absolute temperature and ln denotes the natural logarithm.

The K_{D} may also be expressed as the ratio of the dissociation rate constant of a complex, denoted as k_{off}, to the rate of its association, denoted kₒₙ(so that K_{D}=k_{off}/kₒₙ and K_{A}=kₒₙ/k_{off}).

The off-rate k_{off} has units s⁻¹ (where s is the SI unit notation of second). The on-rate kₒₙ has units M⁻¹s⁻¹. The on-rate may vary between 10² M⁻¹s⁻¹ to about 10⁷ M⁻¹s⁻¹, approaching the diffusion-limited association rate constant for bimolecular interactions. The off-rate is related to the half-life of a given molecular interaction by the relation t_{1/2}=ln(2)/k_{off}. The off-rate may vary between 10⁻⁶ s⁻¹ (near irreversible complex with a t_{1/2} of multiple days) to 1 s⁻¹ (t_{1/2}=0.69 s).

The measured K_{D} may correspond to the apparent K_{D} if the measuring process somehow influences the intrinsic binding affinity of the implied molecules for example by artefacts related to the coating on the biosensor of one molecule. Also, an apparent K_{D} may be measured if one molecule contains more than one recognition sites for the other molecule or molecules. In such situation the measured affinity may be affected by the avidity of the interaction by the two molecules.

The dissociation constant (K_{D}) may be the actual or apparent dissociation constant, as will be clear to the skilled person. Methods for determining the K_{D} will be clear to the skilled person, and for example include the techniques mentioned below. In this respect, it will also be clear that it may not be possible to measure dissociation constants of more than 10⁻⁴ moles/litre or 10⁻³ moles/litre (e.g., of 10⁻² moles/litre). Optionally, as will also be clear to the skilled person, the (actual or apparent) K_{D} may be calculated on the basis of the (actual or apparent) association constant (K_{A}), by means of the relationship (K_{D}=1/K_{A}). K_{A}=1/K_{D}-->K_{A}=[AB]/[A].[B].

In the context of the present invention, the term “avidity” refers to the accumulated strength of multiple affinities of individual non-covalent binding interactions, such as between a protein receptor and its ligand, and may also be referred to as “functional affinity”. “Avidity” differs from “affinity”, which, as explained in detail above, describes the binding strength of a single interaction.

The term “half-life” as used here can generally be defined as described in paragraph o) on page 57 of WO 2008/020079 and as mentioned therein refers to the time taken for the serum concentration of the compound or polypeptide to be reduced by 50%, in vivo, for example due to degradation of the sequence or compound and/or clearance or sequestration of the sequence or compound by natural mechanisms. The in vivo half-life of the protein-based carrier building block and/or molecule of the invention can be determined in any manner known per se, such as by pharmacokinetic analysis. Suitable techniques will be clear to the person skilled in the art and may for example generally be as described in paragraph o) on page 57 of WO 2008/020079. As also mentioned in paragraph o) on page 57 of WO 2008/020079, the half-life can be expressed using parameters such as the t_{1/2}-alpha, t_{1/2}-beta and the area under the curve (AUC). In this respect it should be noted that the term “half-life” as used herein in particular refers to the t_{1/2}-beta or terminal half-life (in which the t_{1/2}-alpha and/or the AUC or both may be kept out of considerations). Reference is for example made to the standard handbooks, such as Kenneth, A et a: Chemical Stability of Pharmaceuticals: A Handbook for Pharmacists and Peters et al, Pharmacokinetic analysis: A Practical Approach (1996). Reference is also made to “Pharmacokinetics”, M Gibaldi & D Perron, published by Marcel Dekker, 2nd Rev. edition (1982). Similarly, the terms “increase in half-life” or “increased half-life” are also as defined in paragraph o) on page 57 of WO 2008/020079 and in particular refer to an increase in the t_{1/2}-beta, either with or without an increase in the t_{1/2}-alpha and/or the AUC or both.

As used herein, the term “hinge region” refers to the portion of a heavy chain molecule that joins the CH1 domain to the CH2 domain. This hinge region consists of approximately 25 residues and is flexible, allowing the two N-terminal antigen binding regions to move independently. The hinge region can be divided into three distinct domains: the upper, middle and lower hinge domains (Roux et al. J. Immunol. 161: 4083 (1998)). The FcRn antagonists in the instant disclosure may contain all or part of a hinge region.

As used herein, the term “CH2 domain” includes the portion of an immunoglobulin heavy chain molecule extending, for example, from approximately positions 244-360 in the Kabat numbering system (Eu positions 231-340). The CH2 domain is unique in that it is not closely linked to another domain. Rather, two N-linked branched carbohydrate chains are sandwiched between the two CH2 domains of an intact native IgG molecule. In one embodiment, a binding polypeptide of the present disclosure comprises a CH2 domain derived from an IgG1 molecule (e.g., a human IgG1 molecule).

As used herein, the term “CH3 domain” includes the portion of a heavy chain immunoglobulin molecule extending approximately 110 residues from the N-terminus of the CH2 domain, e.g., from about positions 361-476 of the Kabat numbering system (Eu positions 341447). The CH3 domain typically forms the C-terminal portion of the antibody. However, in some immunoglobulins, additional domains may extend from the CH3 domain to form the C-terminal portion of the molecule (e.g., the CH4 domain in the U chain of IgM and the E chain of IgE). In one embodiment, a binding polypeptide of the present disclosure comprises a CH3 domain derived from an IgG1 molecule (e.g., a human IgG1 molecule).

As used herein, the term “binding site” comprises a region of a polypeptide which is responsible for selectively binding to a target antigen of interest (e.g., AChR). Binding domains comprise at least one binding site. Exemplary binding domains include an antibody variable domain. Antibody molecules may comprise a single binding site or multiple (e.g., two, three or four) binding sites.

The terms “variable region” and “variable domain” are used herein interchangeable and are intended to have equivalent meaning. The term “variable” refers to the fact that certain portions of the variable domains VH and VL differ extensively in sequence among antibodies and are used in the binding and specificity of each particular antibody for its target antigen. However, the variability is not evenly distributed throughout the variable domains of antibodies. It is concentrated in three segments called “hypervariable loops” in each of the VL domain and the VH domain which form part of the antigen binding site.

The term “EU position” as used herein, refers to the amino acid position in the EU numbering convention for the Fc region described in Edelman, G. M. et al., Proc. Natl. Acad. USA, 63, 78-85 (1969) and Kabat et al., in “Sequences of Proteins of Immunological Interest”, U.S. Dept. Health and Human Services, 5th edition, 1991. Unless otherwise indicated, Fc residue positions referred to herein are in accordance with the EU Ig numbering system.

The term “FcRn binding fragment” refers to a portion of an Fc region that is sufficient to confer FcRn binding.

In the context of the present invention, “acidic pH” or “acidic endosomal pH” refers to an acid physiological pH, such as the pH inside endosomes, which is generally pH<6.8, or <6.5, such as between 5.0 and 6.8, such as about 5.1, 5.2, 5.3, 5.4, 5.5, 5.6, 5.7, 5.8, 5.9, 6.0, 6.1, 6.2, 6.3, 6.4, 6.5, 6.6, or 6.7, or such as about pH 6.4, 6.3, 6.2, 6.1 or about pH 6.0. Hence, “acidic pH” may refer to a pH of between about 6.0 and about 6.5. In the context of the present invention, “neutral pH”, “near neutral pH” or “extracellular physiological pH” refers to the pH of the extracellular space, e.g., a pH of about 7.0 to about 7.5, such as about 7.0, 7.1, 7.2, 7.3, 7.4 or 7.5, preferably about 7.4.

### The FcRn Antagonists of the Present Invention

The present invention provides molecules or polypeptides (also referred to as FcRn antagonists or constructs in the context of the present invention) comprising (a) at least one first polypeptide specifically binding to an epitope on the neonatal Fc receptor (FcRn) and (b) at least a second polypeptide which competes with wild-type IgG1 Fc region for binding to FcRn.

The term “FcRn antagonist” or “FcRn antagonists” as used herein refers to any agent that specifically binds to FcRn and modulates, interferes with, inhibits, and/or blocks the natural function of FcRn to a certain extent. For instance, a “FcRn antagonist” according to the present invention refers to any agent that binds specifically to FcRn and inhibits the binding of immunoglobulin to FcRn, i.e., competes with wild-type IgG1 Fc region for binding to FcRn. The FcRn antagonists of the present invention comprise at least two polypeptides ((a) and (b)), as defined above, each of which specifically binds to an epitope on FcRn. In certain embodiments, the epitope on FcRn to which the first polypeptide binds, is identical to the epitope on FcRn to which the second polypeptide binds. In certain other embodiments, the epitope on FcRn to which the first polypeptide binds, is different from the epitope on FcRn to which the second polypeptide binds. Hence, the FcRn antagonists of the present invention are specific to either one single epitope on FcRn or to two or more different epitopes on the FcRn receptor. In addition, the FcRn antagonists of the present invention comprise at least one polypeptide which competes with the wild-type IgG1 Fc region for binding to FcRn. Hence, the FcRn antagonists of the present invention also compete with the wild-type IgG1 Fc region for binding to FcRn.

The term “a polypeptide which competes with wild-type IgG1 Fc region for binding to FcRn” as used herein, refers to polypeptides that bind to FcRn in a way that reduces or inhibits the binding of wild-type IgG1 Fc region to FcRn, as compared with the binding of wild-type IgG1 Fc region to FcRn in the absence of the polypeptide. Polypeptides that compete with the wild-type IgG1 Fc region for binding to FcRn can be identified by the skilled person by using any suitable method in the art, for example, by using competition ELISA or Biacore assays where binding of the polypeptide to FcRn prevents/reduces the amount of binding of the wild-type IgG1 Fc region to FcRn. Similarly, the term “a polypeptide which does not compete with wild-type IgG1 Fc region for binding to FcRn” as used herein, refers to polypeptides that bind to FcRn in a way that does not significantly affect or does not significantly interfere with the binding of wild-type IgG1 Fc region to FcRn as compared with the binding of wild-type IgG1 Fc region to FcRn in the absence of the polypeptide.

In a preferred embodiment, the at least one first polypeptide (a) which specifically binds to an epitope on the Fc receptor (FcRn) and the second polypeptide (b) which competes with the wild-type IgG1 Fc region for binding to FcRn, comprised in the FcRn antagonists of the present invention, bind synergistically or cooperatively to FcRn. In the context of the present invention, the terms “synergistic binding”, “bind synergistically”, “cooperative binding” or “bind cooperatively” refer to a property of the binding of the combination of the first and second polypeptides when comprised in the FcRn antagonists of the present invention, which binding results in improved effects as compared with the sum of the effects caused by the binding of each of the polypeptides on their own (i.e., not present in combination with each other in the FcRn antagonists of the present invention). Synergistic and/or simultaneous binding of the first and second polypeptides comprised in the FcRn antagonists of the present invention to FcRn may lead to several improved effects.

For instance, synergistic binding of the first and second polypeptides in the FcRn antagonists of the present invention to FcRn may lead to improved serum IgG clearance (in vitro or in vivo) (i.e., improved serum IgG removal) as compared with the IgG clearance (in vitro or in vivo) driven by each of the those first and second polypeptides separately. For example, improved serum IgG clearance may be achieved by the FcRn antagonists of the present invention by cross-linking different FcRn receptors (e.g., when each of the polypeptides comprised in the FcRn antagonists binds to a different FcRn molecule).

For instance, synergistic binding may lead to an extended half-life of the FcRn antagonists of the present invention as compared with the half-life of each of the polypeptides comprised in the FcRn antagonists of the present invention on their own.

For instance, the presence of both the first and second polypeptides in the FcRn antagonists of the present invention may lead to a more efficient competition of the FcRn antagonists of the present invention with the wild-type IgG1 Fc region for binding to FcRn, as compared with the competition of each of those first and second polypeptides individually with the wild-type IgG1 Fc region for binding to FcRn. This would also lead to improved serum IgG clearance of the FcRn antagonists of the present invention.

For instance, the affinity (avidity) of the FcRn antagonists for FcRn binding may be increased as compared to the affinity of each of the individual polypeptides for FcRn that are comprised in the FcRn antagonists of the present invention (avidity effect).

For instance, synergistic binding of the first and second polypeptides comprised in the FcRn antagonists of the present application may allow for a FcRn antagonist with an extended half-life compared to the half-life of each of those first and second polypeptides individually while still allowing efficient blocking of the FcRn receptor. By working with at least two binding units to FcRn (the at least two polypeptides, (a) and (b), as described above), the FcRn antagonists of the present invention may be able to block FcRn (e.g., by efficiently binding to FcRn at extracellular physiological and acidic endosomal pH) in an improved manner due to the avidity effect. In this way, by having at least two polypeptides ((a) and (b)) that produce an avidity effect, the affinity of each of the two polypeptides at extracellular physiological pH can be kept within certain limits, so that the recycling mechanism for extending the half-life is still efficient, while at the same time the FcRn receptor is sufficiently blocked to achieve an antagonistic therapeutic effect (e.g., sufficiently increased IgG clearance).

Hence, in some embodiments, the presence of the first polypeptide and the second polypeptide in the FcRn antagonists of the present invention provides for an increased avidity of the FcRn antagonists for FcRn. Hence, in one embodiment, the FcRn antagonists of the present invention have increased FcRn affinity (avidity) as compared with the sum of the individual FcRn affinities of polypeptides (a) and (b).

Hence, in some embodiments, the presence of the first polypeptide and the second polypeptide in the FcRn antagonists of the present invention provides for an increased clearance of an Fc-containing agent, such as an increased IgG clearance. Hence, in one embodiment, the FcRn antagonists of the present invention provide for an increased clearance of an Fc-containing agent, such as an increased IgG clearance, as compared with the sum of the individual clearance of polypeptides (a) and (b).

In certain embodiments, the at least one first polypeptide (a) which specifically binds to an epitope on the Fc receptor (FcRn); and the second polypeptide (b) that competes with the wild-type IgG1 Fc region for binding to FcRn comprised in the FcRn antagonists of the present invention bind cooperatively to FcRn, resulting in an improved affinity (avidity effect).

In certain embodiments, the FcRn antagonists bind specifically to FcRn with increased avidity for FcRn relative to the affinity of a wild-type IgG1 Fc region.

In another embodiment, the FcRn antagonists of the present invention bind specifically to FcRn with increased avidity at an acidic endosomal pH and at a non-acidic pH (i.e., at a neutral or extracellular physiological pH) for FcRn relative to the affinity of a wild-type IgG1 Fc region and/or relative to the sum of the individual FcRn affinities of polypeptides (a) and (b).

In certain embodiments, the FcRn antagonists of the present invention bind to FcRn with reduced pH dependence relative to a wild-type IgG Fc region. For instance, blockade of FcRn can be increased by binding at extracellular physiological pH, as this ensures that FcRn is already occupied at the cell surface, and that it remains blocked after uptake into the cell.

For example, if the first polypeptide comprised in the FcRn antagonists of the present invention has a high binding affinity for FcRn at extracellular physiological pH, this first binding event could facilitate the binding of the second polypeptide at acidic endosomal pH because the FcRn antagonist is already in proximity to the FcRn receptor. This effect would be independent of the specific epitope of the first polypeptide on FcRn.

For example, increased avidity could also be achieved if both polypeptides can efficiently bind FcRn simultaneously at acidic endosomal pH. For example, if the first polypeptide has a high binding affinity to FcRn at extracellular physiological pH, this first binding event could facilitate binding of the second polypeptide at acidic endosomal pH. This avidity effect would be further enhanced if the first polypeptide remained bound to FcRn at an acidic endosomal pH.

In one embodiment, the first polypeptide (a) which specifically binds to an epitope on the Fc receptor (FcRn), competes with the wild-type IgG1 Fc region for binding to FcRn.

In a preferred embodiment, the first polypeptide (a) specifically binding to an epitope on the Fc receptor (FcRn) does not compete with the wild-type IgG1 Fc region for binding to FcRn.

In certain embodiments, the first polypeptide (a) and the second polypeptide (b) both compete with the wild-type IgG1 Fc region for binding to FcRn at an acidic endosomal pH.

In certain embodiments, the first polypeptide (a) binds to FcRn at extracellular physiological or neutral pH (e.g., about pH 7.4) and/or at acidic endosomal pH (e.g., about pH 6.0) with higher affinity as compared with the binding affinity of the wild-type IgG1 Fc region.

In certain embodiments, the second polypeptide (b) binds to FcRn at extracellular physiological or neutral pH (e.g., about pH 7.4) and/or at acidic endosomal pH (e.g., about pH 6.0) with higher affinity as compared with the binding affinity of the wild-type IgG1 Fc region.

In a preferred embodiment, the at least one first polypeptide (a) which specifically binds to an epitope on the Fc receptor (FcRn) and the second polypeptide (b) which competes with the wild-type IgG1 Fc region for binding to FcRn comprised in the FcRn antagonists of the present application bind cooperatively or synergistically to FcRn, resulting in an increased or at least not substantially reduced half-life of the FcRn antagonists of the present invention as compared with the half-life of the (single) first and/or second polypeptides.

In particular embodiments, the invention provides FcRn antagonists that have a molecular weight of at least about 30 kDa, such as about 40 kDa, or about 50 kDa, or about 165 kDa, or about 175 kDa, or more, such as about 300 kDa. Preferably, the FcRn antagonists of the present invention have a molecular weight of between about 30 kDa and about 165 kDa, and in particular, they have a molecular weight of between 30 kDa and 165 kDa.

The second polypeptide (b) which competes with the wild-type IgG1 Fc region for binding to FcRn comprised in the FcRn antagonists of the present application competes with the wild-type IgG1 Fc region for binding to FcRn and may hence influence the serum clearance of IgG. The term “clearance” as used herein, refers to the amount of a substance (e.g., antibody or polypeptide) cleared (e.g., by biotransformation and/or excretion) from a compartment or fluid (e.g., blood) per unit time. Since the concentration of the antibody in its volume of distribution is most commonly sampled by analysis of blood or plasma, clearances are most commonly described as the “plasma clearance” or “blood clearance”. Suitable techniques of measuring the clearance will be clear to the person skilled in the art. For instance, the amount of antibody in the serum may be measured by liquid chromatography-tandem mass spectrophotometric (LC-MS/MS), and the clearance may be calculated by plotting the concentration-time profile, followed by calculating/estimating the area under the plasma concentration time profile (AUC_{LAST}). The AUC (expressed in mg*h/L) is indicative of the total body exposure to a drug over time following the administration of a dose of the drug. It therefore provides insights into the rate of elimination of the drug from the body and the dose administered. Consequently, it can be used to calculate the clearance rate from the body (% reduction in IgG exposure).

Hence, in one embodiment the FcRn antagonists of the present invention increase the clearance of an Fc-containing agent, such as an IgG. In an embodiment, the FcRn antagonists of the present invention increases the clearance of a circulating IgG in the serum or plasma of a subject. In certain embodiments, the FcRn antagonists of the present invention increase the clearance of IgG by about 50%-100%, preferably by about 70%-90%, such as about 78% or about 80%. In certain embodiments, the FcRn antagonists of the present invention increase the clearance of IgG by more than 30-times, such as 45 or 51 times.

In addition, additive or synergistic binding effects between the polypeptides (a) and (b) may further improve serum IgG clearance driven by the FcRn antagonists of the present invention (i.e., improved serum IgG removal) as compared with the IgG clearance driven by each of the polypeptides comprised in the FcRn antagonists of the present invention.

Hence, in certain embodiments, the FcRn antagonists of the present invention increase serum IgG clearance as compared with the IgG clearance driven by each of the polypeptides comprised in the FcRn antagonists of the present invention in isolation. In other embodiments, the FcRn antagonists of the present invention increase serum IgG clearance as compared with the IgG clearance driven by polypeptide (b), such as TP061, in isolation. In other embodiments, the FcRn antagonists of the present invention increases serum IgG clearance as compared with the IgG clearance driven by polypeptide (b), such as TP061, in isolation by 1.5-5 times, such as 1.6 or 1.8 times. In other embodiments, the FcRn antagonists of the present invention increases serum IgG clearance as compared with the IgG clearance driven by polypeptide (b), such as TP061, in isolation by more than 10%, such as 14% or 16%.

In addition, additive or synergistic binding effects of polypeptides (a) and (b) comprised in the FcRn antagonists of the present invention to FcRn may further decrease serum IgG half-life as driven by the FcRn antagonists of the present invention (i.e., improved serum IgG removal) as compared with the IgG half-life driven by each of the polypeptides (a) and (b) in their individual form.

In other embodiments, the FcRn antagonists of the present invention decrease the half-life of an Fc-containing agent, such as an IgG. In preferred embodiments, the FcRn antagonists of the present invention decrease the half-life of an Fc-containing agent, such as an IgG, as compared with the IgG half-life decrease driven by each of the polypeptides comprised in the FcRn antagonists of the present invention in isolation. In other embodiments, the FcRn antagonists of the present invention decrease the half-life of serum IgG as compared with the IgG decrease driven by polypeptide (b), such as TP061, in isolation.

### The First Polypeptide (a) Comprised in the FcRn Antagonists of the Present Invention

As described above, the FcRn antagonists of the present invention comprise (a) at least one first polypeptide specifically binding to an epitope on FcRn. This first polypeptide is not limited as long as it specifically binds to an epitope on FcRn. For instance, the first polypeptide may compete with the wild-type IgG1 Fc region for binding to FcRn, i.e., the presence of the first polypeptide may cause a decrease in the binding of wild-type IgG1 Fc region to FcRn as compared with the binding of the wild-type IgG1 Fc region to FcRn in the absence of the first polypeptide. In a preferred embodiment, the first polypeptide (a) comprised in the FcRn antagonists of the present technology does not compete with wild-type IgG1 Fc region for binding to FcRn. In one embodiment, the first polypeptide is not an FcRn antagonist as such in its isolated form.

In an embodiment, the at least one first polypeptide (a) specifically binding to an epitope on the Fc receptor (FcRn) comprised in the FcRn antagonists of the present invention comprises or consists of an Affibody® (affibody molecule), a single-chain variable fragment (scFv), a Fab, a Designed Ankyrin Repeat Protein (DARPin®), a Nanofitin® (aka affitin) or an immunoglobulin variable domain sequence (ISVD).

Affitins are artificial proteins with the ability to selectively bind antigens. They are structurally derived from the DNA-binding protein Sac7d, found in Sulfolobus acidocaldarius. Due to their small size and high solubility, they can be easily produced in large amounts using bacterial expression systems (see, e.g., Kalichuk V. et al., “A novel, smaller scaffold for Affitins: Showcase with binders specific for EpCAM”, Biotechnol Bioeng. 2018; 115(2):290-299). In a preferred embodiment, the at least one first polypeptide (a) specifically binding to an epitope on the Fc receptor (FcRn) comprised in the FcRn antagonists of the present invention comprises or consists of a Nanofitin® (aka affitin). In this case, the affitin preferably does not interfere (e.g., not compete) with the binding of wild-type IgG1 Fc region to FcRn.

In one embodiment, the at least one first polypeptide (a) specifically binding to an epitope on the Fc receptor (FcRn) comprised in the FcRn antagonists of the present invention comprises or consists of a scFv. A scFv is a fusion protein of the variable regions of the heavy (VH) and light chains (VL) of immunoglobulins, connected with a short linker peptide of ten to about 25 amino acids. This protein generally retains the specificity of the original immunoglobulin, despite removal of the constant regions and the introduction of the linker. In this case, the scFv preferably does not interfere (e.g., not compete) with the binding of wild-type IgG1 Fc region to FcRn.

In another preferred embodiment, the at least one first polypeptide (a) specifically binding to an epitope on the Fc receptor (FcRn) comprised in the FcRn antagonists of the present invention comprises or consists of an immunoglobulin single variable domain (ISVD). Hence, in a preferred embodiment, the at least one first polypeptide (a) specifically binds to an epitope on FcRn which is different from the epitopes bound by the known natural FcRn ligands (i.e., serum albumin and IgG). This polypeptide (a) would thus not interfere (e.g., not compete) with the binding of wild-type IgG1 Fc region to FcRn.

In a preferred embodiment, the at least one first polypeptide (a) comprised in the FcRn antagonists of the present invention comprises or consists of an antibody or fragment thereof.

The term “antibody” as used herein refers to an intact antibody, or a fragment thereof, such as a binding fragment thereof, that competes with the intact antibody for specific binding. Hence, the term “antibody” as used herein refers to any functional antibody that is capable of specifically binding to the antigen of interest. An antibody fragment or fragment of an antibody as used herein refers to a portion of an antibody that retains the capability of the antibody to specifically bind to the antigen. Antibody fragments include, but are not limited to Fc regions, Fc domains, F(ab), F(ab′), F(ab′)2, Fv, single-chain antibodies, (scFv's), single-domain antibodies, diabodies or any other portion(s) of the antibody that retain the capability to compete with the intact antibody for specific binding.

For instance, the at least one first polypeptide (a) may comprise or consist of a Fc domain or a fragment thereof.

As used in the present description, the term “Fc,” “Fc domain,” “Fc region,” or “Fc fragment” is used interchangeably and is defined as the portion of a heavy chain constant region beginning in the hinge region just N-terminal of the papain cleavage site (i.e., residue 216 in IgG, taking the first residue of heavy chain constant region to be 114) and ending at the C-terminus of the heavy chain. Accordingly, a complete Fc, Fc domain, Fc region, or Fc fragment comprises at least a hinge region or a part of it, a CH2 domain, and a CH3 domain. For instance, a Fc domain may comprise at least a hinge region or a part of it, two CH2 domains, and two CH3 domains. Sequence alignment of portions (CH2 and CH3 domains, residues 231 to 447, EU numbering) of exemplary human IgG1, IgG2, IgG3, and IgG4 Fc domains is shown in FIG. 32 of WO 2021/016571. The term encompasses native/wildtype Fc and Fc variants as described herein and includes molecules in monomeric or multimeric (e.g., dimeric) form, whether digested from whole antibody or produced by other means such as recombinant technology. See, e.g., Ying et al., JBC (2013) 288:25154-164; and Yang et al., JBC (2019) 294:10638-48.

The original immunoglobulin source of the native Fc is typically of human origin and can be any of the immunoglobulins, such as IgG1 and IgG2. Native Fc molecules are made up of monomeric polypeptides that can be linked into dimeric or multimeric forms by covalent (i.e., disulfide bonds) and non-covalent association. The number of intermolecular disulfide bonds between monomeric subunits of native Fc molecules ranges from 1 to 4 depending on class (e.g., IgG, IgA, and IgE) or subclass (e.g., IgG1, IgG2, IgG3, IgA1, and IgA2). One example of a native Fc is a disulfide-bonded dimer resulting from papain digestion of an IgG. The term “native Fc,” as used herein, is generic to the monomeric, dimeric, and multimeric forms.

The term “Fc,” “Fc domain,” “Fc region,” or “Fc fragment” used in the present description encompasses also “Fc variants” “modified Fc” or “modified Fc domains”, i.e., molecules or sequences that are modified from a native/wildtype Fc but still comprise a binding site for the FcRn. A Fc variant or a modified Fc domain also can be shorter or longer than a native Fc (e.g., shorter or longer than a sequence spanning residues 216 to 447 of human IgG, Eu numbering); for example, the Fc variant or modified Fc may lack certain N-terminal and/or C-terminal amino acid residues of the native Fc, or may contain additional amino acid residues at the N-terminus and/or C-terminus compared to a native Fc. A modified Fc domain itself does not include an antigen-binding domain of an antibody or an antibody variant, or a target-binding domain of an immunoadhesin. The term encompasses a molecule or sequence that is humanized from a non-human native Fc. Furthermore, a native Fc comprises regions that can be removed because they provide structural features or biological activities that are not required for the FcRn antagonists (e.g., antibody-like binding polypeptides) as described in WO 2021/016571. Thus, the term encompasses a molecule or sequence that lacks one or more native Fc sites or residues, or in which one or more Fc sites or residues have been modified, that affect or are involved in: (1) disulfide bond formation, (2) incompatibility with a selected host cell, (3) N-terminal heterogeneity upon expression in a selected host cell, (4) glycosylation, (5) interaction with complement, (6) binding to an Fc receptor other than a salvage receptor (an FcγR), or (7) antibody-dependent cellular cytotoxicity (ADCC) or complement-dependent cytotoxicity (CDC).

In certain embodiments, the Fc domain comprised in the FcRn antagonist of the present invention (either as the at least one first polypeptide (a) and/or as the at least one second polypeptide (b)) may comprise one or more amino acid mutations (e.g., substitutions) which alter the effector functions (e.g., ADCC or CDC function) of the Fc domain, as compared to a corresponding wildtype molecule. In certain embodiments, the Fc domain comprised in the FcRn antagonist of the present invention may comprise one or more amino acid mutations (e.g., substitutions) which provide one or more desired biochemical characteristics such as the ability to remain monomeric, the ability to noncovalently dimerize, an increased ability to localize at a target site, and glycosylation patterns, as compared to the corresponding wildtype molecule. For example, the modified Fc domain may have reduced glycosylation (e.g., N- or O-linked glycosylation). Exemplary amino acid substitutions which confer reduced or altered glycosylation are disclosed in WO 2005/018572. In some embodiments, the Fc domain is modified to eliminate glycosylation (e.g., “agly” antibodies).

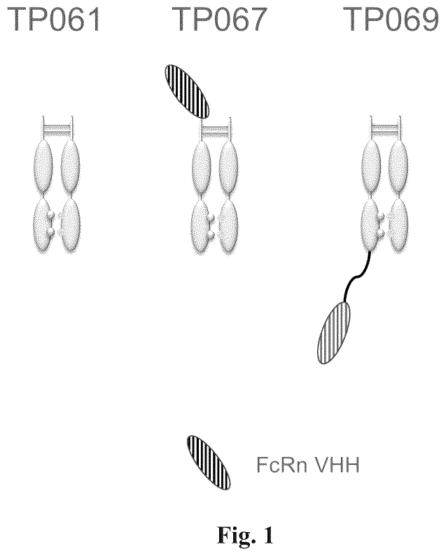

In one preferred embodiment, the Fc region comprised in the FcRn antagonist of the present invention (either as the at least one first polypeptide (a) and/or as the at least one second polypeptide (b)) is in a dimeric form and comprises at least a hinge region or a part of it, two CH2 domains, and two CH3 domains, i.e., two polypeptide fragments, see, e.g., FIG. 1. Hence, in this embodiment, the at least one first polypeptide (a) and/or as the at least one second polypeptide (b), if comprising or consisting of a Fc region, may actually comprise at least two polypeptide chains or fragments, wherein at least part of both of them conform the Fc region. The two polypeptide chains or fragments may be the same (and then, the Fc domain would be a homodimer) or may be different (and then, the Fc domain would be a heterodimer). Both two polypeptide chains or fragments may be linked to each other, e.g., by disulfide bridges, as the skilled person would understand.

Hence, in this preferred embodiment, the FcRn antagonist of the present invention comprises at least two polypeptides (a) and/or at least two polypeptides (b), i.e., the two polypeptides which conform the dimeric Fc domain. The Fc domain is preferably dimeric, such as homodimeric or heterodimeric, more preferably heterodimeric. In this preferred embodiment, at least one and preferably both of the CH3 domains comprised in the Fc domain comprised in the FcRn antagonists of the present invention (either as the first polypeptide (a) and/or the second polypeptide (b), see below), comprise knob in hole mutations. Hence, in this preferred embodiment, the Fc domain is heterodimeric. These mutations serve to engineer an interface between a first and second polypeptide (CH3 domains, in this specific case) for hetero-oligomerization using the “knobs and holes” technology as described in, for example, U.S. Pat. No. 8,216,805, WO 1996/27011, Ridgway, J B et al., “‘Knobs-into-holes’ engineering of antibody CH3 domains for heavy chain heterodimerization”, Protein engineering, 1996, 9, 7:617-21 or in Merchant et al., “An efficient route to human bispecific IgG”, Nature Biotechnology, 1998, 16: 677-681. The preferred interface comprises at least a part of the CH3 domain of the Fc domain. “Protuberances” are constructed by replacing small amino acid side chains from the interface of the first polypeptide (the first CH3 in this case) with larger side chains (e.g., tyrosine or tryptophan). Compensatory “cavities” of identical or similar size to the protuberances are optionally created on the interface of the second polypeptide (the second CH3 in this case) by replacing large amino acid side chains with smaller ones (e.g., alanine or threonine). Where a suitably positioned and dimensioned protuberance or cavity exists at the interface of either the first or second polypeptide, it is only necessary to engineer a corresponding cavity or protuberance, respectively, at the adjacent interface. See U.S. Pat. No. 8,216,805, WO 1996/27011, Ridgway, J B et al., “‘Knobs-into-holes’ engineering of antibody CH3 domains for heavy chain heterodimerization”, Protein engineering, 1996, 9, 7:617-21 or Merchant et al., “An efficient route to human bispecific IgG”, Nature Biotechnology, 1998, 16: 677-681 for further details. In a preferred embodiment, the amino acids HY in the CH3 domain of Fc domains are mutated into RF, such as at positions 435 and 436, (H435R and Y436F in CH3 domain as described by Jendeberg, L. et al. (1997, J. Immunological Meth., 201:25-34)). Hence, in this preferred embodiment, the Fc domain is heterodimeric. But the Fc domain may also be homodimeric (i.e., both polypeptide chains or fragments conforming the Fc region are the same).

In a preferred embodiment, the at least one first polypeptide (a) comprised in the FcRn antagonists of the present invention comprises or consists of an ISVD. Preferably, the ISVD does not compete with wild-type IgG1 Fc region for binding to FcRn and/or is not an FcRn antagonist.

The term “immunoglobulin single variable domain” (ISVD), interchangeably used with “single variable domain”, defines immunoglobulin molecules wherein the antigen binding site is present on, and formed by, a single immunoglobulin domain. This sets ISVDs apart from “conventional” immunoglobulins (e.g., monoclonal antibodies) or their fragments (such as Fab, Fab′, F(ab′)₂, scFv, di-scFv), wherein two immunoglobulin domains, in particular two variable domains, interact to form an antigen binding site. Typically, in conventional immunoglobulins, a heavy chain variable domain (V_{H}) and a light chain variable domain (V_{L}) interact to form an antigen binding site. In this case, the complementarity determining regions (CDRs) of both V_{H} and V_{L} will contribute to the antigen binding site, i.e., a total of 6 CDRs will be involved in antigen binding site formation.

In view of the above definition, the antigen-binding domain of a conventional 4-chain antibody (such as an IgG, IgM, IgA, IgD or IgE molecule; known in the art) or of a Fab fragment, a F(ab′)₂ fragment, an Fv fragment such as a disulphide linked Fv or a scFv fragment, or a diabody (all known in the art) derived from such conventional 4-chain antibody, would normally not be regarded as an immunoglobulin single variable domain, as, in these cases, binding to the respective epitope of an antigen would normally not occur by one (single) immunoglobulin domain but by a pair of (associating) immunoglobulin domains such as light and heavy chain variable domains, i.e., by a V_{H}-V_{L} pair of immunoglobulin domains, which jointly bind to an epitope of the respective antigen.

In contrast, ISVDs are capable of specifically binding to an epitope of the antigen without pairing with an additional immunoglobulin variable domain. The binding site of an immunoglobulin single variable domain is formed by a single V_{H}, a single V_{HH} or single V_{L} domain.

In the context of the present invention, the single variable domain may be a light chain variable domain sequence (e.g., a V_{L}-sequence) or a suitable fragment thereof; or a heavy chain variable domain sequence (e.g., a V_{H}-sequence or V_{HH} sequence) or a suitable fragment thereof; as long as it is capable of forming a single antigen binding unit (i.e., a functional antigen binding unit that essentially consists of the single variable domain, such that the single antigen binding domain does not need to interact with another variable domain to form a functional antigen binding unit).

An ISVD can for example be a heavy chain ISVD, such as a V_{H}, V_{HH}, including a camelized V_{H} or humanized V_{HH}. Preferably, it is a V_{HH}, including a camelized V_{H} or humanized V_{HH}. Heavy chain ISVDs can be derived from a conventional four-chain antibody or from a heavy chain antibody.

For example, the ISVD may be a single domain antibody (or an amino acid sequence that is suitable for use as a single domain antibody), a “dAb” or dAb (or an amino acid sequence that is suitable for use as a dAb) or a Nanobody® ISVD (as defined herein and including but not limited to a V_{HH}); other single variable domains, or any suitable fragment of any one thereof. Preferably, the ISVD is a V_{H}, a humanized V_{H}, a human V_{H}, a V_{HH}, a humanized V_{HH} or a camelized V_{H}. More preferably, the ISVD is a Nanobody® ISVD (such as a V_{HH}, including a humanized V_{HH} or camelized V_{H}) or a suitable fragment thereof. Nanobody® is a registered trademark from Ablynx N.V.

“V_{HH} domains”, also known as V_{HH}S, V_{HH} antibody fragments, and V_{HH} antibodies, have originally been described as the antigen binding immunoglobulin variable domain of “heavy chain antibodies” (i.e., of “antibodies devoid of light chains”; Hamers-Casterman et al. Nature 363: 446-448, 1993). The term “V_{HH} domain” has been chosen in order to distinguish these variable domains from the heavy chain variable domains that are present in conventional 4-chain antibodies (which are referred to herein as “V_{H} domains”) and from the light chain variable domains that are present in conventional 4-chain antibodies (which are referred to herein as “V_{L} domains”). For a further description of V_{HH}s, reference is made to the review article by Muyldermans et al. 2001 (Reviews in Molecular Biotechnology 74: 277-302, 2001).

Typically, the generation of immunoglobulins involves the immunization of experimental animals, fusion of immunoglobulin producing cells to create hybridomas and screening for the desired specificities. Alternatively, immunoglobulins can be generated by screening of naïve or synthetic libraries, e.g., by phage display.

The generation of immunoglobulin sequences, such as Nanobody® V_{HH}s, has been described extensively in various publications, among which WO 94/04678, Hamers-Casterman et al. 1993 and Muyldermans et al. 2001 (Reviews in Molecular Biotechnology 74: 277-302, 2001) can be exemplified. In these methods, camelids are immunized with the target antigen in order to induce an immune response against said target antigen. The repertoire of V_{HH}S obtained from said immunization is further screened for V_{HH}S that bind the target antigen.

In these instances, the generation of antibodies requires purified antigen for immunization and/or screening. Antigens can be purified from natural sources, or in the course of recombinant production.

Immunization and/or screening for immunoglobulin sequences can be performed using peptide fragments of such antigens. The present technology may use immunoglobulin sequences of different origin, comprising mouse, rat, rabbit, donkey, human and camelid immunoglobulin sequences. The technology also includes fully human, humanized or chimeric sequences. For example, the invention comprises camelid immunoglobulin sequences and humanized camelid immunoglobulin sequences, or camelized domain antibodies, e.g. camelized dAb as described by Ward et al (see for example WO 94/04678 and Riechmann, Febs Lett., 339:285-290, 1994 and Prot. Eng., 9:531-537, 1996). Moreover, the invention also uses fused immunoglobulin sequences, e.g. forming a multivalent and/or multispecific construct (for multivalent and multispecific polypeptides containing one or more V_{HH} domains and their preparation, reference is also made to Conrath et al., J. Biol. Chem., Vol. 276, 10. 7346-7350, 2001, as well as to for example WO 96/34103 and WO 99/23221), and immunoglobulin sequences comprising tags or other functional moieties, e.g., toxins, labels, radiochemicals, etc., which are derivable from the immunoglobulin sequences of the present technology.

A “humanized V_{HH}” comprises an amino acid sequence that corresponds to the amino acid sequence of a naturally occurring V_{HH} domain, but that has been “humanized”, i.e., by replacing one or more amino acid residues in the amino acid sequence of said naturally occurring V_{HH} sequence (and in particular in the framework sequences) by one or more of the amino acid residues that occur at the corresponding position(s) in a V_{H} domain from a conventional 4-chain antibody from a human being (e.g., indicated above). This can be performed in a manner known per se, which will be clear to the skilled person, for example on the basis of the further description herein and the prior art (e.g., WO 2008/020079). Again, it should be noted that such humanized V_{HH}S can be obtained in any suitable manner known per se and thus are not strictly limited to polypeptides that have been obtained using a polypeptide that comprises a naturally occurring V_{HH} domain as a starting material.

A “camelized V_{H}” comprises an amino acid sequence that corresponds to the amino acid sequence of a naturally occurring V_{H} domain, but that has been “camelized”, i.e. by replacing one or more amino acid residues in the amino acid sequence of a naturally occurring V_{H} domain from a conventional 4-chain antibody by one or more of the amino acid residues that occur at the corresponding position(s) in a V_{HH} domain of a heavy chain antibody. This can be performed in a manner known per se, which will be clear to the skilled person, for example on the basis of the further description herein and the prior art (e.g., WO 2008/020079). Such “camelizing” substitutions are preferably inserted at amino acid positions that form and/or are present at the V_{H}-V_{L} interface, and/or at the so-called Camelidae hallmark residues, as defined herein (see for example WO 94/04678 and Davies and Riechmann (1994 and 1996), supra). Preferably, the V_{H} sequence that is used as a starting material or starting point for generating or designing the camelized V_{H} is preferably a V_{H} sequence from a mammal, more preferably the V_{H} sequence of a human being, such as a V_{H}3 sequence. However, it should be noted that such camelized V_{H} can be obtained in any suitable manner known per se and thus are not strictly limited to polypeptides that have been obtained using a polypeptide that comprises a naturally occurring V_{H} domain as a starting material.

The structure of an ISVD sequence can be considered to be comprised of four framework regions (“FRs”), which are referred to in the art and herein as “Framework region 1” (“FR1”); as “Framework region 2” (“FR2”); as “Framework region 3” (“FR3”); and as “Framework region 4” (“FR4”), respectively; which framework regions are interrupted by three complementary determining regions (“CDRs”), which are referred to in the art and herein as “Complementarity Determining Region 1” (“CDR1”); as “Complementarity Determining Region 2” (“CDR2”); and as “Complementarity Determining Region 3” (“CDR3”), respectively.

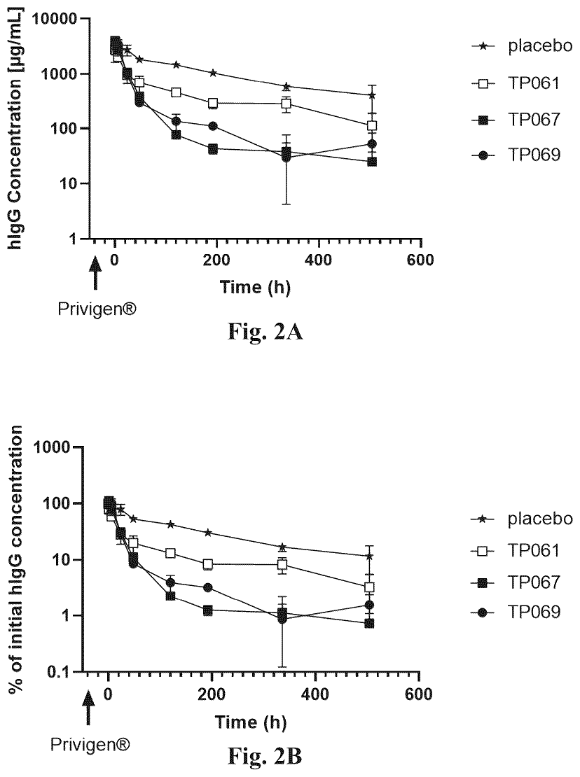

As further described in paragraph q) on pages 58 and 59 of WO 08/020079, the amino acid residues of an immunoglobulin single variable domain can be numbered according to the general numbering for V_{H} domains given by Kabat et al. (“Sequence of proteins of immunological interest”, US Public Health Services, NIH Bethesda, MD, Publication No. 91), as applied to V_{HH} domains from Camelids in the article of Riechmann and Muyldermans, 2000 (J. Immunol. Methods 240 (1-2): 185-195; see for example FIG. 2 of this publication). It should be noted that—as is well known in the art for V_{H} domains and for V_{HH} domains—the total number of amino acid residues in each of the CDRs may vary and may not correspond to the total number of amino acid residues indicated by the Kabat numbering (that is, one or more positions according to the Kabat numbering may not be occupied in the actual sequence, or the actual sequence may contain more amino acid residues than the number allowed for by the Kabat numbering). This means that, generally, the numbering according to Kabat may or may not correspond to the actual numbering of the amino acid residues in the actual sequence. The total number of amino acid residues in a V_{H} domain and a V_{HH} domain will usually be in the range of from 110 to 120, often between 112 and 115. It should however be noted that smaller and longer sequences may also be suitable for the purposes described herein.

In the present application, unless indicated otherwise, CDR sequences were determined according to the AbM numbering as described in Kontermann and Dubel (Eds. 2010, Antibody Engineering, vol 2, Springer Verlag Heidelberg Berlin, Martin, Chapter 3, pp. 33-51). According to this method, FR1 comprises the amino acid residues at positions 1-25, CDR1 comprises the amino acid residues at positions 26-35, FR2 comprises the amino acids at positions 36-49, CDR2 comprises the amino acid residues at positions 50-58, FR3 comprises the amino acid residues at positions 59-94, CDR3 comprises the amino acid residues at positions 95-102, and FR4 comprises the amino acid residues at positions 103-113.

Determination of CDR regions may also be done according to different methods. In the CDR determination according to Kabat, FR1 of an immunoglobulin single variable domain comprises the amino acid residues at positions 1-30, CDR1 of an immunoglobulin single variable domain comprises the amino acid residues at positions 31-35, FR2 of an immunoglobulin single variable domain comprises the amino acids at positions 36-49, CDR2 of an immunoglobulin single variable domain comprises the amino acid residues at positions 50-65, FR3 of an immunoglobulin single variable domain comprises the amino acid residues at positions 66-94, CDR3 of an immunoglobulin single variable domain comprises the amino acid residues at positions 95-102, and FR4 of an immunoglobulin single variable domain comprises the amino acid residues at positions 103-113.

In such an immunoglobulin sequence, the framework sequences may be any suitable framework sequences, and examples of suitable framework sequences will be clear to the skilled person, for example on the basis the standard handbooks and the further disclosure and prior art mentioned herein.

The framework sequences are preferably (a suitable combination of) immunoglobulin framework sequences or framework sequences that have been derived from immunoglobulin framework sequences (for example, by humanization or camelization). For example, the framework sequences may be framework sequences derived from a light chain variable domain (e.g., a V_{L}-sequence) and/or from a heavy chain variable domain (e.g., a V_{H}-sequence or V_{HH} sequence). In one particularly preferred aspect, the framework sequences are either framework sequences that have been derived from a V_{HH}-sequence (in which said framework sequences may optionally have been partially or fully humanized) or are conventional V_{H} sequences that have been camelized (as defined herein).

In particular, the framework sequences present in the ISVD sequence used in the invention may contain one or more of hallmark residues (as defined herein), such that the ISVD sequence is a Nanobody® molecule, such as a V_{HH}, including a humanized V_{HH} or camelized V_{H}. Some preferred, but non-limiting examples of (suitable combinations of) such framework sequences will become clear from the further disclosure herein.

It should be noted that the invention is not limited as to the origin of the ISVD sequence (or of the nucleotide sequence used to express it), nor as to the way that the ISVD sequence or nucleotide sequence is (or has been) generated or obtained. Thus, the ISVD sequences may be naturally occurring sequences (from any suitable species) or synthetic or semi-synthetic sequences. In a specific but non-limiting aspect, the ISVD sequence is a naturally occurring sequence (from any suitable species) or a synthetic or semi-synthetic sequence, including but not limited to “humanized” (as defined herein) immunoglobulin sequences (such as partially or fully humanized mouse or rabbit immunoglobulin sequences, and in particular partially or fully humanized V_{HH} sequences), “camelized” (as defined herein) immunoglobulin sequences, as well as immunoglobulin sequences that have been obtained by techniques such as affinity maturation (for example, starting from synthetic, random or naturally occurring immunoglobulin sequences), CDR grafting, veneering, combining fragments derived from different immunoglobulin sequences, PCR assembly using overlapping primers, and similar techniques for engineering immunoglobulin sequences well known to the skilled person; or any suitable combination of any of the foregoing.

Similarly, nucleotide sequences may be naturally occurring nucleotide sequences or synthetic or semi-synthetic sequences, and may for example be sequences that are isolated by PCR from a suitable naturally occurring template (e.g., DNA or RNA isolated from a cell), nucleotide sequences that have been isolated from a library (and in particular, an expression library), nucleotide sequences that have been prepared by introducing mutations into a naturally occurring nucleotide sequence (using any suitable technique known per se, such as mismatch PCR), nucleotide sequence that have been prepared by PCR using overlapping primers, or nucleotide sequences that have been prepared using techniques for DNA synthesis known per se.

For a general description of ISVDs, reference is made to the further description below, as well as to the prior art cited herein. In this respect, it should however be noted that this description and the prior art mainly described ISVDs of the so-called “V_{H}3 class” (i.e. ISVDs with a high degree of sequence homology to human germline sequences of the V_{H}3 class such as DP-47, DP-51 or DP-29). It should however be noted that the invention in its broadest sense can generally use any type of ISVD, and for example also uses the ISVDs belonging to the so-called “V_{H}4 class” (i.e. ISVDs with a high degree of sequence homology to human germline sequences of the V_{H}4 class such as DP-78), as for example described in WO 2007/118670.

Generally, ISVDs (in particular V_{HH} sequences, including (partially) humanized V_{HH} sequences and camelized V_{H} sequences) can be characterized by the presence of one or more “Hallmark residues” (as described herein) in one or more of the framework sequences (again as further described herein). Thus, generally, a ISVD can be defined as an immunoglobulin sequence with the (general) structure

FR1-CDR1-FR2-CDR2-FR3-CDR3-FR4

in which FR1 to FR4 refer to framework regions 1 to 4, respectively, and in which CDR1 to CDR3 refer to the complementarity determining regions 1 to 3, respectively, and in which one or more of the Hallmark residues are as further defined herein.

In particular, an ISVD can be an immunoglobulin sequence with the (general) structure

FR1-CDR1-FR2-CDR2-FR3-CDR3-FR4

in which FR1 to FR4 refer to framework regions 1 to 4, respectively, and in which CDR1 to CDR3 refer to the complementarity determining regions 1 to 3, respectively, and in which the framework sequences are as further defined herein.

More in particular, an ISVD can be an immunoglobulin sequence with the (general) structure

FR1-CDR1-FR2-CDR2-FR3-CDR3-FR4

in which FR1 to FR4 refer to framework regions 1 to 4, respectively, and in which CDR1 to CDR3 refer to the complementarity determining regions 1 to 3, respectively, and in which:
one or more of the amino acid residues at positions 11, 37, 44, 45, 47, 83, 84, 103, 104 and 108 according to the Kabat numbering are chosen from the Hallmark residues mentioned in Table 2 below.

TABLE 2
Hallmark Residues in Nanobody ® ISVDs

| Position | Human V_{H}3 | Hallmark Residues |
|---|---|---|
| 11 | L, V; | L, S, V, M, W, F, T, Q, E, A, R, G, K, Y, N, P, |
| | predominantly | I; preferably L |
| | L | |
| 37 | V, I, F; | F⁽¹⁾, Y, V, L, A, H, S, I, W, C, N, G, D, T, P, |
| | usually V | preferably F⁽¹⁾ or Y |
| 44⁽⁸⁾ | G | E⁽³⁾, Q⁽³⁾, G⁽²⁾, D, A, K, R, L, P, S, V, H, T, N, |
| | | W, M, I; preferably G⁽²⁾, E⁽³⁾ or Q⁽³⁾; most |
| | | preferably G⁽²⁾ or Q⁽³⁾ |
| 45⁽⁸⁾ | L | L⁽²⁾, R⁽³⁾, P, H, F, G, Q, S, E, T, Y, C, I, D, V; |
| | | preferably L⁽²⁾ or R⁽³⁾ |
| 47⁽⁸⁾ | W, Y | F⁽¹⁾, L⁽¹⁾ or W⁽²⁾ G, I, S, A, V, M, R, Y, E, P, T, |
| | | C, H, K, Q, N, D; preferably W⁽²⁾, L⁽¹⁾ or F⁽¹⁾ |
| 83 | R or K; | R, K⁽⁵⁾, T, E⁽⁵⁾, Q, N, S, I, V, G, M, L, A, D, Y, |
| | usually R | H; preferably K or R; most preferably K |
| 84 | A, T, D; | p⁽⁵⁾, S, H, L, A, V, I, T, F, D, R, Y, N, Q, G, E; |
| | predominantly | preferably P |
| | A | |
| 103 | W | W⁽⁴⁾, R⁽⁶⁾, G, S, K, A, M, Y, L, F, T, N, V, Q, |
| | | P⁽⁶⁾, E, C; preferably W |
| 104 | G | G, A, S, T, D, P, N, E, C, L; preferably G |
| 108 | L, M or T; | Q, L⁽⁷⁾, R, P, E, K, S, T, M, A, H; preferably Q |
| | predominantly | or L⁽⁷⁾ |
| | L | |

Notes:
In particular, but not exclusively, in combination with KERE or KQRE at positions 43-46.
Usually as GLEW at positions 44-47.
Usually as KERE or KQRE at positions 43-46, e.g., as KEREL, KEREF, KOREL, KQREF, KEREG, KQREW or KQREG at positions 43-47. Alternatively, also sequences such as TERE (for example TEREL), TORE (for example TQREL), KECE (for example KECEL or KECER), KQCE (for example KQCEL), RERE (for example REREG), RQRE (for example RQREL, RQREF or RQREW), QERE (for example QEREG), QQRE, (for example QQREW, QQREL or QQREF), KGRE (for example KGREG), KDRE (for example KDREV) are possible. Some other possible, but less preferred sequences include for example DECKL and NVCEL.
With both GLEW at positions 44-47 and KERE or KQRE at positions 43-46.
Often as KP or EP at positions 83-84 of naturally occurring V_{HH} domains.
In particular, but not exclusively, in combination with GLEW at positions 44-47.
With the proviso that when positions 44-47 are GLEW, position 108 is always Q in (non-humanized) VHH sequences that also contain a W at 103.
The GLEW group also contains GLEW-like sequences at positions 44-47, such as for example GVEW, EPEW, GLER, DQEW, DLEW, GIEW, ELEW, GPEW, EWLP, GPER, GLER and ELEW

Thus, a Nanobody® ISVD can be defined as an amino acid sequence with the (general) structure

FR1-CDR1-FR2-CDR2-FR3-CDR3-FR4

in which FR1 to FR4 refer to framework regions 1 to 4, respectively, and in which CDR1 to CDR3 refer to the complementarity determining regions 1 to 3, respectively, and in which one or more of the amino acid residues at positions 11, 37, 44, 45, 47, 83, 84, 103, 104 and 108 according to the Kabat numbering are chosen from the Hallmark residues mentioned in Table 2.

In one embodiment, the ISVD comprised in the FcRn antagonists of the present invention derives from an ISVD, such as from a heavy-chain ISVD, preferably from a Nanobody® ISVD, which has been further engineered/modified to include mutations which prevent/remove binding of pre-existing antibodies/factors. Examples of such mutations are described, e.g., in WO 2012/175741 and WO 2015/173325. For instance, to prevent/remove binding of pre-existing antibodies/factors, the amino acid at position 11 (according to Kabat) may be Val or Leu, preferably Val; and/or the amino acid at position 89 (according to Kabat) may be preferably Val, Thr or Leu, preferably Leu; and/or the amino acid at position 110 (according to Kabat) may be preferably Thr, Lys or Gln, preferably Thr; and/or the amino acid at position 112 (according to Kabat) may be Ser, Lys or Gln, preferably Ser; and/or the ISVD-based building block may contain a C-terminal extension of 1-5 amino acids chosen from any naturally occurring amino acid.

Hence, in a preferred embodiment, the FcRn antagonists of the present invention comprise:
a) at least one immunoglobulin single variable domain (ISVD) specifically binding to an epitope on Fc receptor (FcRn), wherein the ISVD does not compete with wild-type IgG1 Fc region for binding to FcRn; and
b) at least one polypeptide which competes with wild-type IgG1 Fc region for binding to FcRn.

In a preferred embodiment, the at least one ISVD specifically binding to the epitope on FcRn comprised as the first polypeptide in the FcRn antagonists of the present invention does not compete with wild-type human serum albumin (HSA) for binding to FcRn and/or is not an FcRn antagonist on its own. Hence, in this specific embodiment, the FcRn antagonist of the present invention is an antagonist because the at least one second polypeptide (b) is an FcRn antagonist both on its own and in connection with the at least one first polypeptide (i.e., the second polypeptide (b) competes with wild-type IgG1 Fc region for binding to FcRn).

In some embodiments, the ISVD is a (single) domain antibody, a Nanobody® V_{HH}, a V_{HH}, a humanized V_{HH}, or a camelized V_{H}, preferably a Nanobody®.

In another preferred embodiment, the epitope on FcRn to which the ISVD comprised as the first polypeptide (a) in the FcRn antagonists of the present invention binds comprises at least one of the following amino acid residues 1A, 2E, 3S, 4H, 5L, 32P, 97E, 98L, 99G, 100P, 101D, 102N, 103T, 164R, 167L, 168E, 171R, 174L, 175E, 177K, 204Y, 205P, 206P, 207E, 208L, 209Q 255Q, 256H, 257A, 259L, 260A, 261Q, and/or 262P, amino acid residues being numbered according to SEQ ID NO: 1.

In particular embodiments, the epitope as disclosed herein is a linear epitope, comprising a stretch of continuous amino acid residues of the FcRn primary sequence. In particular embodiments, the epitope as disclosed herein is a conformational epitope, comprising at least two discontinuous amino acid residues and/or stretches of amino acid residues of the FcRn primary sequence that are close to each other in the FcRn tertiary structure and that form a specific binding surface with a three-dimensional structure for the polypeptides of the present invention.

Typically, binding units used in the present technology (such as the ISVD comprised in the FcRn antagonist of the present invention as the first polypeptide) will bind to their targets with a dissociation constant (K_{D}) of 10⁻⁵ to 10⁻¹² moles/liter or less, and preferably 10⁻⁷ to 10⁻¹² moles/liter or less and more preferably 10⁻⁸ to 10⁻¹² moles/liter (i.e., with an association constant (K_{A}) of 10⁵ to 10¹² liter/moles or more, and preferably 10⁷ to 10¹² liter/moles or more and more preferably 10⁸ to 10¹² liter/moles). Any K_{D} value greater than 10⁻⁴ mol/liter (or any KA value lower than 10⁴ liters/mol) is generally considered to indicate non-specific binding. The K_{D} for biological interactions, such as the binding of immunoglobulin sequences to an antigen, which are considered specific are typically in the range of 10⁻⁵ moles/liter (10000 nM or 10 μM) to 10⁻¹² moles/liter (0.001 nM or 1 pM) or less. Accordingly, specific/selective binding may mean that—using the same measurement method, e.g., SPR—a binding unit (or polypeptide comprising the same) binds to FcRn with a K_{D} value of 10⁻⁵ to 10⁻¹² moles/liter or less and binds to related targets with a K_{D} value greater than 10⁻⁴ moles/liter. Thus, the ISVD comprised in the FcRn antagonist of the present invention as the first polypeptide in this specific embodiment preferably exhibits at least half the binding affinity, more preferably at least the same binding affinity, to human FcRn as compared to an ISVD consisting of the amino acid of SEQ ID NOs: 14 or 15, or of SEQ ID NO: 185, or of SEQ ID NOs: 186 or 187 wherein the binding affinity is measured using the same method, such as SPR. Specific binding to a certain target from a certain species does not exclude that the binding unit can also specifically bind to the analogous target from a different species. For example, specific binding to human FcRn does not exclude that the binding unit (or a polypeptide comprising the same) can also specifically bind to FcRn from cynomolgus monkeys.

Specific binding of a binding unit to its designated target can be determined in any suitable manner known per se, including, for example, Scatchard analysis and/or competitive binding assays, such as radioimmunoassays (RIA), enzyme immunoassays (EIA) and sandwich competition assays, and the different variants thereof known per se in the art; as well as the other techniques mentioned herein. The dissociation constant may be the actual or apparent dissociation constant, as will be clear to the skilled person. Methods for determining the dissociation constant will be clear to the skilled person, and for example include the techniques mentioned below. In this respect, it will also be clear that it may not be possible to measure dissociation constants of more than 10⁻⁴ moles/liter or 10⁻³ moles/liter (e.g., of 10⁻² moles/liter). Optionally, as will also be clear to the skilled person, the (actual or apparent) dissociation constant may be calculated on the basis of the (actual or apparent) association constant (K_{A}), by means of the relationship [K_{D}=1/K_{A}]. The affinity of a molecular interaction between two molecules can be measured via different techniques known per se, such as the well-known surface plasmon resonance (SPR) biosensor technique (see for example Ober et al., 2001, Intern. Immunology 13: 30 1551-1559). The term “surface plasmon resonance”, as used herein, refers to an optical phenomenon that allows for the analysis of real-time biospecific interactions by detection of alterations in protein concentrations within a biosensor matrix, where one molecule is immobilized on the biosensor chip and the other molecule is passed over the immobilized molecule under flow conditions yielding kₒₙ, k_{off} measurements and hence K_{D} (or K_{A}) values. This can for example be performed using the well-known BIAcore® system (BIAcore International AB, a GE Healthcare company, Uppsala, Sweden and Piscataway, NJ). For further descriptions, see Jonsson et al. (1993, Ann. Biol. Clin. 51: 19-26), Jonsson et al. (1991 Biotechniques 11: 620-627), Johnsson et al. (1995, J. Mol. Recognit. 8: 125-131), and Johnnson et al. (1991, Anal. Biochem. 198: 268-277). Another well-known biosensor technique to determine affinities of biomolecular interactions is bio-layer interferometry (BLI) (see for example Abdiche et al., 2008, Anal. Biochem. 377: 209-217). The term “bio-layer Interferometry” or “BLI”, as used herein, refers to a label-free optical technique that analyzes the interference pattern of light reflected from two surfaces: an internal reference layer (reference beam) and a layer of immobilized protein on the biosensor tip (signal beam). A change in the number of molecules bound to the tip of the biosensor causes a shift in the interference pattern, reported as a wavelength shift (nm), the magnitude of which is a direct measure of the number of molecules bound to the biosensor tip surface.

Since the interactions can be measured in real-time, association and dissociation rates and affinities can be determined. BLI can for example be performed using the well-known Octet® Systems (ForteBio, a division of Pall Life Sciences, Menlo Park, USA). Alternatively, affinities can be measured in Kinetic Exclusion Assay (KinExA) (see for example Drake et al. 2004, Anal. Biochem., 328: 35-43), using the KinExA® platform (Sapidyne Instruments Inc, Boise, USA). The term “KinExA”, as used herein, refers to a solution-based method to measure true equilibrium binding affinity and kinetics of unmodified molecules. Equilibrated solutions of an antibody/antigen complex are passed over a column with beads precoated with antigen (or antibody), allowing the free antibody (or antigen) to bind to the coated molecule. Detection of the antibody (or antigen) thus captured is accomplished with a fluorescently labelled protein binding the antibody (or antigen). The GYROLAB® immunoassay system provides a platform for automated bioanalysis and rapid sample turnaround (Fraley et al., 2013, Bioanalysis 5: 1765-74).

In another embodiment, the FcRn epitope to which the at least one ISVD comprised in the FcRn antagonists of the present invention as the first polypeptide (a) binds, comprises at least one of the following combinations of amino acid residues:
i) 4H and 5L, and/or
ii) 98L, 99G, 100P, 101D and 102N, and/or
iii) 167L, 171R, 174L, 175E and 177K, and/or
iv) 255Q, 256H, 257A, 259L, 260A and 262P,
   wherein the amino acid residues are numbered according to SEQ ID NO: 1.

In another embodiment, the FcRn epitope to which the at least one ISVD comprised in the FcRn antagonists of the present invention as the first polypeptide (a) binds, comprises at least one of the following combinations of amino acid residues:
i) 2E, 3S, 4H and 5L, and/or
ii) 97E, 98L, 99G, 100P, 101D and 102N, and/or
iii) 98L, 99G, 100P, 101D, 102N and 103T, and/or
iv) 167L, 168E, 171R, 174L, 175E and 177K, and/or
v) 205P, 206P and 207E, and/or
vi) 255Q, 256H, 257A, 259L, 260A, 261Q, and 262P,
   wherein the amino acid residues are numbered according to SEQ ID NO: 1.

In another embodiment, the FcRn epitope to which the at least one ISVD comprised in the FcRn antagonists of the present invention as the first polypeptide (a) binds, comprises at least one of the following combinations of amino acid residues:
i) 1A, 2E, 3S, 4H and 5L, and/or
ii) 164R, 167L, 168E, 171R, 174L, 175E and 177K, and/or
iii) 204Y, 205P, 206P and 230E, and/or
iv) 205P, 206P, 207E and 208L,
   wherein the amino acid residues are numbered according to SEQ ID NO: 1.

In another embodiment, the FcRn epitope to which the at least one ISVD comprised in the FcRn antagonists of the present invention as the first polypeptide (a) binds, comprises at least the following amino acid residues 4H, 5L, 98L, 99G, 100P, 101D, 102N, 167L, 171R, 174L, 175E, 177K, 207E, 255Q, 256H, 257A, 259L, 260A, and 262P, wherein the amino acid residues are numbered according to SEQ ID NO: 1.

In another embodiment, the FcRn epitope to which the at least one ISVD comprised in the FcRn antagonists of the present invention as the first polypeptide (a) binds, comprises at least the following amino acid residues 2E, 3S, 4H, 5L, 32P, 97E, 98L, 99G, 100P, 101D, 102N, 103T, 167L, 168E, 171R, 174L, 175E, 177K, 205P, 206P, 207E, 255Q, 256H, 257A, 259L, 260A, 261Q, and 262P, wherein the amino acid residues are numbered according to SEQ ID NO: 1.

In another embodiment, the FcRn epitope to which the at least one ISVD comprised in the FcRn antagonists of the present invention as the first polypeptide (a) binds, comprises at least the following amino acid residues 1A, 2E, 3S, 4H, 5L, 32P, 97E, 98L, 99G, 100P, 101D, 102N, 103T, 164R, 167L, 168E, 171R, 174L, 175E, 177K, 204Y, 205P, 206P, 207E, 208L, 209Q, 255Q, 256H, 257A, 259L, 260A, 261Q, and 262P, wherein the amino acid residues are numbered according to SEQ ID NO: 1.

In another embodiment, the at least one ISVD comprised in the FcRn antagonists of the present invention as the first polypeptide (a) specifically binds to the FcRn epitope in a pH-dependent manner, such that the binding affinity at an acidic endosomal pH, such as a pH between 5.0 and 6.8, is at least three times higher than the binding affinity at an extracellular physiological pH, such as a pH of about 7.4.

In another embodiment, the at least one ISVD comprised in the FcRn antagonists of the present invention as the first polypeptide (a) specifically binds to amino acid residues on FcRn that are not involved in binding of FcRn to serum albumin and/or that are not involved in binding of FcRn to IgG. Hence, in this specific embodiment, the at least one ISVD comprised in the FcRn antagonists of the present invention as the first polypeptide (a) specifically binds to an epitope in FcRn characterized in that it is different from the epitope on FcRn against which serum albumin and/or IgG is directed.

In another embodiment, the at least one ISVD comprised in the FcRn antagonists of the present invention as the first polypeptide (a) consists of 4 framework regions (FR1 to FR4 respectively) and 3 complementarity determining regions (CDR1 to CDR3 respectively) and is characterized in that:
CDR1 (according to Kabat) has the amino acid sequence of SEQ ID NO: 2 [=X₁ X₂ X₃ M Y] or
CDR1 (according to AbM) has the amino acid sequence of SEQ ID NO: 5 [=Xₐ X_{b} X_{c} F Xₑ X₁ X₂ X₃ M Y], wherein
Xₐ is A, D, E, F, G, H, K, L, M, N, Q, S, T, W, or Y,
X_{b} is F, I, L, M, V, or Y,
X_{c} is A, E, G, I, L, M, N, P, S, T, V, W, or Y,
Xₑ is D, E, G, K, M, N, P, Q, S or T,
X₁ is A, D, E, G, K, N, Q, S, T, or V,
X₂ is A, F, L, M, N, Q, S, T, V, or Y, and
X₃ is A, D, E, G, H, M, N, Q, S, T, or V,
and
CDR2 (according to Kabat) has the amino acid sequence of SEQ ID NO: 3 [=A I X₃ X₄ G G G X₈ X₉ X₁₀ Y A D S V K G] or CDR2 (according to AbM) has the amino acid sequence of SEQ ID NO: 6 [=A I X₃ X₄ G G G X₈ X₉ X₁₀], wherein
X₃ is A, D, E, G, H, P, Q, R, S, T, or V,
X₄ is A, D, E, G, K, M, N, P, Q, R, S, T, V, W, or Y,
X₈ is A, D, G, H, K, L, M, Q, S, T, or V,
X₉ is A, D, E, F, G, H, I, K, L, M, N, P, Q, R, S, T, V, W, or Y, and
X₁₀ is A, D, E, H, S, T, V, or Y,
and
CDR3 (according to Kabat) has the amino acid sequence of SEQ ID NO: 4 [=D X₂ X₃ X₄ T X₆ X₇ T X₉ Y X₁₁ X₁₂] or CDR3 (according to AbM) has the amino acid sequence of SEQ ID NO: 7 [=D X₂ X₃ X₄ T X₆ X₇ T X₉ Y X₁₁ X₁₂], wherein
X₂ is A, D, E, F, G, I, L, M, Q, S, T, V, W, or Y,
X₃ is L, or N,
X₄ is F, L, W, or Y,
X₆ is A, D, E, K, L, M, Q, S, or W,
X₇ is L, M, or V,
X₉ is A, D, E, F, G, H, K, L, M, Q, R, S, T, V, W or Y,
X₁₁ is A, E, L, M, Q, S, T, V, or W, and
X₁₂ is T or Y,
provided that the amino acids
X₁ and X₃ of SEQ ID NO: 2 and X₆ of SEQ ID NO: 4 are not simultaneously E, D, and D, respectively, or
Xₐ, X₁ and X₃ of SEQ ID NO: 5 and X₆ of SEQ ID NO: 7 are not simultaneously T, E, D, and D, respectively, or
X₁ and X₃ of SEQ ID NO: 2 and X₉ of SEQ ID NO: 4 are not simultaneously E, D, and K, respectively, or
Xₐ, X₁ and X₃ of SEQ ID NO: 5 and X₉ of SEQ ID NO: 7 are not simultaneously T, E, D, and K, respectively, or
X₃ of SEQ ID NO: 2 and X₉ of SEQ ID NO: 4 are not simultaneously D and K, respectively, or
Xₐ and X₃ of SEQ ID NO: 5 and X₉ of SEQ ID NO: 7 are not simultaneously T, D and K, respectively, or
X₁ and X₃ of SEQ ID NO: 2, X₄ of SEQ ID NO: 3, and X₆ and X₉ of SEQ ID NO: 4 are not simultaneously E, D, Q, D, and K, respectively, or
Xₐ, X₁ and X₃ of SEQ ID NO: 5, X₄ of SEQ ID NO: 6, and X₆ and X₉ of SEQ ID NO: 7 are not simultaneously T, E, D, Q, D, and K, respectively, or
X₁ of SEQ ID NO: 2, X₄ of SEQ ID NO: 3, and X₆ and X₉ of SEQ ID NO: 4 are not simultaneously E, Q, D, and K, respectively, or
Xₐ and X₁ of SEQ ID NO: 5, X₄ of SEQ ID NO: 6, and X₆ and X₉ of SEQ ID NO: 7 are not simultaneously T, E, Q, D, and K, respectively, or
X₃ of SEQ ID NO: 2 and X₆ of SEQ ID NO: 4 are not simultaneously D, and D, respectively, or
Xₐ and X₃ of SEQ ID NO: 5 and X₆ of SEQ ID NO: 7 are not simultaneously T, D, and D, respectively, or
X₁ and X₃ of SEQ ID NO: 2, X₄ of SEQ ID NO: 3, and X₆ of SEQ ID NO: 4 are not simultaneously E, D, Q, and D, respectively, or
Xₐ, X₁ and X₃ of SEQ ID NO: 5, X₄ of SEQ ID NO: 6, and X₆ of SEQ ID NO: 7 are not simultaneously T, E, D, Q, and D, respectively, or
X₁ of SEQ ID NO: 2, and X₆ and X₉ of SEQ ID NO: 4 are not simultaneously E, D, and K, respectively, or
X₃ of SEQ ID NO: 2, X₄ of SEQ ID NO: 3, and X₆ and X₉ of SEQ ID NO: 4 are not simultaneously D, Q, D, and K, respectively, or
X₁ and X₃ of SEQ ID NO: 2, X₄ of SEQ ID NO: 3, and X₉ of SEQ ID NO: 4 are not simultaneously E, D, Q, and K, respectively, or
Xₐ, X₁ and X₃ of SEQ ID NO: 5, X₄ of SEQ ID NO: 6, and X₉ of SEQ ID NO: 7 are not simultaneously T, E, D, Q, and K, respectively, or
X₃ of SEQ ID NO: 2, and X₆ and X₉ of SEQ ID NO: 4 are not simultaneously D, D, and K, respectively, or
X₃ of SEQ ID NO: 2, and X₉ of SEQ ID NO: 4 are not simultaneously D, and S, respectively, or Xₐ and X₃ of SEQ ID NO: 5, and X₉ of SEQ ID NO: 7 are not simultaneously T, D, and S, respectively, or
X₁ and X₃ of SEQ ID NO: 2, and X₆ and X₉ of SEQ ID NO: 4 are not simultaneously E, D, D, and S, respectively, or
X₆ and X₉ of SEQ ID NO: 4 are not simultaneously D, and K, respectively, or
Xₐ of SEQ ID NO: 5, and X₆ and X₉ of SEQ ID NO: 7 are not simultaneously T, D, and K, respectively, or
X₁ of SEQ ID NO: 2, X₄ of SEQ ID NO: 3, and X₆ and X₉ of SEQ ID NO: 4 are not simultaneously E, Q, D, and K, respectively, or
X₃ of SEQ ID NO: 2, X₄ of SEQ ID NO: 3, and X₆ and X₉ of SEQ ID NO: 4 are not simultaneously D, Q, D, and S, respectively, or
Xₐ and X₃ of SEQ ID NO: 5, X₄ of SEQ ID NO: 6, and X₆ and X₉ of SEQ ID NO: 7 are not simultaneously T, D, Q, D, and S, respectively, or
X₁ of SEQ ID NO: 2, X₄ of SEQ ID NO: 3, X₆ and X₉ of SEQ ID NO: 4 are not simultaneously E, Q D, and S, respectively, or
Xₐ and X₁ of SEQ ID NO: 5, X₄ of SEQ ID NO: 6, X₆ and X₉ of SEQ ID NO: 7 are not simultaneously T, E, Q, D, and S, respectively, or
X₁ of SEQ ID NO: 2, and X₆ and X₉ of SEQ ID NO: 4 are not simultaneously E, D, and K, respectively, or
Xₐ and X₁ of SEQ ID NO: 5, and X₆ and X₉ of SEQ ID NO: 7 are not simultaneously T, E, D, and K, respectively, or
X₃ of SEQ ID NO: 2, and X₆ and X₉ of SEQ ID NO: 4 are not simultaneously D, D, and K, respectively, or
Xₐ and X₃ of SEQ ID NO: 5, and X₆ and X₉ of SEQ ID NO: 7 are not simultaneously T, D, D, and K, respectively, or
X₁ and X₃ of SEQ ID NO: 2, and X₆ and X₉ of SEQ ID NO: 4 are not simultaneously E, D, D, and K, respectively, or
X₃ of SEQ ID NO: 2, and X₆ and X₉ of SEQ ID NO: 4 are not simultaneously D, D, and S, respectively, or,
X₁ and X₃ of SEQ ID NO: 2, X₄ of SEQ ID NO: 3, and X₆ and X₉ of SEQ ID NO: 4 are not simultaneously E, D, Q, D, and S, respectively, or
Xₐ, X₁ and X₃ of SEQ ID NO: 5, X₄ of SEQ ID NO: 6, and X₆ and X₉ of SEQ ID NO: 7 are not simultaneously T, E, D, Q, D, and S, respectively, or
X₁ and X₃ of SEQ ID NO: 2, X₄ of SEQ ID NO: 3, and X₆ and X₉ of SEQ ID NO: 4 are not simultaneously E, D, Q, D, and K, respectively, or
Xₐ, X₁ and X₃ of SEQ ID NO: 5, and X₆ and X₉ of SEQ ID NO: 7 are not simultaneously T, E, D, D, and S, respectively, or
X₃ of SEQ ID NO: 2, X₆ and X₉ of SEQ ID NO: 4 are not simultaneously D, D, and S, respectively, or
Xₐ and X₃ of SEQ ID NO: 5, X₆ and X₉ of SEQ ID NO: 7 are not simultaneously T, D, D, and S, respectively, or
X₁ and X₃ of SEQ ID NO: 2, and X₉ of SEQ ID NO: 4 are not simultaneously E, D, and S, respectively, or
X₄ of SEQ ID NO: 3, and X₉ of SEQ ID NO: 4 are not simultaneously Q, and S, respectively or
Xₐ of SEQ ID NO: 5, X₄ of SEQ ID NO: 6, and X₉ of SEQ ID NO: 7 are not simultaneously T, Q, and S, respectively.

In another embodiment, the at least one ISVD comprised in the FcRn antagonists of the present invention as the first polypeptide (a) consists of 4 framework regions (FR1 to FR4 respectively) and 3 complementarity determining regions (CDR1 to CDR3 respectively) and is characterized in that:
CDR1 (according to Kabat) has the amino acid sequence of SEQ ID NO: 2 [=X₁ X₂ X₃ M Y] or
CDR1 (according to AbM) has the amino acid sequence of SEQ ID NO: 5 [=Xₐ X_{b} X_{c} F Xₑ X₁ X₂X₃ M Y], wherein
Xₐ is G, or T,
X_{b} is F,
X_{c} is T,
Xₑ is S,
X₁ is S, D, or E,
X₂ is Y, and
X₃ is A, or D,
and
CDR2 (according to Kabat) has the amino acid sequence of SEQ ID NO: 3 [=A I X₃ X₄ G G G X₈ X₉ X₁₀ Y A D S V K G] or CDR2 (according to AbM) has the amino acid sequence of SEQ ID NO: 6 [=A I X₃ X₄ G G G X₈ X₉ X₁₀], wherein
X₃ is S,
X₄ is Q, or S,
X₈ is S,
X₉ is T, and
X₁₀ is D,
and
CDR3 (according to Kabat) has the amino acid sequence of SEQ ID NO: 4 [=D X₂ X₃ X₄ T X₆ X₇ T X₉ Y X₁₁ X₁₂] or CDR3 (according to AbM) has the amino acid sequence of SEQ ID NO: 7 [=D X₂ X₃ X₄ T X₆ X₇ T X₉ Y X₁₁ X₁₂], wherein
X₂ is T,
X₃ is L,
X₄ is Y,
X₆ is D, E, or S,
X₇ is L,
X₉ is K, R, S, or W,
X₁₁ is S, and
X₁₂ is Y,
provided that the amino acids
X₁ and X₃ of SEQ ID NO: 2 and X₆ of SEQ ID NO: 4 are not simultaneously E, D, and D, respectively, or
Xₐ, X₁ and X₃ of SEQ ID NO: 5 and X₆ of SEQ ID NO: 7 are not simultaneously T, E, D, and D, respectively, or
X₁ and X₃ of SEQ ID NO: 2 and X₉ of SEQ ID NO: 4 are not simultaneously E, D, and K, respectively, or
Xₐ, X₁ and X₃ of SEQ ID NO: 5 and X₉ of SEQ ID NO: 7 are not simultaneously T, E, D, and K, respectively, or
X₃ of SEQ ID NO: 2 and X₉ of SEQ ID NO: 4 are not simultaneously D and K, respectively, or
Xₐ and X₃ of SEQ ID NO: 5 and X₉ of SEQ ID NO: 7 are not simultaneously T, D and K, respectively, or
X₁ and X₃ of SEQ ID NO: 2, X₄ of SEQ ID NO: 3, and X₆ and X₉ of SEQ ID NO: 4 are not simultaneously E, D, Q, D, and K, respectively, or
Xₐ, X₁ and X₃ of SEQ ID NO: 5, X₄ of SEQ ID NO: 6, and X₆ and X₉ of SEQ ID NO: 7 are not simultaneously T, E, D, Q, D, and K, respectively, or
X₁ of SEQ ID NO: 2, X₄ of SEQ ID NO: 3, and X₆ and X₉ of SEQ ID NO: 4 are not simultaneously E, Q, D, and K, respectively, or
Xₐ and X₁ of SEQ ID NO: 5, X₄ of SEQ ID NO: 6, and X₆ and X₉ of SEQ ID NO: 7 are not simultaneously T, E, Q, D, and K, respectively, or
X₃ of SEQ ID NO: 2 and X₆ of SEQ ID NO: 4 are not simultaneously D, and D, respectively, or
Xₐ and X₃ of SEQ ID NO: 5 and X₆ of SEQ ID NO: 7 are not simultaneously T, D, and D, respectively, or
X₁ and X₃ of SEQ ID NO: 2, X₄ of SEQ ID NO: 3, and X₆ of SEQ ID NO: 4 are not simultaneously E, D, Q, and D, respectively, or
Xₐ, X₁ and X₃ of SEQ ID NO: 5, X₄ of SEQ ID NO: 6, and X₆ of SEQ ID NO: 7 are not simultaneously T, E, D, Q, and D, respectively, or
X₁ of SEQ ID NO: 2, and X₆ and X₉ of SEQ ID NO: 4 are not simultaneously E, D, and K, respectively, or
X₃ of SEQ ID NO: 2, X₄ of SEQ ID NO: 3, and X₆ and X₉ of SEQ ID NO: 4 are not simultaneously D, Q, D, and K, respectively, or
X₁ and X₃ of SEQ ID NO: 2, X₄ of SEQ ID NO: 3, and X₉ of SEQ ID NO: 4 are not simultaneously E, D, Q, and K, respectively, or
Xₐ, X₁ and X₃ of SEQ ID NO: 5, X₄ of SEQ ID NO: 6, and X₉ of SEQ ID NO: 7 are not simultaneously T, E, D, Q, and K, respectively, or
X₃ of SEQ ID NO: 2, and X₆ and X₉ of SEQ ID NO: 4 are not simultaneously D, D, and K, respectively, or
X₃ of SEQ ID NO: 2, and X₉ of SEQ ID NO: 4 are not simultaneously D, and S, respectively, or
Xₐ and X₃ of SEQ ID NO: 5, and X₉ of SEQ ID NO: 7 are not simultaneously T, D, and S, respectively, or
X₁ and X₃ of SEQ ID NO: 2, and X₆ and X₉ of SEQ ID NO: 4 are not simultaneously E, D, D, and S, respectively, or
X₆ and X₉ of SEQ ID NO: 4 are not simultaneously D, and K, respectively, or
Xₐ of SEQ ID NO: 5, and X₆ and X₉ of SEQ ID NO: 7 are not simultaneously T, D, and K, respectively, or
X₁ of SEQ ID NO: 2, X₄ of SEQ ID NO: 3, and X₆ and X₉ of SEQ ID NO: 4 are not simultaneously E, Q, D, and K, respectively, or
X₃ of SEQ ID NO: 2, X₄ of SEQ ID NO: 3, and X₆ and X₉ of SEQ ID NO: 4 are not simultaneously D, Q, D, and S, respectively, or
Xₐ and X₃ of SEQ ID NO: 5, X₄ of SEQ ID NO: 6, and X₆ and X₉ of SEQ ID NO: 7 are not simultaneously T, D, Q, D, and S, respectively, or
X₁ of SEQ ID NO: 2, X₄ of SEQ ID NO: 3, X₆ and X₉ of SEQ ID NO: 4 are not simultaneously E, Q D, and S, respectively, or
Xₐ and X₁ of SEQ ID NO: 5, X₄ of SEQ ID NO: 6, X₆ and X₉ of SEQ ID NO: 7 are not simultaneously T, E, Q, D, and S, respectively, or
X₁ of SEQ ID NO: 2, and X₆ and X₉ of SEQ ID NO: 4 are not simultaneously E, D, and K, respectively, or
Xₐ and X₁ of SEQ ID NO: 5, and X₆ and X₉ of SEQ ID NO: 7 are not simultaneously T, E, D, and K, respectively, or
X₃ of SEQ ID NO: 2, and X₆ and X₉ of SEQ ID NO: 4 are not simultaneously D, D, and K, respectively, or
Xₐ and X₃ of SEQ ID NO: 5, and X₆ and X₉ of SEQ ID NO: 7 are not simultaneously T, D, D, and K, respectively, or
X₁ and X₃ of SEQ ID NO: 2, and X₆ and X₉ of SEQ ID NO: 4 are not simultaneously E, D, D, and K, respectively, or
X₃ of SEQ ID NO: 2, and X₆ and X₉ of SEQ ID NO: 4 are not simultaneously D, D, and S, respectively, or,
X₁ and X₃ of SEQ ID NO: 2, X₄ of SEQ ID NO: 3, and X₆ and X₉ of SEQ ID NO: 4 are not simultaneously E, D, Q, D, and S, respectively, or
Xₐ, X₁ and X₃ of SEQ ID NO: 5, X₄ of SEQ ID NO: 6, and X₆ and X₉ of SEQ ID NO: 7 are not simultaneously T, E, D, Q, D, and S, respectively, or
X₁ and X₃ of SEQ ID NO: 2, X₄ of SEQ ID NO: 3, and X₆ and X₉ of SEQ ID NO: 4 are not simultaneously E, D, Q, D, and K, respectively, or
Xₐ, X₁ and X₃ of SEQ ID NO: 5, and X₆ and X₉ of SEQ ID NO: 7 are not simultaneously T, E, D, D, and S, respectively, or
X₃ of SEQ ID NO: 2, X₆ and X₉ of SEQ ID NO: 4 are not simultaneously D, D, and S, respectively, or
Xₐ and X₃ of SEQ ID NO: 5, X₆ and X₉ of SEQ ID NO: 7 are not simultaneously T, D, D, and S, respectively, or
X₁ and X₃ of SEQ ID NO: 2, and X₉ of SEQ ID NO: 4 are not simultaneously E, D, and S, respectively, or
X₄ of SEQ ID NO: 3, and X₉ of SEQ ID NO: 4 are not simultaneously Q, and S, respectively or
Xₐ of SEQ ID NO: 5, X₄ of SEQ ID NO: 6, and X₉ of SEQ ID NO: 7 are not simultaneously T, Q, and S, respectively.

In another embodiment, the at least one ISVD comprised in the FcRn antagonists of the present invention as the first polypeptide (a) consists of 4 framework regions (FR1 to FR4 respectively) and 3 complementarity determining regions (CDR1 to CDR3 respectively) and is characterized in that:
CDR1 (according to Kabat) has the amino acid sequence of SEQ ID NO: 2 [=X₁ X₂ X₃ M Y] or
CDR1 (according to AbM) has the amino acid sequence of SEQ ID NO: 5 [=Xₐ X_{b} X_{c} F Xₑ X₁ X₂ X₃ M Y], wherein
Xₐ is A, D, E, F, G, H, K, L, M, N, Q, S, T, W, or Y,
X_{b} is F, I, L, M, V, or Y,
X_{c} is A, E, G, I, L, M, N, P, S, T, V, W, or Y,
Xₑ is D, E, G, K, M, N, P, Q, S or T,
X₁ is A, D, E, G, K, N, Q, S, T, or V,
X₂ is A, F, L, M, N, Q, S, T, V, or Y, and
X₃ is A, D, E, G, H, M, N, Q, S, T, or V,
and
CDR2 (according to Kabat) has the amino acid sequence of SEQ ID NO: 9 [=A I S S G G G S T D Y A D S V K G] or CDR2 (according to AbM) has the amino acid sequence of SEQ ID NO: 12 [=A I S S G G G S T D],
and
CDR3 (according to Kabat) has the amino acid sequence of SEQ ID NO: 10 [=D T L Y T S L T S Y S Y] or CDR3 (according to AbM) has the amino acid sequence of SEQ ID NO: 13 [=D T L Y T S L T S Y S Y].

In particular embodiments, the present invention provides FcRn antagonists comprising at least one ISVD specifically binding to the epitope on FcRn as disclosed herein, wherein at least one ISVD consists of 4 framework regions (FR1 to FR4 respectively) and 3 complementarity determining regions (CDR1 to CDR3 respectively) and is characterized in that:
CDR1 (according to Kabat) has the amino acid sequence of SEQ ID NO: 8 [=S Y A M Y] or CDR1 (according to AbM) has the amino acid sequence of SEQ ID NO: 11 [=G F T F S S Y A M Y]
and
CDR2 (according to Kabat) has the amino acid sequence of SEQ ID NO: 3 [=A I X₃ X₄ G G G X₈ X₉ X₁₀ Y A D S V K G] or CDR2 (according to AbM) has the amino acid sequence of SEQ ID NO: 6 [=A I X₃ X₄ G G G X₈ X₉ X₁₀], wherein
X₃ is A, D, E, G, H, P, Q, R, S, T, or V,
X₄ is A, D, E, G, K, M, N, P, Q, R, S, T, V, W, or Y,
X₈ is A, D, G, H, K, L, M, Q, S, T, or V,
X₉ is A, D, E, F, G, H, I, K, L, M, N, P, Q, R, S, T, V, W, or Y, and
X₁₀ is A, D, E, H, S, T, V, or Y,
and
CDR3 (according to Kabat) has the amino acid sequence of SEQ ID NO: 10 [=D T L Y T S L T S Y S Y] or CDR3 (according to AbM) has the amino acid sequence of SEQ ID NO: 13 [=D T L Y T S L T S Y S Y].

In another embodiment, the at least one ISVD comprised in the FcRn antagonists of the present invention as the first polypeptide (a) consists of 4 framework regions (FR1 to FR4 respectively) and 3 complementarity determining regions (CDR1 to CDR3 respectively) and is characterized in that:
CDR1 (according to Kabat) has the amino acid sequence of SEQ ID NO: 8 [=S Y A M Y] or CDR1 (according to AbM) has the amino acid sequence of SEQ ID NO: 11 [=G F T F S S Y A M Y]
and
CDR2 (according to Kabat) has the amino acid sequence of SEQ ID NO: 9 [=A I S S G G G S T D Y A D S V K G] or CDR2 (according to AbM) has the amino acid sequence of SEQ ID NO: 12 [=A I S S G G G S T D],
and
CDR3 (according to Kabat) has the amino acid sequence of SEQ ID NO: 4 [=D X₂ X₃ X₄ T X₆ X₇ T X₉ Y X₁₁ X₁₂] or CDR3 (according to AbM) has the amino acid sequence of SEQ ID NO: 7[=D X₂ X₃ X₄ T X₆ X₇ T X₉ Y X₁₁ X₁₂], wherein
X₂ is A, D, E, F, G, I, L, M, Q, S, T, V, W, or Y,
X₃ is L, or N,
X₄ is F, L, W, or Y,
X₆ is A, D, E, K, L, M, Q, S, or W,
X₇ is L, M, or V,
X₉ is A, D, E, F, G, H, K, L, M, Q, R, S, T, V, W or Y,
X₁₁ is A, E, L, M, Q, S, T, V, or W, and
X₁₂ is T or Y,
provided that the amino acids X₆ and X₉ of SEQ ID NO: 4 are not simultaneously D, and K, respectively, or X₆ and X₉ of SEQ ID NO: 7 are not simultaneously D, and K, respectively.

In another embodiment, the at least one ISVD comprised in the FcRn antagonists of the present invention as the first polypeptide (a) consists of 4 framework regions (FR1 to FR4 respectively) and 3 complementarity determining regions (CDR1 to CDR3 respectively) and is characterized in that:
CDR1 (according to Kabat) has the amino acid sequence of SEQ ID NO: 2 [=X₁ X₂ X₃ M Y] or CDR1 (according to AbM) has the amino acid sequence of SEQ ID NO: 5 [=Xₐ X_{b} X_{c} F Xₑ X₁ X₂ X₃ M Y], wherein
Xₐ is G, or T,
X₁ is S, D, or E,
X₂ is Y, and
X₃ is A, or D,
and
CDR2 (according to Kabat) has the amino acid sequence of SEQ ID NO: 9 [=A I S S G G G S T D Y A D S V K G] or CDR2 (according to AbM) has the amino acid sequence of SEQ ID NO: 12 [=A I S S G G G S T D],
and
CDR3 (according to Kabat) has the amino acid sequence of SEQ ID NO: 10 [=D T L Y T S L T S Y S Y] or CDR3 (according to AbM) has the amino acid sequence of SEQ ID NO: 13 [=D T L Y T S L T S Y S Y].

In another embodiment, the at least one ISVD comprised in the FcRn antagonists of the present invention as the first polypeptide (a) consists of 4 framework regions (FR1 to FR4 respectively) and 3 complementarity determining regions (CDR1 to CDR3 respectively) and is characterized in that:
CDR1 (according to Kabat) has the amino acid sequence of SEQ ID NO: 8 [=S Y A M Y] or CDR1 (according to AbM) has the amino acid sequence of SEQ ID NO: 11 [=G F T F S S Y A M Y]
and
CDR2 (according to Kabat) has the amino acid sequence of SEQ ID NO: 3 [=A I X₃ X₄ G G G X₈ X₉ X₁₀ Y A D S V K G] or CDR2 (according to AbM) has the amino acid sequence of SEQ ID NO: 6 [=A I X₃ X₄ G G G X₈ X₉ X₁₀], wherein
X₃ is S,
X₄ is Q, or S,
X₈ is S,
X₉ is T, and
X₁₀ is D,
and
CDR3 (according to Kabat) has the amino acid sequence of SEQ ID NO: 10 [=D T L Y T S L T S Y S Y] or CDR3 (according to AbM) has the amino acid sequence of SEQ ID NO: 13 [=D T L Y T S L T S Y S Y].

In another embodiment, the at least one ISVD comprised in the FcRn antagonists of the present invention as the first polypeptide (a) consists of 4 framework regions (FR1 to FR4 respectively) and 3 complementarity determining regions (CDR1 to CDR3 respectively) and is characterized in that:
CDR1 (according to Kabat) has the amino acid sequence of SEQ ID NO: 8 [=S Y A M Y] or CDR1 (according to AbM) has the amino acid sequence of SEQ ID NO: 11 [=G F T F S S Y A M Y]
and
CDR2 (according to Kabat) has the amino acid sequence of SEQ ID NO: 9 [=A I S S G G G S T D Y A D S V K G] or CDR2 (according to AbM) has the amino acid sequence of SEQ ID NO: 12 [=A I S S G G G S T D],
and
CDR3 (according to Kabat) has the amino acid sequence of SEQ ID NO: 4 [=D X₂ X₃ X₄ T X₆ X₇ T X₉ Y X₁₁ X₁₂] or CDR3 (according to AbM) has the amino acid sequence of SEQ ID NO: 7[=D X₂ X₃ X₄ T X₆ X₇ T X₉ Y X₁₁ X₁₂], wherein
X₂ is T,
X₃ is L,
X₄ is Y,
X₆ is D, E, or S,
X₇ is L,
X₉ is K, R, S, or W,
X₁₁ is S, and
X₁₂ is Y,
provided that the amino acids X₆ and X₉ of SEQ ID NO: 4 are not simultaneously D, and K, respectively, or X₆ and X₉ of SEQ ID NO: 7 are not simultaneously D, and K, respectively.

In another embodiment, the at least one ISVD comprised in the FcRn antagonists of the present invention as the first polypeptide (a) consists of 4 framework regions (FR1 to FR4 respectively) and 3 complementarity determining regions (CDR1 to CDR3 respectively) and is characterized in that:
CDR1 (according to Kabat) has the amino acid sequence of SEQ ID NO: 8 [=S Y A M Y] or CDR1 (according to AbM) has the amino acid sequence of SEQ ID NO: 11 [=G F T F S S Y A M Y], wherein
S at position 1 of SEQ ID NO: 8 or at position 6 of SEQ ID NO: 11 has been changed into A, D, E, G, K, N, Q, T, or V, and/or
Y at position 2 of SEQ ID NO: 8 or at position 7 of SEQ ID NO: 11 has been changed into A, F, L, M, N, Q, S, T, or V, and/or
A at position 3 of SEQ ID NO: 8 or at position 8 of SEQ ID NO: 11 has been changed into D, E, G, H, M, N, Q, S, T, or V and
CDR2 (according to Kabat) has the amino acid sequence of SEQ ID NO: 9 [=A I S S G G G S T D Y A D S V K G] or CDR2 (according to AbM) has the amino acid sequence of SEQ ID NO: 12 [=A I S S G G G S T D], and
CDR3 (according to Kabat) has the amino acid sequence of SEQ ID NO: 10 [=D T L Y T S L T S Y S Y] or CDR3 (according to AbM) has the amino acid sequence of SEQ ID NO: 13 [=D T L Y T S L T S Y S Y].

In another embodiment, the at least one ISVD comprised in the FcRn antagonists of the present invention as the first polypeptide (a) consists of 4 framework regions (FR1 to FR4 respectively) and 3 complementarity determining regions (CDR1 to CDR3 respectively) and is characterized in that:
CDR1 (according to Kabat) has the amino acid sequence of SEQ ID NO: 8 [=S Y A M Y] or CDR1 (according to AbM) has the amino acid sequence of SEQ ID NO: 11 [=G F T F S S Y A M Y]
and
CDR2 (according to Kabat) has the amino acid sequence of SEQ ID NO: 9 [=A I S S G G G S T D Y A D S V K G] or CDR2 (according to AbM) has the amino acid sequence of SEQ ID NO: 12 [=A I S S G G G S T D], wherein
S at position 3 of SEQ ID NO: 9 or at position 3 of SEQ ID NO: 12 has been changed into A, D, E, G, H, P, Q, R, T, or V, and/or
S at position 4 of SEQ ID NO: 9 or at position 4 of SEQ ID NO: 12 has been changed into A, D, E, G, K, M, N, P, Q, R, T, V, W, or Y and/or
S at position 8 of SEQ ID NO: 9 or at position 8 of SEQ ID NO: 12 has been changed into A, D, G, H, K, L, M, Q, T, or V, and/or
T at position 9 of SEQ ID NO: 9 or at position 9 of SEQ ID NO: 12 has been changed into is A, D, E, F, G, H, I, K, L, M, N, P, Q, R, S, V, W, or Y and/or
D at position 10 of SEQ ID NO: 9 or at position 10 of SEQ ID NO: 12 has been changed into A, E, H, S, T, V, or Y, and
CDR3 (according to Kabat) has the amino acid sequence of SEQ ID NO: 10 [=D T L Y T S L T S Y S Y] or CDR3 (according to AbM) has the amino acid sequence of SEQ ID NO: 13 [=D T L Y T S L T S Y S Y].

In another embodiment, the at least one ISVD comprised in the FcRn antagonists of the present invention as the first polypeptide (a) consists of 4 framework regions (FR1 to FR4 respectively) and 3 complementarity determining regions (CDR1 to CDR3 respectively) and is characterized in that:
CDR1 (according to Kabat) has the amino acid sequence of SEQ ID NO: 8 [=S Y A M Y] or CDR1 (according to AbM) has the amino acid sequence of SEQ ID NO: 11 [=G F T F S S Y A M Y]
and
CDR2 (according to Kabat) has the amino acid sequence of SEQ ID NO: 9 [=A I S S G G G S T D Y A D S V K G] or CDR2 (according to AbM) has the amino acid sequence of SEQ ID NO: 12 [=A I S S G G G S T D]
and
CDR3 (according to Kabat) has the amino acid sequence of SEQ ID NO: 10 [=D T L Y T S L T S Y S Y] or CDR3 (according to AbM) has the amino acid sequence of SEQ ID NO: 13 [=D T L Y T S L T S Y S Y], wherein
T at position 2 of SEQ ID NO: 10 or at position 2 of SEQ ID NO: 13 has been changed into A, D, E, F, G, I, L, M, Q, S, V, W, or Y, and/or
L at position 3 of SEQ ID NO: 10 or at position 3 of SEQ ID NO: 13 has been changed into N, and/or
Y at position 4 of SEQ ID NO: 10 or at position 4 of SEQ ID NO: 13 has been changed into F, L, or W, and/or
S at position 6 of SEQ ID NO: 10 or at position 6 of SEQ ID NO: 13 has been changed into A, D, E, K, L, M, Q, or W, and/or
L at position 7 of SEQ ID NO: 10 or at position 7 of SEQ ID NO: 13 has been changed into M, or V, and/or
S at position 9 of SEQ ID NO: 10 or at position 9 of SEQ ID NO: 13 has been changed into A, D, E, F, G, H, K, L, M, Q, R, T, V, W or Y, and/or
S at position 11 of SEQ ID NO: 10 or at position 11 of SEQ ID NO: 13 has been changed into A, E, L, M, Q, T, V, or W, and/or
Y at position 12 of SEQ ID NO: 10 or at position 12 of SEQ ID NO: 13 has been changed into T, provided that the amino acids at positions 6 and 9 of SEQ ID NO: 10 or at positions 6 and 9 of SEQ ID NO: 13 are not simultaneously changed into D, and K, respectively.

In another embodiment, the at least one ISVD comprised in the FcRn antagonists of the present invention as the first polypeptide (a) consists of 4 framework regions (FR1 to FR4 respectively) and 3 complementarity determining regions (CDR1 to CDR3 respectively) and is characterized in that:
CDR1 (according to Kabat) has the amino acid sequence of SEQ ID NO: 8 [=S Y A M Y] or CDR1 (according to AbM) has the amino acid sequence of SEQ ID NO: 11 [=G F T F S S Y A M Y], wherein
S at position 1 of SEQ ID NO: 8 or at position 6 of SEQ ID NO: 11 has been changed into D or E, and/or
A at position 3 of SEQ ID NO: 8 or at position 8 of SEQ ID NO: 11 has been changed into D, and
CDR2 (according to Kabat) has the amino acid sequence of SEQ ID NO: 9 [=A I S S G G G S T D Y A D S V K G] or CDR2 (according to AbM) has the amino acid sequence of SEQ ID NO: 12 [=A I S S G G G S T D], wherein
S at position 4 of SEQ ID NO: 9 or at position 4 of SEQ ID NO: 12 has been changed into Q, and
CDR3 (according to Kabat) has the amino acid sequence of SEQ ID NO: 10 [=D T L Y T S L T S Y S Y] or CDR3 (according to AbM) has the amino acid sequence of SEQ ID NO: 13 [=D T L Y T S L T S Y S Y], wherein
S at position 6 of SEQ ID NO: 10 or at position 6 of SEQ ID NO: 13 has been changed into D, or E, and/or
S at position 9 of SEQ ID NO: 10 or at position 9 of SEQ ID NO: 13 has been changed into K, R, or W,
provided that the amino acids at positions 6 and 9 of SEQ ID NO: 10 or at positions 6 and 9 of SEQ ID NO: 13 are not simultaneously changed into D, and K, respectively.

In another embodiment, the at least one ISVD comprised in the FcRn antagonists of the present invention as the first polypeptide (a) consists of 4 framework regions (FR1 to FR4 respectively) and 3 complementarity determining regions (CDR1 to CDR3 respectively) and is characterized in that: CDR1 (according to Kabat) has the amino acid sequence of SEQ ID NO: 8 [=S Y A M Y] or CDR1 (according to AbM) has the amino acid sequence of SEQ ID NO: 11 [=G F T F S S Y A M Y], wherein
S at position 1 of SEQ ID NO: 8 or at position 6 of SEQ ID NO: 11 has been changed into D or E, and/or
A at position 3 of SEQ ID NO: 8 or at position 8 of SEQ ID NO: 11 has been changed into D, and CDR2 (according to Kabat) has the amino acid sequence of SEQ ID NO: 9 [=A I S S G G G S T D Y A D S V K G] or CDR2 (according to AbM) has the amino acid sequence of SEQ ID NO: 12 [=A I S S G G G S T D], and
CDR3 (according to Kabat) has the amino acid sequence of SEQ ID NO: 10 [=D T L Y T S L T S Y S Y] or CDR3 (according to AbM) has the amino acid sequence of SEQ ID NO: 13 [=D T L Y T S L T S Y S Y].

In another embodiment, the at least one ISVD comprised in the FcRn antagonists of the present invention as the first polypeptide (a) consists of 4 framework regions (FR1 to FR4 respectively) and 3 complementarity determining regions (CDR1 to CDR3 respectively) and is characterized in that: CDR1 (according to Kabat) has the amino acid sequence of SEQ ID NO: 8 [=S Y A M Y] or CDR1 (according to AbM) has the amino acid sequence of SEQ ID NO: 11 [=G F T F S S Y A M Y], and
CDR2 (according to Kabat) has the amino acid sequence of SEQ ID NO: 9 [=A I S S G G G S T D Y A D S V K G] or CDR2 (according to AbM) has the amino acid sequence of SEQ ID NO: 12 [=A I S S G G G S T D], wherein
S at position 4 of SEQ ID NO: 9 or at position 4 of SEQ ID NO: 12 has been changed into Q, and CDR3 (according to Kabat) has the amino acid sequence of SEQ ID NO: 10 [=D T L Y T S L T S Y S Y] or CDR3 (according to AbM) has the amino acid sequence of SEQ ID NO: 13 [=D T L Y T S L T S Y S Y].

In another embodiment, the at least one ISVD comprised in the FcRn antagonists of the present invention as the first polypeptide (a) consists of 4 framework regions (FR1 to FR4 respectively) and 3 complementarity determining regions (CDR1 to CDR3 respectively) and is characterized in that: CDR1 (according to Kabat) has the amino acid sequence of SEQ ID NO: 8 [=S Y A M Y] or CDR1 (according to AbM) has the amino acid sequence of SEQ ID NO: 11 [=G F T F S S Y A M Y],
and
CDR2 (according to Kabat) has the amino acid sequence of SEQ ID NO: 9 [=A I S S G G G S T D Y A D S V K G] or CDR2 (according to AbM) has the amino acid sequence of SEQ ID NO: 12 [=A I S S G G G S T D], and
CDR3 (according to Kabat) has the amino acid sequence of SEQ ID NO: 10 [=D T L Y T S L T S Y S Y] or CDR3 (according to AbM) has the amino acid sequence of SEQ ID NO: 13 [=D T L Y T S L T S Y S Y], wherein
S at position 6 of SEQ ID NO: 10 or at position 6 of SEQ ID NO: 13 has been changed into D, or E, and/or
S at position 9 of SEQ ID NO: 10 or at position 9 of SEQ ID NO: 13 has been changed into K, R, or W,
provided that the amino acids at positions 6 and 9 of SEQ ID NO: 10 or at positions 6 and 9 of SEQ ID NO: 13 are not simultaneously changed into D, and K, respectively.

In another embodiment, the at least one ISVD comprised in the FcRn antagonists of the present invention as the first polypeptide (a) consists of 4 framework regions (FR1 to FR4 respectively) and 3 complementarity determining regions (CDR1 to CDR3 respectively), and is characterized in that:
a) CDR1 (according to Kabat) has the amino acid sequence of SEQ ID NO: 8 [=S Y A M Y] and/or has an amino acid sequence having 4, 3, 2 or only 1 “amino acid difference(s)” (as defined herein) with the sequence of SEQ ID NO: 8; and
b) CDR2 (according to Kabat) has the amino acid sequence of SEQ ID NO: 9 [=A I S S G G G S T D Y A D S V K G] and/or has an amino acid sequence having 4, 3, 2 or only 1 “amino acid difference(s)” (as defined herein) with the sequence of SEQ ID NO: 9; and
c) CDR3 (according to Kabat) has the amino acid sequence of SEQ ID NO: 10 [=D T L Y T S L T S Y S Y] and/or has an amino acid sequence having 4, 3, 2 or only 1 “amino acid difference(s)” (as defined herein) with the sequence of SEQ ID NO: 10.

In another embodiment, the at least one ISVD comprised in the FcRn antagonists of the present invention as the first polypeptide (a) consists of 4 framework regions (FR1 to FR4 respectively) and 3 complementarity determining regions (CDR1 to CDR3 respectively), and is characterized in that:
a) CDR1 (according to AbM) has the amino acid sequence of SEQ ID NO: 11 [=G F T F S S Y A M Y] and/or has an amino acid sequence having 4, 3, 2 or only 1 “amino acid difference(s)” (as defined herein) with the sequence of SEQ ID NO: 11; and
b) CDR2 (according to AbM) has the amino acid sequence of SEQ ID NO: 12 [=A I S S G G G S T D] and/or has an amino acid sequence having 4, 3, 2 or only 1 “amino acid difference(s)” (as defined herein) with the sequence of SEQ ID NO: 12; and
c) CDR3 (according to AbM) has the amino acid sequence of SEQ ID NO: 13 [=D T L Y T S L T S Y S Y], and/or has an amino acid sequence having 4, 3, 2 or only 1 “amino acid difference(s)” (as defined herein) with the sequence of SEQ ID NO: 13.

In another embodiment, the at least one ISVD comprised in the FcRn antagonists of the present invention as the first polypeptide (a) has the sequence of SEQ ID NO: 14, SEQ ID NO: 15 or SEQ ID NO: 185.

In another embodiment, the at least one ISVD comprised in the FcRn antagonists of the present invention as the first polypeptide (a) has at least 80%, such as 90%, or even 95% sequence identity with the amino acid sequence of SEQ ID NO: 14, SEQ ID NO: 15 or SEQ ID NO: 185.

In another embodiment, the at least one ISVD comprised in the FcRn antagonists of the present invention as the first polypeptide (a) has the sequence SEQ ID NO.: 186 or SEQ ID NO: 187.

In another embodiment, the at least one ISVD comprised in the FcRn antagonists of the present invention as the first polypeptide (a) has at least 80%, such as 90%, or even 95% sequence identity with the amino acid sequence of SEQ ID NO: 185, SEQ ID NO.: 186 or SEQ ID NO: 187.

In particular embodiments, the polypeptides as described herein comprising the ISVD with the one or more CDRs with 1, 2, 3, or 4 amino acid(s) differences bind to FcRn with about the same affinity compared to the binding by the amino acid sequence or polypeptide comprising the CDRs without the 4, 3, 2, or 1 amino acid(s) difference, said affinity as measured by surface plasmon resonance.

Compared to the sequence of SEQ ID NO: 14, 15 and/or 185, the ISVD comprised in the FcRn antagonists of the invention as the first polypeptide (a) preferably also contain (at least): one or more humanizing substitutions; and/or one or more mutations (i.e., amino acid substitutions, deletions or additions, and in particular substitutions) that reduce the binding by pre-existing antibodies; and may optionally contain one or more further mutations (e.g., to improve chemical stability of the FcRn antagonist) as described herein.

Compared to the sequence of SEQ ID NO: 186 and/or 187, the ISVD comprised in the FcRn antagonists of the invention as the first polypeptide (a) preferably also contain (at least): one or more humanizing substitutions; and/or one or more mutations (i.e., amino acid substitutions, deletions or additions, and in particular substitutions) that reduce the binding by pre-existing antibodies; and may optionally contain one or more further mutations (e.g., to improve chemical stability of the FcRn antagonist) as described herein.

For suitable humanizing substitutions (and suitable combinations thereof), reference is for example made to WO 09/138519 (or in the prior art cited in WO 09/138519) and WO 08/020079 (or in the prior art cited in WO 08/020079), as well as Tables A-3 to A-8 from WO 08/020079 (which are lists showing possible humanizing substitutions). Some preferred but non-limiting examples of such humanizing substitutions are Q108L and A14P or a suitable combination thereof. Such humanizing substitutions may also be suitably combined with one or more other mutations as described herein (such as with one or more mutations that reduce binding by pre-existing antibodies).

For suitable mutations that can reduce the binding by pre-existing antibodies (and suitable combinations of such mutations), reference is for example made to WO 2012/175741 and WO 2015/173325 and also to for example WO 2013/024059 and WO 2016/118733.

Amino acid sequence modifications of the polypeptides or ISVDs described herein are contemplated. For example, it may be desirable to improve the binding affinity and/or other biological properties of the polypeptides or ISVDs. Amino acid sequence variants of the polypeptides or ISVDs as described herein are prepared by introducing appropriate nucleotide changes into the nucleic acid encoding the polypeptides or ISVDs, or by peptide synthesis.

Such modifications include, for example, deletions from, and/or insertions into, and/or substitutions of, residues within the polypeptides or ISVDs as described herein. Any combination of deletion, insertion, and substitution is made to arrive at the final construct, provided that the final construct possesses the desired characteristics. The amino acid changes also may alter post-translational processes of the binding molecules, such as changing the number or position of glycosylation sites. Preferably, 1, 2, 3, 4, 5, 6, 7, 3, 9, or 10 amino acids may be substituted in a CDR, while 1, 2, 3, 4, 5, 6, 7, 8, 9, 10, 11, 12, 13, 14, 15, 16, 17, 18, 19, 20, or 25 amino acids may be substituted in the framework regions (FRs). The substitutions are preferably conservative substitutions as described herein. Additionally or alternatively, 1, 2, 3, 4, 5, or 6 amino acids may be inserted or deleted in each of the CDRs (of course, dependent on their length), while 1, 2, 3, 4, 5, 6, 7, 8, 9, 10, 11, 12, 13, 14, 15, 16, 17, 18, 19, 20, or 25 amino acids may be inserted or deleted in each of the FRs.

A useful method for identification of certain residues or regions of polypeptides or ISVDs as described herein, that are preferred locations for mutagenesis is called “alanine scanning mutagenesis” as described by Cunningham and Wells 1939 (Science 244: 1081-1085). Here, a residue or group of target residues within the binding molecule is/are identified (e.g., charged residues such as Arg, Asp, His, Lys, and Glu) and replaced by a neutral or negatively charged amino acid (most preferably alanine or polyalanine) to affect the interaction of the amino acids with the epitope. Those amino acid locations demonstrating functional sensitivity to the substitutions then are refined by introducing further or other variants at, or for, the sites of substitution. Thus, while the site for introducing an amino acid sequence variation is predetermined, the nature of the mutation per se needs not to be predetermined. For example, to analyze the performance of a mutation at a given site, alanine scanning or random mutagenesis is conducted at a target codon or region and the expressed binding molecule variants are screened for the desired activity.

Preferably, amino acid sequence insertions include amino- and/or carboxyl-terminal fusions ranging in length from 1, 2, 3, 4, 5, 6, 7, 8, 9 or 10 residues to polypeptides containing a hundred or more residues.

Another type of variant is an amino acid substitution variant. These variants have preferably (at least) 1, 2, 3, 4, 5, 6, 7, 8, 9 or 10 amino acid residues in the amino acid sequences, ISVDs or polypeptides replaced by a different residue. The sites of greatest interest for substitution mutagenesis include the CDRs, in particular the hypervariable regions, but FR alterations are also contemplated. For example, if a CDR sequence encompasses 6 amino acids, it is envisaged that one, two or three of these amino acids are substituted. Similarly, if a CDR sequence encompasses 15 amino acids it is envisaged that one, two, three, four, five or six of these amino acids are substituted.

Generally, if amino acids are substituted in one or more or all of the CDRs, it is preferred that the then-obtained “substituted” sequence is at least 60%, more preferably 65%, even more preferably 70%, particularly preferably 75%, more particularly preferably 80% or even more than 90% identical to the “original” CDR sequence. This means that it is dependent of the length of the CDR to which degree it is identical to the “substituted” sequence. For example, a CDR having 5 amino acids is preferably 80% identical to its substituted sequence in order to have at least one amino acid substituted. Accordingly, the CDRs of the amino acid sequences, ISVDs or polypeptides may have different degrees of identity to their substituted sequences, e.g., CDR1 may have 80%, while CDR3 may have 90%.

Preferred amino acid substitutions are conservative substitutions. Such conservative substitutions preferably are substitutions in which one amino acid within the following groups (a)-(e) is substituted by another amino acid residue within the same group: (a) small aliphatic, nonpolar or slightly polar residues: Ala, Ser, Thr, Pro and Gly; (b) polar, negatively charged residues and their (uncharged) amides: Asp, Asn, Glu and Gln; (c) polar, positively charged residues: His, Arg and Lys; (d) large aliphatic, nonpolar residues: Met, Leu, lie, Val and Cys; and (e) aromatic residues: Phe, Tyr and Trp. Further preferred conservative substitutions are as follows: Ala into Gly or into Ser; Arg into Lys; Asn into Gln or into His; Asp into Glu; Cys into Ser; Gln into Asn; Glu into Asp; Gly into Ala or into Pro; His into Asn or into Gln; Ile into Leu or into Val; Leu into lie or into Val; Lys into Arg, into Gln or into Glu; Met into Leu, into Tyr or into lie; Phe into Met, into Leu or into Tyr; Ser into Thr; Thr into Ser; Trp into Tyr; Tyr into Trp; and/or Phe into Val, into lie or into Leu. However, any substitution (including non-conservative substitution) is envisaged as long as the polypeptide retains its capability to specifically bind to the epitope on FcRn as described herein, specifically bind to FcRn at acidic pH (such as pH 5.0 to 6.8) with an affinity as described herein (e.g. with a K_{D} between 10⁻⁶ and 10⁻¹¹ M) and/or its CDRs have an identity of at least 60%, more preferably 65%, even more preferably 70%, particularly preferably 75%, more particularly preferably 80% to the “original” CDR sequence.

Preferred CDR sequences comprised in the ISVDs comprised in the FcRn antagonists of the present invention as the first polypeptide (a) are depicted as SEQ ID NO's 8 to 13 and 67 to 68 in Table 3. Sequence analysis further revealed that there are a number of possible sequence variations in the CDRs (see SEQ ID NO's 2 to 7 in Table 3).

TABLE 3
Sequence IDs of preferred ISVDs and CDR sequences

| | SEQ ID | |
|---|---|---|
| Nanobody® ISVD ID | NO. | Sequence |
| T0263018B11- | 14 | EVQLVESGGGVVQPGGSLRLSCAASGFTFSSYAMYWVRQAPG |
| parent | | KGLEWVSAISSGGGSTDYADSVKGRFTISRDNSKNTLYLQMNSL |
| | | RPEDTALYYCAADTLYTSLTWYSYWGQGTLVTVSS |
| T0263018B11- | 15 | EVQLVESGGGVVQPGGSLRLSCAASGFTFSSYAMYWVRQAPG |
| W100cS | | KGLEWVSAISSGGGSTDYADSVKGRFTISRDNSKNTLYLQMNSL |
| | | RPEDTALYYCAADTLYTSLTSYSYWGQGTLVTVSS |
| T0263018B11- | 11 | GFTFSSYAMY |
| parent/W100cS/(Y79S, | | |
| T100bE, W100cS) | | |
| (CDR1 - AbM) | | |
| T0263018B11- | 12 | AISSGGGSTD |
| parent/W100cS/(Y79S, | | |
| T100bE, W100cS) | | |
| (CDR2 - AbM) | | |
| T0263018B11- | 68 | DTLYTSLTWYSY |
| parent (CDR3 - AbM) | | |
| T0263018B11- | 13 | DTLYTSLTSYSY |
| W100cS (CDR3 - | | |
| AbM) | | |
| T0263018B11- | 8 | SYAMY |
| parent/W100cS/(Y79S, | | |
| T100bE, W100cS) | | |
| (CDR1 - Kabat) | | |
| T0263018B11- | 9 | AISSGGGSTDYADSVKG |
| parent/W100cS/(Y79S, | | |
| T100bE, W100cS) | | |
| (CDR2 - Kabat) | | |
| T0263018B11- | 67 | DTLYTSLTWYSY |
| parent (CDR3 | | |
| - | | |
| Kabat) | | |
| T0263018B11- | 10 | DTLYTSLTSYSY |
| W100cS (CDR3 - | | |
| Kabat) | | |
| CDR1 (variable | 2 | X₁ X₂ X₃ M Y, wherein |
| sequence -Kabat) | | X₁ is A, D, E, G, K, N, Q, S, T, or V, |
| | | X₂ is A, F, L, M, N, Q, S, T, V, or Y, and |
| | | X₃ is A, D, E, G, H, M, N, Q, S, T, or V |
| CDR2 (variable | 3 | A I X₃ X₄ G G G X₈ X₉ X₁₀ Y A D S V K G, wherein |
| sequence -Kabat) | | X₃ is A, D, E, G, H, P, Q, R, S, T, or V, |
| | | X₄ is A, D, E, G, K, M, N, P, Q, R, S, T, V, W, or Y, |
| | | X₈ is A, D, G, H, K, L, M, Q, S, T, or V, |
| | | X₉ is A, D, E, F, G, H, I, K, L, M, N, P, Q, R, S, T, V, W, or Y, |
| | | and |
| | | X₁₀ is A, D, E, H, S, T, V, or Y, |
| CDR3 (variable | 4 | D X₂ X₃ X₄ T X₆ X₇ T X₉ Y X₁₁ X₁₂, wherein |
| sequence -Kabat) | | X₂ is A, D, E, F, G, I, L, M, Q, S, T, V, W, or Y, |
| | | X₃ is L, or N, |
| | | X₄ is F, L, W, or Y, |
| | | X₆ is A, D, E, K, L, M, Q, S, or W, |
| | | X₇ is L, M, or V, |
| | | X₉ is A, D, E, F, G, H, K, L, M, Q, R, S, T, V, W or Y, |
| | | X₁₁ is A, E, L, M, Q, S, T, V, or W, and |
| | | X₁₂ is T or Y |
| CDR1 (variable | 5 | Xₐ X_{b} X_{c} F Xₑ X₁ X₂ X₃ M Y, wherein |
| sequence - AbM) | | Xₐ is A, D, E, F, G, H, K, L, M, N, Q, S, T, W, or Y, |
| | | X_{b} is F, I, L, M, V, or Y, |
| | | X_{c} is A, E, G, I, L, M, N, P, S, T, V, W, or Y, |
| | | Xₑ is D, E, G, K, M, N, P, Q, S or T, |
| | | X₁ is A, D, E, G, K, N, Q, S, T, or V, |
| | | X₂ is A, F, L, M, N, Q, S, T, V, or Y, and |
| | | X₃ is A, D, E, G, H, M, N, Q, S, T, or V |
| CDR2 (variable | 6 | A I X₃ X₄ G G G X₈ X₉ X₁₀, wherein |
| sequence - AbM) | | X₃ is A, D, E, G, H, P, Q, R, S, T, or V, |
| | | X₄ is A, D, E, G, K, M, N, P, Q, R, S, T, V, W, or Y, |
| | | X₈ is A, D, G, H, K, L, M, Q, S, T, or V, |
| | | X₉ is A, D, E, F, G, H, I, K, L, M, N, P, Q, R, S, T, V, W, or Y, |
| | | and |
| | | X₁₀ is A, D, E, H, S, T, V, or Y |
| CDR3 (variable | 7 | D X₂ X₃ X₄ T X₆ X₇ T X₉ Y X₁₁ X₁₂, wherein |
| sequence - AbM) | | X₂ is A, D, E, F, G, I, L, M, Q, S, T, V, W, or Y, |
| | | X₃ is L, or N, |
| | | X₄ is F, L, W, or Y, |
| | | X₆ is A, D, E, K, L, M, Q, S, or W, |
| | | X₇ is L, M, or V, |
| | | X₉ is A, D, E, F, G, H, K, L, M, Q, R, S, T, V, W or Y, |
| | | X₁₁ is A, E, L, M, Q, S, T, V, or W, and |
| | | X₁₂ is T or Y |
| T0263018B11 | 185 | DVQLVESGGGVVQPGGSLRLSCAASGFTFSSYAMYWVRQAPG |
| (E1D, W100cS) | | KGLEWVSAISSGGGSTDYADSVKGRFTISRDNSKNTLYLQMNSL |
| | | RPEDTALYYCAADTLYTSLTSYSYWGQGTLVTVSS |
| T0263018B11 | 186 | EVQLVESGGGVVQPGGSLRLSCAASGFTFSSYAMYWVRQAPG |
| (Y79S, T100bE, | | KGLEWVSAISSGGGSTDYADSVKGRFTISRDNSKNTLSLQMNSL |
| W100cs) | | RPEDTALYYCAADTLYTSLESYSYWGQGTLVTVSS |
| T0263018B11 | 187 | DVQLVESGGGVVQPGGSLRLSCAASGFTFSSYAMYWVRQAPG |
| (E1D, Y79S, T100bE, | | KGLEWVSAISSGGGSTDYADSVKGRFTISRDNSKNTLSLQMNSL |
| W100cS) | | RPEDTALYYCAADTLYTSLESYSYWGQGTLVTVSS |
| T0263018B11 | 189 | DTLYTSLESYSY |
| (Y79S, T100bE, | | |
| W100cS) (CDR3 - AbM) | | |
| T0263018B11 | 189 | DTLYTSLESYSY |
| (Y79S, T100bE, | | |
| W100cS) (CDR3 - Kabat) | | |

The ISVDs comprised in the FcRn antagonists as polypeptide (a) bind to a specific unique epitope on FcRn, which is an epitope that is different from the epitopes on FcRn bound by the natural ligands of FcRn, i.e. IgG.

As stated above, in some embodiments the ISVDs comprised in the FcRn antagonists of present invention as polypeptide (a) are such that when these are bound to or otherwise associated with an FcRn molecule, the binding by the FcRn molecule to IgG is not (significantly) affected, reduced or inhibited (i.e., the ISVD does not compete with wild-type IgG1 Fc region for binding to FcRn).

Hence, in some embodiments, the ISVDs comprised in the FcRn antagonists of present invention as polypeptide (a) specifically binds to amino acid residues on FcRn that are not involved in binding of FcRn to serum albumin and/or to IgG. The involvement of an amino acid residue on FcRn in binding to at least one ISVD, to serum albumin and/or to IgG can be determined, for example, by determining the interaction of the amino acid residue on FcRn with an amino acid residue on the at least one ISVD, with an amino acid residue on serum albumin and/or with an amino acid residue on IgG, e.g. by crystallographic studies. In this particular embodiment, the FcRn antagonists comprise at least one ISVD as the first polypeptide (a) which specifically binds to an epitope on FcRn that comprises amino acid residues that are not comprised in the epitope on FcRn bound by serum albumin and/or in the epitope on FcRn bound by IgG. In this particular embodiment, the FcRn antagonists comprise at least one ISVD which specifically binds to amino acid residues on FcRn that are not bound by serum albumin and/or by IgG.

In other embodiments, the ISVDs comprised in the FcRn antagonists of present invention as polypeptide (a) are preferably also such that they compete with the polypeptide with the amino acid sequence of SEQ ID NO's: 14 and/or 15 for binding to FcRn and/or that they “cross-block”(as defined herein) the binding of the polypeptide with the amino acid sequence of SEQ ID NO's: 14 and/or 15 to FcRn.

In other embodiments, the ISVDs comprised in the FcRn antagonists of present invention as polypeptide (a) are preferably also such that they compete with the polypeptide with the amino acid sequence of SEQ ID NO's: 186 and/or 187 for binding to FcRn and/or that they “cross-block” (as defined herein) the binding of the polypeptide with the amino acid sequence of SEQ ID NO's: 186 and/or 187 to FcRn.

The terms “cross-block”, “cross-blocked” and “cross-blocking” are used interchangeably herein to mean the ability of an immunoglobulin single variable domain or polypeptide to interfere with the binding of a ligand to its target, such as a natural ligand to its receptor(s). The extent to which an immunoglobulin single variable domain or polypeptide of the invention is able to interfere with the binding of another compound such as the natural ligand to its target and therefore whether it can be said to cross-block according to the invention, can be determined using competition binding assays. One particularly suitable quantitative cross-blocking assay uses a FACS- or an ELISA-based approach or Alphascreen to measure competition between the labelled (e.g., His tagged or biotinylated) immunoglobulin single variable domain or polypeptide according to the invention and the other binding agent in terms of their binding to the target. Suitable FACS-, ELISA- or Alphascreen-displacement-based assays for determining whether a binding molecule cross-blocks or is capable of cross-blocking a polypeptide are well known. It will be appreciated that these assays can be used with any of the immunoglobulin single variable domains or other binding agents described herein. Thus, in general, a cross-blocking polypeptide according to the invention is for example one which will bind to the target in the above cross-blocking assay such that, during the assay and in the presence of a second polypeptide or in the presence of the natural ligand, the recorded displacement of the immunoglobulin single variable domain or polypeptide according to the invention is between 60% and 100% (e.g., in ELISA/Alphascreen based competition assay) or between 80% to 100% (e.g., in FACS based competition assay) of the maximum theoretical displacement (e.g., displacement by cold (e.g., unlabeled) immunoglobulin single variable domain or polypeptide that needs to be cross-blocked) by the to be tested potentially cross-blocking agent that is present in an amount of 0.01 mM or less.

In particular embodiments, the ISVDs comprised in the FcRn antagonists of present invention as polypeptide (a) are such that they bind to essentially the same amino acid residues and/or epitope on FcRn as the amino acid residues and/or epitope bound by the polypeptide with the amino acid sequence of SEQ ID NOs: 14, 15, 185, 186 and/or 187, and even more preferably such that they share essentially the same amino acid interactions as the polypeptide with the amino acid sequence of SEQ ID NOs: 14, 15, 185, 186 and/or 187. For this purpose, according to a specific but non-limiting aspect, FcRn antagonists according to the present invention preferably either have the same CDRs as the sequence of SEQ ID NOs: 14, 15, 185, 186 and/or 187, or compared to the sequence of SEQ ID NOs: 14, 15, 185, 186 and/or 187 preferably contain within their CDRs only such mutations (such as conservative amino acid substitutions) that still allow them to undergo the same or essentially the same amino acid interactions with FcRn as the polypeptide with the sequence of SEQ ID NOs: 14, 15, 185, 186 and/or 187.

In particular embodiments, the ISVDs comprised in the FcRn antagonists of present invention as polypeptide (a) specifically bind to the above-described epitope on FcRn with an affinity (KA) of between 10⁶ M⁻¹ and 10¹¹ M⁻¹.

In particular embodiments, the ISVDs comprised in the FcRn antagonists of present invention as polypeptide (a) specifically bind to the above-described epitope on FcRn with a dissociation constant (K_{D}) of between 10⁻⁶ nM and 10⁻¹¹ M or less. Preferably, the K) is determined by Kinexa, BLI or SPR, for instance as determined by SPR.

In particular embodiments, the ISVDs comprised in the FcRn antagonists of present invention as polypeptide (a) specifically bind to the above-described epitope on FcRn with an on-rate constant (kₒₙ) selected from the group consisting of at least about 10² M⁻¹s⁻¹, of at least about 10³ M⁻¹s⁻¹, at least about 10⁴ M⁻¹s⁻¹, at least about 10⁵ M⁻¹s⁻¹, at least about 10⁶ M⁻¹s⁻¹, at least about 10⁷ M⁻¹s⁻¹, and at least about 10⁸ M⁻¹s⁻¹, preferably as measured by surface plasmon resonance or BLI.

In particular embodiments, the ISVDs comprised in the FcRn antagonists of present invention as polypeptide (a) specifically bind to the above-described epitope on FcRn with an off-rate constant (k_{off}) selected from the group consisting of at most about 10⁻¹s⁻¹, at most about 10⁻²s⁻¹, at most about 10⁻³s⁻¹, of at most about 10⁻⁴s⁻¹, at most about 10⁻⁵s⁻¹, and at most about 10⁻⁶s⁻¹, preferably as measured by surface plasmon resonance or BLI.

The ISVDs comprised in the FcRn antagonists of present invention as polypeptide (a) are, in certain embodiments, such that they are cross-reactive between human FcRn and FcRn from at least one, preferably from at least two, more preferably from at least three and up to essentially all of the following species of mammal: mouse, dog, rat, rabbit, guinea pig, pig, sheep, cow and cynomolgus monkey.

When an ISVD is said to exhibit “(improved) cross-reactivity for binding to human and non-human primate FcRn” compared to another ISVD, it means that for said ISVD the ratio of the binding activity (such as expressed in terms of K_{D} or k_{off}) for human FcRn and for non-human primate FcRn is lower than that same ratio calculated for the other ISVD in the same assay. Good cross-reactivity for binding to human and non-human primate FcRn allows for the assessment of toxicity of an FcRn antagonists according to the present invention in preclinical studies conducted on non-human primates.

In particular embodiments, the ISVDs comprised in the FcRn antagonists of present invention as polypeptide (a) are such that they are (at least) cross-reactive between human FcRn and cynomolgus monkey FcRn, and preferably also between either human FcRn and/or cynomolgus monkey FcRn on the one hand, and at least one, preferably both of rat FcRn and pig FcRn on the other hand. For the sake of convenience, in the sequence of FcRn, the stretches of amino acids that are assumed to be part of the putative epitope of the polypeptides of the invention have been highlighted. Without being limited to any specific mechanism or hypothesis, it is assumed that the polypeptides of the invention are (essentially) capable of binding to (one or more amino acid residues within) the corresponding stretches of amino acid residues that are present within the amino acid sequence of those mammalian FcRn proteins, with which the polypeptides of the invention are cross-reactive.

Generally, an ISVDs comprised in the FcRn antagonists of present invention as polypeptide (a) can be considered to be cross-reactive between human FcRn and FcRn from one of the above mentioned other species when it can bind to human FcRn with an affinity less than 500 nM, preferably less than 200 nM, more preferably less than 10 nM; and also to FcRn from those above-mentioned species with an affinity less than 500 nM, preferably less than 200 nM, more preferably less than 10 nM, again both as determined using SPR.

In particular embodiments, the ISVDs comprised in the FcRn antagonists of present invention as polypeptide (a) specifically bind to the unique epitope on FcRn as disclosed herein in a pH-dependent manner, such that their binding affinity at an acidic pH, in particular at an acidic pH of between 5.0 and 6.8, is at least three times higher than the binding affinity at a neutral or physiological pH of 7.4. In these particular embodiments, the ISVDs comprised in the FcRn antagonists of the present invention show conditional specific binding to FcRn and as a result, by making use of the in vivo FcRn-mediated recycling mechanism, have a prolonged in vivo serum half-life. The ISVDs comprised in the FcRn antagonists of the present invention may therefore be used to extend the in vivo half-life of therapeutic targets or therapeutic molecules of interest to which they are suitably linked, bound or fused.

The pH-dependent interaction of the ISVDs with FcRn in certain particular embodiments of the present invention ensures that these polypeptides are optimally and rapidly captured in the acidic environment of the endosomes and efficiently released after recycling when encountering pH 7.4 again (i.e. outside the endosomes).

In this way, the present inventors have been able to demonstrate that the FcRn antagonists according to a certain aspect of the present invention, by specifically binding to FcRn in a pH-dependent manner, show an increased half-life in vivo.

Thus, in particular embodiments, the FcRn antagonists according to the invention that comprise at least one ISVD binding to the unique epitope on FcRn (as the first polypeptide, (a)) according to the invention and as disclosed herein, can have an increased half-life, compared to a known FcRn antagonist as described in the prior art.

Also, FcRn antagonists provided by the invention preferably have a half-life (defined as t½ beta) in man that is more than 1 hour, preferably more than 2 hours, more preferably of more than 6 hours, such as of more than 12 hours, and for example of about one day, two days, one week, two weeks and up to or even beyond the half-life of IgG in man (estimated to be around 19-21 days), although the latter may be less critical.

The ISVDs comprised in the FcRn antagonists of present invention as polypeptide (a) are preferably also such that either:
they have a serum half-life in man (expressed as t½ beta) that is more than 6 hours, preferably more than 12 hours, more preferably of more than 24 hours, even more preferably more than 72 hours; for example of about one week, two weeks and up to the half-life of IgG in man (estimated to be around 19-21 days); and/or such that
when they are linked to another moiety or entity (e.g., comprised in the FcRn antagonist of the present invention, linked to polypeptide (b), as defined herein, they confer to the resulting FcRn antagonist of the invention a serum half-life which is equal to or increased as compared with the half-life of polypeptide (b) on its own; for instance the half-life in man of the FcRn antagonist comprising at least one ISVD as polypeptide (a) (expressed as t½ beta) may be more than 6 hours, preferably more than 12 hours, such as more than 18 hours, more preferably of more than 24 hours, such as more than 29 hours, even more preferably more than 72 hours; for example of about one week, two weeks, and up to or even beyond the half-life of IgG in man.

In particular embodiments, at an acidic pH of between 5.0 and 6.8, the ISVDs comprised in the FcRn antagonists of present invention as polypeptide (a) specifically bind to FcRn with an affinity (K_{A}) of between 10⁻³ nM⁻¹ and 10² nM⁻¹. The affinity (K_{A}) of these ISVDs for FcRn at acidic pH, preferably at a pH of between 5.0 and 6.8 is at least three times higher than the affinity (K_{A}) of the same polypeptides for FcRn at neutral or physiologic pH of 7.4.

In further particular embodiments, at an acidic pH of between 5.0 and 6.8, the ISVDs comprised in the FcRn antagonists of present invention as polypeptide (a) bind to FcRn with an affinity that is at least ten times higher than the affinity (K_{A}) for FcRn of the same polypeptides at neutral or physiologic pH of 7.4. In yet further particular embodiments, at an acidic pH of between 5.0 and 6.8, the ISVDs comprised in the FcRn antagonists of present invention as polypeptide (a) bind to FcRn with an affinity that is at least fifty times higher, such as at least hundred times higher than the affinity (K_{A}) for FcRn of the same polypeptides at neutral or physiologic pH of 7.4.

In certain particular embodiments, the present invention provides an FcRn antagonists as described herein characterized in that the ISVDs comprised in the FcRn antagonists of present invention as polypeptide (a) binds to FcRn at neutral or physiologic pH of 7.4 with an affinity that is at least three times, such as at least ten times, such as at least fifty times, such as at least hundred times lower than the affinity with which the at least one ISVD binds to FcRn at acidic pH of between 5.0 and 6.8.

In a particular embodiment, at physiologic pH such as at a pH of 7.4, the at least one ISVD binds to FcRn with a KA value lower than 104 liters/mol.

In certain particular embodiments, the present invention provides FcRn antagonists as described herein characterized in that the ISVDs comprised in the FcRn antagonists of present invention as polypeptide (a) does not detectably, selectively or specifically bind to FcRn, or exhibits no or essentially no binding to FcRn at neutral or physiologic pH, such as at a pH of 7.4.

In particular embodiments, at an acidic pH of between 5.0 and 6.8, the ISVDs comprised in the FcRn antagonists of present invention as polypeptide (a) specifically bind to FcRn with a dissociation constant (K_{D}) of between 10⁻⁶ M and 10⁻¹¹ M or less. Preferably, the K_{D} is determined by Kinexa, BLI or SPR, for instance as determined by SPR.

In particular embodiments, at an acidic pH of between 5.0 and 6.8, the ISVDs comprised in the FcRn antagonists of present invention as polypeptide (a) specifically bind to FcRn with a dissociation constant (K_{D}) of between 10³ nM and 10⁻² nM or less. The dissociation constant (K_{D}) of these ISVDs for FcRn at acidic pH, preferably at a pH of between 5.0 and 6.8 is at least three times better (i.e., lower value) than the dissociation constant (K_{D}) of the same ISVDs for FcRn at neutral or physiologic pH of about 7.4. In further particular embodiments, at an acidic pH of between 5.0 and 6.8, the ISVDs comprised in the FcRn antagonists of present invention as polypeptide (a) bind to FcRn with an affinity that is at least ten times higher/better than the affinity (K_{D}) for FcRn of the same ISVD at neutral or physiologic pH of about 7.4. In yet further particular embodiments, at an acidic pH of between 5.0 and 6.8, the ISVDs according to the invention bind to FcRn with an affinity that is at least fifty times higher, such as at least hundred times higher/better than the affinity (K_{D}) for FcRn of the same ISVDs at neutral or physiologic pH of about 7.4.

In certain particular embodiments, the present invention provides FcRn antagonists as described herein characterized in that the ISVD comprised in the FcRn antagonists of present invention as polypeptide (a) binds to FcRn at neutral or physiologic pH of 7.4 with an affinity that is at least three times, such as at least ten times, such as at least fifty times, such as at least hundred times lower/worse than the affinity with which the at least one ISVD binds to FcRn at acidic pH of between 5.0 and 6.8.

In a particular embodiment, at physiologic pH such as at a pH of 7.4, the at least one ISVD binds to FcRn with a K_{D} value greater than 10⁻⁴ mol/liter.

In certain particular embodiments, the present invention provides FcRn antagonists as described herein characterized in that the ISVD comprised in the FcRn antagonists of present invention as polypeptide (a) does not detectably, selectively or specifically bind to FcRn, or exhibits no or essentially no binding to FcRn at neutral or physiologic pH, such as at a pH of 7.4.

Accordingly, the present invention relates to FcRn antagonists as described herein, comprising at least one ISVD as the first polypeptide (a) that binds to FcRn with an average K_{D} value of between 1000 nM and 10 pM, such as at an average K_{D} value of 900 nM or less, even more preferably at an average K_{D} value of 800 nM or less, such as less than 700, 600, 500, 400, 300, 200, 100, 50 nM or even less, such as less than 40, 30, 20, 10, 5, 1 nM such as less than 500, 400, 300, 200, 100, 90, 80, 70, 60, 50, 40, 30, 20 pM, or even less, such as less than 10 pM. Preferably, the K_{D} is determined by Kinexa, BLI or surface plasmon resonance (SPR), for instance as determined by SPR. Preferably, the average K_{D} is measured by SPR on recombinant protein.

The present invention also relates to FcRn antagonists as described herein, comprising at least one ISVD as the first polypeptide (a) binding to FcRn at acidic pH with an EC50 value of between 1000 nM and 1 pM, such as at an average EC50 value of 1000 nM or less, even more preferably at an average EC50 value of 900 nM or less, such as less than 800, 700, 600, 500, 400, 300, 200, 100, 50 nM or even less, such as less than 40, 30, 20, 10, 5 or 1 nM or even less, such as less than 500, 400, 300, 200, 100, 90, 80, 70, 60, 50, 40, 30, 20, 10, 5 pM, or even less, such as less than 4 pM.

In particular embodiments, at an acidic pH of between 5.0 and 6.8, the ISVDs comprised in the FcRn antagonists of the present invention, as the first polypeptide (a), specifically bind to FcRn with an on rate constant (kₒₙ) selected from the group consisting of at least about 10²M⁻¹s⁻¹, of at least about 10³M⁻¹s⁻¹, at least about 10⁴M⁻¹s⁻¹, at least about 10⁵M⁻¹s⁻¹, at least about 10⁶M⁻¹s⁻¹, at least about 10⁷M⁻¹s⁻¹, and at least about 10⁸M⁻¹s⁻¹, preferably as measured by surface plasmon resonance or BLI.

In particular embodiments, at an acidic pH of between 5.0 and 6.8, the ISVDs comprised in the FcRn antagonists of the present invention, as the first polypeptide (a), specifically bind to FcRn with an off rate constant (k_{off}) selected from the group consisting of at most about 10⁻¹s⁻¹, at most about 10⁻²s⁻¹, at most about 10⁻³s⁻¹, of at most about 10⁻⁴s⁻¹, at most about 10⁻⁵s⁻¹, and at most about 10⁻⁶s⁻¹, preferably as measured by surface plasmon resonance or BLI.

In particular embodiments, at an acidic pH of between 5.0 and 6.8, the ISVDs comprised in the FcRn antagonists of the present invention, as the first polypeptide (a), specifically bind to FcRn with an off rate constant (k_{off}) selected from the group consisting of at most about 10⁻¹s⁻¹, at most about 10⁻²s⁻¹, at most about 10⁻³s⁻¹, of at most about 10⁻⁴s⁻¹, at most about 10⁻⁵s⁻¹, and at most about 10⁻⁶s⁻¹. The off-rate constant (k_{off}) of these amino acid sequences and polypeptides for FcRn at acidic pH, preferably at a pH of between 5.0 and 6.8 is at least three times lower than the off rate constant (k_{off}) of the same amino acid sequences and polypeptides for FcRn at neutral or physiologic pH of 7.4. In further particular embodiments, at an acidic pH of between 5.0 and 6.8, the polypeptides according to the invention bind to FcRn with off rate constant (k_{off}) that is at least ten times lower than the off rate constant (k_{off}) for FcRn of the same polypeptides at neutral or physiologic pH of 7.4. In yet further particular embodiments, at an acidic pH of between 5.0 and 6.8, the polypeptides according to the invention bind to FcRn with off rate constant (k_{off}) that is at least fifty times lower, such as at least hundred times lower than the off-rate constant (k_{off}) for FcRn of the same polypeptides at neutral or physiologic pH of 7.4.

In certain particular embodiments, the present invention provides FcRn antagonists as described herein characterized in that the ISVDs comprised in the FcRn antagonists of the present invention, as the first polypeptide (a), binds to FcRn at neutral or physiologic pH of 7.4 with an off rate constant (k_{off}) that is at least three times, such as at least ten times, such as at least fifty times, such as at least hundred times higher than the off rate constant (k_{off}) with which the ISVD binds to FcRn at acidic pH of between 5.0 and 6.8.

In certain particular embodiments, the present invention provides FcRn antagonists as described herein characterized in that the ISVDs comprised in the FcRn antagonists of the present invention, as the first polypeptide (a), does not detectably, selectively or specifically bind to FcRn, or exhibits no or essentially no binding to FcRn at neutral or physiologic pH, such as at a pH of 7.4.

In particular embodiments, the ISVDs comprised in the FcRn antagonists of the present invention, as the first polypeptide (a), have a high affinity for a particular epitope on FcRn at an acidic pH of between 5.0 and 6.8, while showing a reduced binding or not showing any detectable, selective and/or specific binding to FcRn at physiological or neutral pH of 7.4.

### The Second Polypeptide (b) Comprised in the FcRn Antagonists of the Present Invention

As described above, the FcRn antagonists of the present invention comprise (b) at least a second polypeptide which competes with wild-type IgG1 Fc region for binding to FcRn. Any polypeptide which competes with the wild-type IgG1 Fc region for binding to FcRn may be used as the at least one second polypeptide comprised in the FcRn antagonists disclosed herein. This at least one second polypeptide is not limited as long as it specifically binds to an epitope on FcRn and competes with wild-type IgG1 Fc region for binding to FcRn.

In a preferred embodiment, the at least one second polypeptide (b) comprised in the FcRn antagonists of the present invention binds specifically to FcRn with increased affinity relative to wild-type IgG1 Fc region binding to FcRn. In a more preferred embodiment, the at least one second polypeptide (b) comprised in the FcRn antagonists of the present invention has increased FcRn binding affinities at both acidic pH and extracellular physiological pH as compared to wild-type IgG1 Fc region binding to FcRn.

In another embodiment, the at least one second polypeptide (b) comprised in the FcRn antagonists of the present invention specifically binds to FcRn with reduced pH dependence relative to a wild-type IgG1 Fc region.

In further embodiments, the at least one second polypeptide (b) comprised in the FcRn antagonists of the present invention has altered affinity (increased or decreased) for CD16a as compared to a wild-type IgG1 Fc region.

In one embodiment, the at least one second polypeptide (b) comprised in the FcRn antagonists of the present invention comprises or consists of an antibody or a fragment thereof. For instance, the antibody or a fragment thereof is a monoclonal antibody, a fusion antibody, a humanized antibody, a human antibody or a single chain antibody. In one embodiment, the at least one second polypeptide (b) comprised in the FcRn antagonists of the present invention comprises or consists of an ISVD.

In an embodiment, the at least one second polypeptide (b) comprised in the FcRn antagonists of the present invention comprises or consists of an Affibody® (affibody molecule), a single-chain variable fragment (scFv), a Fab, a Designed Ankyrin Repeat Protein (DARPin®), a Nanofitin® (aka affitin) or an immunoglobulin variable domain sequence (ISVD).

Affibody molecules are a class of engineered affinity proteins with proven potential for therapeutic, diagnostic and biotechnological applications. Affibody molecules are small (6.5 kDa) single domain proteins that can be isolated for high affinity and specificity to any given protein target (from FEBS Letters, Volume 584, Issue 12, 18 Jun. 2010, Pages 2670-2680).

Affitins are artificial proteins with the ability to selectively bind antigens. They are structurally derived from the DNA-binding protein Sac7d, found in Sulfolobus acidocaldarius. Due to their small size and high solubility, they can be easily produced in large amounts using bacterial expression systems (see, e.g., Kalichuk V. et al., “A novel, smaller scaffold for Affitins: Showcase with binders specific for EpCAM”, Biotechnol Bioeng. 2018; 115(2):290-299). In a preferred embodiment, the at least one second polypeptide (b) comprised in the FcRn antagonists of the present invention comprises or consists of a Nanofitin® (aka affitin).

In a preferred embodiment, the at least one second polypeptide (b) comprised in the FcRn antagonists of the present invention comprises or consists of an Fc domain or a fragment thereof, as defined herein above in the present description in the context of the first polypeptide (a).

In certain embodiments, the Fc domain or FcRn-binding fragment thereof binds specifically to FcRn with increased affinity relative to a wild-type IgG1 Fc region. In preferred embodiments, the Fc domain comprised in the FcRn antagonists of the present invention is in a dimeric form, i.e., it comprises at least a hinge region or part of it, two CH2 domains, and two CH3 domains, i.e., two polypeptide fragments, see, e.g., FIG. 1. Hence, in this preferred embodiment, the FcRn antagonists of the present invention comprise at least two polypeptides (b), i.e., the two polypeptides which conform the dimeric Fc domain. Hence, in a preferred embodiment, the Fc domain is dimeric, more preferably heterodimeric, as defined above.

In certain embodiments, the Fc region is an IgG Fc region. Fc domains from any IgG subtype can be used to generate the Fc domain or FcRn-binding fragment thereof comprised in the FcRn antagonist of the present invention. In some embodiments, the Fc domain or FcRn-binding fragment thereof is derived from a human IgG1, IgG2, IgG3, or IgG4 Fc domain and comprise the substitutions described herein relative to the wildtype origin. In certain other embodiments, the Fc domain or FcRn-binding fragment thereof is an artificial Fc derived from more than one IgG subtype. In other embodiments, the Fc domain or FcRn-binding fragment thereof comprises a chimeric hinge (i.e., a hinge comprising hinge portions derived from hinge domains of different antibody isotypes, e.g., an upper hinge domain from an IgG4 molecule and an IgG 1 middle hinge domain). In certain embodiments, the Fc domain or FcRn-binding fragment thereof is an IgG 1 Fc region. In certain embodiments, the Fc domain or FcRn-binding fragment thereof is a human IgG Fc region. In certain embodiments, the Fc domain or FcRn-binding fragment thereof is a human IgG 1 Fc region. In certain embodiments, the Fc domain or FcRn-binding fragment thereof is a chimeric Fc region.

In certain embodiments, the Fc domain comprises amino acid alterations, substitutions, insertions and/or deletions that confer the desired characteristics.

Useful Fc domains FcRn-binding or fragments thereof being comprised in the FcRn antagonists of the present invention are described in WO 2015/100299 and in WO 2019/110823.

In some embodiments, the Fc domain or FcRn-binding fragment thereof comprises at least one, preferably all, of the following amino acids at the following positions:
a) a tyrosine (Y) at amino acid position 252,
b) a threonine (T) at amino acid position 254,
c) a glutamic acid (E) at amino acid position 256,
d) a lysine (K) at amino acid position 433,
e) a phenylalanine (F) at amino acid position 434, and/or
f) a tyrosine (Y) at amino acid position 436;
   according to EU numbering.

Non-limiting examples of amino acid sequences that can be used in the Fc domain or FcRn-binding fragment thereof are set forth in Table 1 of WO 2015/100299 (SEQ ID NO: 22-24 in the present description). In certain embodiments, the amino acid sequence of the Fc domain or FcRn-binding fragment thereof comprises the amino acid sequence set forth in SEQ ID NO: 22. In certain embodiments, the amino acid sequence of the Fc domain or FcRn-binding fragment thereof comprises or consists of the amino acid sequence set forth in SEQ ID NO: 22, 23, or 24. In one embodiment, if the Fc domain is in a dimeric form, i.e., comprises at least a hinge region or part of it, two CH2 domains, and two CH3 domains, i.e., two polypeptide fragments, the Fc domain may comprise two polypeptides comprising or consisting of the amino acid sequence set forth in SEQ ID NO: 22. Hence, if the Fc domain comprises at least a hinge region or part of it, two CH2 domains, and two CH3 domains, i.e., two polypeptide fragments, the Fc domain may comprise two polypeptides comprising or consisting of the amino acid sequence set forth in SEQ ID NO: 23. Hence, if the Fc domain comprises at least a hinge region or part of it, two CH2 domains, and two CH3 domains, i.e., two polypeptide fragments, the Fc domain may comprise two polypeptides comprising or consisting of the amino acid sequence set forth in SEQ ID NO: 24. In these embodiments, at least one and preferably both of the CH3 domains comprised in the Fc domain comprised in the FcRn antagonists of the present invention may comprise knob in hole mutations, as defined above. Hence, if the Fc domain comprises at least a hinge region or part of it, two CH2 domains, and two CH3 domains, i.e., two polypeptide fragments, the Fc domain may comprise two polypeptides comprising or consisting of the amino acid sequences set forth in SEQ ID NO: 119 and 120. Additionally, or alternatively, the amino acids HY in the CH3 domains of Fc domain may be mutated into RF, such as at positions 435 and 436, (H435R and Y436F in CH3 domain, as described by Jendeberg, L. et al. (1997, J. Immunological Meth., 201:25-34)).

In certain embodiments, the Fc domain is a homodimer, wherein the amino acid sequence of each of the polypeptides comprised in the Fc domain consists of SEQ ID NO: 22. In certain embodiments, the Fc domain is a homodimer, wherein the amino acid sequence of each of the polypeptides comprised in the Fc domain consists of SEQ ID NO: 23. In certain embodiments, the Fc domain is a homodimer, wherein the amino acid sequence of each of the polypeptides comprised in the Fc domain consists of SEQ ID NO: 24. In certain embodiments, the Fc domain is a heterodimer, wherein the amino acid sequences of each of the polypeptides comprised in the Fc domain comprises or consists of SEQ ID NO: 119 and 120.

In certain embodiments, the amino acid sequence of the Fc domains of the variant Fc region comprises or consists of the amino acid sequence set forth in SEQ ID NO: 22.

In certain embodiments, the amino acid sequence of the Fc domains of the variant Fc region comprises or consists of the amino acid sequence set forth in SEQ ID NO: 23.

In certain embodiments, the amino acid sequence of the Fc domains of the variant Fc region comprises or consists of the amino acid sequence set forth in SEQ ID NO: 24.

In certain embodiments, the amino acid sequence of the Fc domains of the variant Fc region comprises or consists of the amino acid sequences set forth in SEQ ID NO: 119 and/or 120.

In certain embodiments, the FcRn antagonist comprises a variant Fc domain or fragment thereof that does not comprise an N-linked glycan at EU position 297. In certain embodiments, the FcRn antagonist comprises a variant Fc region that comprises an afucosylated N-linked glycan at EU position 297. In certain embodiments, the FcRn antagonist comprises a variant Fc domain or fragment thereof that comprises an N-linked glycan having a bisecting GlcNac at EU position 297.

Further Fc domains or fragments thereof being comprised in the FcRn antagonists of the present invention are described in WO 2021/016571.

In some embodiments, the Fc domain or FcRn-binding fragment thereof comprised in the FcRn antagonist of the present invention may comprise an amino acid substitution selected from M252, 1253, S254, T256, K288, T307, K322, E380, L432, N434, or Y436, and any combinations thereof. Unless otherwise indicated, all Fc residue positions described herein are according to the EU numbering system. In some embodiments, the Fc domain or FcRn-binding fragment thereof may comprise a double amino acid substitution at any two amino acid positions selected from M252, 1253, S254, T256, K288, T307, K322, E380, L432, N434, and Y436. In some embodiments, the Fc domain or FcRn-binding fragment thereof may comprise a triple amino acid substitution at any three amino acid positions selected from M252, 1253, S254, T256, K288, T307, K322, E380, L432, N434, and Y436. In some embodiments, the Fc domain or FcRn-binding fragment thereof may comprise a quadruple amino acid substitution at any four amino acid positions selected from M252, 1253, S254, T256, K288, T307, K322, E380, L432, N434, and Y436. In some embodiments, it may be desirable for a Fc domain or FcRn-binding fragment thereof to comprise an amino acid substitution at any of the amino acid positions selected from M252, 1253, S254, T256, K288, T307, K322, E380, L432, or Y436, and any combinations thereof, wherein amino acid position N434 is not substituted (i.e., amino acid position N434 is wildtype).

For instance, in other embodiments, the Fc domain or FcRn-binding fragment thereof may comprise an amino acid substitution selected from M252Y (i.e., a tyrosine at amino acid position 252), T256D, T256E, K288D, K288N, T307A, T307E, T307F, T307M, T307Q, T307W, E380C, N434F, N434P, N434Y, Y436H, Y436N, or Y436W, and any combinations thereof.

In some embodiments, the Fc domain or FcRn-binding fragment thereof may comprise a double amino acid substitution selected from M252, wherein the substitution is M252Y; T256, wherein the substitution is T256D, or T256E; K288, wherein the substitution is K288D, or K288N; T307, wherein the substitution is T307A, T307E, T307F, T307M, T307Q, or T307W; E380, wherein the substitution is E380C; N434, wherein the substitution is N434F, N434P, or N434Y; Y436, wherein the substitution is Y436H, Y436N, or Y436W. In some embodiments, the Fc domain or FcRn-binding fragment thereof may comprise a triple amino acid substitution selected from M252, wherein the substitution is M252Y; T256, wherein the substitution is T256D, or T256E; K288, wherein the substitution is K288D, or K288N; T307, wherein the substitution is T307A, T307E, T307F, T307M, T307Q, or T307W; E380, wherein the substitution is E380C; N434, wherein the substitution is N434F, N434P, or N434Y; Y436, wherein the substitution is Y436H, Y436N, or Y436W. In some embodiments, the Fc domain or FcRn-binding fragment thereof may comprise a quadruple amino acid substitution selected from M252, wherein the substitution is M252Y; T256, wherein the substitution is T256D, or T256E; K288, wherein the substitution is K288D, or K288N; T307, wherein the substitution is T307A, T307E, T307F, T307M, T307Q, or T307W; E380, wherein the substitution is E380C; N434, wherein the substitution is N434F, N434P, or N434Y; Y436, wherein the substitution is Y436H, Y436N, or Y436W. In some embodiments, it may be desirable for a Fc domain or FcRn-binding fragment thereof to comprise an amino acid substitution at any of the amino acid positions selected from M252Y, T256D, T256E, K288D, K288N, T307A, T307E, T307F, T307M, T307Q, T307W, E380C, Y436H, Y436N, or Y436W, and any combinations thereof, wherein amino acid position N434 is not substituted with a phenylalanine (F) or a tyrosine (Y). In some embodiments, it may be desirable for a Fc domain or FcRn-binding fragment thereof to comprise an amino acid substitution at any of the amino acid positions selected from M252Y, T256D, T256E, K288D, K288N, T307A, T307E, T307F, T307M, T307Q, T307W, E380C, Y436H, Y436N, or Y436W, and any combinations thereof, wherein amino acid position N434 is not substituted with a tyrosine (Y). In some embodiments, it may be desirable for a Fc domain or FcRn-binding fragment thereof to comprise an amino acid substitution at any of the amino acid positions selected from M252Y, T256D, T256E, K288D, K288N, T307A, T307E, T307F, T307M, T307Q, T307W, E380C, Y436H, Y436N, or Y436W, and any combinations thereof, wherein amino acid position N434 is not substituted (i.e., amino acid position N434 is wildtype).

In certain embodiments, the Fc domain or FcRn-binding fragment thereof may comprise an amino acid substitution selected from M252, T256, T307, or N434, and any combinations thereof. In certain embodiments, the Fc domain or FcRn-binding fragment thereof may comprise a double amino acid substitution at any two amino acid positions selected from M252, T256, T307, and N434. In certain embodiments, the Fc domain or FcRn-binding fragment thereof may comprise a triple amino acid substitution at any three amino acid positions selected from M252, T256, T307, and N434. In certain embodiments, the Fc domain or FcRn-binding fragment thereof may comprise a quadruple amino acid substitution at amino acid positions M252, T256, T307, and N434. In some embodiments, it may be desirable for a Fc domain or FcRn-binding fragment thereof to comprise an amino acid substitution selected from M252, T256, or T307, and any combinations thereof, wherein amino acid position N434 is not substituted (i.e., amino acid position N434 is wildtype).

In exemplary embodiments, the Fc domain or FcRn-binding fragment thereof may comprise an amino acid substitution selected from M252, wherein the substitution is M252Y; T256, wherein the substitution is T256D, or T256E; T307, wherein the substitution is T307Q, or T307W; or N434, wherein the substitution is N434F, or N434Y, and any combinations thereof. In certain embodiments, the Fc domain or FcRn-binding fragment thereof may comprise a double amino acid substitution at any two amino acid positions selected from M252, wherein the substitution is M252Y; T256, wherein the substitution is T256D, or T256E; T307, wherein the substitution is T307Q, or T307W; or N434, wherein the substitution is N434F, or N434Y. In certain embodiments, the Fc domain or FcRn-binding fragment thereof may comprise a triple amino acid substitution at any three amino acid positions selected from M252, wherein the substitution is M252Y; T256, wherein the substitution is T256D, or T256E; T307, wherein the substitution is T307Q, or T307W; or N434, wherein the substitution is N434F, or N434Y. In certain embodiments, the Fc domain or FcRn-binding fragment thereof may comprise a quadruple amino acid substitution at amino acid positions selected from M252, wherein the substitution is M252Y; T256, wherein the substitution is T256D, or T256E; T307, wherein the substitution is T307Q, or T307W; or N434, wherein the substitution is N434F, or N434Y. In some embodiments, it may be desirable for a Fc domain or FcRn-binding fragment thereof to comprise an amino acid substitution selected from M252Y, T256D, T256E, T307Q, or T307W, and any combinations thereof, wherein amino acid position N434 is not substituted with a phenylalanine (F) or a tyrosine (Y). In some embodiments, it may be desirable for a Fc domain or FcRn-binding fragment thereof to comprise an amino acid substitution selected from M252Y, T256D, T256E, T307Q, or T307W, and any combinations thereof, wherein amino acid position N434 is not substituted with a tyrosine (Y).

In some embodiments, it may be desirable for a Fc domain or FcRn-binding fragment thereof to comprise an amino acid substitution selected from M252Y, T256D, T256E, T307Q or T307W, and any combinations thereof, wherein amino acid position N434 is not substituted (i.e., amino acid position N434 is wild type).

In certain embodiments, the Fc domain or FcRn-binding fragment thereof may comprise an amino acid substitution selected from T256D, or T256E, and/or T307W, or T307Q, and further comprises an amino acid substitution selected from N434F, or N434Y, or M252Y. In some embodiments, it may be desirable for a Fc domain or FcRn-binding fragment thereof to comprise an amino acid substitution selected from T256D, or T256E, and/or T307W, or T307Q and further comprise the amino acid substitution M252Y, wherein amino acid position N434 is not substituted with a phenylalanine (F) or a tyrosine (Y). In some embodiments, it may be desirable for a Fc domain or FcRn-binding fragment thereof to comprise an amino acid substitution selected from T256D, or T256E, and/or T307W, or T307Q, and further comprise the amino acid substitution M252Y, wherein amino acid position N434 is not substituted with a tyrosine (Y). In some embodiments, it may be desirable for a Fc domain or FcRn-binding fragment thereof to comprise an amino acid substitution selected from T256D, or T256E, and/or T307W, or T307Q, and further comprise the amino acid substitution M252Y, wherein amino acid position N434 is not substituted (i.e., amino acid position N434 is wild type).

In some embodiments, the Fc domain or FcRn-binding fragment thereof may comprise the amino acid substitutions shown in FIG. 33 of WO 2021/016571. For example, the Fc domain or FcRn-binding fragment thereof may comprise double amino acid substitutions M252Y/N434Y (YY); or triple amino acid substitutions selected from M252Y/T307W/N434Y (YWY), M252Y/T256D/N434Y (YDY), and T256D/307W/N434Y (DWY).

In some embodiments, the Fc domain or FcRn-binding fragment thereof may comprise a quadruple amino acid substitution selected from M252Y/T256D/T307Q/N434F (YDQF), M252Y/T256D/T307W/N434F (YDWF), M252Y/T256D/T307Q/N434Y (YDQY), M252Y/T256E/T307Q/N434Y (YEQY), M252Y/T256D/T307W/N434Y (YDWY), and M252Y/T256E/T307W/N434Y (YEWY).

In other embodiments, the Fc domain or FcRn-binding fragment thereof comprises a combination of the following four amino acid residues:
a) a tyrosine (Y) at amino acid position 252,
b) an aspartic acid (D) or a glutamic acid (E) at amino acid position 256,
c) a tryptophan (W) or a glutamine (Q) at amino acid position 307, and
d) a phenylalanine (F) or a tyrosine (Y) at amino acid position 434;
   according to Eu numbering.

In certain embodiments, the amino acid sequence of the Fc domain or FcRn-binding fragment thereof comprises or consists of the amino acid sequence set forth in SEQ ID NO: 25 with at least one of the above-recited amino acid substitutions.

In certain embodiments, the amino acid sequence of the Fc domains of the variant Fc region comprises or consists of the amino acid sequence set forth in SEQ ID NO: 26 with the recited amino acid substitutions.

In certain embodiments, the amino acid sequence of the Fc domains of the variant Fc region comprises or consists of the amino acid sequence set forth in SEQ ID NO: 27 with the recited amino acid substitutions.

In certain embodiments, the amino acid sequence of the Fc domains of the variant Fc region comprises or consists of the amino acid sequence set forth in SEQ ID NO: 61 with the recited amino acid substitutions.

Hence, if the Fc domain is in a dimeric form, i.e., comprises at least a hinge region or part of it, two CH2 domains, and two CH3 domains, i.e., two polypeptide fragments, the Fc domain may comprise two polypeptides comprising or consisting of the amino acid sequence set forth in SEQ ID NO: 25 with at least one of the above-recited amino acid substitutions. Hence, if the Fc domain comprises at least a hinge region or part of it, two CH2 domains, and two CH3 domains, i.e., two polypeptide fragments, the Fc domain may comprise two polypeptides comprising or consisting of the amino acid sequence set forth in SEQ ID NO: 26 with at least one of the above-recited amino acid substitutions. Hence, if the Fc domain comprises at least a hinge region or part of it, two CH2 domains, and two CH3 domains, i.e., two polypeptide fragments, the Fc domain may comprise two polypeptides comprising or consisting of the amino acid sequence set forth in SEQ ID NO: 27 with at least one of the above-recited amino acid substitutions. Hence, if the Fc domain comprises at least a hinge region or part of it, two CH2 domains, and two CH3 domains, i.e., two polypeptide fragments, the Fc domain may comprise two polypeptides comprising or consisting of the amino acid sequence set forth in SEQ ID NO: 61 with at least one of the above-recited amino acid substitutions. In these embodiments, at least one and preferably both of the CH3 domains comprised in the Fc domain comprised in the FcRn antagonists of the present invention may comprise knob in hole mutations, as defined above. Additionally or alternatively, the amino acids HY in the CH3 domains of Fc domain may be mutated into RF, such as at positions 435 and 436, (H435R and Y436F in CH3 domain, as described by Jendeberg, L. et al. (1997, J. Immunological Meth., 201:25-34)).

In other embodiments, the amino acid sequence of the Fc domain or FcRn-binding fragment thereof comprises or consists of the amino acid sequence set forth in any one of SEQ ID NOs: 25, 26, 27 or 61, with at least one of the above-recited amino acid substitutions and further comprising a linker sequence, e.g., as shown in Table 4, at the N- and/or C-terminal region of the sequence. In a preferred embodiment, the linker comprises or consists of SEQ ID NO: 41, 42, 43, 44 or 66. In a further preferred embodiment, the linker is comprised at the N-terminal region of the Fc domain or FcRn-binding fragment as set forth in any one of SEQ ID NOs: 25, 26, 27 or 61, with at least one of the above-recited amino acid substitutions. In a further preferred embodiment, the linker comprises or consists of SEQ ID NO: 66.

In other embodiments, the Fc domain or fragment thereof comprises a combination of the following four amino acid residues:
a) a tyrosine (Y) at amino acid position 252,
b) an aspartic acid (D) or a glutamic acid (E) at amino acid position 256,
c) a tryptophan (W) or a glutamine (Q) at amino acid position 307, and
d) a phenylalanine (F) or a tyrosine (Y) at amino acid position 434;
   according to Eu numbering.

In other embodiments, the Fc domain or fragment thereof comprises a combination of amino acid residues selected from the group consisting of:
a) a tyrosine (Y) at amino acid position 252, an aspartic acid (D) at amino acid position 256, a glutamine (Q) at amino acid position 307, and a tyrosine (Y) at amino acid position 434;
b) a tyrosine (Y) at amino acid position 252, a glutamic acid (E) at amino acid position 256, a tryptophan (W) at amino acid position 307, and a tyrosine (Y) at amino acid position 434;
c) a tyrosine (Y) at amino acid position 252, a glutamic acid (E) at amino acid position 256, a glutamine (Q) at amino acid position 307, and a tyrosine (Y) at amino acid position 434;
d) a tyrosine (Y) at amino acid position 252, an aspartic acid (D) at amino acid position 256, a glutamine (Q) at amino acid position 307, and a phenylalanine (F) at amino acid position 434;
e) a tyrosine (Y) at amino acid position 252, an aspartic acid (D) at amino acid position 256, a tryptophan (W) at amino acid position 307, and a tyrosine (Y) at amino acid position 434; and
f) a tyrosine (Y) at amino acid position 252, an aspartic acid (D) at amino acid position 256, a tryptophan (W) at amino acid position 307, and a phenylalanine (F) at amino acid position 434.

Hence, if the Fc domain is in a dimeric form, i.e., comprises or consists of at least a hinge region or part of it, two CH2 domains, and two CH3 domains, i.e., two polypeptide fragments, the Fc domain may comprise or consist of two polypeptides comprising or consisting of the amino acid sequence set forth in SEQ ID NO: 123. In these embodiments, at least one and preferably both of the CH3 domains comprised in the Fc domain comprised in the FcRn antagonists of the present invention may comprise knob in hole mutations, as defined above. Hence, if the Fc domain is in a dimeric form, i.e., comprises at least a hinge region or part of it, two CH2 domains, and two CH3 domains, i.e., two polypeptide fragments, the Fc domain may comprise or consist of two polypeptides comprising or consisting of the amino acid sequence set forth in SEQ ID NO: 121 and 122. Additionally, or alternatively, the amino acids HY in the CH3 domains of Fc domain may be mutated into RF, such as at positions 435 and 436, (H435R and Y436F in CH3 domain, as described by Jendeberg, L. et al. (1997, J. Immunological Meth., 201:25-34)).

In certain embodiments, the amino acid sequences of the Fc domains of the variant Fc region comprises or consists of the amino acid sequence set forth in SEQ ID NO: 121 and/or 122.

In certain embodiments, the amino acid sequence of the Fc domains of the variant Fc region comprises or consists of the amino acid sequence set forth in SEQ ID NO: 123.

In other embodiments, the amino acid sequence of the Fc domain or FcRn-binding fragment thereof comprises or consists of the amino acid sequence set forth in any one of SEQ ID NOs: 121-123, with at least one of the above-recited amino acid substitutions and further comprising a linker sequence, e.g., as shown in Table 4, at the N- and/or C-terminal region of the sequence. In a preferred embodiment, the linker comprises or consists of SEQ ID NO: 41, 42, 43, 44 or 66. In a further preferred embodiment, the linker is comprised at the N-terminal region of the Fc domain or FcRn-binding fragment as set forth in any one of SEQ ID NOs: 120-123. In certain embodiments, the Fc domain is a homodimer, wherein the amino acid sequence of each of the polypeptides comprised in the Fc domain comprises or consists of SEQ ID NO: 123. In certain embodiments, the Fc domain is a heterodimer, wherein the amino acid sequences of each of the polypeptides comprised in the Fc domain comprises or consists of SEQ ID NO: 121 and/or 122.

Hence, preferably, the FcRn antagonist of the present invention comprises two polypeptide fragments (chains). In one embodiment, the FcRn antagonist of the present invention is in a dimeric form, preferably in a heterodimeric form. One of the polypeptide chains or fragments may comprise at least a hinge region or a part of it, a CH2 domain, and a CH3 domain. The other polypeptide chain or fragment may comprise at least a hinge region or a part of it, a CH2 domain, and a CH3 domain, which may be the same or different as the other polypeptide chain or fragment, as described in detail above. For instance, one of the polypeptide chains or fragments may comprise SEQ ID NO.: 121, and the other polypeptide chain or fragment may comprise SEQ ID NO.: 122. For instance, one of the polypeptide chains or fragments may comprise SEQ ID NO.: 119, and the other polypeptide chain or fragment may comprise SEQ ID NO.: 120. For instance, both polypeptide chains or fragments may comprise SEQ ID NO.: 123. For instance, both polypeptide chains or fragments may comprise SEQ ID NO.: 23. One of the polypeptide chains or fragments may comprise, at its N- and/or C-terminus, covalently linked directly or by means of a linker, at least one first polypeptide (a) specifically binding to an epitope on FcRn.

In certain embodiments, the Fc domain may be mutated to decrease effector function using techniques known in the art. In some embodiments, the modified Fc herein also has altered binding affinity to an Fc-gamma receptors (FcγR). The FcγRs belong to a family that includes several members, e.g., FcγRI, FcγRIIa, FcγRIIb, FcγRIIIa, and FcγRIIIb. In some embodiments, the modified Fc herein, while having enhanced FcRn binding affinities, has reduced FcγRIIIa binding affinity, compared to a wildtype Fc domain. In certain embodiments, the variant Fc has increased affinity for FcγRIIIa, called CD 16a in this application.

FcRn Antagonists Comprising Fc Domain with YTE-KF Mutations

In one embodiment, the FcRn antagonist of the present invention comprises:
a) At least one first polypeptide specifically binding to an epitope on Fc receptor (FcRn); and
b) At least a second polypeptide which competes with wild-type IgG1 Fc region for binding to FcRn,
   wherein the at least one second polypeptide which competes with wild-type IgG1 Fc region for binding to FcRn comprises or consists of a Fc domain or a fragment thereof which comprises at least one, preferably all, of the following amino acids at the following positions
a) a tyrosine (Y) at amino acid position 252,
b) a threonine (T) at amino acid position 254,
c) a glutamic acid (E) at amino acid position 256,
d) a lysine (K) at amino acid position 433,
e) a phenylalanine (F) at amino acid position 434, and/or
f) a tyrosine (Y) at amino acid position 436;
   according to EU numbering.

In certain embodiments, the at least one second polypeptide which competes with wild-type IgG1 Fc region for binding to FcRn comprises or consists of a Fc domain which is a homodimer, wherein the amino acid sequence of each of the polypeptides comprised in the Fc domain consists of SEQ ID NO: 22. In certain embodiments, the Fc domain is a homodimer, wherein the amino acid sequence of each of the polypeptides comprised in the Fc domain consists of SEQ ID NO: 23. In certain embodiments, the Fc domain is a homodimer, wherein the amino acid sequence of each of the polypeptides comprised in the Fc domain consists of SEQ ID NO: 24. In certain embodiments, the Fc domain is a heterodimer, wherein the amino acid sequences of each of the polypeptides comprised in the Fc domain comprises or consists of SEQ ID NO: 119 and 120.

In certain embodiments, the amino acid sequence of the Fc domains of the variant Fc region comprises or consists of the amino acid sequence set forth in SEQ ID NO: 22 (CPPCPAPELLGGPSVFLFPPKPKDTLYITREPEVTCVVVDVSHEDPEVKFNWYVDGVEVHNAKTKPREEQ YNSTYRVVSVLTVLHQDWLNGKEYKCKVSNKALPAPIEKTISKAKGQPREPQVYTLPPSRDELTKNQVSLT CLVKGFYPSDIAVEWESNGQPENNYKTTPPVLDSDGSFFLYSKLTVDKSRWQQGNVFSCSVMHEALKFH YTQKSLSLSPG).

In certain embodiments, the amino acid sequence of the Fc domains of the variant Fc region comprises or consists of the amino acid sequence set forth in

SEQ ID NO: 23
(DKTHTCPPCPAPELLGGPSVFLFPPKPKDTLYITREPEVTCVVVDVSHE
DPEVKFNWYVDGVEVHNAKTKPREEQYNSTYRVVSVLTVLHQDWLNGKEY
KCKVSNKALPAPIEKTISKAKGQPREPQVYTLPPSRDELTKNQVSLTCLV
KGFYPSDIAVEWESNGQPENNYKTTPPVLDSDGSFFLYSKLTVDKSRWQQ
GNVFSCSVMHEALKFHYTQKSLSLSPGK).

In certain embodiments, the amino acid sequence of the Fc domains of the variant Fc region comprises or consists of the amino acid sequence set forth in

SEQ ID NO: 24
(DKTHTCPPCPAPELLGGPSVFLFPPKPKDTLYITREPEVTCVVVDVSHE
DPEVKFNWYVDGVEVHNAKTKPREEQYNSTYRVVSVLTVLHQDWLNGKEY
KCKVSNKALPAPIEKTISKAKGQPREPQVYTLPPSRDELTKNQVSLTCLV
KGFYPSDIAVEWESNGQPENNYKTTPPVLDSDGSFFLYSKLTVDKSRWQQ
GNVFSCSVMHEALKFHYTQKSLSLSPG).

In certain embodiments, the amino acid sequence of the Fc domains of the variant Fc region comprises or consists of the amino acid sequences set forth in SEQ ID NO: 119 and/or 120.

Sequences 119 and 120 are as follows:

| | SEQ | |
|---|---|---|
| Descrip- | ID | |
| tion | NO | Sequence |
| (YTEKF, | 119 | DKTHTCPPCPAPELLGGPSVFLFPPKPKDTLYITR |
| knob) | | EPEVTCVVVDVSHEDPEVKFNWYVDGVEVHNAKTK |
| | | PREEQYNSTYRVVSVLTVLHQDWLNGKEYKCKVSN |
| | | KALPAPIEKTISKAKGQPREPQVYTLPPCRDELTK |
| | | NQVSLWCLVKGFYPSDIAVEWESNGQPENNYKTTP |
| | | PVLDSDGSFFLYSKLTVDKSRWQQGNVFSCSVMHE |
| | | ALKFHYTQKSLSLSPG |
| (YTEKF, | 120 | DKTHTCPPCPAPELLGGPSVFLFPPKPKDTLYITR |
| hole) | | EPEVTCVVVDVSHEDPEVKFNWYVDGVEVHNAKTK |
| | | PREEQYNSTYRVVSVLTVLHQDWLNGKEYKCKVSN |
| | | KALPAPIEKTISKAKGQPREPQVCTLPPSRDELTK |
| | | NQVSLSCAVKGFYPSDIAVEWESNGQPENNYKTTP |
| | | PVLDSDGSFFLVSKLTVDKSRWQQGNVFSCSVMHE |
| | | ALKFHYTQKSLSLSPG |

In this embodiment, the at least one first polypeptide (a) can be any polypeptide as described herein, see, e.g., section “The first polypeptide (a) comprised in the FcRn antagonists of the present invention”.

The Fc domain or fragment thereof as described above may be covalently linked directly or by means of a linker to the at least one first polypeptide (a) specifically binding to an epitope on FcRn. In one embodiment, the Fc domain or fragment thereof as described above is covalently linked by means of a linker to the at least one first polypeptide (a) specifically binding to an epitope on FcRn. The linker may be one as shown in Table 4. In a preferred embodiment, the linker is a 35GS linker (see, e.g., SEQ ID NO.: 39) or a hinge linker, such as a short G1 hinge linker (e.g., SEQ ID NO.: 66), which may be part of the Fc domain. The Fc domain or fragment thereof as described above may be covalently linked directly or by means of a linker to the at least one first polypeptide (a) at the N- or C-terminal region of the Fc domain or fragment thereof as described above.

Fc domains with the above amino acids (YTE-KF) have been described above in this description and equally apply to the present embodiments.

In one embodiment, the at least one first polypeptide (a) specifically binding to an epitope on FcRn does not compete with wild-type IgG1 Fc region for binding to FcRn. Preferably, the at least one first polypeptide (a) of this embodiment comprises or consists of an ISVD, as defined herein. Hence, in a preferred embodiment, the FcRn antagonists of the present invention comprise:
a) at least one ISVD specifically binding to an epitope on Fc receptor (FcRn), wherein the ISVD does not compete with wild-type IgG1 Fc region for binding to FcRn, as described herein; and
b) at least one polypeptide which competes with wild-type IgG1 Fc region for binding to FcRn, wherein the at least one second polypeptide which competes with wild-type IgG1 Fc region for binding to FcRn is a Fc domain or a fragment thereof which comprises at least one, preferably all, of the following amino acids at the following positions
   a) a tyrosine (Y) at amino acid position 252,
   b) a threonine (T) at amino acid position 254,
   c) a glutamic acid (E) at amino acid position 256,
   d) a lysine (K) at amino acid position 433,
   e) a phenylalanine (F) at amino acid position 434, and/or
   f) a tyrosine (Y) at amino acid position 436;
   according to EU numbering,
      preferably wherein the at least one second polypeptide which competes with wild-type IgG1 Fc region for binding to FcRn comprises or consists of a Fc domain which is a homodimer, wherein the amino acid sequence of each of the polypeptides comprised in the Fc domain consists of SEQ ID NO: 22, SEQ ID NO: 23 or SEQ ID NO: 24, or wherein the Fc domain is a heterodimer, wherein the amino acid sequences of each of the polypeptides comprised in the Fc domain comprises or consists of SEQ ID NO: 119 and 120.

The Fc domain or fragment thereof as described above may be covalently linked directly or by means of a linker to the at least one first polypeptide (a) specifically binding to an epitope on FcRn. In one embodiment, the Fc domain or fragment thereof as described above is covalently linked by means of a linker to the at least one first polypeptide (a) specifically binding to an epitope on FcRn. The linker may be one as shown in Table 4. In a preferred embodiment, the linker is a 35GS linker (see, e.g., SEQ ID NO.: 39) or a hinge linker, such as a short G1 hinge linker (e.g., SEQ ID NO.: 66), which may be part of the Fc domain. The Fc domain or fragment thereof as described above may be covalently linked directly or by means of a linker to the at least one first polypeptide (a) at the N- or C-terminal region of the Fc domain or fragment thereof as described above.

In one embodiment, the at least one first polypeptide (a) specifically binding to an epitope on the Fc receptor (FcRn) comprised in the FcRn antagonists of the present invention is an ISVD that comprises 4 framework regions (FR1 to FR4, respectively) and three complementarity determining regions (CDR1 to CDR3, respectively), wherein the ISVD comprises a CDR1 (AbM numbering) that is the amino acid sequence of SEQ ID NO: 11, a CDR2 (AbM numbering) that is the amino acid sequence of SEQ ID NO: 12 and a CDR3 (AbM numbering) that is the amino acid sequence of SEQ ID NO: 13.

If the CDR definitions according to Kabat are used, then in this embodiment the at least one first polypeptide (a) specifically binding to an epitope on the Fc receptor (FcRn) comprised in the FcRn antagonists of the present invention is an ISVD that comprises 4 framework regions (FR1 to FR4, respectively) and three complementarity determining regions (CDR1 to CDR3, respectively), wherein the ISVD comprises a CDR1 (Kabat numbering) that is the amino acid sequence of SEQ ID NO: 8, a CDR2 (Kabat numbering) that is the amino acid sequence of SEQ ID NO: 9 and a CDR3 (Kabat numbering) that is the amino acid sequence of SEQ ID NO: 10.

In a preferred embodiment, the at least one first polypeptide (a) specifically binding to an epitope on the Fc receptor (FcRn) comprised in the FcRn antagonists of the present invention is an ISVD which comprises or consists of SEQ ID NO.: 15. If polypeptide (a) is located at the N-terminus of the FcRn antagonist, then the ISVD may comprise a E1D mutation. In this particular case, the ISVD preferably comprises or consists of SEQ ID NO.: 185.

In a preferred embodiment, the FcRn antagonists of the present invention comprise:
a) at least one immunoglobulin single variable domain (ISVD) specifically binding to an epitope on Fc receptor (FcRn), wherein the ISVD does not compete with wild-type IgG1 Fc region for binding to FcRn, preferably wherein the ISVD comprises or consist of T0263018B11(E1D,W100cS) (SEQ ID NO.: 185) or T0263018B11(W100cS) (SEQ ID NO.: 15); and
b) at least one polypeptide which competes with wild-type IgG1 Fc region for binding to FcRn, wherein the at least one second polypeptide which competes with wild-type IgG1 Fc region for binding to FcRn is a Fc domain or a fragment thereof which comprises at least one, preferably all, of the following amino acids at the following positions:
   a) a tyrosine (Y) at amino acid position 252,
   b) a threonine (T) at amino acid position 254,
   c) a glutamic acid (E) at amino acid position 256,
   d) a lysine (K) at amino acid position 433,
   e) a phenylalanine (F) at amino acid position 434, and/or
   f) a tyrosine (Y) at amino acid position 436;
   according to EU numbering,
      preferably wherein the at least one second polypeptide which competes with wild-type IgG1 Fc region for binding to FcRn comprises or consists of a Fc domain which is a heterodimer, wherein the amino acid sequences of each of the polypeptides comprised in the Fc domain comprises or consists of SEQ ID NO: 119 and 120.

SEQ ID NO.: 15 (T0263018B11-W100cS) is as follows:
EVQLVESGGGVVQPGGSLRLSCAASGFTFSSYAMYWVRQAPGKGLEWVSA
ISSGGGSTDYADSVKGRFTISRDNSKNTLYLQMNSLRPEDTALYYCAADT
LYTSLTSYSYWGQGTLVTVSS
SEQ ID NO.: 185 (T0263018B11(E1D, W100cS)) is as
follows:
DVQLVESGGGVVQPGGSLRLSCAASGFTFSSYAMYWVRQAPGKGLEWVSA
ISSGGGSTDYADSVKGRFTISRDNSKNTLYLQMNSLRPEDTALYYCAADT
LYTSLTSYSYWGQGTLVTVSS

In one embodiment, the FcRn antagonist of the present invention comprises two polypeptide fragments (chains). Hence, in one embodiment, the FcRn antagonist of the present invention is in a dimeric form, preferably in a heterodimeric form. One of the polypeptide chains or fragments may comprise at least a hinge region or a part of it, a CH2 domain, and a CH3 domain. The other polypeptide chain or fragment may comprise at least a hinge region or a part of it, a CH2 domain, and a CH3 domain. For instance, one of the polypeptide chains or fragments may comprise SEQ ID NO.: 119, and the other polypeptide chain or fragment may comprise SEQ ID NO.: 120. One of the polypeptide chains or fragments may comprise, at its N- and/or C-terminus, covalently linked directly or by means of a linker, at least one first polypeptide (a) specifically binding to an epitope on FcRn, as described herein. For instance, one of the polypeptide chains or fragments may comprise, at its N- and/or C-terminus, covalently linked directly or by means of a linker, at least one ISVD specifically binding to an epitope on Fc receptor (FcRn), wherein the ISVD does not compete with wild-type IgG1 Fc region for binding to FcRn, preferably wherein the ISVD comprises or consist of T0263018B11(E1D,W100cS) (SEQ ID NO.: 185), or wherein the ISVD comprises or consist of T0263018B11(W100cS) (SEQ ID NO.: 15).

In another embodiment, one of the polypeptide chains or fragments may comprise, at its N-terminus, covalently linked directly or by means of a linker, preferably directly linked or linked by means of an IgG1 hinge, see Table 4, e.g., SEQ ID NO.: 66, which may be part of the Fc domain, at least one ISVD specifically binding to an epitope on Fc receptor (FcRn), wherein the ISVD does not compete with wild-type IgG1 Fc region for binding to FcRn, preferably wherein the ISVD comprises or consist of T0263018B11(E1D,W100cS) (SEQ ID NO.: 185).

For instance, in another embodiment, the FcRn antagonist is heterodimeric and it comprises or consists of:
one polypeptide chain or fragment which comprises a N-terminal ISVD specifically binding to an epitope on Fc receptor (FcRn), wherein the ISVD does not compete with wild-type IgG1 Fc region for binding to FcRn, preferably wherein the ISVD comprises or consist of T0263018B11(E1D,W100cS), defined by SEQ ID NO.: 185, followed by a C-terminal polypeptide defined by SEQ ID NO.: 119; and
a second polypeptide chain or fragment which comprises or consists of SEQ ID NO.: 120.

In another embodiment, one of the polypeptide chains or fragments may comprise, at its C-terminus, covalently linked by means of a linker, preferably a 35GS linker as defined herein, at least one ISVD specifically binding to an epitope on Fc receptor (FcRn), wherein the ISVD does not compete with wild-type IgG1 Fc region for binding to FcRn, preferably wherein the ISVD comprises or consist of T0263018B11(W100cS) (SEQ ID NO.: 15).

For instance, in one embodiment, the FcRn antagonist is heterodimeric and it comprises or consists of:
one polypeptide chain or fragment which comprises a N-terminal polypeptide defined by SEQ ID NO.: 119, followed by a 35GS linker (SEQ ID NO.: 39) and a C-terminal ISVD specifically binding to an epitope on Fc receptor (FcRn), wherein the ISVD does not compete with wild-type IgG1 Fc region for binding to FcRn, preferably wherein the ISVD comprises or consist of T0263018B11(W100cS) (SEQ ID NO.: 15); and
a second polypeptide chain or fragment which comprises or consists of SEQ ID NO.: 120.

In a preferred embodiment, the FcRn antagonist is TP067, which comprises two chains:
Chain 1: T0263018B11(E1D,W100cS) (SEQ ID NO.: 185)-human IgG1 Fc YTE-KF (SEQ ID NO.: 119); and
Chain 2: human IgG1 Fc YTE-KF (SEQ ID NO.: 120).

In another preferred embodiment, the FcRn antagonist is TP069, which comprises two chains:
Chain 1: human IgG1 Fc YTE-KF (SEQ ID NO.: 119)-35GS-T0263018B11(W100cS) (SEQ ID NO.: 15); and
Chain 2: human IgG1 Fc YTE-KF (SEQ ID NO.: 120).
   FcRn Antagonists Comprising Fc Domain with YDQY Mutations

In one embodiment, the FcRn antagonist of the present invention comprises:
a) At least one first polypeptide specifically binding to an epitope on Fc receptor (FcRn); and
b) At least a second polypeptide which competes with wild-type IgG1 Fc region for binding to FcRn,
   wherein the at least one second polypeptide which competes with wild-type IgG1 Fc region for binding to FcRn comprises or consists of a Fc domain or a fragment thereof which comprises a quadruple amino acid substitution selected from M252Y/T256D/T307Q/N434F (YDQF), M252Y/T256D/T307W/N434F (YDWF), M252Y/T256D/T307Q/N434Y (YDQY), M252Y/T256E/T307Q/N434Y (YEQY), M252Y/T256D/T307W/N434Y (YDWY), and M252Y/T256E/T307W/N434Y (YEWY). Preferably, the at least one second polypeptide which competes with wild-type IgG1 Fc region for binding to FcRn comprises the quadruple amino acid substitution M252Y/T256D/T307Q/N434Y (YDQY). Even more preferably, the Fc domain or a fragment thereof comprises or consists of SEQ ID NO: 121-123, preferably comprises or consists of SEQ ID NO: 123 for a homodimer Fc-domain or comprises or consists of 121 and 122 for a heterodimeric Fc-domain.

SEQ ID NO.: 121-123 are as follows (IgG1 Hinge underlined):

| | SEQ | |
|---|---|---|
| | ID | |
| Description | NO | Sequence |
| Fc regions | 121 | DKTHTCPPCPAPELLGGPSVFLFPPKPKDTLY |
| (YDQY, | | ISRDPEVTCVVVDVSHEDPEVKFNWYVDGVEV |
| knob) | | HNAKTKPREEQYNSTYRVVSVLQVLHQDWLNG |
| | | KEYKCKVSNKALPAPIEKTISKAKGQPREPQV |
| | | YTLPPCRDELTKNQVSLWCLVKGFYPSDIAVE |
| | | WESNGQPENNYKTTPPVLDSDGSFFLYSKLTV |
| | | DKSRWQQGNVFSCSVMHEALHYHYTQKSLSLS |
| | | PG |
| Fc regions | 122 | DKTHTCPPCPAPELLGGPSVFLFPPKPKDTLY |
| (YDQY, | | ISRDPEVTCVVVDVSHEDPEVKFNWYVDGVEV |
| hole) | | HNAKTKPREEQYNSTYRVVSVLQVLHQDWLNG |
| | | KEYKCKVSNKALPAPIEKTISKAKGQPREPQV |
| | | CTLPPSRDELTKNQVSLSCAVKGFYPSDIAVE |
| | | WESNGQPENNYKTTPPVLDSDGSFFLVSKLTV |
| | | DKSRWQQGNVFSCSVMHEALHYHYTQKSLSLS |
| | | PG |
| Fc regions | 123 | DKTHTCPPCPAPELLGGPSVFLFPPKPKDTLY |
| (YDQY, | | ISRDPEVTCVVVDVSHEDPEVKFNWYVDGVEV |
| no KIH) | | HNAKTKPREEQYNSTYRVVSVLQVLHQDWLNG |
| | | KEYKCKVSNKALPAPIEKTISKAKGQPREPQV |
| | | YTLPPSRDELTKNQVSLTCLVKGFYPSDIAVE |
| | | WESNGQPENNYKTTPPVLDSDGSFFLYSKLTV |
| | | DKSRWQQGNVFSCSVMHEALHYHYTQKSLSLS |
| | | PG |

In this embodiment, the at least one first polypeptide (a) can be any polypeptide as described herein, see, e.g., section “The first polypeptide (a) comprised in the FcRn antagonists of the present invention”. The second polypeptide (b) which competes with wild-type IgG1 Fc region for binding to FcRn comprises or consists of a Fc domain or a fragment thereof which comprises a quadruple amino acid substitution selected from M252Y/T256D/T307Q/N434F (YDQF), M252Y/T256D/T307W/N434F (YDWF), M252Y/T256D/T307Q/N434Y (YDQY), M252Y/T256E/T307Q/N434Y (YEQY), M252Y/T256D/T307W/N434Y (YDWY), and M252Y/T256E/T307W/N434Y (YEWY), preferably the quadruple amino acid substitution M252Y/T256D/T307Q/N434Y (YDQY), more preferably the Fc domain or a fragment thereof comprises or consists of SEQ ID NO: 121-123, preferably comprises or consists of SEQ ID NO: 123 for a homodimer Fc-domain or comprises or consists of 121 and 122 for a heterodimeric Fc-domain.

The Fc domain or fragment thereof as described above may be covalently linked directly or by means of a linker to the at least one first polypeptide (a) specifically binding to an epitope on FcRn. In one embodiment, the Fc domain or fragment thereof as described above is covalently linked by means of a linker to the at least one first polypeptide (a) specifically binding to an epitope on FcRn. The linker may be one as shown in Table 4. In a preferred embodiment, the linker is a 35GS linker (see, e.g., SEQ ID NO.: 39). The Fc domain or fragment thereof as described above may be covalently linked directly or by means of a linker to the at least one first polypeptide (a) at the N- or C-terminal region of the Fc domain or fragment thereof as described above.

The at least one first polypeptide (a) specifically binding to an epitope on FcRn comprised in the FcRn antagonist of this embodiment may be any polypeptide as described herein, see, e.g., section “The first polypeptide (a) comprised in the FcRn antagonists of the present invention”.

In one embodiment, the at least one first polypeptide (a) specifically binding to an epitope on FcRn does not compete with wild-type IgG1 Fc region for binding to FcRn. Preferably, the at least one first polypeptide (a) of this embodiment comprises or consists of an ISVD, as defined herein. Hence, in a preferred embodiment, the FcRn antagonists of the present invention comprise:
a) at least one immunoglobulin single variable domain (ISVD) specifically binding to an epitope on Fc receptor (FcRn), wherein the ISVD does not compete with wild-type IgG1 Fc region for binding to FcRn, as described herein; and
b) at least one polypeptide which competes with wild-type IgG1 Fc region for binding to FcRn, wherein the at least one second polypeptide which competes with wild-type IgG1 Fc region for binding to FcRn is a Fc domain or a fragment thereof which comprises a quadruple amino acid substitution selected from M252Y/T256D/T307Q/N434F (YDQF), M252Y/T256D/T307W/N434F (YDWF), M252Y/T256D/T307Q/N434Y (YDQY), M252Y/T256E/T307Q/N434Y (YEQY), M252Y/T256D/T307W/N434Y (YDWY), and M252Y/T256E/T307W/N434Y (YEWY), preferably the quadruple amino acid substitution M252Y/T256D/T307Q/N434Y (YDQY), more preferably the Fc domain or a fragment thereof comprises or consists of SEQ ID NO: 121-123, preferably comprises or consists of SEQ ID NO: 123 for a homodimer Fc-domain or comprises or consists of 121 and 122 for a heterodimeric Fc-domain.

In one embodiment, the at least one first polypeptide (a) specifically binding to an epitope on the Fc receptor (FcRn) comprised in the FcRn antagonists of the present invention is an ISVD that comprises 4 framework regions (FR1 to FR4, respectively) and three complementarity determining regions (CDR1 to CDR3, respectively), wherein the ISVD comprises a CDR1 (AbM numbering) that is the amino acid sequence of SEQ ID NO: 11, a CDR2 (AbM numbering) that is the amino acid sequence of SEQ ID NO: 12 and a CDR3 (AbM numbering) that is the amino acid sequence of SEQ ID NO: 189.

If the CDR definitions according to Kabat are used, then in this embodiment the at least one first polypeptide (a) specifically binding to an epitope on the Fc receptor (FcRn) comprised in the FcRn antagonists of the present invention is an ISVD that comprises 4 framework regions (FR1 to FR4, respectively) and three complementarity determining regions (CDR1 to CDR3, respectively), wherein the ISVD comprises a CDR1 (Kabat numbering) that is the amino acid sequence of SEQ ID NO: 8, a CDR2 (Kabat numbering) that is the amino acid sequence of SEQ ID NO: 9 and a CDR3 (Kabat numbering) that is the amino acid sequence of SEQ ID NO: 189.

In a preferred embodiment, the at least one first polypeptide (a) specifically binding to an epitope on the Fc receptor (FcRn) comprised in the FcRn antagonists of the present invention is an ISVD which comprises or consists of SEQ ID NO.: 186. If polypeptide (a) is located at the N-terminus of the FcRn antagonist, then the ISVD may comprise a E1D mutation. In this particular case, the ISVD preferably comprises or consists of SEQ ID NO.: 187.

In another embodiment, the at least one first polypeptide (a) specifically binding to an epitope on the Fc receptor (FcRn) comprised in the FcRn antagonists of the present invention is an ISVD that comprises 4 framework regions (FR1 to FR4, respectively) and three complementarity determining regions (CDR1 to CDR3, respectively), wherein the ISVD comprises a CDR1 (AbM numbering) that is the amino acid sequence of SEQ ID NO: 128, a CDR2 (AbM numbering) that is the amino acid sequence of SEQ ID NO: 129 and a CDR3 (AbM numbering) that is the amino acid sequence of SEQ ID NO: 130.

If the CDR definitions according to Kabat are used, then in this embodiment the at least one first polypeptide (a) specifically binding to an epitope on the Fc receptor (FcRn) comprised in the FcRn antagonists of the present invention is an ISVD that comprises 4 framework regions (FR1 to FR4, respectively) and three complementarity determining regions (CDR1 to CDR3, respectively), wherein the ISVD comprises a CDR1 (Kabat numbering) that is the amino acid sequence of SEQ ID NO: 136, a CDR2 (Kabat numbering) that is the amino acid sequence of SEQ ID NO: 137 and a CDR3 (Kabat numbering) that is the amino acid sequence of SEQ ID NO: 130.

In a preferred embodiment, the at least one first polypeptide (a) specifically binding to an epitope on the Fc receptor (FcRn) comprised in the FcRn antagonists of the present invention is an ISVD which comprises or consists of SEQ ID NO.: 131. If polypeptide (a) is located at the N-terminus of the FcRn antagonist, then the ISVD may comprise a E1D mutation.

In another embodiment, the at least one first polypeptide (a) specifically binding to an epitope on the Fc receptor (FcRn) comprised in the FcRn antagonists of the present invention is an ISVD that comprises 4 framework regions (FR1 to FR4, respectively) and three complementarity determining regions (CDR1 to CDR3, respectively), wherein the ISVD comprises a CDR1 (AbM numbering) that is the amino acid sequence of SEQ ID NO: 141, a CDR2 (AbM numbering) that is the amino acid sequence of SEQ ID NO: 142 and a CDR3 (AbM numbering) that is the amino acid sequence of SEQ ID NO: 143.

If the CDR definitions according to Kabat are used, then in this embodiment the at least one first polypeptide (a) specifically binding to an epitope on the Fc receptor (FcRn) comprised in the FcRn antagonists of the present invention is an ISVD that comprises 4 framework regions (FR1 to FR4, respectively) and three complementarity determining regions (CDR1 to CDR3, respectively), wherein the ISVD comprises a CDR1 (Kabat numbering) that is the amino acid sequence of SEQ ID NO: 145, a CDR2 (Kabat numbering) that is the amino acid sequence of SEQ ID NO: 146 and a CDR3 (Kabat numbering) that is the amino acid sequence of SEQ ID NO: 143.

In a preferred embodiment, the at least one first polypeptide (a) specifically binding to an epitope on the Fc receptor (FcRn) comprised in the FcRn antagonists of the present invention is an ISVD which comprises or consists of SEQ ID NO.: 144. If polypeptide (a) is located at the N-terminus of the FcRn antagonist, then the ISVD may comprise a E1D mutation.

In a preferred embodiment, the FcRn antagonists of the present invention comprise:
a) at least one immunoglobulin single variable domain (ISVD) specifically binding to an epitope on Fc receptor (FcRn), wherein the ISVD does not compete with wild-type IgG1 Fc region for binding to FcRn, preferably wherein the ISVD comprises or consist of T0263018B11(Y79S,T100bE,W100cS) (SEQ ID NO.: 186), or wherein the ISVD comprises or consist of T0263091D07 (SEQ ID NO: 131), or wherein the ISVD comprises or consist of T0263204B12 (SEQ ID NO: 144); and
b) at least one polypeptide which competes with wild-type IgG1 Fc region for binding to FcRn, wherein the at least one second polypeptide which competes with wild-type IgG1 Fc region for binding to FcRn is a Fc domain or a fragment thereof which comprises a quadruple amino acid substitution selected from M252Y/T256D/T307Q/N434F (YDQF), M252Y/T256D/T307W/N434F (YDWF), M252Y/T256D/T307Q/N434Y (YDQY), M252Y/T256E/T307Q/N434Y (YEQY), M252Y/T256D/T307W/N434Y (YDWY), and M252Y/T256E/T307W/N434Y (YEWY), preferably the quadruple amino acid substitution M252Y/T256D/T307Q/N434Y (YDQY), more preferably the Fc domain or a fragment thereof comprises or consists of SEQ ID NO: 121-123, preferably comprises or consists of SEQ ID NO: 123 for a homodimer Fc-domain or comprises or consists of 121 and 122 for a heterodimeric Fc-domain.

SEQ ID NO.: 186 (T0263018B11
(Y79S, T100bE, W100cS)) is as follows:
EVQLVESGGGVVQPGGSLRLSCAASGFTFSSYAMYWVRQAPGKGL
EWVSAISSGGGSTDYADSVKGRFTISRDNSKNTLSLQMNSLRPED
TALYYCAADTLYTSLESYSYWGQGTLVTVSS

If the ISVD T0263018B11(Y79S,T100bE,W100cS) is located at the N-terminus of a polypeptide, it may preferably comprise a E1D mutation with respect to SEQ ID NO.: 186, see SEQ ID NO.: 187: DVQLVESGGGVVQPGGSLRLSCAASGFTFSSYAMYWVRQAPGKGLEWVSAISSGGGSTDYADSVKGRF TISRDNSKNTLSLQMNSLRPEDTALYYCAADTLYTSLESYSYWGQGTLVTVSS

SEQ ID NO.: 131 (T0263091D07) is as follows:
EVQLVESGGGVVQPGGSLRLSCAASGFSFSDYYMYWVRQAPGKGL
EWVSAISSGGSSTYYADSVKGRFTISRDNSKNTVYLQMNSLRPED
TALYYCAADYLSVPDPSYEYWGQGTLVTVSS
SEQ ID NO.: 144 (T0263204B12) is as follows:
EVQLVESGGGLVQPGGSLRLSCAASGFTLDYYAIGWFRQAPGKER
EGVLCISSSGDSTYYADSVKGRFTISRDNAKNTVYLQMDSLNPED
TAVYYCAVDPPSYWTGTGCLYGYRYWGQGTLVTVSS

In one embodiment, the FcRn antagonist of the present invention comprises two polypeptide fragments (chains). Hence, in one embodiment, the FcRn antagonist of the present invention is in a dimeric form, preferably in a heterodimeric form. One of the polypeptide chains or fragments may comprise at least a hinge region or a part of it, a CH2 domain, and a CH3 domain. The other polypeptide chain or fragment may comprise at least a hinge region or a part of it, a CH2 domain, and a CH3 domain. For instance, one of the polypeptide chains or fragments may comprise SEQ ID NO.: 121, and the other polypeptide chain or fragment may comprise SEQ ID NO.: 122. One of the polypeptide chains or fragments may comprise, at its N- and/or C-terminus, covalently linked directly or by means of a linker, at least one first polypeptide (a) specifically binding to an epitope on FcRn, as described herein. For instance, one of the polypeptide chains or fragments may comprise, at its N- and/or C-terminus, covalently linked directly or by means of a linker, at least one ISVD specifically binding to an epitope on Fc receptor (FcRn), wherein the ISVD does not compete with wild-type IgG1 Fc region for binding to FcRn, preferably wherein the ISVD comprises or consist of T0263018B11(Y79S,T100bE,W100cS) (SEQ ID NO.: 186), or wherein the ISVD comprises or consist of T0263091D07 (SEQ ID NO: 131), or wherein the ISVD comprises or consists of T0263204B12 (SEQ ID NO: 144).

In one embodiment, one of the polypeptide chains or fragments may comprise, at its C-terminus, covalently linked by means of a linker, preferably a 35GS linker as defined herein, at least one ISVD specifically binding to an epitope on Fc receptor (FcRn), wherein the ISVD does not compete with wild-type IgG1 Fc region for binding to FcRn, preferably wherein the ISVD comprises or consist of T0263018B11(Y79S,T100bE,W100cS) (SEQ ID NO.: 186), or wherein the ISVD comprises or consist of T0263091D07 (SEQ ID NO: 131), or wherein the ISVD comprises or consists of T0263204B12 (SEQ ID NO: 144).

In another embodiment, one of the polypeptide chains or fragments may comprise, at its N-terminus, covalently linked directly or by means of a linker, preferably directly linked or linked by means of an IgG1 hinge, see Table 4, e.g., SEQ ID NO.: 66, which may be part of the Fc domain, at least one ISVD specifically binding to an epitope on Fc receptor (FcRn), wherein the ISVD does not compete with wild-type IgG1 Fc region for binding to FcRn, preferably wherein the ISVD comprises or consist of T0263018B11(Y79S,T100bE,W100cS) (SEQ ID NO.: 186).

For instance, in one embodiment, the FcRn antagonist is heterodimeric and it comprises or consists of:
one polypeptide chain or fragment which comprises a N-terminal polypeptide defined by SEQ ID NO.: 121, followed by a 35GS linker (SEQ ID NO.: 39) and a C-terminal ISVD specifically binding to an epitope on Fc receptor (FcRn), wherein the ISVD does not compete with wild-type IgG1 Fc region for binding to FcRn, preferably wherein the ISVD comprises or consist of T0263018B11(Y79S,T100bE,W100cS) (SEQ ID NO.: 186); and
a second polypeptide chain or fragment which comprises or consists of SEQ ID NO.: 122.

For instance, in another embodiment, the FcRn antagonist is heterodimeric and it comprises or consists of:
one polypeptide chain or fragment which comprises a N-terminal polypeptide defined by SEQ ID NO.: 121, followed by a 35GS linker (SEQ ID NO.: 39) and a C-terminal ISVD specifically binding to an epitope on Fc receptor (FcRn), wherein the ISVD does not compete with wild-type IgG1 Fc region for binding to FcRn, preferably wherein the ISVD comprises or consist of T0263091D07 (SEQ ID NO: 131); and
a second polypeptide chain or fragment which comprises or consists of SEQ ID NO.: 122.

For instance, in another embodiment, the FcRn antagonist is heterodimeric and it comprises or consists of:
one polypeptide chain or fragment which comprises a N-terminal polypeptide defined by SEQ ID NO.: 121, followed by a 35GS linker (SEQ ID NO.: 39) and a C-terminal ISVD specifically binding to an epitope on Fc receptor (FcRn), wherein the ISVD does not compete with wild-type IgG1 Fc region for binding to FcRn, preferably wherein the ISVD comprises or consist of T0263204B12 (SEQ ID NO: 144); and
a second polypeptide chain or fragment which comprises or consists of SEQ ID NO.: 122.

For instance, in another embodiment, the FcRn antagonist is heterodimeric and it comprises or consists of:
one polypeptide chain or fragment which comprises a N-terminal ISVD specifically binding to an epitope on Fc receptor (FcRn), wherein the ISVD does not compete with wild-type IgG1 Fc region for binding to FcRn, preferably wherein the ISVD comprises or consist of T0263018B11(Y79S,T100bE,W100cS) E1D, defined by SEQ ID NO.: 187, followed by a C-terminal polypeptide defined by SEQ ID NO.: 121; and
a second polypeptide chain or fragment which comprises or consists of SEQ ID NO.: 122.

In a preferred embodiment, the first and/or the second polypeptide chain or fragment as defined above comprises a C-terminal alanine. The linker may be one as shown in Table 4. In a preferred embodiment, the linker is a 35GS linker (see, e.g., SEQ ID NO.: 39).

In a preferred embodiment, the FcRn antagonist is TPP-122907, which comprises two chains:
Chain 1: human IgG1 Fc YDQY (SEQ ID NO.: 121)-35GS-T0263018B11(Y79S,T100bE,W100cS) (SEQ ID NO.:186)-A; and
Chain 2: human IgG1 Fc YDQY (SEQ ID NO.: 122).

In another preferred embodiment, the FcRn antagonist is TPP-122908, which comprises two chains:
Chain 1: human IgG1 FcYDQY (SEQ ID NO.: 121)-35GS-T0263091D07 (SEQ ID NO.:131)-A; and
Chain 2: human IgG1 Fc YDQY (SEQ ID NO.: 122).

In another preferred embodiment, the FcRn antagonist is TPP-122909, which comprises two chains:
Chain 1: human IgG1 FcYDQY (SEQ ID NO.: 121)-35GS-T0263204B12 (SEQ ID NO.:144)-A; and
Chain 2: human IgG1 Fc YDQY (SEQ ID NO.: 122).

In another preferred embodiment, the FcRn antagonist is TPP-122905, which comprises two chains:
Chain 1: T0263018B11(E1D,Y79S,T100bE,W100cS) (SEQ ID NO.: 187)-human IgG1 Fc YDQY (SEQ ID NO.: 121); and
Chain 2: human IgG1 Fc YDQY (SEQ ID NO.: 122).

In another embodiment, the FcRn antagonist of the present invention comprises:
a) At least one first polypeptide specifically binding to an epitope on Fc receptor (FcRn), as defined herein;
b) At least a second polypeptide which competes with wild-type IgG1 Fc region for binding to FcRn; and
c) At least one further group, residue, moiety or binding unit, optionally linked via one or more linkers, such as peptide linkers, as defined above, in which said one or more other groups, residues, moieties or binding units provide the antagonist of the present technology with increased (in vivo) half-life, compared to the corresponding antagonist without said one or more other groups, residues, moieties or binding units (“(in vivo) half-life extending moiety”, or “half-life extending (HLE) moiety”), as described herein,
   wherein the at least one second polypeptide which competes with wild-type IgG1 Fc region for binding to FcRn comprises or consists of a Fc domain or a fragment thereof which comprises a quadruple amino acid substitution selected from M252Y/T256D/T307Q/N434F (YDQF), M252Y/T256D/T307W/N434F (YDWF), M252Y/T256D/T307Q/N434Y (YDQY), M252Y/T256E/T307Q/N434Y (YEQY), M252Y/T256D/T307W/N434Y (YDWY), and M252Y/T256E/T307W/N434Y (YEWY), preferably the quadruple amino acid substitution M252Y/T256D/T307Q/N434Y (YDQY), more preferably the Fc domain or a fragment thereof comprises or consists of SEQ ID NO: 121-123, preferably comprises or consists of SEQ ID NO: 123 for a homodimer Fc-domain or comprises or consists of 121 and 122 for a heterodimeric Fc-domain.

In one embodiment, the at least one further group, residue, moiety or binding unit (c) is an ISVD, for instance a human serum albumin (HSA)-binding ISVD, as described herein. Preferably, the at least one further group, residue, moiety or binding unit (c) is an ISVD that binds to human serum albumin and comprises a CDR1 (Abm) that is the amino acid sequence of SEQ ID NO: 19, a CDR2 (Abm) that is the amino acid sequence of SEQ ID NO: 20 and a CDR3 (Abm) that is the amino acid sequence of SEQ ID NO: 21, or, using the Kabat definition, a CDR1 (Kabat) that is the amino acid sequence of SEQ ID NO: 16, a CDR2 (Kabat) that is the amino acid sequence of SEQ ID NO: 17 and a CDR3 (Kabat) that is the amino acid sequence of SEQ ID NO: 18. Preferably, the ISVD that binds to human serum albumin can be selected from the ISVDs of Table 5. More preferably, the ISVD comprises or consists of SEQ ID NO.: 58.

In one embodiment, the at least one first polypeptide (a) specifically binding to an epitope on FcRn does not compete with wild-type IgG1 Fc region for binding to FcRn. Preferably, the at least one first polypeptide (a) of this embodiment comprises or consists of an ISVD, as defined herein. Preferably, the ISVD comprises or consist of T0263018B11(Y79S,T100bE,W100cS) (SEQ ID NO.: 186).

In another embodiment, the at least one polypeptide which competes with wild-type IgG1 Fc region for binding to FcRn is a Fc domain or a fragment thereof which comprises a quadruple amino acid substitution selected from M252Y/T256D/T307Q/N434F (YDQF), M252Y/T256D/T307W/N434F (YDWF), M252Y/T256D/T307Q/N434Y (YDQY), M252Y/T256E/T307Q/N434Y (YEQY), M252Y/T256D/T307W/N434Y (YDWY), and M252Y/T256E/T307W/N434Y (YEWY). Preferably, the at least one second polypeptide which competes with wild-type IgG1 Fc region for binding to FcRn comprises the quadruple amino acid substitution M252Y/T256D/T307Q/N434Y (YDQY). More preferably, the Fc domain or a fragment thereof comprises or consists of SEQ ID NO: 121-123, preferably comprises or consists of SEQ ID NO: 123 for a homodimer Fc-domain or comprises or consists of 121 and 122 for a heterodimeric Fc-domain.

Hence, in a preferred embodiment, the FcRn antagonists of the present invention comprise:
a) At least one first polypeptide specifically binding to an epitope on Fc receptor (FcRn), which comprises or consists of an ISVD, preferably wherein the ISVD comprises or consist of T0263018B11(Y79S,T100bE,W100cS) (SEQ ID NO.: 186);
b) At least a second polypeptide which competes with wild-type IgG1 Fc region for binding to FcRn, which comprises or consists of a Fc domain or a fragment thereof which comprises a quadruple amino acid substitution selected from M252Y/T256D/T307Q/N434F (YDQF), M252Y/T256D/T307W/N434F (YDWF), M252Y/T256D/T307Q/N434Y (YDQY), M252Y/T256E/T307Q/N434Y (YEQY), M252Y/T256D/T307W/N434Y (YDWY), and M252Y/T256E/T307W/N434Y (YEWY), preferably which comprises the quadruple amino acid substitution M252Y/T256D/T307Q/N434Y (YDQY), more preferably the Fc domain or a fragment thereof comprises or consists of SEQ ID NO: 121-123, preferably comprises or consists of SEQ ID NO: 123 for a homodimer Fc-domain or comprises or consists of 121 and 122 for a heterodimeric Fc-domain; and
c) At least one further group, residue, moiety or binding unit, which is an ISVD, for instance a human serum albumin (HSA)-binding ISVD, preferably wherein the ISVD binds to human serum albumin and is selected from the ISVDs of Table 5, preferably wherein the ISVD comprises or consists of SEQ ID NO.: 58.

The three components of the FcRn antagonists in this embodiment (a, b and c) may be covalently linked among them directly or by means of a linker. In a preferred embodiment, a), b) and c) are covalently linked to each other by means of a linker, preferably a peptide linker, such as a linker as shown in Table 4. Preferably, the linker is a 35GS linker (see, e.g., SEQ ID NO.: 39). Hence, in a preferred embodiment, a), b) and c) are linked to each other by means of a 35GS liner (SEQ ID NO.: 39).

In one embodiment, the FcRn antagonist of the present invention is in a dimeric form, preferably in a heterodimeric form. For instance, one of the polypeptide chains or fragments may comprise SEQ ID NO.: 121, and the other polypeptide chain or fragment may comprise SEQ ID NO.: 122. One of the polypeptide chains or fragments may comprise, at its N- and/or C-terminus, covalently linked directly or by means of a linker, at least one first polypeptide (a) specifically binding to an epitope on FcRn and/or at least one further group, residue, moiety or binding unit, optionally linked via one or more linkers, such as peptide linkers, as defined above. For instance, one of the polypeptide chains or fragments may comprise, at its N- and/or C-terminus, covalently linked directly or by means of a linker, at least one ISVD specifically binding to an epitope on Fc receptor (FcRn), wherein the ISVD does not compete with wild-type IgG1 Fc region for binding to FcRn, preferably wherein the ISVD comprises or consist of T0263018B11(Y79S,T100bE,W100cS) (SEQ ID NO.: 186). For instance, the other polypeptide chain or fragment may comprise, at its N- and/or C-terminus, covalently linked directly or by means of a linker, at least one further group, residue, moiety or binding unit, optionally linked via one or more linkers, such as peptide linkers, as defined above.

In one embodiment, one of the polypeptide chains or fragments may comprise, at its C-terminus, covalently linked by means of a linker, preferably a 35GS linker as defined herein, at least one ISVD specifically binding to an epitope on Fc receptor (FcRn), wherein the ISVD does not compete with wild-type IgG1 Fc region for binding to FcRn, preferably wherein the ISVD comprises or consist of T0263018B11(Y79S,T100bE,W100cS) (SEQ ID NO.: 186). The other polypeptide chain or fragment may comprise at its C-terminus, covalently linked by means of a linker, preferably a 35GS linker as defined herein, at least one further group, residue, moiety or binding unit, as defined above, preferably an albumin-binding ISVD, more preferably an ISVD that binds to human serum albumin and comprises a CDR1 (Abm) that is the amino acid sequence of SEQ ID NO: 19, a CDR2 (Abm) that is the amino acid sequence of SEQ ID NO: 20 and a CDR3 (Abm) that is the amino acid sequence of SEQ ID NO: 21, or, using the Kabat definition, a CDR1 (Kabat) that is the amino acid sequence of SEQ ID NO: 16, a CDR2 (Kabat) that is the amino acid sequence of SEQ ID NO: 17 and a CDR3 (Kabat) that is the amino acid sequence of SEQ ID NO: 18, even more preferably an albumin-binding ISVD comprising or consisting of SEQ ID NO.: 58.

For instance, in one embodiment, the FcRn antagonist is heterodimeric and it comprises or consists of:
one polypeptide chain or fragment which comprises a N-terminal polypeptide defined by SEQ ID NO.: 121, followed by a 35GS linker (SEQ ID NO.: 39) and a C-terminal ISVD specifically binding to an epitope on Fc receptor (FcRn), wherein the ISVD does not compete with wild-type IgG1 Fc region for binding to FcRn, preferably wherein the ISVD comprises or consist of T0263018B11(Y79S,T100bE,W100cS) (SEQ ID NO.: 186); and
a second polypeptide chain or fragment which comprises a N-terminal polypeptide chain or fragment which comprises or consists of SEQ ID NO.: 122 followed by a 35GS linker (SEQ ID NO.: 39) and a C-terminal albumin-binding ISVD, preferably an ISVD that binds to human serum albumin and comprises a CDR1 (Abm) that is the amino acid sequence of SEQ ID NO: 19, a CDR2 (Abm) that is the amino acid sequence of SEQ ID NO: 20 and a CDR3 (Abm) that is the amino acid sequence of SEQ ID NO: 21, or, using the Kabat definition, a CDR1 (Kabat) that is the amino acid sequence of SEQ ID NO: 16, a CDR2 (Kabat) that is the amino acid sequence of SEQ ID NO: 17 and a CDR3 (Kabat) that is the amino acid sequence of SEQ ID NO: 18, more preferably an albumin-binding ISVD selected from Table 5, even more preferably comprising or consisting of SEQ ID NO.: 58.

In a preferred embodiment, the first and/or the second polypeptide chain or fragment as defined above comprises a C-terminal alanine. The linker may be one as shown in Table 4. In a preferred embodiment, the linker is a 35GS linker (see, e.g., SEQ ID NO.: 39).

In another preferred embodiment, the FcRn antagonist is TPP-122910, which comprises two chains:
Chain 1: human IgG1 Fc YDQY (SEQ ID NO.: 121)-35GS-T0263018B11(Y79S,T100bE,W100cS) (SEQ ID NO.:186)-A; and
Chain 2: human IgG1 Fc YDQY (SEQ ID NO.: 122)-35GS-ALB23002 (SEQ ID NO.:58)-A.

In another embodiment, the FcRn antagonist of the present invention comprises:
a) Two first polypeptides specifically binding to an epitope on Fc receptor (FcRn), as defined herein;
b) At least a second polypeptide which competes with wild-type IgG1 Fc region for binding to FcRn.

Preferably, the at least one second polypeptide which competes with wild-type IgG1 Fc region for binding to FcRn comprises or consists of a Fc domain or a fragment thereof which comprises a quadruple amino acid substitution selected from M252Y/T256D/T307Q/N434F (YDQF), M252Y/T256D/T307W/N434F (YDWF), M252Y/T256D/T307Q/N434Y (YDQY), M252Y/T256E/T307Q/N434Y (YEQY), M252Y/T256D/T307W/N434Y (YDWY), and M252Y/T256E/T307W/N434Y (YEWY), preferably the quadruple amino acid substitution M252Y/T256D/T307Q/N434Y (YDQY), more preferably the Fc domain or a fragment thereof comprises or consists of SEQ ID NO: 121-123, preferably comprises or consists of SEQ ID NO: 123 for a homodimer Fc-domain or comprises or consists of 121 and 122 for a heterodimeric Fc-domain.

In one embodiment, at least one of the at least two first polypeptides (a) specifically binding to an epitope on FcRn does not compete with wild-type IgG1 Fc region for binding to FcRn. Preferably, both of the at least two first polypeptides (a) specifically binding to an epitope on FcRn does not compete with wild-type IgG1 Fc region for binding to FcRn do not compete with wild-type IgG1 Fc region for binding to FcRn. Preferably, the at least two first polypeptides (a) of this embodiment comprise or consist of an ISVD, as defined herein. Preferably, the ISVD comprises or consist of T0263018B11(Y79S,T100bE,W100cS) (SEQ ID NO.: 186).

In another embodiment, the at least one polypeptide which competes with wild-type IgG1 Fc region for binding to FcRn is a Fc domain or a fragment thereof which comprises a quadruple amino acid substitution selected from M252Y/T256D/T307Q/N434F (YDQF), M252Y/T256D/T307W/N434F (YDWF), M252Y/T256D/T307Q/N434Y (YDQY), M252Y/T256E/T307Q/N434Y (YEQY), M252Y/T256D/T307W/N434Y (YDWY), and M252Y/T256E/T307W/N434Y (YEWY). Preferably, the at least one second polypeptide which competes with wild-type IgG1 Fc region for binding to FcRn comprises the quadruple amino acid substitution M252Y/T256D/T307Q/N434Y (YDQY). More preferably, the Fc domain or a fragment thereof comprises or consists of SEQ ID NO: 123 for a homodimer Fc-domain.

Hence, in a preferred embodiment, the FcRn antagonist of the present invention comprises:
a) At least two first polypeptides specifically binding to an epitope on Fc receptor (FcRn), each of which comprises or consists of an ISVD, preferably wherein each ISVD comprises or consists of T0263018B11(Y79S,T100bE,W100cS) (SEQ ID NO.: 186);
b) At least a second polypeptide which competes with wild-type IgG1 Fc region for binding to FcRn, which comprises or consists of a Fc domain or a fragment thereof which comprises a quadruple amino acid substitution selected from M252Y/T256D/T307Q/N434F (YDQF), M252Y/T256D/T307W/N434F (YDWF), M252Y/T256D/T307Q/N434Y (YDQY), M252Y/T256E/T307Q/N434Y (YEQY), M252Y/T256D/T307W/N434Y (YDWY), and M252Y/T256E/T307W/N434Y (YEWY), preferably which comprises the quadruple amino acid substitution M252Y/T256D/T307Q/N434Y (YDQY), more preferably the Fc domain or a fragment thereof comprises or consists of SEQ ID NO: 121-123, preferably comprises or consists of SEQ ID NO: 123 for a homodimer Fc-domain.

The three components of the FcRn antagonists in this embodiment (two first polypeptides (a) and one polypeptide (b)) may be covalently linked among them directly or by means of a linker. In a preferred embodiment, the two first polypeptides (a) and the one polypeptide (b) are covalently linked to each other by means of a linker, preferably a peptide linker, such as a linker as shown in Table 4. Preferably, the linker is a 35GS linker (see, e.g., SEQ ID NO.: 39). Hence, in a preferred embodiment, the two first polypeptides (a) and the one polypeptide (b) are linked to each other by means of a 35GS liner (SEQ ID NO.: 39).

In one embodiment, the FcRn antagonist of the present invention is in a dimeric form, preferably in a homodimeric form. For instance, both polypeptide chains or fragments may comprise SEQ ID NO.: 123. Both polypeptide chains or fragments may comprise, at their N- and/or C-terminus, covalently linked directly or by means of a linker, at least one first polypeptide (a) specifically binding to an epitope on FcRn, as defined herein. For instance, both of the polypeptide chains or fragments may comprise, at their N- and/or C-terminus, preferably at their C-terminus, covalently linked directly or by means of a linker, at least one ISVD specifically binding to an epitope on Fc receptor (FcRn), wherein the ISVD does not compete with wild-type IgG1 Fc region for binding to FcRn, preferably wherein the ISVD comprises or consist of T0263018B11(Y79S,T100bE,W100cS) (SEQ ID NO.: 186).

In one embodiment, both polypeptide chains or fragments comprise, at its C-terminus, covalently linked by means of a linker, preferably a 35GS linker as defined herein, at least one ISVD specifically binding to an epitope on Fc receptor (FcRn), wherein the ISVD does not compete with wild-type IgG1 Fc region for binding to FcRn, preferably wherein the ISVD comprises or consist of T0263018B11(Y79S,T100bE,W100cS) (SEQ ID NO.: 186).

For instance, in one embodiment, the FcRn antagonist is homodimeric and it comprises or consists of two polypeptide chains or fragments which each comprises a N-terminal polypeptide defined by SEQ ID NO.: 123, followed by a 35GS linker (SEQ ID NO.: 39) and a C-terminal ISVD specifically binding to an epitope on Fc receptor (FcRn), wherein the ISVD does not compete with wild-type IgG1 Fc region for binding to FcRn, preferably wherein the ISVD comprises or consist of T0263018B11(Y79S,T100bE,W100cS) (SEQ ID NO.: 186).

In a preferred embodiment, one or both polypeptide chains or fragments as defined above comprise a C-terminal alanine.

In another preferred embodiment, the FcRn antagonist is TPP-122903, which comprises two chains:
Chain 1: human IgG1 Fc YDQY (SEQ ID NO.: 123)-35GS-T0263018B11(Y79S,T100bE,W100cS) (SEQ ID NO.:186)-A; and
Chain 2: human IgG1 Fc YDQY (SEQ ID NO.: 123)-35GS-T0263018B11(Y79S,T100bE,W100cS) (SEQ ID NO.:186)-A.

In another embodiment, the FcRn antagonist of the present invention comprises:
a) Two first polypeptides specifically binding to an epitope on Fc receptor (FcRn), as defined herein;
b) At least a second polypeptide which competes with wild-type IgG1 Fc region for binding to FcRn.

Preferably, the at least one second polypeptide which competes with wild-type IgG1 Fc region for binding to FcRn comprises or consists of a Fc domain or a fragment thereof which comprises a quadruple amino acid substitution selected from M252Y/T256D/T307Q/N434F (YDQF), M252Y/T256D/T307W/N434F (YDWF), M252Y/T256D/T307Q/N434Y (YDQY), M252Y/T256E/T307Q/N434Y (YEQY), M252Y/T256D/T307W/N434Y (YDWY), and M252Y/T256E/T307W/N434Y (YEWY), preferably the quadruple amino acid substitution M252Y/T256D/T307Q/N434Y (YDQY), more preferably the Fc domain or a fragment thereof comprises or consists of SEQ ID NO: 121-123, preferably comprises or consists of SEQ ID NO: 123 for a homodimer Fc-domain (two times SEQ ID NO.: 123) or comprises or consists of 121 and 122 for a heterodimeric Fc-domain.

In one embodiment, at least one of the at least two first polypeptides (a) specifically binding to an epitope on FcRn does not compete with wild-type IgG1 Fc region for binding to FcRn.

Preferably, both of the at least two first polypeptides (a) specifically binding to an epitope on FcRn does not compete with wild-type IgG1 Fc region for binding to FcRn do not compete with wild-type IgG1 Fc region for binding to FcRn. Preferably, the at least two first polypeptides (a) of this embodiment comprise or consist of an ISVD, as defined herein. Preferably, the ISVD comprises or consist of T0263091D07 (SEQ ID NO.: 131).

In another embodiment, the at least one polypeptide which competes with wild-type IgG1 Fc region for binding to FcRn is a Fc domain or a fragment thereof which comprises a quadruple amino acid substitution selected from M252Y/T256D/T307Q/N434F (YDQF), M252Y/T256D/T307W/N434F (YDWF), M252Y/T256D/T307Q/N434Y (YDQY), M252Y/T256E/T307Q/N434Y (YEQY), M252Y/T256D/T307W/N434Y (YDWY), and M252Y/T256E/T307W/N434Y (YEWY). Preferably, the at least one second polypeptide which competes with wild-type IgG1 Fc region for binding to FcRn comprises the quadruple amino acid substitution M252Y/T256D/T307Q/N434Y (YDQY). More preferably, the Fc domain or a fragment thereof comprises or consists of SEQ ID NO: 123 for a homodimer Fc-domain (two polypeptides comprising or consisting of SEQ ID NO.: 123).

Hence, in a preferred embodiment, the FcRn antagonist of the present invention comprises:
a) At least two first polypeptides specifically binding to an epitope on Fc receptor (FcRn), each of which comprises or consists of an ISVD, preferably wherein each ISVD comprises or consists of T0263091D07 (SEQ ID NO.: 131);
b) At least a second polypeptide which competes with wild-type IgG1 Fc region for binding to FcRn, which comprises or consists of a Fc domain or a fragment thereof which comprises a quadruple amino acid substitution selected from M252Y/T256D/T307Q/N434F (YDQF), M252Y/T256D/T307W/N434F (YDWF), M252Y/T256D/T307Q/N434Y (YDQY), M252Y/T256E/T307Q/N434Y (YEQY), M252Y/T256D/T307W/N434Y (YDWY), and M252Y/T256E/T307W/N434Y (YEWY), preferably which comprises the quadruple amino acid substitution M252Y/T256D/T307Q/N434Y (YDQY), more preferably the Fc domain or a fragment thereof comprises or consists of SEQ ID NO: 121-123, preferably comprises or consists of SEQ ID NO: 123 for a homodimer Fc-domain (two polypeptides comprising or consisting of SEQ ID NO.: 123).

The three components of the FcRn antagonists in this embodiment (two first polypeptides (a) and one polypeptide (b)) may be covalently linked among them directly or by means of a linker. In a preferred embodiment, the two first polypeptides (a) and the one polypeptide (b) are covalently linked to each other by means of a linker, preferably a peptide linker, such as a linker as shown in Table 4. Preferably, the linker is a 35GS linker (see, e.g., SEQ ID NO.: 39). Hence, in a preferred embodiment, the two first polypeptides (a) and the one polypeptide (b) are linked to each other by means of a 35GS liner (SEQ ID NO.: 39).

In one embodiment, the FcRn antagonist of the present invention is in a dimeric form, preferably in a homodimeric form. For instance, both polypeptide chains or fragments may comprise SEQ ID NO.: 123. Both polypeptide chains or fragments may comprise, at their N- and/or C-terminus, covalently linked directly or by means of a linker, at least one first polypeptide (a) specifically binding to an epitope on FcRn, as defined herein. For instance, both of the polypeptide chains or fragments may comprise, at their N- and/or C-terminus, preferably at their C-terminus, covalently linked directly or by means of a linker, at least one ISVD specifically binding to an epitope on Fc receptor (FcRn), wherein the ISVD does not compete with wild-type IgG1 Fc region for binding to FcRn, preferably wherein the ISVD comprises or consist of T0263091D07 (SEQ ID NO.: 131).

In one embodiment, both polypeptide chains or fragments comprise, at its C-terminus, covalently linked by means of a linker, preferably a 35GS linker as defined herein, at least one ISVD specifically binding to an epitope on Fc receptor (FcRn), wherein the ISVD does not compete with wild-type IgG1 Fc region for binding to FcRn, preferably wherein the ISVD comprises or consist of T0263091D07 (SEQ ID NO.: 131).

For instance, in one embodiment, the FcRn antagonist is homodimeric and it comprises or consists of two polypeptide chains or fragments which each comprises a N-terminal polypeptide defined by SEQ ID NO.: 123, followed by a 35GS linker (SEQ ID NO.: 39) and a C-terminal ISVD specifically binding to an epitope on Fc receptor (FcRn), wherein the ISVD does not compete with wild-type IgG1 Fc region for binding to FcRn, preferably wherein the ISVD comprises or consist of T0263091D07 (SEQ ID NO.: 131).

In a preferred embodiment, one or both polypeptide chains or fragments as defined above comprise a C-terminal alanine.

In another preferred embodiment, the FcRn antagonist is TPP-122904, which comprises two chains:
Chain 1: human IgG1 FcYDQY (SEQ ID NO.: 123)-35GS-T0263091D07 (SEQ ID NO.: 131)-A; and
Chain 2: human IgG1 FcYDQY (SEQ ID NO.: 123)-35GS-T0263091D07 (SEQ ID NO.: 131)-A.

In another embodiment, the at least one first polypeptide (a) comprises or consists of a single-chain variable fragment (scFv), preferably wherein the scFv does not compete with wild-type IgG1 Fc region for binding to FcRn, as defined herein. Hence, in a preferred embodiment, the FcRn antagonists of the present invention comprise:
a) at least one scFv specifically binding to an epitope on Fc receptor (FcRn), wherein the scFv preferably does not compete with wild-type IgG1 Fc region for binding to FcRn, as described herein; and
b) at least one polypeptide which competes with wild-type IgG1 Fc region for binding to FcRn, wherein the at least one second polypeptide which competes with wild-type IgG1 Fc region for binding to FcRn is a Fc domain or a fragment thereof which comprises a quadruple amino acid substitution selected from M252Y/T256D/T307Q/N434F (YDQF), M252Y/T256D/T307W/N434F (YDWF), M252Y/T256D/T307Q/N434Y (YDQY), M252Y/T256E/T307Q/N434Y (YEQY), M252Y/T256D/T307W/N434Y (YDWY), and M252Y/T256E/T307W/N434Y (YEWY), preferably the quadruple amino acid substitution M252Y/T256D/T307Q/N434Y (YDQY), more preferably the Fc domain or a fragment thereof comprises or consists of SEQ ID NO: 121-123, preferably comprises or consists of SEQ ID NO: 123 for a homodimer Fc-domain or comprises or consists of 121 and 122 for a heterodimeric Fc-domain.

In a preferred embodiment, the FcRn antagonists of the present invention comprise:
a) at least one scFv specifically binding to an epitope on Fc receptor (FcRn), wherein the scFv preferably does not compete with wild-type IgG1 Fc region for binding to FcRn, preferably wherein the scFv comprises or consists of SEQ ID NO.: 201); and
b) at least one polypeptide which competes with wild-type IgG1 Fc region for binding to FcRn, wherein the at least one second polypeptide which competes with wild-type IgG1 Fc region for binding to FcRn is a Fc domain or a fragment thereof which comprises a quadruple amino acid substitution selected from M252Y/T256D/T307Q/N434F (YDQF), M252Y/T256D/T307W/N434F (YDWF), M252Y/T256D/T307Q/N434Y (YDQY), M252Y/T256E/T307Q/N434Y (YEQY), M252Y/T256D/T307W/N434Y (YDWY), and M252Y/T256E/T307W/N434Y (YEWY), preferably the quadruple amino acid substitution M252Y/T256D/T307Q/N434Y (YDQY), more preferably the Fc domain or a fragment thereof comprises or consists of SEQ ID NO: 121-123, preferably comprises or consists of SEQ ID NO: 123 for a homodimer Fc-domain or comprises or consists of 121 and 122 for a heterodimeric Fc-domain.

SEQ ID NO.: 201 is as follows:
DIQLTQSPSSLSASVGDRVTLTCQATQDIDNNLNWYQQKPGKAPK
LLIYDASNLETGVPSRFSGSGSGTDFTFTISDLQPEDVATYYCQQ
YYNLPLTFGGGTKVDIKRSRGGGGSGGGGSGGGGSLEMAEVQLVQ
SGAEVKKPGASVKVSCKASGYTFTSYDINWVRQATGQGLEWMGWM
NPNSGNTGYAQKFQGRVTMTRNTSISTAYMELSSLRSEDTAVYYC
ARGVDLGDGWGQGTLVTVSS

In one embodiment, the FcRn antagonist of the present invention comprises two polypeptide fragments (chains). Hence, in one embodiment, the FcRn antagonist of the present invention is in a dimeric form, preferably in a heterodimeric form. One of the polypeptide chains or fragments may comprise at least a hinge region or a part of it, a CH2 domain, and a CH3 domain. The other polypeptide chain or fragment may comprise at least a hinge region or a part of it, a CH2 domain, and a CH3 domain. For instance, one of the polypeptide chains or fragments may comprise SEQ ID NO.: 121, and the other polypeptide chain or fragment may comprise SEQ ID NO.: 122. One of the polypeptide chains or fragments may comprise, at its N- and/or C-terminus, covalently linked directly or by means of a linker, at least one first polypeptide (a) specifically binding to an epitope on FcRn. For instance, one of the polypeptide chains or fragments may comprise, at its N- and/or C-terminus, covalently linked directly or by means of a linker, at least one scFv specifically binding to an epitope on Fc receptor (FcRn), wherein the scFv preferably does not compete with wild-type IgG1 Fc region for binding to FcRn, preferably wherein the scFv comprises or consists of SEQ ID NO.: 201).

In one embodiment, one of the polypeptide chains or fragments may comprise, at its C-terminus, covalently linked by means of a linker, preferably a 35GS linker as defined herein, at least one scFv specifically binding to an epitope on Fc receptor (FcRn), wherein the scFv preferably does not compete with wild-type IgG1 Fc region for binding to FcRn, preferably wherein the scFv comprises or consists of SEQ ID NO.: 201). For instance, in one embodiment, the FcRn antagonist is heterodimeric and it comprises or consists of:
one polypeptide chain or fragment which comprises a N-terminal polypeptide defined by SEQ ID NO.: 121, followed by a 35GS linker (SEQ ID NO.: 39) and a C-terminal scFv specifically binding to an epitope on Fc receptor (FcRn), wherein the scFv preferably does not compete with wild-type IgG1 Fc region for binding to FcRn, preferably wherein the scFv comprises or consists of SEQ ID NO.: 201); and
a second polypeptide chain or fragment which comprises or consists of SEQ ID NO.: 122.

In one embodiment, the first and/or the second polypeptide chain or fragment as defined above comprises a C-terminal alanine. The linker may be one as shown in Table 4. In a preferred embodiment, the linker is a 35GS linker (see, e.g., SEQ ID NO.: 39).

In a preferred embodiment, the FcRn antagonist is TPP-122912, which comprises two chains:
Chain 1: human IgG1 FcYDQY (SEQ ID NO.: 121)-35GS-FcRn antibody FnAb12 ScFv (SEQ ID NO.: 201); and
Chain 2: human IgG1 Fc YDQY (SEQ ID NO.: 122).

### FcRn Antagonists Comprising Two ISVDs

In another embodiment, the FcRn antagonist of the present invention comprises:
a) At least one first polypeptide specifically binding to an epitope on Fc receptor (FcRn); and
b) At least a second polypeptide which competes with wild-type IgG1 Fc region for binding to FcRn,
   wherein the at least one second polypeptide as defined in (b) comprises or consists of an ISVD.

In this embodiment, the at least one first polypeptide (a) can be any polypeptide as described herein, see, e.g., section “The first polypeptide (a) comprised in the FcRn antagonists of the present invention” and the at least one second polypeptide as defined in (b) comprises or consists of an ISVD.

In certain embodiments, the ISVD comprised in the at least one second polypeptide as defined in (b) comprises 4 framework regions (FR1 to FR4, respectively) and three complementarity determining regions (CDR1 to CDR3, respectively), wherein:
CDR1 (AbM numbering) consists of an amino acid sequence selected from:
   a) the amino acid sequence of GFTFSDYGMG (SEQ ID NO:155);
   b) amino acid sequences that have at least 80% amino acid identity with the amino acid sequence of GFTFSDYGMG (SEQ ID NO:155);
   c) amino acid sequences that have 3, 2, or 1 amino acid difference with the amino acid sequences GFTFSDYGMG (SEQ ID NO:155);
and
CDR2 (AbM numbering) consists of an amino acid sequence selected from:
   a) the amino acid sequence of YISSDGGETS (SEQ ID NO: 156);
   b) amino acid sequences that have at least 80% amino acid identity with the amino acid sequence of YISSDGGETS (SEQ ID NO: 156);
   c) amino acid sequences that have 3, 2, or 1 amino acid difference with the amino acid sequence of YISSDGGETS (SEQ ID NO: 156);
and
CDR3 (AbM numbering) consists of an amino acid sequence selected from:
   a) the amino acid sequence of GGV (SEQ ID NO:157);
   b) amino acid sequences that have at least 80% amino acid identity with the amino acid sequence of GGV (SEQ ID NO:157);
   c) amino acid sequences that have 3, 2, or 1 amino acid difference with the amino acid sequence of GGV (SEQ ID NO:157).

Preferably, the CDR sequences of the at least a second polypeptide (b) which competes with wild-type IgG1 Fc region for binding to FcRn which is an ISVD have at least 90% amino acid sequence identity, more preferably at least 95% amino acid sequence identity, such as 99% amino acid sequence identity or more, or even essentially 100% amino acid sequence identity with the CDR sequences of SEQ ID NOs: 155, 156, and/or 157.

In a further embodiment, the at least a second polypeptide (b) which competes with wild-type IgG1 Fc region for binding to FcRn which is an ISVD comprises CDRs (AbM numbering) with an amino acid sequence that has at least 70% amino acid sequence identity, preferably at least 80% amino acid sequence identity, more preferably at least 90% amino acid sequence identity, such as 95% amino acid sequence identity or 99% amino acid sequence identity or more, or even essentially 100% amino acid sequence identity with the amino acid sequences of the CDRs of the ISVD with the amino acid sequence SEQ ID NO: 158 or 168. Even more preferably, the Fc domain or a fragment thereof comprises or consists of SEQ ID NO: 158 when the ISDV is at the N-terminal position.

The ISVD comprised in the at least one second polypeptide (b) may be covalently linked directly or by means of a linker to the at least one first polypeptide (a) specifically binding to an epitope on FcRn. In one embodiment, the ISVD comprised in the polypeptide (b) as described above is covalently linked by means of a linker to the at least one first polypeptide (a) specifically binding to an epitope on FcRn. The linker may be one as shown in Table 4. In a preferred embodiment, the linker is a 35GS linker (see, e.g., SEQ ID NO.: 39). The ISVD comprised in the at least one second polypeptide (b) as described above may be covalently linked directly or by means of a linker to the at least one first polypeptide (a) at the N- or C-terminal region of the ISVD comprised in the at least one second polypeptide (b) thereof as described above.

The at least one first polypeptide (a) specifically binding to an epitope on FcRn comprised in the FcRn antagonist of this embodiment may be any polypeptide as described herein, see, e.g., section “The first polypeptide (a) comprised in the FcRn antagonists of the present invention”. In one embodiment, the at least one first polypeptide (a) specifically binding to an epitope on FcRn does not compete with wild-type IgG1 Fc region for binding to FcRn. Preferably, the at least one first polypeptide (a) of this embodiment comprises or consists of an ISVD, as defined herein. Hence, in a preferred embodiment, the FcRn antagonists of the present invention comprise:
a) at least one ISVD specifically binding to an epitope on Fc receptor (FcRn), wherein the ISVD does not compete with wild-type IgG1 Fc region for binding to FcRn, as described herein; and
b) at least a second polypeptide which competes with wild-type IgG1 Fc region for binding to FcRn, wherein the at least one second polypeptide as defined in (b) comprises or consists of an ISVD, preferably wherein the ISVD comprised in the at least one second polypeptide as defined in (b) comprises 4 framework regions (FR1 to FR4, respectively) and three complementarity determining regions (CDR1 to CDR3, respectively), wherein the CDR sequences have at least 90% amino acid sequence identity, more preferably at least 95% amino acid sequence identity, such as 99% amino acid sequence identity or more, or even essentially 100% amino acid sequence identity with the CDR sequences of SEQ ID NOs: 155, 156, and/or 157, even more preferably, wherein the ISVD comprises or consists of SEQ ID NO: 158 or 168, preferably wherein the at least a second polypeptide (b) which competes with wild-type IgG1 Fc region for binding to FcRn which is an ISVD consists of SEQ ID NO.: 158.

In one embodiment, the at least one first polypeptide (a) specifically binding to an epitope on the Fc receptor (FcRn) comprised in the FcRn antagonists of the present invention comprises or consists of an ISVD, as described above in this description. Hence, in a preferred embodiment, the at least one first polypeptide (a) specifically binds to an epitope on FcRn which is different from the epitopes bound by the known natural FcRn ligands (i.e., serum albumin and IgG). This polypeptide (a) would thus not interfere (e.g., not compete) with the binding of wild-type IgG1 Fc region to FcRn.

Hence, in one preferred embodiment, both the at least one polypeptide (a) and the at least one polypeptide (b) comprised in the FcRn antagonists of the present invention comprise or consist of ISVDs.

In one embodiment, the at least one first polypeptide (a) specifically binding to an epitope on the Fc receptor (FcRn) comprised in the FcRn antagonists of the present invention is an ISVD that comprises 4 framework regions (FR1 to FR4, respectively) and three complementarity determining regions (CDR1 to CDR3, respectively), wherein the ISVD comprises a CDR1 (AbM numbering) that is the amino acid sequence of SEQ ID NO: 11, a CDR2 (AbM numbering) that is the amino acid sequence of SEQ ID NO: 12 and a CDR3 (AbM numbering) that is the amino acid sequence of SEQ ID NO: 189.

If the CDR definitions according to Kabat are used, then in this embodiment the at least one first polypeptide (a) specifically binding to an epitope on the Fc receptor (FcRn) comprised in the FcRn antagonists of the present invention is an ISVD that comprises 4 framework regions (FR1 to FR4, respectively) and three complementarity determining regions (CDR1 to CDR3, respectively), wherein the ISVD comprises a CDR1 (Kabat numbering) that is the amino acid sequence of SEQ ID NO: 8, a CDR2 (Kabat numbering) that is the amino acid sequence of SEQ ID NO: 9 and a CDR3 (Kabat numbering) that is the amino acid sequence of SEQ ID NO: 189.

In a preferred embodiment, the at least one first polypeptide (a) specifically binding to an epitope on the Fc receptor (FcRn) comprised in the FcRn antagonists of the present invention is an ISVD which comprises or consists of SEQ ID NO.: 186. If polypeptide (a) is located at the N-terminus of the FcRn antagonist, then the ISVD may comprise a E1D mutation. In this particular case, the ISVD preferably comprises or consists of SEQ ID NO.: 187.

In a preferred embodiment, the FcRn antagonist of the present invention comprises:
a) at least one immunoglobulin single variable domain (ISVD) specifically binding to an epitope on Fc receptor (FcRn), wherein the ISVD does not compete with wild-type IgG1 Fc region for binding to FcRn, preferably wherein the ISVD comprises or consist of T0263018B11(Y79S,T100bE,W100cS) (SEQ ID NO.: 186); and
b) At least a second polypeptide which competes with wild-type IgG1 Fc region for binding to FcRn, wherein the at least one second polypeptide as defined in (b) comprises or consists of an immunoglobulin single variable domain (ISVD), preferably, wherein the ISVD comprises or consists of SEQ ID NO: 158 or 168, more preferably SEQ ID NO.: 158.

In one embodiment, the ISVD comprised in the polypeptide (a) and the ISDV comprised in polypeptide (b) are linked, preferably a 35GS linker as defined herein.

In another embodiment, the FcRn antagonist of the present invention comprises:
a) At least one first polypeptide specifically binding to an epitope on Fc receptor (FcRn), as defined herein;
b) At least a second polypeptide which competes with wild-type IgG1 Fc region for binding to FcRn; and
c) At least one further group, residue, moiety or binding unit, optionally linked via one or more linkers, such as peptide linkers, as defined above, in which said one or more other groups, residues, moieties or binding units provide the antagonist of the present technology with increased (in vivo) half-life, compared to the corresponding antagonist without said one or more other groups, residues, moieties or binding units (“(in vivo) half-life extending moiety”, or “half-life extending (HLE) moiety”), as described herein,
   wherein the at least one second polypeptide as defined in (b) comprises or consists of an ISVD, preferably wherein the ISVD comprised in the at least one second polypeptide as defined in (b) comprises 4 framework regions (FR1 to FR4, respectively) and three complementarity determining regions (CDR1 to CDR3, respectively), wherein the CDR sequences have at least 90% amino acid sequence identity, more preferably at least 95% amino acid sequence identity, such as 99% amino acid sequence identity or more, or even essentially 100% amino acid sequence identity with the CDR sequences of SEQ ID NOs: 155, 156, and/or 157, even more preferably, wherein the ISVD comprises or consists of SEQ ID NO: 158 or 168, preferably SEQ ID NO.: 158.

In one embodiment, the at least one further group, residue, moiety or binding unit (c) is an ISVD, for instance a human serum albumin (HSA)-binding ISVD, as described herein. Preferably, the at least one further group, residue, moiety or binding unit (c) is an ISVD that binds to human serum albumin and comprises a CDR1 (Abm) that is the amino acid sequence of SEQ ID NO: 19, a CDR2 (Abm) that is the amino acid sequence of SEQ ID NO: 20 and a CDR3 (Abm) that is the amino acid sequence of SEQ ID NO: 21, or, using the Kabat definition, a CDR1 (Kabat) that is the amino acid sequence of SEQ ID NO: 16, a CDR2 (Kabat) that is the amino acid sequence of SEQ ID NO: 17 and a CDR3 (Kabat) that is the amino acid sequence of SEQ ID NO: 18. Preferably, the ISVD that binds to human serum albumin can be selected from the ISVDs of Table 5. More preferably, the ISVD that binds to human serum albumin comprises or consists of SEQ ID NO.: 58.

In one embodiment, the at least one first polypeptide (a) specifically binding to an epitope on the Fc receptor (FcRn) comprised in the FcRn antagonists of the present invention comprises or consists of an ISVD, as described above in this description. Hence, in a preferred embodiment, the at least one first polypeptide (a) specifically binds to an epitope on FcRn which is different from the epitopes bound by the known natural FcRn ligands (i.e., serum albumin and IgG). This polypeptide (a) would thus not interfere (e.g., not compete) with the binding of wild-type IgG1 Fc region to FcRn.

Hence, in one preferred embodiment, both the at least one polypeptide (a) and the at least one polypeptide (b) comprised in the FcRn antagonists of the present invention comprise or consist of ISVDs.

In one embodiment, the at least one first polypeptide (a) specifically binding to an epitope on the Fc receptor (FcRn) comprised in the FcRn antagonists of the present invention is an ISVD that comprises 4 framework regions (FR1 to FR4, respectively) and three complementarity determining regions (CDR1 to CDR3, respectively), wherein the ISVD comprises a CDR1 (AbM numbering) that is the amino acid sequence of SEQ ID NO: 11, a CDR2 (AbM numbering) that is the amino acid sequence of SEQ ID NO: 12 and a CDR3 (AbM numbering) that is the amino acid sequence of SEQ ID NO: 189.

If the CDR definitions according to Kabat are used, then in this embodiment the at least one first polypeptide (a) specifically binding to an epitope on the Fc receptor (FcRn) comprised in the FcRn antagonists of the present invention is an ISVD that comprises 4 framework regions (FR1 to FR4, respectively) and three complementarity determining regions (CDR1 to CDR3, respectively), wherein the ISVD comprises a CDR1 (Kabat numbering) that is the amino acid sequence of SEQ ID NO: 8, a CDR2 (Kabat numbering) that is the amino acid sequence of SEQ ID NO: 9 and a CDR3 (Kabat numbering) that is the amino acid sequence of SEQ ID NO: 189.

In a preferred embodiment, the at least one first polypeptide (a) specifically binding to an epitope on the Fc receptor (FcRn) comprised in the FcRn antagonists of the present invention is an ISVD which comprises or consists of SEQ ID NO.: 186. If polypeptide (a) is located at the N-terminus of the FcRn antagonist, then the ISVD may comprise a E1D mutation. In this particular case, the ISVD preferably comprises or consists of SEQ ID NO.: 187. Hence, in a preferred embodiment, the FcRn antagonist of the present invention comprises:
a) At least one first polypeptide specifically binding to an epitope on Fc receptor (FcRn), which comprises or consists of an ISVD, preferably wherein the ISVD comprises or consist of T0263018B11(Y79S,T100bE,W100cS) (SEQ ID NO.: 186);
b) At least a second polypeptide which competes with wild-type IgG1 Fc region for binding to FcRn, wherein the at least one second polypeptide as defined in (b) comprises or consists of an immunoglobulin single variable domain (ISVD), preferably wherein the ISVD comprised in the at least one second polypeptide as defined in (b) comprises 4 framework regions (FR1 to FR4, respectively) and three complementarity determining regions (CDR1 to CDR3, respectively), wherein the CDR sequences have at least 90% amino acid sequence identity, more preferably at least 95% amino acid sequence identity, such as 99% amino acid sequence identity or more, or even essentially 100% amino acid sequence identity with the CDR sequences of SEQ ID NOs: 155, 156, and/or 157, even more preferably, wherein the ISVD comprises or consists of SEQ ID NO: 158 or 168, more preferably SEQ ID NO.: 158;
c) At least one further group, residue, moiety or binding unit, which is an ISVD, for instance a human serum albumin (HSA)-binding ISVD, preferably wherein the ISVD binds to human serum albumin and is selected from the ISVDs of Table 5, preferably wherein the ISVD comprises or consists of SEQ ID NO.: 58.

The three components of the FcRn antagonists in this embodiment (a, b and c) may be covalently linked among them directly or by means of a linker. In a preferred embodiment, a), b) and c) are covalently linked to each other by means of a linker, preferably a peptide linker, such as a linker as shown in Table 4. Preferably, the linker is a 35GS linker (see, e.g., SEQ ID NO.: 39). Hence, in a preferred embodiment, a), b) and c) are linked to each other by means of a 35GS liner (SEQ ID NO.: 39).

In another preferred embodiment, the FcRn antagonist is TPP-122947, which comprises or consists of: T0263201B05(E1D)(SEQ ID NO.: 158)-35GS-T0263018B11(Y79S,T100bE,W100cS) (SEQ ID NO. 186)-35GS-ALB23002 (SEQ ID NO. 58)-A.

In a preferred embodiment, the FcRn antagonist comprises a C-terminal alanine.

### Methods for Preparing the FcRn Antagonists of the Present Invention

Another embodiment of the invention relates to a method for producing the FcRn antagonists of the present invention. As described in detail above, the FcRN antagonists according to the present invention comprise at least one first polypeptide (a) specifically binding to an epitope on Fc receptor (FcRn) and at least one second polypeptide (b) which competes with wild-type IgG1 Fc region for binding to FcRn. At least both polypeptides are comprised in the FcRn antagonists of the present invention. The skilled person is aware of means of linking two polypeptides to prepare the FcRn antagonists of the present invention. For instance, the method may comprise the steps of:
a) Providing at least a first (a) and a second (b) polypeptide, as described above;
b) Linking both polypeptides together, directly or by means of a linker, as described below.

For instance, the method may comprise the steps of:
a) Selecting at least a first and a second polypeptide, as described above;
b) Designing a genetic construct which encodes a protein sequence comprising both the first and second polypeptides; and
c) Introducing said genetic construct in an expression system to obtain the FcRn antagonists of the present invention, as described above in the present specification.

In the context of the present invention, the position of each of the polypeptides ((a) and (b)) in the FcRn antagonists of the present invention is not limited. For instance, the first polypeptide (a) may be located in the N-terminal part of the FcRn antagonists, whereas the second polypeptide (b) may be located in the C-terminal part of the FcRn antagonists. In addition, the first polypeptide (a) may be located in the C-terminal part of the FcRn antagonists, whereas the second polypeptide (b) may be located in the N-terminal part of the FcRn antagonists.

In addition, the at least one first polypeptide (a) and the at least one second polypeptide (b) may be directly linked to each other or linked via a linker, such as peptide linkers.

The use of linkers to connect two or more (poly)peptides is well known in the art. One frequently used class of peptidic linkers are known as the “Gly-Ser” or “GS” linkers. These are linkers that essentially consist of glycine (G) and serine (S) residues, and usually comprise one or more repeats of a peptide motif such as the GGGGS (SEQ ID NO: 29) motif (for example, exhibiting the formula (Gly-Gly-Gly-Gly-Ser)ₙ in which n may be 1, 2, 3, 4, 5, 6, 7 or more). Some often used examples of such GS linkers are 9GS linkers (GGGGSGGGS, SEQ ID NO: 32), 15GS linkers (n=3) and 35GS linkers (n=7). Reference is for example made to Chen et al., Adv. Drug Deliv. Rev. 2013 Oct. 15; 65(10): 1357-1369; and Klein et al., Protein Eng. Des. Sel. (2014) 27 (10): 325-330. In particular but non-limiting embodiments, the linker is chosen from the group consisting of linkers of 3A, 3GS, 5GS, 7GS, 9GS, 10GS, 15GS, 18GS, 20GS, 25GS, 30GS and 35GS (SEQ ID NO: 28 to 40).

For instance, when the at least one second polypeptide (b) which competes with wild-type IgG1 Fc region for binding to FcRn comprised in the FcRn antagonist of the present invention comprises or consists of a Fc domain or a fragment thereof, the Fc domain or a fragment thereof may comprise, in its N-terminal part, a sequence comprising or consisting of part of the hinge region. The “hinge region” is a short sequence of the heavy chains (H) of antibodies linking the Fab (Fragment antigen binding) region to the Fc (Fragment crystallizable) region. For instance, the Fc domain or a fragment thereof may comprise, in its N-terminal part, a sequence comprising or consisting of a sequence selected from SEQ ID NO: 41-44 and 66. In a preferred embodiment, the Fc domain or a fragment thereof may comprise, in its N-terminal part, a sequence comprising or consisting of SEQ ID NO: 66.

In other embodiments, when the at least one first polypeptide specifically binding to an epitope on FcRn comprises or consists of a Fc domain or a fragment thereof, the Fc domain or a fragment thereof may comprise, in its N-terminal part, a sequence comprising or consisting of part of the hinge region, as described above.

In these embodiments, when the Fc domain or a fragment thereof is located at the C-terminal part of the FcRn antagonist, the other polypeptide (e.g., the at least one first polypeptide (a) or the at least one second polypeptide (b), respectively) will be located at the N-terminal part of the FcRn antagonist. In this case, both polypeptides may be linked directly or by means of a linker, as described above. If they are linked by means of a linker, in a preferred embodiment, they are linked by means of the hinge region comprised in the Fc domain or fragment thereof, preferably comprising or consisting of SEQ ID NO: 66, as described above.

In the specific embodiment where the at least one first (a) or the at least one second (b) polypeptides comprised in the FcRn antagonists of the present invention is a dimeric Fc domain (i.e., a Fc domain comprising two polypeptides, each comprising at least one CH2 and at least one CH3 domains), the other polypeptide comprised in the FcRn antagonists may be linked (directly or via a linker, as described below) to the N- or C-terminal part of one of the polypeptides comprised in the dimeric Fc domain. For instance, if the at least one second polypeptide (b) is a dimeric Fc domain, the at least one first polypeptide (a) may be linked (directly or via a linker, as described below) to the N-terminal part of one of the polypeptides comprised in the dimeric Fc domain, e.g., to the hinge region or part thereof of one of the polypeptides, see, e.g., SEQ ID NO: 41-44 and 66. For instance, if the at least one second polypeptide (b) is a dimeric Fc domain, the at least one first polypeptide (a) may be linked (directly or via a linker, as described below) to the C-terminal part of one of the polypeptides comprised in the dimeric Fc domain, e.g., via a peptide linker, see, e.g., SEQ ID NO: 28 to 40. See FIG. 1, where the at least one second polypeptide (b) is a dimeric Fc domain and the at least one first polypeptide (a) is an ISVD. In this figure, two orientations of the FcRn antagonist are shown. In the middle of the figure, the at least one first polypeptide (a) is located at the N-terminal part of the FcRn antagonist (of), and it is linked to one of the polypeptides of the dimeric Fc domain through the hinge region. In the right-hand part of the Figure, the first polypeptide (a) is located at the C-terminal part of the FcRn antagonist, and it is linked to the Fc domain (polypeptide (b)) through a peptide linker (e.g., see SEQ ID NO: 28-40 in Table 4).

TABLE 4
Linker sequences (“ID” refers to the
SEQ ID NO as used herein)

| Name | ID | Amino acid sequence |
|---|---|---|
| 3A linker | 28 | AAA |
| 5GS linker | 29 | GGGGS |
| 7GS linker | 30 | SGGSGGS |
| 8GS linker | 31 | GGGGSGGS |
| 9GS linker | 32 | GGGGSGGGS |
| 10GS linker | 33 | GGGGSGGGGS |
| 15GS linker | 34 | GGGGSGGGGSGGGGS |
| 18GS linker | 35 | GGGGSGGGGSGGGGSGGS |
| 20GS linker | 36 | GGGGSGGGGSGGGGSGGGGS |
| 25GS linker | 37 | GGGGSGGGGSGGGGSGGGGS |
| | | GGGGS |
| 30GS linker | 38 | GGGGSGGGGSGGGGSGGGGS |
| | | GGGGSGGGGS |
| 35GS linker | 39 | GGGGSGGGGSGGGGSGGGGS |
| | | GGGGSGGGGSGGGGS |
| 40GS linker | 40 | GGGGSGGGGSGGGGSGGGGS |
| | | GGGGSGGGGSGGGGSGGGGS |
| G1 hinge | 41 | EPKSCDKTHTCPPCP |
| 9GS-G1 hinge | 42 | GGGGSGGGSEPKSCDKTHTC |
| | | PPCP |
| Llama upper long | 43 | EPKTPKPQPAAA |
| hinge region | | |
| G3 hinge | 44 | ELKTPLGDTTHTCPRCPEPKS |
| | | CDTPPPCPRCPEPKSCDTPPP |
| | | CPRCPEPKSCDTPPPCPRCP |
| Short G1 hinge | 66 | DKTHTCPPCP |

### Fusion Protein

The present invention further provides a fusion protein comprising the FcRn antagonists of the present invention. The fusion protein of the present invention may comprise, besides the FcRn antagonists, further groups, residues, moieties or binding units.

For example, such further groups, residues, moieties or binding units may be one or more additional immunoglobulins, so as to form a (fusion) protein (the fusion protein of the present invention). In a preferred but non-limiting aspect, the one or more other groups, residues, moieties or binding units are ISVDs. Even more preferably, the one or more other groups, residues, moieties or binding units are chosen from the group consisting of domain antibodies, ISVDs that are suitable for use as a domain antibody, single domain antibodies, ISVDs that are suitable for use as a single domain antibody, “dAb” 's, ISVDs that are suitable for use as a dAb, V_{HH}s, humanized V_{HH}S, camelized V_{H}S, or Nanobody® V_{HH}s. Alternatively, such groups, residues, moieties or binding units may for example be chemical groups, residues, moieties, which may or may not by themselves be biologically and/or pharmacologically active. For example, and without limitation, such groups may be linked to the FcRn antagonists of the invention, as further described herein. A fusion protein of the invention may also include additional groups with certain functionalities, such as a label, a toxin, one or more linkers, a binding sequence, etc. These additional functionalities include both amino acid-based and non-amino acid-based groups.

Hence, in one embodiment, the fusion protein of the present invention may further comprise (besides the FcRn antagonists of the present invention), one or more ISVDs. Preferably, the ISVD may be a HLE ISVD, a targeting ISVD or a therapeutic ISVD. The one or more ISVDs which may be further comprised (besides the one which may be comprised as the first polypeptide (a) in the FcRn antagonists of the present invention) in the fusion protein of the present invention may thus form a “multivalent” or “multispecific” fusion protein.

Polypeptides that comprise of two or more ISVDs (such as the one which may be comprised as the first polypeptide (a) in the FcRn antagonists of the present invention, and one or more further ISVD(s)) will be referred to herein as “multivalent fusion proteins” or as “multivalent constructs”, and these may provide certain advantages compared to the corresponding monovalent polypeptide. Generally, proteins or polypeptides that comprise a single ISVD (such as the FcRn antagonists of the invention) will be referred to herein as “monovalent” proteins or as “monovalent constructs”.

As also described herein, multivalent proteins of the invention may for example, without limitation, be multispecific (such as bispecific or trispecific) or multiparatopic (such as biparatopic) constructs (or be both multiparatopic and multispecific), and may for example be constructs that comprise at least two binding domains or binding units that are each directed towards a different epitope on the same subunit, constructs that comprise at least two binding domains or binding units that each have a different biological function (for example one binding domain that can block or inhibit receptor-ligand interaction, and one binding domain that does not block or inhibit receptor-ligand interaction), or constructs that comprise at least two binding domains or binding units that are each directed towards a different target.

For a general description of multivalent and multispecific polypeptides containing one or more ISVDs and their preparation, reference is also made to Conrath et al., J. Biol. Chem., Vol. 276, 10. 7346-7350, 2001; Muyldermans, Reviews in Molecular Biotechnology 74 (2001), 277-302; as well as to for example WO 96/34103, WO 99/23221, WO 04/041862, WO 2006/122786, WO 2008/020079, WO 2008/142164 or WO 2009/068627.

The fusion protein of the invention can generally be prepared by a method which comprises at least one step of suitably linking the FcRn antagonists of the present invention to one or more further groups, residues, moieties or binding units, either directly or via one or more suitable linkers, as described herein.

Fusion proteins of the invention can also be prepared by a method which generally comprises at least the steps of providing a nucleic acid that encodes the fusion protein of the invention, expressing said nucleic acid in a suitable manner, and recovering the expressed polypeptide of the invention. Such methods can be performed in a manner known per se, which will be clear to the skilled person, for example on the basis of the methods and techniques further described herein.

It will be appreciated that the order of the FcRn antagonist and further groups, residues, moieties or binding units, if present, in the fusion protein of the invention can be chosen according to the needs of the person skilled in the art, as well as the relative affinities which may depend on the location of the FcRn antagonist and further groups, residues, moieties or binding units, if present, in the fusion protein. Whether the fusion protein comprises one or more linkers to interconnect the FcRn antagonist and optionally further groups, residues, moieties or binding units is a matter of design choice. However, some orientations, with or without linkers, may provide preferred binding characteristics in comparison to other orientations. All different possible orientations are encompassed by the invention.

For instance, the sequence of the fusion protein of the present invention may comprise one or more ISVDs, linked together directly or by means of a linker, as defined herein, followed by the FcRn antagonists of the present invention, directly linked to the one or more ISVDs or linked by means of a linker, as defined herein. In this embodiment, the one or more ISVDs would be located at the N-terminal region of the fusion protein, whereas the chimeric protein would be located at the C-terminal region of the fusion protein.

For instance, the sequence of the fusion protein of the present invention may comprise the FcRn antagonists of the present invention followed by one or more ISVDs, linked together directly or by means of a linker, directly linked to the FcRn antagonists of the invention, or linked by means of a linker, as defined herein. In this embodiment, the one or more ISVDs would be located at the C-terminal region of the fusion protein, whereas the FcRn antagonist would be located at the N-terminal region of the fusion protein.

For instance, the fusion protein of the present invention may additionally comprise a group, residue, moiety or binding unit which provides the FcRn antagonists of the present invention with increased (in vivo) half-life compared to the corresponding FcRn antagonist without said one or more other groups, residues, moieties or binding units (“(in vivo) half-life extending moiety”, or “half-life extending (HLE) moiety”).

Hence, in some embodiments the FcRn antagonist of the present invention comprises:
a) At least one first polypeptide specifically binding to an epitope on Fc receptor (FcRn);
b) At least a second polypeptide which competes with wild-type IgG1 Fc region for binding to FcRn; and
c) At least one further group, residue, moiety or binding unit, optionally linked via one or more linkers, such as peptide linkers, as defined above, in which said one or more other groups, residues, moieties or binding units provide the molecule of the present technology with increased (in vivo) half-life, compared to the corresponding molecule without said one or more other groups, residues, moieties or binding units (“(in vivo) half-life extending moiety”, or “half-life extending (HLE) moiety”), as defined herein.

The term “half-life” as used here can generally be defined as described in paragraph o) on page 57 of WO 2008/020079 and as mentioned therein refers to the time taken for the serum concentration of the compound or polypeptide to be reduced by 50%, in vivo, for example due to degradation of the sequence or compound and/or clearance or sequestration of the sequence or compound by natural mechanisms. The in vivo half-life of the FcRn antagonist of the present invention can be determined in any manner known per se, such as by pharmacokinetic analysis. Suitable techniques will be clear to the person skilled in the art and may for example generally be as described in paragraph o) on page 57 of WO 2008/020079. As also mentioned in paragraph o) on page 57 of WO 2008/020079, the half-life can be expressed using parameters such as the t_{1/2}-alpha, t_{1/2}-beta and the area under the curve (AUC). In this respect it should be noted that the term “half-life” as used herein in particular refers to the t_{1/2}-beta or terminal half-life (in which the t_{1/2}-alpha and/or the AUC or both may be kept out of considerations). Reference is for example made to the standard handbooks, such as Kenneth, A et ah: Chemical Stability of Pharmaceuticals: A Handbook for Pharmacists and Peters et al, Pharmacokinetic analysis: A Practical Approach (1996). Reference is also made to “Pharmacokinetics”, M Gibaldi & D Perron, published by Marcel Dekker, 2nd Rev. edition (1982). Similarly, the terms “increase in half-life” or “increased half-life” are also as defined in paragraph o) on page 57 of WO 2008/020079 and in particular refer to an increase in the t_{1/2}-beta, either with or without an increase in the t_{1/2}-alpha and/or the AUC or both.

(In vivo) half-life can be extended by an increase in the hydrodynamic radius (size) or by a decrease in the molecule's clearance. For instance, (in vivo) half-life extending moieties such as polyethylene glycol or ELNN polypeptides increase the size of the molecules to which they are attached, therefore bypassing renal clearance, and thus increasing the half-life of those molecules. Other (in vivo) half-life extending moieties such as binding units that can bind to, e.g., serum albumin, increase the half-life of the molecules to which they are attached by binding, e.g., to serum albumin. Albumin is the most abundant plasma protein, is highly soluble, very stable and has an extraordinarily long circulatory half-life as a direct result of its size and interaction with the FcRn mediated recycling pathway, see, e.g., Sleep D. et al., “Albumin as a versatile platform for drug half-life extension”, Biochim Biophys Acta, 2013, 1830(12):5526-34.

More specifically, said one or more other groups, residues, moieties or binding units that provide the FcRn antagonist of the present invention with increased half-life can be chosen from the group consisting of a polyethylene glycol (PEG) molecule, ELNN polypeptides or fragments thereof, binding units that can bind to serum albumin, such as human serum albumin. In one embodiment, said one or more other groups, residues, moieties or binding units that provide the molecule of the present technology with increased half-life is a binding unit that can bind to human serum albumin. In one embodiment, the binding unit is an ISVD.

For instance, the HLE moiety is an ISVD, for instance a human serum albumin (HSA)-binding ISVD. For example, WO 2004/041865 describes ISVDs binding to serum albumin (and in particular against HSA) that can be used to increase the half-life of the FcRn antagonists of the present invention.

The international application WO 2006/122787, the content of which is herein incorporated by reference, describes a number of ISVDs against (human) serum albumin. These ISVDs include the ISVDs called Alb-1 (SEQ ID NO: 52 in WO 2006/122787) and humanized variants thereof, such as Alb-8 (SEQ ID NO: 62 in WO 2006/122787). Again, these can be used to extend the half-life of FcRn antagonists of the present invention.

WO 2012/175400, the content of which is herein incorporated by reference, describes a further improved version of Alb-1, called Alb-23.

In one embodiment, the fusion protein of the present invention comprises a serum albumin binding moiety selected from Alb-1, Alb-3, Alb-4, Alb-5, Alb-6, Alb-7, Alb-8, Alb-9, Alb-10 (described in WO 2006/122787) and Alb-23. In one embodiment, the serum albumin binding moiety is Alb-8 or Alb-23 or its variants, as shown on pages 7-9 of WO 2012/175400. In one embodiment, the serum albumin binding moiety is selected from the albumin binders described in WO 2012/175741, WO 2015/173325, WO 2017/080850, WO 2017/085172, WO 2018/104444, WO 2018/134235, and WO 2018/134234, the content of which is herein incorporated by reference. Some serum albumin binders are also shown in Table 5 below.

In one embodiment, the molecule of the present technology comprises a HLE moiety as described in the following item A:
A. An ISVD that binds to human serum albumin and comprises
   i. a CDR1 (Abm) that is the amino acid sequence of SEQ ID NO: 19 (GFTFRSFGMS) or an amino acid sequence with 2 or 1 amino acid difference with SEQ ID NO: 19;
   ii. a CDR2 (Abm) that is the amino acid sequence of SEQ ID NO: 20 (SISGSGSDTL) or an amino acid sequence with 2 or 1 amino acid difference with SEQ ID NO: 20; and
   iii. a CDR3 (Abm) that is the amino acid sequence of SEQ ID NO: 21 (GGSLSR) or an amino acid sequence with 2 or 1 amino acid difference with SEQ ID NO: 21.

In one embodiment, the ISVD comprises a CDR1(Abm) that is the amino acid sequence of SEQ ID NO: 19, a CDR2 (Abm) that is the amino acid sequence of SEQ ID NO: 20 and a CDR3 (Abm) that is the amino acid sequence of SEQ ID NO: 21.

Item A can be also described using the Kabat CDR definition as:
A′. An ISVD that binds to human serum albumin and comprises
   i. a CDR1 that is the amino acid sequence of SEQ ID NO: 16 (SFGMS) or an amino acid sequence with 2 or 1 amino acid difference with SEQ ID NO: 16;
   ii. a CDR2 that is the amino acid sequence of SEQ ID NO: 17 (SISGSGSDTLYADSVKG) or an amino acid sequence with 2 or 1 amino acid difference with SEQ ID NO: 17; and
   iii. a CDR3 that is the amino acid sequence of SEQ ID NO: 18 (GGSLSR) or an amino acid sequence with 2 or 1 amino acid difference with SEQ ID NO: 18.

In one embodiment, the ISVD comprises a CDR1 (Kabat) that is the amino acid sequence of SEQ ID NO: 16, a CDR2 (Kabat) that is the amino acid sequence of SEQ ID NO: 17 and a CDR3 (Kabat) that is the amino acid sequence of SEQ ID NO: 18.

Examples of such an ISVD that binds to human serum albumin have one or more, or all, framework regions as indicated for construct ALB23002 (SEQ ID NO.: 58) in Tables B-1 and B-2 (in addition to the CDRs as defined in the preceding item A and item A′). In one embodiment, it is an ISVD comprising or consisting of the full amino acid sequence of construct ALB23002 (SEQ ID NO: 58).

TABLE B-1
Sequences for CDRs according to AbM CDR
and framework annotation (“ID” refers
to the given SEQ ID NO)

| ID | VHH | ID | FR1 | ID | CDR1 | ID | FR2 | ID | CDR2 | ID | FR3 | ID | CDR3 | ID | FR4 |
|---|---|---|---|---|---|---|---|---|---|---|---|---|---|---|---|
| 58 | ALB | 170 | EVQ | 19 | GFTF | 117 | WV | 20 | SIS | 172 | YAD | 21 | GGSL | 173 | SS |
| | 230 | | LVE | | RSFG | | RQ | | GSG | | SVK | | SR | | QG |
| | 02 | | SGG | | MS | | AP | | SDT | | GRF | | | | TL |
| | | | GVV | | | | GK | | L | | TIS | | | | VT |
| | | | QP | | | | GP | | | | RDN | | | | VS |
| | | | GGS | | | | EW | | | | SKN | | | | S |
| | | | LRL | | | | VS | | | | TLY | | | | |
| | | | SCA | | | | | | | | LQM | | | | |
| | | | AS | | | | | | | | NSL | | | | |
| | | | | | | | | | | | RPE | | | | |
| | | | | | | | | | | | DTA | | | | |
| | | | | | | | | | | | LYY | | | | |
| | | | | | | | | | | | CTI | | | | |

TABLE B-2
Sequences for CDRs according to Kabat CDR and frameworks annotation
(“ID” refers to the given SEQ ID NO)

| ID | VHH | ID | FR1 | ID | CDR1 | ID | FR2 | ID | CDR2 | ID | FR3 | ID | CDR3 | ID | FR4 |
|---|---|---|---|---|---|---|---|---|---|---|---|---|---|---|---|
| 58 | ALB | 174 | EV | 16 | SFG | 171 | WVR | 17 | SISGS | 175 | RFTIS | 18 | GGSL | 173 | SSQG |
| | 230 | | QLV | | MS | | QAP | | GSDT | | RDNS | | SR | | TLVT |
| | 02 | | ESG | | | | GKGP | | LYAD | | KNTL | | | | VSS |
| | | | GG | | | | EWV | | SVKG | | YLQM | | | | |
| | | | W | | | | S | | | | NSLR | | | | |
| | | | QP | | | | | | | | PEDT | | | | |
| | | | GG | | | | | | | | ALYY | | | | |
| | | | SLR | | | | | | | | CTI | | | | |
| | | | LSC | | | | | | | | | | | | |
| | | | AAS | | | | | | | | | | | | |
| | | | GFT | | | | | | | | | | | | |
| | | | FR | | | | | | | | | | | | |

In one embodiment, the fusion protein of the present invention comprises the serum albumin binding moiety Alb23 (SEQ ID NO: 46) as defined in Table 5 below. In one preferred embodiment, the fusion protein of the present invention comprises the serum albumin binding moiety Alb23002 (SEQ ID NO: 58) as defined in Table 5 below. In another preferred embodiment, the fusion protein of the present invention comprises the serum albumin binding moiety Alb23002(E1D) (SEQ ID NO: 60) as defined in Table 5 below.

Examples of such an ISVD that binds to human serum albumin have one or more, or all, framework regions as indicated for construct ALB23002 in Table 5 (in addition to the CDRs as defined in the preceding items A and A′). In one embodiment, it is an ISVD comprising or consisting of the full amino acid sequence of construct ALB23002 (SEQ ID NO: 58, see also Table 5).

Also in another embodiment, the amino acid sequence of an ISVD binding to human serum albumin may have a sequence identity of more than 90%, such as more than 95% or more than 99%, with SEQ ID NO: 58, wherein the CDRs are as defined in the preceding items A and A′. In one embodiment, the ISVD binding to human serum albumin comprises or consists of the amino acid sequence of SEQ ID NO: 58.

When such an ISVD binding to human serum albumin has 2 or 1 amino acid difference in at least one CDR relative to a corresponding reference CDR sequence (items A or A′ above), the ISVD has at least half the binding affinity, or at least the same binding affinity, to human serum albumin compared to construct ALB23002 (SEQ ID NO: 58), wherein the binding affinity is measured using the same method, such as SPR.

TABLE 5
Serum albumin binding ISVD sequences (“ID” refers to the SEQ ID NO as used herein)

| Name | ID | Amino acid sequence |
|---|---|---|
| Alb8 | 45 | EVQLVESGGGLVQPGNSLRLSCAASGFTFSSFGMSWVRQAPGKGLEWVSSI |
| | | SGSGSDTLYADSVKGRFTISRDNAKTTLYLQMNSLRPEDTAVYYCTIGGSL |
| | | SRSSQGTLVTVSS |
| Alb23 | 46 | EVQLLESGGGLVQPGGSLRLSCAASGFTFRSFGMSWVRQAPGKGPEWVSSI |
| | | SGSGSDTLYADSVKGRFTISRDNSKNTLYLQMNSLRPEDTAVYYCTIGGSL |
| | | SRSSQGTLVTVSS |
| Alb129 | 47 | EVQLVESGGGVVQPGNSLRLSCAASGFTFSSFGMSWVRQAPGKGLEWVSSIS |
| | | GSGSDTLYADSVKGRFTISRDNAKTTLYLQMNSLRPEDTATYYCTIGGSLSR |
| | | SSQGTLVTVSSA |
| Alb132 | 48 | EVQLVESGGGVVQPGGSLRLSCAASGFTFRSFGMSWVRQAPGKGPEWVSSI |
| | | SGSGSDTLYADSVKGRFTISRDNSKNTLYLQMNSLRPEDTATYYCTIGGSL |
| | | SRSSQGTLVTVSSA |
| Alb11 | 49 | EVQLVESGGGLVQPGNSLRLSCAASGFTFSSFGMSWVRQAPGKGLEWVSSI |
| | | SGSGSDTLYADSVKGRFTISRDNAKTTLYLQMNSLRPEDTAVYYCTIGGSL |
| | | SRSSQGTLVTVSS |
| Alb11 | 50 | EVQLVESGGGLVQPGNSLRLSCAASGFTFSSFGMSWVRQAPGKGLEWVSSI |
| (S1 | | SGSGSDTLYADSVKGRFTISRDNAKTTLYLQMNSLRPEDTAVYYCTIGGSL |
| 12K)-A | | SRSSQGTLVKVSSA |
| Alb82 | 51 | EVQLVESGGGVVQPGNSLRLSCAASGFTFSSFGMSWVRQAPGKGLEWVSSIS |
| | | GSGSDTLYADSVKGRFTISRDNAKTTLYLQMNSLRPEDTALYYCTIGGSLSR |
| | | SSQGTLVTVSS |
| Alb82-A | 52 | EVQLVESGGGVVQPGNSLRLSCAASGFTFSSFGMSWVRQAPGKGLEWVSS |
| | | ISGSGSDTLYADSVKGRFTISRDNAKTTLYLQMNSLRPEDTALYYCTIGG |
| | | SLSRSSQGTLVTVSSA |
| Alb82- | 53 | EVQLVESGGGVVQPGNSLRLSCAASGFTFSSFGMSWVRQAPGKGLEWVSSIS |
| AA | | GSGSDTLYADSVKGRFTISRDNAKTTLYLQMNSLRPEDTALYYCTIGGSLSR |
| | | SSQGTLVTVSSAA |
| Alb82- | 54 | EVQLVESGGGVVQPGNSLRLSCAASGFTFSSFGMSWVRQAPGKGLEWVSSIS |
| AAA | | GSGSDTLYADSVKGRFTISRDNAKTTLYLQMNSLRPEDTALYYCTIGGSLSR |
| | | SSQGTLVTVSSAAA |
| Alb82-G | 55 | EVQLVESGGGVVQPGNSLRLSCAASGFTFSSFGMSWVRQAPGKGLEWVSSIS |
| | | GSGSDTLYADSVKGRFTISRDNAKTTLYLQMNSLRPEDTALYYCTIGGSLSR |
| | | SSQGTLVTVSSG |
| Alb82- | 56 | EVQLVESGGGVVQPGNSLRLSCAASGFTFSSFGMSWVRQAPGKGLEWVSSIS |
| GG | | GSGSDTLYADSVKGRFTISRDNAKTTLYLQMNSLRPEDTALYYCTIGGSLSR |
| | | SSQGTLVTVSSGG |
| Alb82- | 57 | EVQLVESGGGVVQPGNSLRLSCAASGFTFSSFGMSWVRQAPGKGLEWVSSIS |
| GGG | | GSGSDTLYADSVKGRFTISRDNAKTTLYLQMNSLRPEDTALYYCTIGGSLSR |
| | | SSQGTLVTVSSGGG |
| Alb23002 | 58 | EVQLVESGGGVVQPGGSLRLSCAASGFTFRSFGMSWVRQAPGKGPEWVSSI |
| | | SGSGSDTLYADSVKGRFTISRDNSKNTLYLQMNSLRPEDTALYYCTIGGSL |
| | | SRSSQGTLVTVSS |
| Alb223 | 59 | EVQLVESGGGVVQPGGSLRLSCAASGFTFRSFGMSWVRQAPGKGPEWVSSI |
| | | SGSGSDTLYADSVKGRFTISRDNSKNTLYLQMNSLRPEDTALYYCTIGGSL |
| | | SRSSQGTLVTVSSA |
| Alb23002 | 60 | DVQLVESGGGVVQPGGSLRLSCAASGFTFRSFGMSWVRQAPGKGPEWVSSI |
| (E1D) | | SGSGSDTLYADSVKGRFTISRDNSKNTLYLQMNSLRPEDTALYYCTIGGSL |
| | | SRSSQGTLVTVSS |

The fusion protein of the present invention may additionally comprise (besides the FcRn antagonist) one or more targeting moieties. A “targeting moiety”, as defined herein, is any group, residue, moiety, or binding unit which is capable of being directed through its binding to a target.

Further, the fusion protein of the present invention may additionally comprise one or more therapeutic moieties. A “therapeutic moiety”, as defined herein, is any group, residue, moiety, or binding unit which is capable of exerting a therapeutic activity in the animal and/or human body. The therapeutic moiety may also be in the form of a precursor, which then gets activated to exert its therapeutic activity.

### Nucleic Acid Molecules

The present invention also provides a nucleic acid molecule encoding the FcRn antagonists or fusion protein of the present invention.

A “nucleic acid molecule” (used interchangeably with “nucleic acid”) is a chain of nucleotide monomers linked to each other via a phosphate backbone to form a nucleotide sequence. A nucleic acid may be used to transform/transfect a host cell or host organism, e.g., for expression and/or production of a polypeptide. Suitable (non-human) hosts or host cells for production purposes will be clear to the skilled person, and may for example be any suitable fungal, prokaryotic or eukaryotic cell or cell line or any suitable fungal, prokaryotic or eukaryotic organism. A host or host cell comprising a nucleic acid encoding the protein-based carrier building block and/or the molecule (or part of the molecule) of the present invention is also encompassed by the present invention.

A nucleic acid may be for example DNA, RNA, or a hybrid thereof, and may also comprise (e.g., chemically) modified nucleotides, like PNA. It can be single- or double-stranded. In one embodiment, it is in the form of double-stranded DNA. For example, the nucleotide sequences of the present invention may be genomic DNA, cDNA.

The nucleic acids of the present invention can be prepared or obtained in a manner known per se, and/or can be isolated from a suitable natural source. Nucleotide sequences encoding naturally occurring (poly)peptides can for example be subjected to site-directed mutagenesis, so as to provide a nucleic acid molecule encoding polypeptide with sequence variation. Also, as will be clear to the skilled person, to prepare a nucleic acid, also several nucleotide sequences, such as at least one nucleotide sequence encoding a targeting moiety and for example nucleic acids encoding one or more linkers can be linked together in a suitable manner.

Techniques for generating nucleic acids will be clear to the skilled person and may for instance include, but are not limited to, automated DNA synthesis; site-directed mutagenesis; combining two or more naturally occurring and/or synthetic sequences (or two or more parts thereof), introduction of mutations that lead to the expression of a truncated expression product; introduction of one or more restriction sites (e.g. to create cassettes and/or regions that may easily be digested and/or ligated using suitable restriction enzymes), and/or the introduction of mutations by means of a PCR reaction using one or more “mismatched” primers.

### Vectors

Also provided is a vector comprising the nucleic acid molecule encoding FcRn antagonists or fusion protein of the present invention.

A vector as used herein is a vehicle suitable for carrying genetic material into a cell. A vector includes naked nucleic acids, such as plasmids or mRNAs, or nucleic acids embedded into a bigger structure, such as liposomes or viral vectors.

In some embodiments, vectors comprise at least one nucleic acid that is optionally linked to one or more regulatory elements, such as for example one or more suitable promoter(s), enhancer(s), terminator(s), etc.). In one embodiment, the vector is an expression vector, i.e., a vector suitable for expressing an encoded polypeptide or construct under suitable conditions, e.g. when the vector is introduced into a (e.g., human) cell. DNA-based vectors include the presence of elements for transcription (e.g., a promoter and a polyA signal) and translation (e.g., Kozak sequence).

In one embodiment, in the vector, said at least one nucleic acid and said regulatory elements are “operably linked” to each other, by which is generally meant that they are in a functional relationship with each other. For instance, a promoter is considered “operably linked” to a coding sequence if said promoter is able to initiate or otherwise control/regulate the transcription and/or the expression of a coding sequence (in which said coding sequence should be understood as being “under the control of” said promotor). Generally, when two nucleotide sequences are operably linked, they will be in the same orientation and usually also in the same reading frame. They will usually also be essentially contiguous, although this may also not be required.

In one embodiment, any regulatory elements of the vector are such that they are capable of providing their intended biological function in the intended host cell or host organism.

For instance, a promoter, enhancer or terminator should be “operable” in the intended host cell or host organism, by which is meant that for example said promoter should be capable of initiating or otherwise controlling/regulating the transcription and/or the expression of a nucleotide sequence—e.g. a coding sequence—to which it is operably linked.

### The Host Cell of the Present Invention

The nucleic acids of the invention and/or the genetic constructs of the invention (nucleic acids of the invention) may be used to transform a host cell or host organism, i.e., for expression and/or production of the FcRn antagonists and/or fusion proteins of the invention. The host is preferably a non-human host. Suitable hosts or host cells will be clear to the skilled person, and may for example be any suitable fungal, prokaryotic or eukaryotic cell or cell line or any suitable fungal, prokaryotic or eukaryotic organism, for example: a bacterial strain, including but not limited to gram-negative strains such as strains of Escherichia coli; of Proteus, for example of Proteus mirabilis; of Pseudomonas, for example of Pseudomonas fluorescens; and gram-positive strains such as strains of Bacillus, for example of Bacillus subtilis or of Bacillus brevis; of Streptomyces, for example of Streptomyces lividans; of Staphylococcus, for example of Staphylococcus carnosus; and of Lactococcus, for example of Lactococcus lactis; a fungal cell, including but not limited to cells from species of Trichoderma, for example from Trichoderma reesei; of Neurospora, for example from Neurospora crassa; of Sordaria, for example from Sordaria macrospora; of Aspergillus, for example from Aspergillus niger or from Aspergillus sojae; or from other filamentous fungi; a yeast cell, including but not limited to cells from species of Saccharomyces, for example of Saccharomyces cerevisiae; of Schizosaccharomyces, for example of Schizosaccharomyces pombe; of Pichia, for example of Pichia pastoris or of Pichia methanolica; of Hansenula, for example of Hansenula polymorpha; of Kluyveromyces, for example of Kluyveromyces lactis; of Arxula, for example of Arxula adeninivorans; of Yarrowia, for example of Yarrowia lipolytica; an amphibian cell or cell line, such as Xenopus oocytes; an insect-derived cell or cell line, such as cells/cell lines derived from lepidoptera, including but not limited to Spodoptera SF9 and Sf21 cells or cells/cell lines derived from Drosophila, such as Schneider and Kc cells; a plant or plant cell, for example in tobacco plants; and/or a mammalian cell or cell line, for example a cell or cell line derived from a human, a cell or a cell line from mammals including but not limited to CHO-cells, BHK-cells (for example BHK-21 cells) and human cells or cell lines such as HeLa, COS (for example COS-7) and PER.C6 cells; as well as all other hosts or host cells known per se for the expression and production of antibodies and antibody fragments (including but not limited to (single) domain antibodies and scFv fragments), which will be clear to the skilled person. Reference is also made to the general background art cited hereinabove, as well as to for example WO 94/29457; WO 96/34103; WO 99/42077; Frenken et al. 1998 (Res. Immunol. 149: 589-99); Riechmann and Muyldermans 1999 (J. Immunol. Met. 231: 25-38); van der Linden 2000 (J. Biotechnol. 80: 261-70); Joosten et al. 2003 (Microb. Cell Fact. 2: 1); Joosten et al. 2005 (Appl. Microbiol. Biotechnol. 66: 384-92); and the further references cited herein.

For expression of the FcRn antagonists and/or fusion proteins in a cell, they may also be expressed as so-called “intrabodies”, as for example described in WO 94/02610, WO 95/22618 and U.S. Pat. No. 7,004,940; WO 03/014960; in Cattaneo and Biocca 1997 (Intracellular Antibodies: Development and Applications. Landes and Springer-Verlag); and in Kontermann 2004 (Methods 34: 163-170).

According to one preferred, but non-limiting embodiment of the invention, the FcRn antagonists and/or fusion proteins of the invention are produced in a bacterial cell, in particular a bacterial cell suitable for large scale pharmaceutical production, such as cells of the strains mentioned above.

According to another preferred, but non-limiting embodiment of the invention, the FcRn antagonists and/or fusion proteins of the invention are produced in a yeast cell, in particular a yeast cell suitable for large scale pharmaceutical production, such as cells of the species mentioned above.

According to yet another preferred, but non-limiting embodiment of the invention, the FcRn antagonists and/or fusion proteins of the invention are produced in a mammalian cell, in particular in a human cell or in a cell of a human cell line, and more in particular in a human cell or in a cell of a human cell line that is suitable for large scale pharmaceutical production, such as the cell lines mentioned hereinabove.

Suitable techniques for transforming a host or host cell of the invention will be clear to the skilled person and may depend on the intended host cell/host organism and the genetic construct to be used. Reference is again made to the handbooks and patent applications mentioned above.

After transformation, a step for detecting and selecting those host cells or host organisms that have been successfully transformed with the nucleotide sequence/genetic construct of the invention may be performed. This may for instance be a selection step based on a selectable marker present in the genetic construct of the invention or a step involving the detection of the polypeptide of the invention, e.g., using specific antibodies.

The transformed host cell (which may be in the form or a stable cell line) or host organisms (which may be in the form of a stable mutant line or strain) form further aspects of the present invention.

Preferably, these host cells or host organisms are such that they express, or are (at least) capable of expressing (e.g., under suitable conditions), the FcRn antagonists and/or fusion proteins of the invention (and in case of a host organism: in at least one cell, part, tissue or organ thereof). The invention also includes further generations, progeny and/or offspring of the host cell or host organism of the invention, for instance obtained by cell division or by sexual or asexual reproduction.

Accordingly, in another aspect, the invention relates to a host or host cell that expresses (or that under suitable circumstances is capable of expressing) the FcRn antagonists and/or fusion proteins of the invention; and/or that contains a nucleic acid encoding the same. Some preferred but non-limiting examples of such hosts or host cells can be as generally described in WO 04/041867, WO 04/041865 or WO 09/068627. For example, the FcRn antagonists and/or fusion proteins of the invention may with advantage be expressed, produced or manufactured in mammalian cells, such as Chinese Hamster Overy cells (CHO cells), or in a suitable yeast strain, such as a strain of Pichia pastoris. Reference is also made to WO 04/25591, WO 10/125187, WO 11/003622, and WO 12/056000 which also describes the expression/production in Pichia and other hosts/host cells of immunoglobulin single variable domains and polypeptides comprising the same.

To produce/obtain expression of the FcRn antagonists and/or fusion proteins of the invention, the transformed host cell or transformed host organism may generally be kept, maintained and/or cultured under conditions such that the (desired) FcRn antagonists and/or fusion proteins of the invention are expressed/produced. Suitable conditions will be clear to the skilled person and will usually depend upon the host cell/host organism used, as well as on the regulatory elements that control the expression of the (relevant) nucleotide sequence of the invention. Again, reference is made to the handbooks and patent applications mentioned above in the paragraphs on the genetic constructs of the invention.

Generally, suitable conditions may include the use of a suitable medium, the presence of a suitable source of food and/or suitable nutrients, the use of a suitable temperature, and optionally the presence of a suitable inducing factor or compound (e.g., when the nucleotide sequences of the invention are under the control of an inducible promoter); all of which may be selected by the skilled person. Again, under such conditions, the FcRn antagonists and/or fusion proteins of the invention may be expressed in a constitutive manner, in a transient manner, or only when suitably induced.

It will also be clear to the skilled person that the FcRn antagonists and/or fusion proteins of the invention may (first) be generated in an immature form (as mentioned above), which may then be subjected to post-translational modification, depending on the host cell/host organism used. Also, the FcRn antagonists and/or fusion proteins of the invention may be glycosylated, again depending on the host cell/host organism used.

The FcRn antagonists and/or fusion proteins of the invention may then be isolated from the host cell/host organism and/or from the medium in which said host cell or host organism was cultivated, using protein isolation and/or purification techniques known per se, such as (preparative) chromatography and/or electrophoresis techniques, differential precipitation techniques, affinity techniques (e.g., using a specific, cleavable amino acid sequence fused with the polypeptide or construct of the invention) and/or preparative immunological techniques (i.e., using antibodies against the amino acid sequence to be isolated).

An polypeptide or protein is considered to be “(in) essentially isolated (form)”—for example, compared to its native biological source and/or the reaction medium or cultivation medium from which it has been obtained—when it has been separated from at least one other component with which it is usually associated in said source or medium, such as another protein/polypeptide, another biological component or macromolecule or at least one contaminant, impurity or minor component. In particular, a polypeptide or protein is considered “essentially isolated” when it has been purified at least 2-fold, in particular at least 10-fold, more in particular at least 100-fold, and up to 1000-fold or more. A polypeptide or protein that is “in essentially isolated form” is preferably essentially homogeneous, as determined using a suitable technique, such as a suitable chromatographical technique, such as polyacrylamide-gel electrophoresis.

### Compositions

The invention also relates to a composition comprising the FcRn antagonists and/or fusion proteins of the invention. The composition may be a pharmaceutical composition. The composition may further comprise at least one pharmaceutically acceptable carrier, diluent or excipient and/or adjuvant, and optionally comprise one or more further pharmaceutically active polypeptides and/or compounds.

In the above methods, the FcRn antagonists and/or fusion proteins can be administered in any suitable manner, depending on the specific pharmaceutical formulation or composition to be used. Thus, the FcRn antagonists and/or fusion proteins of the invention and/or the compositions comprising the same can for example be administered orally, intraperitoneally, intravenously, subcutaneously, intramuscularly, or via any other route of administration that circumvents the gastrointestinal tract, intranasally, transdermally, topically, by means of a suppository, by inhalation, again depending on the specific pharmaceutical formulation or composition to be used. The clinician will be able to select a suitable route of administration and a suitable pharmaceutical formulation or composition to be used in such administration, depending on the disease or disorder to be prevented or treated and other factors well known to the clinician.

An effective amount of a molecule as described, or a composition comprising the molecule of the present invention can be administered to a subject in order to provide the intended treatment results.

As used herein, the term “therapy” refers to any protocol, method and/or agent that can be used in the treatment, prevention and/or management of a disease and/or disorder, or symptoms thereof. In certain embodiments, the terms “therapies” and “therapy” refer to a biological therapy, supportive therapy, and/or other therapies useful in the treatment, prevention and/or management of a disease and/or disorder, or one or more symptoms thereof known to one of skill in the art, such as medical personnel.

As used herein, the terms “treat”, “treatment” and “treating” in the context of administering (a) therapy(ies) to a subject refer to the reduction or amelioration of the progression, severity, and/or duration of a diseases or disorder, and/or the amelioration of one or more symptoms thereof resulting from the administration of one or more therapies (including, but not limited to, the administration of one or more prophylactic or therapeutic agents).

The FcRn antagonists, fusion proteins or compositions of the invention and/or the compositions comprising the same are administered according to a regime of treatment that is suitable for preventing and/or treating the disease and/or disorder to be prevented or treated. The clinician will generally be able to determine a suitable treatment regimen, depending on factors such as the stage of the disease and/or disorder to be treated, the severity of the disease and/or disorder to be treated and/or the severity of the symptoms thereof, the specific FcRn antagonists, fusion proteins or compositions of the invention to be used, the specific route of administration and pharmaceutical formulation or composition to be used, the age, gender, weight, diet, general condition of the patient, and similar factors well known to the clinician.

Generally, the treatment regimen will comprise the administration of one or more FcRn antagonists, fusion proteins or compositions of the invention, or of one or more compositions comprising the same, in one or more pharmaceutically effective amounts or doses. The specific amount(s) or doses to be administered can be determined by the clinician, again based on the factors cited above.

Usually, in the above method, a single FcRn antagonist, fusion proteins or compositions of the invention will be used. It is however within the scope of the invention to use two or more FcRn antagonists, fusion proteins or compositions of the invention in combination.

The FcRn antagonists, fusion proteins or compositions of the invention may also be used in combination with one or more further pharmaceutically active compounds or principles, i.e., as a combined treatment regimen, which may or may not lead to a synergistic effect. Again, the clinician will be able to select such further compounds or principles, as well as a suitable combined treatment regimen, based on the factors cited above and his expert judgement.

In FcRn antagonists, compositions or fusion proteins of the invention may be used in combination with other pharmaceutically active compounds or principles that are or can be used for the prevention and/or treatment of the disease and/or disorder cited herein, as a result of which a synergistic effect may or may not be obtained. Examples of such compounds and principles, as well as routes, methods and pharmaceutical formulations or compositions for administering them will be clear to the clinician.

When two or more substances or principles are to be used as part of a combined treatment regimen, they can be administered via the same route of administration or via different routes of administration, at essentially the same time or at different times (e.g., essentially simultaneously, consecutively, or according to an alternating regime). When the substances or principles are to be administered simultaneously via the same route of administration, they may be administered as different pharmaceutical formulations or compositions or part of a combined pharmaceutical formulation or composition, as will be clear to the skilled person.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

Methods and Uses of the FcRn Antagonists, Compositions, and/or Fusion Proteins

In another aspect, the present invention provides FcRn-antagonists, compositions and/or fusion proteins comprising the same for use in medicine. In particular, the present invention provides the use of the FcRn antagonists, fusion proteins and/or compositions of the present invention in therapy, for instance for the treatment of an antibody-mediated disorder in a subject. In certain embodiments, the present invention provides compositions and articles of manufacture (e.g., kits) for use in such treatment methods.

Hence, the present invention provides the use of the FcRn antagonists, fusion proteins and/or compositions in the manufacture of a medicament, in particular a medicament for the treatment of an antibody-mediated disorder in a subject. Hence, the present invention provides methods for treatment antibody-mediated disorders in a subject, wherein the methods comprise the administration of the FcRn antagonists, fusion proteins and/or compositions of the present invention to a subject in need thereof.

As used herein, the terms “antibody-related”, “antibody-mediated”, and “antibody-responsive” disorder, condition, or disease refer to a disorder, condition, or disease that may be ameliorated by removal undesired antibody (e.g., certain autoreactive IgG, or exogenously provided therapeutic or diagnostic IgG that is no longer needed or beneficial to the patient).

In certain embodiments, the antibody-mediated disorder is an autoimmune disease.

In certain embodiments, the autoimmune disease is selected from the group consisting of allogenic islet graft rejection, alopecia areata, ankylosing spondylitis, antiphospholipid syndrome, autoimmune Addison's disease, Alzheimer's disease, antineutrophil cytoplasmic autoantibodies (ANCA), autoimmune diseases of the adrenal gland, autoimmune hemolytic anemia, autoimmune hepatitis, autoimmune myocarditis, autoimmune neutropenia, autoimmune oophoritis and orchitis, autoimmune thrombocytopenia, autoimmune urticaria, Behcet's disease, bullous pemphigoid, cardiomyopathy, Castleman's syndrome, celiacspruce-dermatitis, chronic fatigue immune disfunction syndrome, chronic inflammatory demyelinating polyneuropathy (CIDP), Churg-Strauss syndrome, cicatrical pemphigoid, CREST syndrome, cold agglutinin disease, Crohn's disease, dermatomyositis, dilated cardiomyopathy, discoid lupus, epidermolysis bullosa acquisita, essential mixed cryoglobulinemia, factor VIII deficiency, fibromyalgia-fibromyositis, glomerulonephritis, Grave's disease, Guillain-Barre, Goodpasture's syndrome, graft-versus-host disease (GVHD), Hashimoto's thyroiditis, hemophilia A, idiopathic membranous neuropathy, idiopathic pulmonary fibrosis, idiopathic thrombocytopenia purpura (ITP), IgA neuropathy, IgM polyneuropathies, immune mediated thrombocytopenia, juvenile arthritis, Kawasaki's disease, lichen plantus, lichen sclerosus, lupus erthematosis, Meniere's disease, mixed connective tissue disease, mucous membrane pemphigoid, multiple sclerosis, type 1 diabetes mellitus, Multifocal motor neuropathy (MMN), myasthenia gravis, paraneoplastic bullous pemphigoid, pemphigoid gestationis, pemphigus vulgaris, pemphigus foliaceus, pernicious anemia, polyarteritis nodosa, polychrondritis, polyglandular syndromes, polymyalgia rheumatica, polymyositis and dermatomyositis, primary agammaglobinulinemia, primary biliary cirrhosis, psoriasis, psoriatic arthritis, relapsing polychondritis, Reynauld's phenomenon, Reiter's syndrome, rheumatoid arthritis, sarcoidosis, scleroderma, Sjorgen's syndrome, solid organ transplant rejection, stiff-man syndrome, systemic lupus erythematosus, takayasu arteritis, toxic epidermal necrolysis (TEN), Stevens Johnson syndrome (SJS), temporal arteristis/giant cell arteritis, thrombotic thrombocytopenia purpura, ulcerative colitis, uveitis, dermatitis herpetiformis vasculitis, anti-neutrophil cytoplasmic antibody-associated vasculitides, vitiligo, and Wegner's granulomatosis.

In certain embodiments, the autoimmune disease is selected from the group consisting of graft versus host disease (GVHD), systemic lupus erythematosus (SLE), myasthenia gravis, systemic sclerosis (SSc)/scleroderma, rheumatoid arthritis, psoriatic arthritis, osteoarthritis, diabetes, multiple sclerosis, pemphigus vulgaris, atopic dermatitis, psoriasis, asthma, allergy, idiopathic pulmonary fibrosis (IPF), idiopathic thrombocytopenia purpura (ITP), and hidradenitis suppurativa.

In certain embodiments, the IgG-mediated disorder may be an allergy. The allergy may be, for example, a food allergy, a drug allergy, an environmental allergy and a contact allergy.

In certain embodiments, the FcRn antagonists, fusion proteins and/or compositions of the present invention reduce the serum levels of an Fc-containing agent in subject that has been administered the FcRn antagonists, wherein said FcRn antagonists, fusion proteins and/or compositions are simultaneously or sequentially administered to said subject. Clearance of an Fc-containing agent may be desired in cases where the Fc-containing agent is detrimental (e.g., toxic) to a subject, or wherein the Fc-containing reagent is only desired to be present in a subject for a certain period of time, for example, when the Fc-containing agent is immunogenic or when the Fc-containing agent is an antibody-drug conjugate, respectively. In certain embodiments, clearance of an Fc-containing agent reduces the amount of exposure of the subject to the Fc-containing agent. The level of any Fc-containing agent in the serum may be reduced by employing the FcRn antagonists, fusion proteins and/or compositions described herein. Accordingly, in certain embodiments, an FcRn antagonists, fusion proteins and/or compositions disclosed herein are used to reduce the serum levels of an Fc-containing agent in a subject that has been administered the FcRn antagonists, fusion proteins and/or compositions. For example, a subject administered a therapeutic agent may develop antibodies, e.g., anti-drug antibodies, that reduce the availability and/or efficacy of the administered therapeutic agent. The presence of anti-drug antibodies in a subject may result in unwanted side effects. Accordingly, the FcRn antagonists, fusion proteins and/or compositions disclosed herein remove the anti-drug antibodies that develop in a subject. In another non-limiting example, the FcRn antagonists, fusion proteins and/or compositions disclosed herein can be administered after having administered an effective amount of a radiolabeled antibody to reduce exposure of a non-target tissue to a radiolabeled antibody during diagnostic imaging, comprising administering to a subject a therapeutically effective amount of the FcRn antagonists, fusion proteins and/or compositions described herein.

In certain embodiments, the FcRn antagonists, fusion proteins and/or compositions are administered to the subject simultaneously or sequentially with an additional therapeutic agent. In certain embodiments, the additional therapeutic agent is an anti-inflammatory agent.

Specific Examples of ISVDs Binding to FcRn that are Suitable for Use as First Polypeptides in the FcRn Antagonists of the Present Invention

Examples of ISVD specifically binding to FcRn that are suitable for use as first polypeptides in the FcRn antagonists of the present invention have been provided above in the present description, see, e.g., section “The first polypeptide (a) comprised in the FcRn antagonists of the present invention.

Further specific examples of ISVDs specifically binding to FcRn are ISVDs that comprises 4 framework regions (FR1 to FR4, respectively) and three complementarity determining regions (CDR1 to CDR3, respectively), wherein:
CDR1 (AbM numbering) consists of an amino acid sequence selected from:
   d) the amino acid sequence of GFTFSSYAMY (SEQ ID NO: 11);
   e) amino acid sequences that have at least 80% amino acid identity with the amino acid sequence of GFTFSSYAMY (SEQ ID NO: 11);
   f) amino acid sequences that have 3, 2, or 1 amino acid difference with the amino acid sequences GFTFSSYAMY (SEQ ID NO: 11);
and
CDR2 (AbM numbering) consists of an amino acid sequence selected from:
   g) the amino acid sequence of AISSGGGSTD (SEQ ID NO: 12);
   h) amino acid sequences that have at least 80% amino acid identity with the amino acid sequence of AISSGGGSTD (SEQ ID NO: 12);
   i) amino acid sequences that have 3, 2, or 1 amino acid difference with the amino acid sequence of AISSGGGSTD (SEQ ID NO: 12);
and
CDR3 (AbM numbering) consists of an amino acid sequence selected from:
   j) the amino acid sequence of DTLYTSLESYSY (SEQ ID NO:189);
   k) amino acid sequences that have at least 80% amino acid identity with the amino acid sequence of DTLYTSLESYSY (SEQ ID NO:189);
   l) amino acid sequences that have 3, 2, or 1 amino acid difference with the amino acid sequence of DTLYTSLESYSY (SEQ ID NO:189).

Preferably, the CDR sequences have at least 90% amino acid sequence identity, more preferably at least 95% amino acid sequence identity, such as 99% amino acid sequence identity or more, or even essentially 100% amino acid sequence identity with the CDR sequences of SEQ ID NOs: 11, 12, and/or 189.

In one embodiment, the ISVD comprise CDRs (AbM numbering) with an amino acid sequence that has at least 70% amino acid sequence identity, preferably at least 80% amino acid sequence identity, more preferably at least 90% amino acid sequence identity, such as 95% amino acid sequence identity or 99% amino acid sequence identity or more, or even essentially 100% amino acid sequence identity with the amino acid sequences of the CDRs of the ISVD with the amino acid sequence SEQ ID NO: 186 or 187.

(T0263018B11 (Y79S, T100bE, W100cS))
(CDRs according to Abm are underlined;
CDRs according to Kabat are in italics)
SEQ ID NO: 186
EVQLVESGGGVVQPGGSLRLSCAASGFTFSSYAMYWVRQ
APGKGLEWVSAISSGGGSTDYADSVKGRFTISRDNSKNT
LSLQMNSLRPEDTALYYCAADTLYTSLESYSYWGQGTLV
TVSS
(T0263018B11 (E1D, Y79S, T100bE, W100cS))
(CDRs according to Abm are underlined;
CDRs according to Kabat are in italics)
SEQ ID NO: 187
DVQLVESGGGVVQPGGSLRLSCAASGFTFSSYAMYWVRQ
APGKGLEWVSAISSGGGSTDYADSVKGRFTISRDNSKNT
LSLQMNSLRPEDTALYYCAADTLYTSLESYSYWGQGTLV
TVSS

In one embodiment, the ISVD comprises a CDR1 (AbM numbering) that is the amino acid sequence of SEQ ID NO: 11, a CDR2 (AbM numbering) that is the amino acid sequence of SEQ ID NO: 12 and a CDR3 (AbM numbering) that is the amino acid sequence of SEQ ID NO: 189.

Specific examples of such ISVDs that specifically bind to FcRn have one or more, or all, framework regions as indicated for T0263018B11 (Y79S, T100BE, W100CS) (SEQ ID NO: 186) or for T0263018B11 (E1D, Y79S, T100BE, W100CS) (SEQ ID NO: 187). In one embodiment, the ISVD comprises or consists of the full amino acid sequence of T0263018B11 (Y79S, T100BE, W100CS) (SEQ ID NO: 186) or the full amino acid sequence of T0263018B11 (E1D, Y79S, T100BE, W100CS) (SEQ ID NO: 187).

The SEQ ID NOs for the CDR sequences referred to above are based on the CDR definition according to the AbM definition (see Tables A-3 and A-5). It is noted that the SEQ ID NOs for the CDR sequences defined according to the Kabat definition can likewise be used (see Tables A-4 and A-6). Accordingly, the ISVDs provided by the present technology, specifically binding to FcRn as described above using the AbM definition, can be also described using the Kabat definition.

As such in one embodiment, the ISVDs specifically binding to FcRn are ISVDs that comprise 4 framework regions (FR1 to FR4, respectively) and three complementarity determining regions (CDR1 to CDR3, respectively), wherein:
CDR1 (Kabat numbering) consists of an amino acid sequence selected from:
   a) the amino acid sequence of SYAMY (SEQ ID NO: 8);
   b) amino acid sequences that have at least 80% amino acid identity with the amino acid sequence of SYAMY (SEQ ID NO: 8);
   c) amino acid sequences that have 3, 2, or 1 amino acid difference with the amino acid sequences of SYAMY (SEQ ID NO: 8);
and
CDR2 (Kabat numbering) consists of an amino acid sequence selected from:
   d) the amino acid sequence of AISSGGGSTDYADSVKG (SEQ ID NO: 9);
   e) amino acid sequences that have at least 80% amino acid identity with the amino acid sequence of AISSGGGSTDYADSVKG (SEQ ID NO: 9);
   f) amino acid sequences that have 3, 2, or 1 amino acid difference with the amino acid sequence of AISSGGGSTDYADSVKG (SEQ ID NO: 9);
and
CDR3 (Kabat numbering) consists of an amino acid sequence selected from:
   g) the amino acid sequence of DTLYTSLESYSY (SEQ ID NO: 189);
   h) amino acid sequences that have at least 80% amino acid identity with the amino acid sequence of DTLYTSLESYSY (SEQ ID NO: 189);
   i) amino acid sequences that have 3, 2, or 1 amino acid difference with the amino acid sequence of DTLYTSLESYSY (SEQ ID NO: 189).

Preferably, the CDR sequences have at least 90% amino acid sequence identity, more preferably at least 95% amino acid sequence identity, such as 99% amino acid sequence identity or more, or even essentially 100% amino acid sequence identity with the CDR sequences of SEQ ID NOs: 8, 9, and/or 189.

In one embodiment, the ISVD comprise CDRs (Kabat numbering) with an amino acid sequence that has at least 70% amino acid sequence identity, preferably at least 80% amino acid sequence identity, more preferably at least 90% amino acid sequence identity, such as 95% amino acid sequence identity or 99% amino acid sequence identity or more, or even essentially 100% amino acid sequence identity with the amino acid sequences of the CDRs of the ISVD with the amino acid sequence of SEQ ID NOs: 186 or 187.

In one embodiment, the ISVD comprises a CDR1 (Kabat numbering) that is the amino acid sequence of SEQ ID NO: 8, a CDR2 (Kabat numbering) that is the amino acid sequence of SEQ ID NO: 9 and a CDR3 (Kabat numbering) that is the amino acid sequence of SEQ ID NO: 189.

Specific examples of such ISVDs that specifically bind to FcRn have one or more, or all, framework regions as indicated for T0263018B11 (Y79S, T100BE, W100CS) (SEQ ID NO: 186) or for T0263018B11 (E1D, Y79S, T100BE, W100CS) (SEQ ID NO: 187). In one embodiment, the ISVD comprises or consists of the full amino acid sequence T0263018B11 (Y79S, T100BE, W100CS) (SEQ ID NO: 186) or the full amino acid sequence T0263018B11 (E1D, Y79S, T100BE, W100CS) (SEQ ID NO: 187) (see Tables A-3 to A-6).

In another embodiment, the ISVD specifically binding to human FcRn may have a sequence identity of more than 90%, such as more than 95% or even more than 99%, with SEQ ID NO: 186, wherein the CDRs are as defined above. In one embodiment, the ISVD specifically binding to FcRn comprises or consists of the amino acid sequence of SEQ ID NO: 186 or of SEQ ID NO: 187.

In another embodiment, when such an ISVD specifically binding to FcRn has 3, 2 or 1 amino acid difference in at least one CDR relative to a corresponding reference CDR sequence (above), the ISVD has at least (essentially) the same binding affinity to human FcRn compared to the VHHs with a sequence comprising or consisting of SEQ ID NO: 186 or of SEQ ID NO: 187, wherein the binding affinity is measured using the same method, such as, e.g., SPR.

In another embodiment, when such an ISVD specifically binding to FcRn has 3, 2 or 1 amino acid difference in at least one CDR relative to a corresponding reference CDR sequence (above), the ISVD has at least (essentially) the same binding affinity to cyno FcRn compared to one of the VHHs with a sequence comprising or consisting of SEQ ID NO: 186 or of SEQ ID NO: 187, wherein the binding affinity is measured using the same method, such as, e.g., SPR.

In another embodiment, when the CDRs of such an ISVD specifically binding to FcRn have at least 90% amino acid sequence identity, at least 95% amino acid sequence identity, such as 99% amino acid sequence identity or more, with a corresponding reference CDR sequence (above), the ISVD has at least (essentially) the same binding affinity to human FcRn compared to a VHH with SEQ ID NO: 186 or with SEQ ID NO: 187, wherein the binding affinity is measured using the same method, such as, e.g., SPR.

In another embodiment, when the CDRs of such an ISVD specifically binding to FcRn have at least 90% amino acid sequence identity, at least 95% amino acid sequence identity, such as 99% amino acid sequence identity or more, with a corresponding reference CDR sequence (above), the ISVD has at least (essentially) the same binding affinity to cyno FcRn compared to a VHH with SEQ ID NO: 186 or with SEQ ID NO: 187, wherein the binding affinity is measured using the same method, such as, e.g., SPR.

Further specific examples of ISVDs specifically binding to FcRn are ISVDs that comprises 4 framework regions (FR1 to FR4, respectively) and three complementarity determining regions (CDR1 to CDR3, respectively), wherein:
CDR1 (AbM numbering) consists of an amino acid sequence selected from:
a) the amino acid sequence of GFSFSDYYMY (SEQ ID NO:128);
b) amino acid sequences that have at least 80% amino acid identity with the amino acid sequence of GFSFSDYYMY (SEQ ID NO: 128);
c) amino acid sequences that have 3, 2, or 1 amino acid difference with the amino acid sequences GFSFSDYYMY (SEQ ID NO: 128);
   and
CDR2 (AbM numbering) consists of an amino acid sequence selected from:
a) the amino acid sequence of AISSGGSSTY (SEQ ID NO: 129);
b) amino acid sequences that have at least 80% amino acid identity with the amino acid sequence of AISSGGSSTY (SEQ ID NO:129);
c) amino acid sequences that have 3, 2, or 1 amino acid difference with the amino acid sequence of AISSGGSSTY (SEQ ID NO: 129);
   and
CDR3 (AbM numbering) consists of an amino acid sequence selected from:
a) the amino acid sequence of DYLSVPDPSYEY (SEQ ID NO:130);
b) amino acid sequences that have at least 80% amino acid identity with the amino acid sequence of DYLSVPDPSYEY (SEQ ID NO:130);
c) amino acid sequences that have 3, 2, or 1 amino acid difference with the amino acid sequence of DYLSVPDPSYEY (SEQ ID NO:130).

Preferably, the CDR sequences have at least 90% amino acid sequence identity, more preferably at least 95% amino acid sequence identity, such as 99% amino acid sequence identity or more, or even essentially 100% amino acid sequence identity with the CDR sequences of SEQ ID NOs: 128, 129, and/or 130.

In one embodiment, the ISVD comprise CDRs (AbM numbering) with an amino acid sequence that has at least 70% amino acid sequence identity, preferably at least 80% amino acid sequence identity, more preferably at least 90% amino acid sequence identity, such as 95% amino acid sequence identity or 99% amino acid sequence identity or more, or even essentially 100% amino acid sequence identity with the amino acid sequences of the CDRs of the ISVD with the amino acid sequence SEQ ID NO: 131.

(T0263091D07) (CDRs according to Abm are
underlined; CDRs according to
Kabat are in italics)
SEQ ID NO: 131
EVQLVESGGGVVQPGGSLRLSCAASGFSFSDYYMYWVRQAPG
KGLEWVSAISSGGSSTYYADSVKGRFTISRDNSKNTVYLQMN
SLRPEDTALYYCAADYLSVPDPSYEYWGQGTLVTVSS

In one embodiment, the ISVD comprises a CDR1 (AbM numbering) that is the amino acid sequence of SEQ ID NO: 128, a CDR2 (AbM numbering) that is the amino acid sequence of SEQ ID NO: 129 and a CDR3 (AbM numbering) that is the amino acid sequence of SEQ ID NO: 130.

Specific examples of such ISVDs that specifically bind to FcRn have one or more, or all, framework regions as indicated for T0263091D07 (SEQ ID NO: 131). In one embodiment, the ISVD comprises or consists of the full amino acid sequence of T0263091D07 (SEQ ID NO: 131).

The SEQ ID NOs for the CDR sequences referred to above are based on the CDR definition according to the AbM definition (see Table A-11). It is noted that the SEQ ID NOs for the CDR sequences defined according to the Kabat definition can likewise be used (see Table A-12). Accordingly, the ISVDs provided by the present technology, specifically binding to FcRn as described above using the AbM definition, can be also described using the Kabat definition.

As such in one embodiment, the ISVDs specifically binding to FcRn are ISVDs that comprise 4 framework regions (FR1 to FR4, respectively) and three complementarity determining regions (CDR1 to CDR3, respectively), wherein:
CDR1 (Kabat numbering) consists of an amino acid sequence selected from:
a) the amino acid sequence of DYYMY (SEQ ID NO: 136);
b) amino acid sequences that have at least 80% amino acid identity with the amino acid sequence of DYYMY (SEQ ID NO: 136);
c) amino acid sequences that have 3, 2, or 1 amino acid difference with the amino acid sequences of DYYMY (SEQ ID NO: 136);
   and
CDR2 (Kabat numbering) consists of an amino acid sequence selected from:
a) the amino acid sequence of AISSGGSSTYYADSVKG (SEQ ID NO: 137);
b) amino acid sequences that have at least 80% amino acid identity with the amino acid sequence of AISSGGSSTYYADSVKG (SEQ ID NO: 137);
c) amino acid sequences that have 3, 2, or 1 amino acid difference with the amino acid sequence of AISSGGSSTYYADSVKG (SEQ ID NO: 137);
   and
CDR3 (Kabat numbering) consists of an amino acid sequence selected from:
a) the amino acid sequence of DYLSVPDPSYEY (SEQ ID NO: 130);
b) amino acid sequences that have at least 80% amino acid identity with the amino acid sequence of DYLSVPDPSYEY (SEQ ID NO: 130);
c) amino acid sequences that have 3, 2, or 1 amino acid difference with the amino acid sequence of DYLSVPDPSYEY (SEQ ID NO: 130).

Preferably, the CDR sequences have at least 90% amino acid sequence identity, more preferably at least 95% amino acid sequence identity, such as 99% amino acid sequence identity or more, or even essentially 100% amino acid sequence identity with the CDR sequences of SEQ ID NOs: 136, 137, and/or 130.

In one embodiment, the ISVD comprise CDRs (Kabat numbering) with an amino acid sequence that has at least 70% amino acid sequence identity, preferably at least 80% amino acid sequence identity, more preferably at least 90% amino acid sequence identity, such as 95% amino acid sequence identity or 99% amino acid sequence identity or more, or even essentially 100% amino acid sequence identity with the amino acid sequences of the CDRs of the ISVD with the amino acid sequence of SEQ ID NOs: 131.

In one embodiment, the ISVD comprises a CDR1 (Kabat numbering) that is the amino acid sequence of SEQ ID NO: 136, a CDR2 (Kabat numbering) that is the amino acid sequence of SEQ ID NO: 137 and a CDR3 (Kabat numbering) that is the amino acid sequence of SEQ ID NO: 130.

Specific examples of such ISVDs that specifically bind to FcRn have one or more, or all, framework regions as indicated for T0263091D07 (SEQ ID NO: 131). In one embodiment, the ISVD comprises or consists of the full amino acid sequence of T0263091D07 (SEQ ID NO: 131) (see Table A-11, Table A-12).

In another embodiment, the ISVD specifically binding to human FcRn may have a sequence identity of more than 90%, such as more than 95% or even more than 99%, with SEQ ID NO: 131, wherein the CDRs are as defined above. In one embodiment, the ISVD specifically binding to FcRn comprises or consists of the amino acid sequence of SEQ ID NO: 131.

In another embodiment, when such an ISVD specifically binding to FcRn has 3, 2 or 1 amino acid difference in at least one CDR relative to a corresponding reference CDR sequence (above), the ISVD has at least (essentially) the same binding affinity to human FcRn compared to the VHHs with a sequence comprising or consisting of SEQ ID NO: 131, wherein the binding affinity is measured using the same method, such as, e.g., SPR.

In another embodiment, when such an ISVD specifically binding to FcRn has 3, 2 or 1 amino acid difference in at least one CDR relative to a corresponding reference CDR sequence (above), the ISVD has at least (essentially) the same binding affinity to cyno FcRn compared to one of the VHHs with a sequence comprising or consisting of SEQ ID NO: 131, wherein the binding affinity is measured using the same method, such as, e.g., SPR.

In another embodiment, when the CDRs of such an ISVD specifically binding to FcRn have at least 90% amino acid sequence identity, at least 95% amino acid sequence identity, such as 99% amino acid sequence identity or more, with a corresponding reference CDR sequence (above), the ISVD has at least (essentially) the same binding affinity to human FcRn compared to a VHH with SEQ ID NO: 131, wherein the binding affinity is measured using the same method, such as, e.g., SPR.

In another embodiment, when the CDRs of such an ISVD specifically binding to FcRn have at least 90% amino acid sequence identity, at least 95% amino acid sequence identity, such as 99% amino acid sequence identity or more, with a corresponding reference CDR sequence (above), the ISVD has at least (essentially) the same binding affinity to cyno FcRn compared to a VHH with SEQ ID NO: 131, wherein the binding affinity is measured using the same method, such as, e.g., SPR.

Further specific examples of ISVDs specifically binding to FcRn are ISVDs that comprises 4 framework regions (FR1 to FR4, respectively) and three complementarity determining regions (CDR1 to CDR3, respectively), wherein:
CDR1 (AbM numbering) consists of an amino acid sequence selected from:
   a) the amino acid sequence of GFTLDYYAIG (SEQ ID NO: 141);
   b) amino acid sequences that have at least 80% amino acid identity with the amino acid sequence of GFTLDYYAIG (SEQ ID NO: 141);
   c) amino acid sequences that have 3, 2, or 1 amino acid difference with the amino acid sequences GFTLDYYAIG (SEQ ID NO: 141);
and
CDR2 (AbM numbering) consists of an amino acid sequence selected from:
   a) the amino acid sequence of CISSSGDSTY (SEQ ID NO: 142);
   b) amino acid sequences that have at least 80% amino acid identity with the amino acid sequence of CISSSGDSTY (SEQ ID NO: 142);
   c) amino acid sequences that have 3, 2, or 1 amino acid difference with the amino acid sequence of CISSSGDSTY (SEQ ID NO: 142);
and
CDR3 (AbM numbering) consists of an amino acid sequence selected from:
   a) the amino acid sequence of DPPSYWTGTGCLYGYRY (SEQ ID NO:143);
   b) amino acid sequences that have at least 80% amino acid identity with the amino acid sequence of DPPSYWTGTGCLYGYRY (SEQ ID NO:143);
   c) amino acid sequences that have 3, 2, or 1 amino acid difference with the amino acid sequence of DPPSYWTGTGCLYGYRY (SEQ ID NO:143).

Preferably, the CDR sequences have at least 90% amino acid sequence identity, more preferably at least 95% amino acid sequence identity, such as 99% amino acid sequence identity or more, or even essentially 100% amino acid sequence identity with the CDR sequences of SEQ ID NOs: 141, 142, and/or 143.

In one embodiment, the ISVD comprise CDRs (AbM numbering) with an amino acid sequence that has at least 70% amino acid sequence identity, preferably at least 80% amino acid sequence identity, more preferably at least 90% amino acid sequence identity, such as 95% amino acid sequence identity or 99% amino acid sequence identity or more, or even essentially 100% amino acid sequence identity with the amino acid sequences of the CDRs of the ISVD with the amino acid sequence SEQ ID NO: 144.

(T0263204B12) (CDRs according to Abm
are underlined; CDRs according to
Kabat are in italics)
SEQ ID NO: 144
EVQLVESGGGLVQPGGSLRLSCAASGFTLDYYAIGWFRQAPGK
EREGVLCISSSGDSTYYADSVKGRFTISRDNAKNTVYLQMDSL
NPEDTAVYYCAVDPPSYWTGTGCLYGYRYWGQGTLVTVSS

In one embodiment, the ISVD comprises a CDR1 (AbM numbering) that is the amino acid sequence of SEQ ID NO: 141, a CDR2 (AbM numbering) that is the amino acid sequence of SEQ ID NO: 142 and a CDR3 (AbM numbering) that is the amino acid sequence of SEQ ID NO: 143.

Specific examples of such ISVDs that specifically bind to FcRn have one or more, or all, framework regions as indicated for T0263204B12 (SEQ ID NO: 144). In one embodiment, the ISVD comprises or consists of the full amino acid sequence of T0263204B12 (SEQ ID NO: 144).

The SEQ ID NOs for the CDR sequences referred to above are based on the CDR definition according to the AbM definition (see Table A-13). It is noted that the SEQ ID NOs for the CDR sequences defined according to the Kabat definition can likewise be used (see Table A-14). Accordingly, the ISVDs provided by the present technology, specifically binding to FcRn as described above using the AbM definition, can be also described using the Kabat definition.

As such in one embodiment, the ISVDs specifically binding to FcRn are ISVDs that comprise 4 framework regions (FR1 to FR4, respectively) and three complementarity determining regions (CDR1 to CDR3, respectively), wherein:
CDR1 (Kabat numbering) consists of an amino acid sequence selected from:
   a) the amino acid sequence of YYAIG (SEQ ID NO: 145);
   b) amino acid sequences that have at least 80% amino acid identity with the amino acid sequence of YYAIG (SEQ ID NO: 145);
   c) amino acid sequences that have 3, 2, or 1 amino acid difference with the amino acid sequences of YYAIG (SEQ ID NO: 145);
and
CDR2 (Kabat numbering) consists of an amino acid sequence selected from:
   a) the amino acid sequence of CISSSGDSTYYADSVKG (SEQ ID NO:146);
   b) amino acid sequences that have at least 80% amino acid identity with the amino acid sequence of CISSSGDSTYYADSVKG (SEQ ID NO:146);
   c) amino acid sequences that have 3, 2, or 1 amino acid difference with the amino acid sequence of CISSSGDSTYYADSVKG (SEQ ID NO:146);
and
CDR3 (Kabat numbering) consists of an amino acid sequence selected from:
   a) the amino acid sequence of DPPSYWTGTGCLYGYRY (SEQ ID NO: 143);
   b) amino acid sequences that have at least 80% amino acid identity with the amino acid sequence of DPPSYWTGTGCLYGYRY (SEQ ID NO: 143);
   c) amino acid sequences that have 3, 2, or 1 amino acid difference with the amino acid sequence of DPPSYWTGTGCLYGYRY (SEQ ID NO: 143).

Preferably, the CDR sequences have at least 90% amino acid sequence identity, more preferably at least 95% amino acid sequence identity, such as 99% amino acid sequence identity or more, or even essentially 100% amino acid sequence identity with the CDR sequences of SEQ ID NOs: 145, 146, and/or 143.

In one embodiment, the ISVD comprise CDRs (Kabat numbering) with an amino acid sequence that has at least 70% amino acid sequence identity, preferably at least 80% amino acid sequence identity, more preferably at least 90% amino acid sequence identity, such as 95% amino acid sequence identity or 99% amino acid sequence identity or more, or even essentially 100% amino acid sequence identity with the amino acid sequences of the CDRs of the ISVD with the amino acid sequence of SEQ ID NOs: 144.

In one embodiment, the ISVD comprises a CDR1 (Kabat numbering) that is the amino acid sequence of SEQ ID NO: 145, a CDR2 (Kabat numbering) that is the amino acid sequence of SEQ ID NO: 146 and a CDR3 (Kabat numbering) that is the amino acid sequence of SEQ ID NO: 143.

Specific examples of such ISVDs that specifically bind to FcRn have one or more, or all, framework regions as indicated for T0263204B12 (SEQ ID NO: 144). In one embodiment, the ISVD comprises or consists of the full amino acid sequence of T0263204B12 (SEQ ID NO: 144) (see Table A-12, Table A-13).

In another embodiment, the ISVD specifically binding to human FcRn may have a sequence identity of more than 90%, such as more than 95% or even more than 99%, with SEQ ID NO: 144, wherein the CDRs are as defined above. In one embodiment, the ISVD specifically binding to FcRn comprises or consists of the amino acid sequence of SEQ ID NO: 144.

In another embodiment, when such an ISVD specifically binding to FcRn has 3, 2 or 1 amino acid difference in at least one CDR relative to a corresponding reference CDR sequence (above), the ISVD has at least (essentially) the same binding affinity to human FcRn compared to the VHHs with a sequence comprising or consisting of SEQ ID NO: 144, wherein the binding affinity is measured using the same method, such as, e.g., SPR.

In another embodiment, when such an ISVD specifically binding to FcRn has 3, 2 or 1 amino acid difference in at least one CDR relative to a corresponding reference CDR sequence (above), the ISVD has at least (essentially) the same binding affinity to cyno FcRn compared to one of the VHHs with a sequence comprising or consisting of SEQ ID NO: 144, wherein the binding affinity is measured using the same method, such as, e.g., SPR.

In another embodiment, when the CDRs of such an ISVD specifically binding to FcRn have at least 90% amino acid sequence identity, at least 95% amino acid sequence identity, such as 99% amino acid sequence identity or more, with a corresponding reference CDR sequence (above), the ISVD has at least (essentially) the same binding affinity to human FcRn compared to a VHH with SEQ ID NO: 144, wherein the binding affinity is measured using the same method, such as, e.g., SPR.

In another embodiment, when the CDRs of such an ISVD specifically binding to FcRn have at least 90% amino acid sequence identity, at least 95% amino acid sequence identity, such as 99% amino acid sequence identity or more, with a corresponding reference CDR sequence (above), the ISVD has at least (essentially) the same binding affinity to cyno FcRn compared to a VHH with SEQ ID NO: 144, wherein the binding affinity is measured using the same method, such as, e.g., SPR.

Further specific examples of ISVDs specifically binding to FcRn are ISVDs that comprises 4 framework regions (FR1 to FR4, respectively) and three complementarity determining regions (CDR1 to CDR3, respectively), wherein:
CDR1 (AbM numbering) consists of an amino acid sequence selected from:
   a) the amino acid sequence of GFTFSDYGMG (SEQ ID NO:155);
   b) amino acid sequences that have at least 80% amino acid identity with the amino acid sequence of GFTFSDYGMG (SEQ ID NO:155);
   c) amino acid sequences that have 3, 2, or 1 amino acid difference with the amino acid sequences GFTFSDYGMG (SEQ ID NO:155);
and
CDR2 (AbM numbering) consists of an amino acid sequence selected from:
   a) the amino acid sequence of YISSDGGETS (SEQ ID NO: 156);
   b) amino acid sequences that have at least 80% amino acid identity with the amino acid sequence of YISSDGGETS (SEQ ID NO: 156);
   c) amino acid sequences that have 3, 2, or 1 amino acid difference with the amino acid sequence of YISSDGGETS (SEQ ID NO: 156);
and
CDR3 (AbM numbering) consists of an amino acid sequence selected from:
   a) the amino acid sequence of GGV (SEQ ID NO:157);
   b) amino acid sequences that have at least 80% amino acid identity with the amino acid sequence of GGV (SEQ ID NO:157);
   c) amino acid sequences that have 3, 2, or 1 amino acid difference with the amino acid sequence of GGV (SEQ ID NO:157).

Preferably, the CDR sequences have at least 90% amino acid sequence identity, more preferably at least 95% amino acid sequence identity, such as 99% amino acid sequence identity or more, or even essentially 100% amino acid sequence identity with the CDR sequences of SEQ ID NOs: 155, 156, and/or 157.

In one embodiment, the ISVD comprise CDRs (AbM numbering) with an amino acid sequence that has at least 70% amino acid sequence identity, preferably at least 80% amino acid sequence identity, more preferably at least 90% amino acid sequence identity, such as 95% amino acid sequence identity or 99% amino acid sequence identity or more, or even essentially 100% amino acid sequence identity with the amino acid sequences of the CDRs of the ISVD with the amino acid sequence SEQ ID NO: 158 or 168.

(T0263201B05(E1D)) (CDRs according to
Abm are underlined; CDRs according
to Kabat are in italics)
SEQ ID NO: 158
DVQLVESGGGLVQPGGSLRLSCAASGFTFSDYGMGWVRQ
APGKGLEVVSYISSDGGETSYADSVKGRFTISRDNAKNM
LYLQMSSLKLEDTALYYCAKGGVRGQGTLVTVSS
(T0263201B05) (CDRs according to Abm
are underlined; CDRs according to
Kabat are in italics)
SEQ ID NO: 168
EVQLVESGGGLVQPGGSLRLSCAASGFTFSDYGMGWVRQ
APGKGLEVVSYISSDGGETSYADSVKGRFTISRDNAKNM
LYLQMSSLKLEDTALYYCAKGGVRGQGTLVTVSS

In one embodiment, the ISVD comprises a CDR1 (AbM numbering) that is the amino acid sequence of SEQ ID NO: 155, a CDR2 (AbM numbering) that is the amino acid sequence of SEQ ID NO: 156 and a CDR3 (AbM numbering) that is the amino acid sequence of SEQ ID NO: 157.

Specific examples of such ISVDs that specifically bind to FcRn have one or more, or all, framework regions as indicated for T0263201B05(E1D) (SEQ ID NO: 158) or for T0263201B05 (SEQ ID NO: 168). In one embodiment, the ISVD comprises or consists of the full amino acid sequence of T0263201B05(E1D) (SEQ ID NO: 158) or the full amino acid sequence of T0263201B05 (SEQ ID NO: 168).

The SEQ ID NOs for the CDR sequences referred to above are based on the CDR definition according to the AbM definition (see Tables A-7 and A-9). It is noted that the SEQ ID NOs for the CDR sequences defined according to the Kabat definition can likewise be used (see Tables A-8 and A-10). Accordingly, the ISVDs provided by the present technology, specifically binding to FcRn as described above using the AbM definition, can be also described using the Kabat definition.

As such in one embodiment, the ISVDs specifically binding to FcRn are ISVDs that comprise 4 framework regions (FR1 to FR4, respectively) and three complementarity determining regions (CDR1 to CDR3, respectively), wherein:
CDR1 (Kabat numbering) consists of an amino acid sequence selected from:
   a) the amino acid sequence of DYGMG (SEQ ID NO: 159);
   b) amino acid sequences that have at least 80% amino acid identity with the amino acid sequence of DYGMG (SEQ ID NO: 159);
   c) amino acid sequences that have 3, 2, or 1 amino acid difference with the amino acid sequences of DYGMG (SEQ ID NO: 159);
and
CDR2 (Kabat numbering) consists of an amino acid sequence selected from:
   a) the amino acid sequence of YISSDGGETSYADSVKG (SEQ ID NO: 160);
   b) amino acid sequences that have at least 80% amino acid identity with the amino acid sequence of YISSDGGETSYADSVKG (SEQ ID NO: 160);
   c) amino acid sequences that have 3, 2, or 1 amino acid difference with the amino acid sequence of YISSDGGETSYADSVKG (SEQ ID NO: 160);
and
CDR3 (Kabat numbering) consists of an amino acid sequence selected from:
   a) the amino acid sequence of GGV (SEQ ID NO: 157);
   b) amino acid sequences that have at least 80% amino acid identity with the amino acid sequence of GGV (SEQ ID NO: 157);
   c) amino acid sequences that have 3, 2, or 1 amino acid difference with the amino acid sequence of GGV (SEQ ID NO: 157).

Preferably, the CDR sequences have at least 90% amino acid sequence identity, more preferably at least 95% amino acid sequence identity, such as 99% amino acid sequence identity or more, or even essentially 100% amino acid sequence identity with the CDR sequences of SEQ ID NOs: 159, 160, and/or 157.

In one embodiment, the ISVD comprise CDRs (Kabat numbering) with an amino acid sequence that has at least 70% amino acid sequence identity, preferably at least 80% amino acid sequence identity, more preferably at least 90% amino acid sequence identity, such as 95% amino acid sequence identity or 99% amino acid sequence identity or more, or even essentially 100% amino acid sequence identity with the amino acid sequences of the CDRs of the ISVD with the amino acid sequence of SEQ ID NOs: 158 or 168.

In one embodiment, the ISVD comprises a CDR1 (Kabat numbering) that is the amino acid sequence of SEQ ID NO: 159, a CDR2 (Kabat numbering) that is the amino acid sequence of SEQ ID NO: 160 and a CDR3 (Kabat numbering) that is the amino acid sequence of SEQ ID NO: 157.

Specific examples of such ISVDs that specifically bind to FcRn have one or more, or all, framework regions as indicated for T0263201B05(E1D) (SEQ ID NO: 158) or for T0263201B05 (SEQ ID NO: 168). In one embodiment, the ISVD comprises or consists of the full amino acid sequence T0263201B05(E1D) (SEQ ID NO: 158) or the full amino acid sequence T0263201B05 (SEQ ID NO: 168) (see Tables A-7 to A-10).

In another embodiment, the ISVD specifically binding to human FcRn may have a sequence identity of more than 90%, such as more than 95% or even more than 99%, with SEQ ID NO: 158, wherein the CDRs are as defined above. In one embodiment, the ISVD specifically binding to FcRn comprises or consists of the amino acid sequence of SEQ ID NO: 158 or of SEQ ID NO: 168.

In another embodiment, when such an ISVD specifically binding to FcRn has 3, 2 or 1 amino acid difference in at least one CDR relative to a corresponding reference CDR sequence (above), the ISVD has at least (essentially) the same binding affinity to human FcRn compared to the VHHs with a sequence comprising or consisting of SEQ ID NO: 158 or of SEQ ID NO: 168, wherein the binding affinity is measured using the same method, such as, e.g., SPR.

In another embodiment, when such an ISVD specifically binding to FcRn has 3, 2 or 1 amino acid difference in at least one CDR relative to a corresponding reference CDR sequence (above), the ISVD has at least (essentially) the same binding affinity to cyno FcRn compared to one of the VHHs with a sequence comprising or consisting of SEQ ID NO: 158 or of SEQ ID NO: 168, wherein the binding affinity is measured using the same method, such as, e.g., SPR.

In another embodiment, when the CDRs of such an ISVD specifically binding to FcRn have at least 90% amino acid sequence identity, at least 95% amino acid sequence identity, such as 99% amino acid sequence identity or more, with a corresponding reference CDR sequence (above), the ISVD has at least (essentially) the same binding affinity to human FcRn compared to a VHH with SEQ ID NO: 158 or with SEQ ID NO: 168, wherein the binding affinity is measured using the same method, such as, e.g., SPR.

In another embodiment, when the CDRs of such an ISVD specifically binding to FcRn have at least 90% amino acid sequence identity, at least 95% amino acid sequence identity, such as 99% amino acid sequence identity or more, with a corresponding reference CDR sequence (above), the ISVD has at least (essentially) the same binding affinity to cyno FcRn compared to a VHH with SEQ ID NO: 158 or with SEQ ID NO: 168, wherein the binding affinity is measured using the same method, such as, e.g., SPR.

Specific Examples of ISVDs Binding to FcRn that are Suitable for Use as Second Polypeptides in the FcRn Antagonists of the Present Invention

Specific examples of ISVDs specifically binding to FcRn are ISVDs that comprises 4 framework regions (FR1 to FR4, respectively) and three complementarity determining regions (CDR1 to CDR3, respectively), wherein:
CDR1 (AbM numbering) consists of an amino acid sequence selected from:
a) the amino acid sequence of GFTFSDYGMG (SEQ ID NO: 155);
b) amino acid sequences that have at least 80% amino acid identity with the amino acid sequence of GFTFSDYGMG (SEQ ID NO: 155);
c) amino acid sequences that have 3, 2, or 1 amino acid difference with the amino acid sequences GFTFSDYGMG (SEQ ID NO: 155);
   and
CDR2 (AbM numbering) consists of an amino acid sequence selected from:
a) the amino acid sequence of YISSDGGETS (SEQ ID NO: 156);
b) amino acid sequences that have at least 80% amino acid identity with the amino acid sequence of YISSDGGETS (SEQ ID NO: 156);
c) amino acid sequences that have 3, 2, or 1 amino acid difference with the amino acid sequence of YISSDGGETS (SEQ ID NO: 156);
   and
CDR3 (AbM numbering) consists of an amino acid sequence selected from:
a) the amino acid sequence of GGV (SEQ ID NO:157);
b) amino acid sequences that have at least 80% amino acid identity with the amino acid sequence of GGV (SEQ ID NO:157);
c) amino acid sequences that have 3, 2, or 1 amino acid difference with the amino acid sequence of GGV (SEQ ID NO:157).

Preferably, the CDR sequences have at least 90% amino acid sequence identity, more preferably at least 95% amino acid sequence identity, such as 99% amino acid sequence identity or more, or even essentially 100% amino acid sequence identity with the CDR sequences of SEQ ID NOs: 155, 156, and/or 157.

In one embodiment, the ISVD comprise CDRs (AbM numbering) with an amino acid sequence that has at least 70% amino acid sequence identity, preferably at least 80% amino acid sequence identity, more preferably at least 90% amino acid sequence identity, such as 95% amino acid sequence identity or 99% amino acid sequence identity or more, or even essentially 100% amino acid sequence identity with the amino acid sequences of the CDRs of the ISVD with the amino acid sequence SEQ ID NO: 158 or 168.

(T0263201B05(E1D)) (CDRs according to
Abm are underlined; CDRs according
to Kabat are in italics)
SEQ ID NO: 158
DVQLVESGGGLVQPGGSLRLSCAASGFTFSDYGMGWVRQAPG
KGLEVVSYISSDGGETSYADSVKGRFTISRDNAKNMLYLQMS
SLKLEDTALYYCAKGGVRGQGTLVTVSS
(T0263201B05) (CDRs according to Abm
are underlined; CDRs according to
Kabat are in italics)
SEQ ID NO: 168
EVQLVESGGGLVQPGGSLRLSCAASGFTFSDYGMGWVRQAPG
KGLEVVSYISSDGGETSYADSVKGRFTISRDNAKNMLYLQMS
SLKLEDTALYYCAKGGVRGQGTLVTVSS

In one embodiment, the ISVD comprises a CDR1 (AbM numbering) that is the amino acid sequence of SEQ ID NO: 155, a CDR2 (AbM numbering) that is the amino acid sequence of SEQ ID NO: 156 and a CDR3 (AbM numbering) that is the amino acid sequence of SEQ ID NO: 157.

Specific examples of such ISVDs that specifically bind to FcRn have one or more, or all, framework regions as indicated for T0263201B05(E1D) (SEQ ID NO: 158) or for T0263201B05 (SEQ ID NO: 168). In one embodiment, the ISVD comprises or consists of the full amino acid sequence of T0263201B05(E1D) (SEQ ID NO: 158) or the full amino acid sequence of T0263201B05 (SEQ ID NO: 168).

The SEQ ID NOs for the CDR sequences referred to above are based on the CDR definition according to the AbM definition (see Tables A-7 and A-9). It is noted that the SEQ ID NOs for the CDR sequences defined according to the Kabat definition can likewise be used (see Tables A-8 and A-10). Accordingly, the ISVDs provided by the present technology, specifically binding to FcRn as described above using the AbM definition, can be also described using the Kabat definition.

As such in one embodiment, the ISVDs specifically binding to FcRn are ISVDs that comprise 4 framework regions (FR1 to FR4, respectively) and three complementarity determining regions (CDR1 to CDR3, respectively), wherein:
CDR1 (Kabat numbering) consists of an amino acid sequence selected from:
a) the amino acid sequence of DYGMG (SEQ ID NO: 159);
b) amino acid sequences that have at least 80% amino acid identity with the amino acid sequence of DYGMG (SEQ ID NO: 159);
c) amino acid sequences that have 3, 2, or 1 amino acid difference with the amino acid sequences of DYGMG (SEQ ID NO: 159);
   and
CDR2 (Kabat numbering) consists of an amino acid sequence selected from:
a) the amino acid sequence of YISSDGGETSYADSVKG (SEQ ID NO: 160);
b) amino acid sequences that have at least 80% amino acid identity with the amino acid sequence of YISSDGGETSYADSVKG (SEQ ID NO: 160);
c) amino acid sequences that have 3, 2, or 1 amino acid difference with the amino acid sequence of YISSDGGETSYADSVKG (SEQ ID NO: 160);
   and
CDR3 (Kabat numbering) consists of an amino acid sequence selected from:
a) the amino acid sequence of GGV (SEQ ID NO: 157);
b) amino acid sequences that have at least 80% amino acid identity with the amino acid sequence of GGV (SEQ ID NO: 157);
c) amino acid sequences that have 3, 2, or 1 amino acid difference with the amino acid sequence of GGV (SEQ ID NO: 157).

Preferably, the CDR sequences have at least 90% amino acid sequence identity, more preferably at least 95% amino acid sequence identity, such as 99% amino acid sequence identity or more, or even essentially 100% amino acid sequence identity with the CDR sequences of SEQ ID NOs: 159, 160, and/or 157.

In one embodiment, the ISVD comprise CDRs (Kabat numbering) with an amino acid sequence that has at least 70% amino acid sequence identity, preferably at least 80% amino acid sequence identity, more preferably at least 90% amino acid sequence identity, such as 95% amino acid sequence identity or 99% amino acid sequence identity or more, or even essentially 100% amino acid sequence identity with the amino acid sequences of the CDRs of the ISVD with the amino acid sequence of SEQ ID NOs: 158 or 168.

In one embodiment, the ISVD comprises a CDR1 (Kabat numbering) that is the amino acid sequence of SEQ ID NO: 159, a CDR2 (Kabat numbering) that is the amino acid sequence of SEQ ID NO: 160 and a CDR3 (Kabat numbering) that is the amino acid sequence of SEQ ID NO: 157.

Specific examples of such ISVDs that specifically bind to FcRn have one or more, or all, framework regions as indicated for T0263201B05(E1D) (SEQ ID NO: 158) or for T0263201B05 (SEQ ID NO: 168). In one embodiment, the ISVD comprises or consists of the full amino acid sequence T0263201B05(E1D) (SEQ ID NO: 158) or the full amino acid sequence T0263201B05 (SEQ ID NO: 168) (see Tables A-7 to A-10).

In another embodiment, the ISVD specifically binding to human FcRn may have a sequence identity of more than 90%, such as more than 95% or even more than 99%, with SEQ ID NO: 158, wherein the CDRs are as defined above. In one embodiment, the ISVD specifically binding to FcRn comprises or consists of the amino acid sequence of SEQ ID NO: 158 or of SEQ ID NO: 168.

In another embodiment, when such an ISVD specifically binding to FcRn has 3, 2 or 1 amino acid difference in at least one CDR relative to a corresponding reference CDR sequence (above), the ISVD has at least (essentially) the same binding affinity to human FcRn compared to the VHHs with a sequence comprising or consisting of SEQ ID NO: 158 or of SEQ ID NO: 168, wherein the binding affinity is measured using the same method, such as, e.g., SPR.

In another embodiment, when such an ISVD specifically binding to FcRn has 3, 2 or 1 amino acid difference in at least one CDR relative to a corresponding reference CDR sequence (above), the ISVD has at least (essentially) the same binding affinity to cyno FcRn compared to one of the VHHs with a sequence comprising or consisting of SEQ ID NO: 158 or of SEQ ID NO: 168, wherein the binding affinity is measured using the same method, such as, e.g., SPR.

In another embodiment, when the CDRs of such an ISVD specifically binding to FcRn have at least 90% amino acid sequence identity, at least 95% amino acid sequence identity, such as 99% amino acid sequence identity or more, with a corresponding reference CDR sequence (above), the ISVD has at least (essentially) the same binding affinity to human FcRn compared to a VHH with SEQ ID NO: 158 or with SEQ ID NO: 168, wherein the binding affinity is measured using the same method, such as, e.g., SPR.

In another embodiment, when the CDRs of such an ISVD specifically binding to FcRn have at least 90% amino acid sequence identity, at least 95% amino acid sequence identity, such as 99% amino acid sequence identity or more, with a corresponding reference CDR sequence (above), the ISVD has at least (essentially) the same binding affinity to cyno FcRn compared to a VHH with SEQ ID NO: 158 or with SEQ ID NO: 168, wherein the binding affinity is measured using the same method, such as, e.g., SPR.

SEQUENCES
C-terminal fusion construct without knobs/
holes mutation in Fc region Sequence of Fc
YTE-KF (SEQ ID NO: (23) or 24), with sequence
of Fc YTE-KF hinge underlined, sequence of
the 35 GS linker in italics (SEQ ID NO: 39),
sequence of T0263018B11-W100cS (FcRn ISVD,
SEQ ID NO: 15) in bold
SEQ ID NO: 62
DKTHTCPPCPAPELLGGPSVFLFPPKPKDTLYITREPEVTCVVVD
VSHEDPEVKFNWYVDGVEVHNAKTKPREEQYNSTYRVVSVLTVLH
QDWLNGKEYKCKVSNKALPAPIEKTISKAKGQPREPQVYTLPPSR
DELTKNQVSLTCLVKGFYPSDIAVEWESNGQPENNYKTTPPVLDS
DGSFFLYSKLTVDKSRWQQGNVFSCSVMHEALKFHYTQKSLSLSP
G(K)GGGGSGGGGSGGGGSGGGGSGGGGSGGGGSGGGGSEVQLVE
SGGGVVQPGGSLRLSCAASGFTFSSYAMYWVRQAPGKGLEWVSAI
SSGGGSTDYADSVKGRFTISRDNSKNTLYLQMNSLRPEDTALYYC
AADTLYTSLTSYSYWGQGTLVTVSS
C-terminal fusion construct (TP069)
(chain 1-knobs) Sequence of Fc YTE-KF (knobs)
(SEQ ID NO: 119, with sequence of Fc YTE-KF
hinge underlined, sequence of the 35 GS
linker in italics (SEQ ID NO: 39), sequence
of T0263018B11-W100cS
(FcRn ISVD, SEQ ID NO: 15) in bold
SEQ ID NO: 124
DKTHTCPPCPAPELLGGPSVFLFPPKPKDTLYITREPEVTCVVVD
VSHEDPEVKFNWYVDGVEVHNAKTKPREEQYNSTYRVVSVLTVLH
QDWLNGKEYKCKVSNKALPAPIEKTISKAKGQPREPQVYTLPPCR
DELTKNQVSLWCLVKGFYPSDIAVEWESNGQPENNYKTTPPVLDS
DGSFFLYSKLTVDKSRWQQGNVFSCSVMHEALKFHYTQKSLSLSP
GGGGGSGGGGSGGGGSGGGGSGGGGSGGGGSGGGGSEVQLVESGG
GVVQPGGSLRLSCAASGFTFSSYAMYWVRQAPGKGLEWVSAISSG
GGSTDYADSVKGRFTISRDNSKNTLYLQMNSLRPEDTALYYCAAD
TLYTSLTSYSYWGQGTLVTVSS
C-terminal fusion construct (TP069)
(Chain 2-holes) Sequence of Fc YTE-KF (hole)
(SEQ ID NO: 120, with sequence of Fc YTE-KF
hinge underlined
SEQ ID NO: 125 or 120
DKTHTCPPCPAPELLGGPSVFLFPPKPKDTLYITREPEVTCVVVD
VSHEDPEVKFNWYVDGVEVHNAKTKPREEQYNSTYRVVSVLTVLH
QDWLNGKEYKCKVSNKALPAPIEKTISKAKGQPREPQVCTLPPSR
DELTKNQVSLSCAVKGFYPSDIAVEWESNGQPENNYKTTPPVLDS
DGSFFLVSKLTVDKSRWQQGNVFSCSVMHEALKFHYTQKSLSLSP
G
N-terminal fusion construct without knobs/
holes mutation in Fc region Sequence of
T0263018B11 (E1D, W100cS) in bold (FcRn ISVD,
SEQ ID NO: 185), sequence of Fc YTE-KF
(SEQ ID NO: (23) or 24) with sequence of
Fc YTE-KF hinge underlined
SEQ ID NO: 63
DVQLVESGGGVVQPGGSLRLSCAASGFTFSSYAMYWVRQAPGKGL
EWVSAISSGGGSTDYADSVKGRFTISRDNSKNTLYLQMNSLRPED
TALYYCAADTLYTSLTSYSYWGQGTLVTVSSDKTHTCPPCPAPEL
LGGPSVFLFPPKPKDTLYITREPEVTCVVVDVSHEDPEVKFNWYV
DGVEVHNAKTKPREEQYNSTYRVVSVLTVLHQDWLNGKEYKCKVS
NKALPAPIEKTISKAKGQPREPQVYTLPPSRDELTKNQVSLTCLV
KGFYPSDIAVEWESNGQPENNYKTTPPVLDSDGSFFLYSKLTVDK
SRWQQGNVFSCSVMHEALKFHYTQKSLSLSPG(K)
N-terminal fusion construct (TP067)
(Chain 1-knobs) Sequence of T0263018B11
(E1D, W100cS) in bold (FcRn ISVD,
SEQ ID NO: 185), sequence of Fc YTE-KF
(knob) (SEQ ID NO: 119) with sequence of
Fc YTE-KF hinge underlined
SEQ ID NO: 126
DVQLVESGGGVVQPGGSLRLSCAASGFTFSSYAMYWVRQAPGKGL
EWVSAISSGGGSTDYADSVKGRFTISRDNSKNTLYLQMNSLRPED
TALYYCAADTLYTSLTSYSYWGQGTLVTVSSDKTHTCPPCPAPEL
LGGPSVFLFPPKPKDTLYITREPEVTCVVVDVSHEDPEVKFNWYV
DGVEVHNAKTKPREEQYNSTYRVVSVLTVLHQDWLNGKEYKCKVS
NKALPAPIEKTISKAKGQPREPQVYTLPPCRDELTKNQVSLWCLV
KGFYPSDIAVEWESNGQPENNYKTTPPVLDSDGSFFLYSKLTVDK
SRWQQGNVFSCSVMHEALKFHYTQKSLSLSPG
N-terminal fusion construct (TP067)
(Chain 2-holes)
Sequence of Fc YTE-KF (hole) (SEQ ID NO: 120)
with sequence of Fc YTE-KF hinge underlined
SEQ ID NO: 127
DKTHTCPPCPAPELLGGPSVFLFPPKPKDTLYITREPEVTCVVVD
VSHEDPEVKFNWYVDGVEVHNAKTKPREEQYNSTYRVVSVLTVLH
QDWLNGKEYKCKVSNKALPAPIEKTISKAKGQPREPQVCTLPPSR
DELTKNQVSLSCAVKGFYPSDIAVEWESNGQPENNYKTTPPVLDS
DGSFFLVSKLTVDKSRWQQGNVFSCSVMHEALKFHYTQKSLSLSP
G

TABLE A-1

| Name | ID | Amino acid sequence |
|---|---|---|
| Human FcRn | 1 | AESHLSLLYHLTAVSSPAPGTPAFWVSGWLGPQQYLSYNSL |
| | | RGEAEPCGAWVWENQVSWYWEKETTDLRIKEKLFLEAFKA |
| | | LGGKGPYTLQGLLGCELGPDNTSVPTAKFALNGEEFMNFD |
| | | LKQGTWGGDWPEALAISQRWQQQDKAANKELTFLLFSCPH |
| | | RLREHLERGRGNLEWKEPPSMRLKARPSSPGFSVLTCSAF |
| | | SFYPPELQLRFLRNGLAAGTGQGDFGPNSDGSFHASSSLT |
| | | VKSGDEHHYCCIVQHAGLAQPLRVELESPAKSS |
| CDR1 (variable sequence | 2 | X₁ X₂ X₃ M Y, wherein |
| -Kabat) | | X₁ is A, D, E, G, K, N, Q, S, T, or V, |
| | | X₂ is A, F, L, M, N, Q, S, T, V, or Y, and |
| | | X₃ is A, D, E, G, H, M, N, Q, S, T, or V |
| CDR2 (variable sequence | 3 | A I X₃ X₄ G G G X₈ X₉ X₁₀ Y A D S V K G, wherein |
| -Kabat) | | X₃ is A, D, E, G, H, P, Q, R, S, T, or V, |
| | | X₄ is A, D, E, G, K, M, N, P, Q, R, S, T, V, W, or Y, |
| | | X₈ is A, D, G, H, K, L, M, Q, S, T, or V, |
| | | X₉ is A, D, E, F, G, H, I, K, L, M, N, |
| | | P, Q, R, S, T, V, W, or Y, |
| | | and |
| | | X₁₀ is A, D, E, H, S, T, V, or Y, |
| CDR3 (variable sequence | 4 | D X₂ X₃ X₄ T X₆ X₇ T X₉ Y X₁₁ X₁₂, wherein |
| -Kabat) | | X₂ is A, D, E, F, G, I, L, M, Q, S, T, V, W, or Y, |
| | | X₃ is L, or N, |
| | | X₄ is F, L, W, or Y, |
| | | X₆ is A, D, E, K, L, M, Q, S, or W, |
| | | X₇ is L, M, or V, |
| | | X₉ is A, D, E, F, G, H, K, L, M, Q, R, S, T, V, W or Y, |
| | | X₁₁ is A, E, L, M, Q, S, T, V, or W, and |
| | | X₁₂ is T or Y |
| CDR1 (variable sequence | 5 | Xₐ X_{b} X_{c} F Xₑ X₁ X₂ X₃ M Y, wherein |
| -AbM) | | Xₐ is A, D, E, F, G, H, K, L, M, N, Q, S, T, W, or Y, |
| | | X_{b} is F, I, L, M, V, or Y, |
| | | X_{c} is A, E, G, I, L, M, N, P, S, T, V, W, or Y, |
| | | Xₑ is D, E, G, K, M, N, P, Q, S or T, |
| | | X₁ is A, D, E, G, K, N, Q, S, T, or V, |
| | | X₂ is A, F, L, M, N, Q, S, T, V, or Y, and |
| | | X₃ is A, D, E, G, H, M, N, Q, S, T, or V |
| CDR2 (variable sequence | 6 | A I X₃ X₄ G G G X₈ X₉ X₁₀, wherein |
| -AbM) | | X₃ is A, D, E, G, H, P, Q, R, S, T, or V, |
| | | X₄ is A, D, E, G, K, M, N, P, Q, R, S, T, V, W, or Y, |
| | | X₈ is A, D, G, H, K, L, M, Q, S, T, or V, |
| | | X₉ is A, D, E, F, G, H, I, K, L, M, N, P, Q, R, S, T, |
| | | V, W, or Y, and |
| | | X₁₀ is A, D, E, H, S, T, V, or Y |
| CDR3 (variable sequence | 7 | D X₂ X₃ X₄ T X₆ X₇ T X₉ Y X₁₁ X₁₂, wherein |
| -AbM) | | X₂ is A, D, E, F, G, I, L, M, Q, S, T, V, W, or Y, |
| | | X₃ is L, or N, |
| | | X₄ is F, L, W, or Y, |
| | | X₆ is A, D, E, K, L, M, Q, S, or W, |
| | | X₇ is L, M, or V, |
| | | X₉ is A, D, E, F, G, H, K, L, M, Q, R, S, T, V, W or Y, |
| | | X₁₁ is A, E, L, M, Q, S, T, V, or W, and |
| | | X₁₂ is T or Y |
| T0263018B11- | 8 | SYAMY |
| parent/W100cS/ | | |
| (Y79S, T100bE, W100cS) | | |
| (CDR1-Kabat) | | |
| T0263018B11- | 9 | AISSGGGSTDYADSVKG |
| parent/W100cS/(Y79S, | | |
| T100bE, W100cS) (CDR2- | | |
| Kabat) | | |
| T0263018B11-W100cS | 10 | DTLYTSLTSYSY |
| (CDR3-Kabat) | | |
| T0263018B11- | 11 | GFTFSSYAMY |
| parent/W100cS/ | | |
| (Y79S, T100bE, W100cS) | | |
| (CDR1-AbM) | | |
| T0263018B11- | 12 | AISSGGGSTD |
| parent/W100cS/(Y79S, | | |
| T100bE, W100cS) (CDR2- | | |
| AbM) | | |
| T0263018B11-W100cS | 13 | DTLYTSLTSYSY |
| (CDR3-AbM) | | |
| T0263018B11-parent | 14 | EVQLVESGGGVVQPGGSLRLSCAASGFTFSSYAMYWVRQAP |
| | | GKGLEWVSAISSGGGSTDYADSVKGRFTISRDNSKNTLYL |
| | | QMNSLRPEDTALYYCAADTLYTSLTWYSYWGQGTLVTVSS |
| T0263018B11-W100cS | 15 | EVQLVESGGGVVQPGGSLRLSCAASGFTFSSYAMYWVRQAP |
| | | GKGLEWVSAISSGGGSTDYADSVKGRFTISRDNSKNTLYL |
| | | QMNSLRPEDTALYYCAADTLYTSLTSYSYWGQGTLVTVSS |
| ISVD binding to serum | 16 | SFGMS |
| albumin CDR1 (CDR1 | | |
| (kabat) of ALB23002) | | |
| ISVD binding to serum | 17 | SISGSGSDTLYADSVKG |
| albumin CDR2 (CDR2 | | |
| (kabat) of ALB23002) | | |
| ISVD binding to serum | 18 | GGSLSR |
| albumin CDR3 (CDR3 | | |
| (kabat) of ALB23002) | | |
| ISVD binding to serum | 19 | GFTFRSFGMS |
| albumin CDR1 (CDR1 | | |
| (AbM) of ALB23002) | | |
| ISVD binding to serum | 20 | SISGSGSDTL |
| albumin CDR2 (CDR2 | | |
| (AbM) of ALB23002) | | |
| ISVD binding to serum | 21 | GGSLSR |
| albumin CDR3 (CDR3 | | |
| (AbM) of ALB23002) | | |
| T0263018B11-parent | 67 | DTLYTSLTWYSY |
| (CDR3-Kabat) | | |
| T0263018B11-parent | 68 | DTLYTSLTWYSY |
| (CDR3-AbM) | | |
| T0263018B11 | 185 | DVQLVESGGGVVQPGGSLRLSCAASGFTFSSYAMYWVRQAP |
| (E1D, W100cS) | | GKGLEWVSAISSGGGSTDYADSVKGRFTISRDNSKNTLYL |
| | | QMNSLRPEDTALYYCAADTLYTSLTSYSYWGQGTLVTVSS |
| T0263018B11- | 186 | EVQLVESGGGVVQPGGSLRLSCAASGFTFSSYAMYWVRQAP |
| (Y79S, T100bE, W100cS) | | GKGLEWVSAISSGGGSTDYADSVKGRFTISRDNSKNTLSLQ |
| | | MNSLRPEDTALYYCAADTLYTSLESYSYWGQGTLVTVSS |
| T0263018B11- | 187 | DVQLVESGGGVVQPGGSLRLSCAASGFTFSSYAMYWVRQAP |
| (E1D, Y79S, T100bE, | | GKGLEWVSAISSGGGSTDYADSVKGRFTISRDNSKNTLSL |
| W100cS) | | QMNSLRPEDTALYYCAADTLYTSLESYSYWGQGTLVTVSS |
| T0263201B05(E1D) | 158 | DVQLVESGGGLVQPGGSLRLSCAASGFTFSDYGMGWVRQA |
| | | PGKGLEVVSYISSDGGETSYADSVKGRFTISRDNAKNMLY |
| | | LQMSSLKLEDTALYYCAKGGVRGQGTLVTVSS |
| T0263201B05 | 168 | EVQLVESGGGLVQPGGSLRLSCAASGFTFSDYGMGWVRQA |
| | | PGKGLEVVSYISSDGGETSYADSVKGRFTISRDNAKNMLY |
| | | LQMSSLKLEDTALYYCAKGGVRGQGTLVTVSS |
| RSV001A04(L11V, | 200 | EVQLVESGGGVVQAGGSLSISCAASGGSLSNYVLGWFRQA |
| V89L, Q108L) | | PGKEREFVAAINWRGDITIGPPNVEGRFTISRDNAKNTGY |
| | | LQMNSLAPDDTALYYCGAGTPLNPGAYIYDWSYDYWGRGT |
| | | LVTVSS |
| T0263091D07 | 131 | EVQLVESGGGVVQPGGSLRLSCAASGFSFSDYYMYWVRQAP |
| | | GKGLEWVSAISSGGSSTYYADSVKGRFTISRDNSKNTVYLQ |
| | | MNSLRPEDTALYYCAADYLSVPDPSYEYWGQGTLVTVSS |
| T0263204B12 | 144 | EVQLVESGGGLVQPGGSLRLSCAASGFTLDYYAIGWFRQAP |
| | | GKEREGVLCISSSGDSTYYADSVKGRFTISRDNAKNTVYLQ |
| | | MDSLNPEDTAVYYCAVDPPSYWTGTGCLYGYRYWGQGTLVT |
| | | VSS |
| FcRn antibody FnAb12 | 201 | DIQLTQSPSSLSASVGDRVTLTCQATQDIDNNLNWYQQKPG |
| ScFv | | KAPKLLIYDASNLETGVPSRFSGSGSGTDFTFTISDLQPED |
| | | VATYYCQQYYNLPLTFGGGTKVDIKRSRGGGGSGGGGSGGG |
| | | GSLEMAEVQLVQSGAEVKKPGASVKVSCKASGYTFTSYDIN |
| | | WVRQATGQGLEWMGWMNPNSGNTGYAQKFQGRVTMTRNTSI |
| | | STAYMELSSLRSEDTAVYYCARGVDLGDGWGQGTLVTVSS |

TABLE A-2
Fc regions

| Name | ID | Amino acid sequence |
|---|---|---|
| Amino acid | 22 | CPPCPAPELLGGPSVFLFPPKPKDTLYITREPEVTCVVVDVSHED |
| sequences of Fc | | PEVKFNWYVDGVEVHNAKTKPREEQYNSTYRVVSVLTVLHQDWLN |
| regions | | GKEYKCKVSNKALPAPIEKTISKAKGQPREPQVYTLPPSRDELTK |
| (WO 2015/100299) | | NQVSLTCLVKGFYPSDIAVEWESNGQPENNYKTTPPVLDSDGSFF |
| | | LYSKLTVDKSRWQQGNVFSCSVMHEALKFHYTQKSLSLSPG |
| Amino acid | 23 | DKTHTCPPCPAPELLGGPSVFLFPPKPKDTLYITREPEVTCVVVD |
| sequence of Fc | | VSHEDPEVKFNWYVDGVEVHNAKTKPREEQYNSTYRVVSVLTVLH |
| regions | | QDWLNGKEYKCKVSNKALPAPIEKTISKAKGQPREPQVYTLPPSR |
| (WO 2015/100299) | | DELTKNQVSLTCLVKGFYPSDIAVEWESNGQPENNYKTTPPVLDS |
| (YTEKF) no KIH | | DGSFFLYSKLTVDKSRWQQGNVFSCSVMHEALKFHYTQKSLSLSP |
| as in TP061 | | GK |
| Amino acid | 24 | DKTHTCPPCPAPELLGGPSVFLFPPKPKDTLYITREPEVTCVVVD |
| sequences of Fc | | VSHEDPEVKFNWYVDGVEVHNAKTKPREEQYNSTYRVVSVLTVLH |
| regions | | QDWLNGKEYKCKVSNKALPAPIEKTISKAKGQPREPQVYTLPPSR |
| (WO 2015/100299) | | DELTKNQVSLTCLVKGFYPSDIAVEWESNGQPENNYKTTPPVLDS |
| | | DGSFFLYSKLTVDKSRWQQGNVFSCSVMHEALKFHYTQKSLSLSP |
| | | G |
| Amino acid | 25 | APELLGGPSVFLFPPKPKDTLMISRTPEVTCVVVDVSHEDPEVKF |
| sequences of Fc | | NWYVDGVEVHNAKTKPREEQYNSTYRVVSVLTVLHQDWLNGKEYK |
| regions | | CKVSNKALPAPIEKTISKAKGQPREPQVYTLPPSRDELTKNQVSL |
| (WO 2021/016571) | | TCLVKGFYPSDIAVEWESNGQPENNYKTTPPVLDSDGSFFLYSKL |
| | | TVDKSRWQQGNVFSCSVMHEALHNHYTQKSLSLSPGK |
| Amino acid | 26 | APEFLGGPSVFLFPPKPKDTLMISRTPEVTCVVVDVSQEDPEVQF |
| sequences of Fc | | NWYVDGVEVHNAKTKPREEQFNSTYRVVSVLTVLHQDWLNGKEYK |
| regions | | CKVSNKGLPSSIEKTISKAKGQPREPQVYTLPPSQEEMTKNQVSL |
| (WO 2021/016571) | | TCLVKGFYPSDIAVEWESNGQPENNYKTTPPVLDSDGSFFLYSRL |
| | | TVDKSRWQEGNVFSCSVMHEALHNHYTQKSLSLSLGK |
| Amino acid | 27 | APPVAGPSVFLFPPKPKDTLMISRTPEVTCVVVDVSHEDPEVQFN |
| sequences of Fc | | WYVDGVEVHNAKTKPREEQFNSTFRVVSVLTVVHQDWLNGKEYKC |
| regions | | KVSNKGLPAPIEKTISKTKGQPREPQVYTLPPSREEMTKNQVSLT |
| (WO 2021/016571) | | CLVKGFYPSDIAVEWESNGQPENNYKTTPPMLDSDGSFFLYSKLT |
| | | VDKSRWQQGNVFSCSVMHEALHNHYTQKSLSLSPGK |
| Amino acid | 61 | APELLGGPSVFLFPPKPKDTLMISRTPEVTCVVVDVSHEDPEVQF |
| sequences of Fc | | KWYVDGVEVHNAKTKPREEQYNSTFRVVSVLTVLHQDWLNGKEYK |
| regions | | CKVSNKALPAPIEKTISKTKGQPREPQVYTLPPSREEMTKNQVSL |
| (WO 2021/016571) | | TCLVKGFYPSDIAVEWESSGQPENNYNTTPPMLDSDGSFFLYSKL |
| | | TVDKSRWQQGNIFSCSVMHEALHNRFTQKSLSLSPGK |
| Fc region YTE-KF | 119 | DKTHTCPPCPAPELLGGPSVFLFPPKPKDTLYITREPEVTCVVVD |
| mutations (knob) | | VSHEDPEVKFNWYVDGVEVHNAKTKPREEQYNSTYRVVSVLTVLH |
| | | QDWLNGKEYKCKVSNKALPAPIEKTISKAKGQPREPQVYTLPPCR |
| | | DELTKNQVSLWCLVKGFYPSDIAVEWESNGQPENNYKTTPPVLDS |
| | | DGSFFLYSKLTVDKSRWQQGNVFSCSVMHEALKFHYTQKSLSLSP |
| | | G |
| Fc region YTE-KF | 120 | DKTHTCPPCPAPELLGGPSVFLFPPKPKDTLYITREPEVTCVVVD |
| mutations (hole) | | VSHEDPEVKFNWYVDGVEVHNAKTKPREEQYNSTYRVVSVLTVLH |
| | | QDWLNGKEYKCKVSNKALPAPIEKTISKAKGQPREPQVCTLPPSR |
| | | DELTKNQVSLSCAVKGFYPSDIAVEWESNGQPENNYKTTPPVLDS |
| | | DGSFFLVSKLTVDKSRWQQGNVFSCSVMHEALKFHYTQKSLSLSP |
| | | G |
| Fc region | 121 | DKTHTCPPCPAPELLGGPSVFLFPPKPKDTLYISRDPEVTCVVVD |
| YDQY mutations | | VSHEDPEVKFNWYVDGVEVHNAKTKPREEQYNSTYRVVSVLQVLH |
| (knob) | | QDWLNGKEYKCKVSNKALPAPIEKTISKAKGQPREPQVYTLPPCR |
| | | DELTKNQVSLWCLVKGFYPSDIAVEWESNGQPENNYKTTPPVLDS |
| | | DGSFFLYSKLTVDKSRWQQGNVFSCSVMHEALHYHYTQKSLSLSP |
| | | G |
| Fc region | 122 | DKTHTCPPCPAPELLGGPSVFLFPPKPKDTLYISRDPEVTCVVVD |
| YDQY mutations | | VSHEDPEVKFNWYVDGVEVHNAKTKPREEQYNSTYRVVSVLQVLH |
| (hole) | | QDWLNGKEYKCKVSNKALPAPIEKTISKAKGQPREPQVCTLPPSR |
| | | DELTKNQVSLSCAVKGFYPSDIAVEWESNGQPENNYKTTPPVLDS |
| | | DGSFFLVSKLTVDKSRWQQGNVFSCSVMHEALHYHYTQKSLSLSP |
| | | G |
| Fc region | 123 | DKTHTCPPCPAPELLGGPSVFLFPPKPKDTLYISRDPEVTCVVVD |
| YDQY mutations | | VSHEDPEVKFNWYVDGVEVHNAKTKPREEQYNSTYRVVSVLQVLH |
| (no KIH) | | QDWLNGKEYKCKVSNKALPAPIEKTISKAKGQPREPQVYTLPPSR |
| | | DELTKNQVSLTCLVKGFYPSDIAVEWESNGQPENNYKTTPPVLDS |
| | | DGSFFLYSKLTVDKSRWQQGNVFSCSVMHEALHYHYTQKSLSLSP |
| | | G |

TABLE A-3
Sequences for CDRs according to AbM numbering and frameworks
(“ID” refers to the given SEQ ID NO)

| ID | V_{HH} | ID | FR1 | ID | CDR1 | ID | FR2 | ID | CDR2 | ID | FR3 | ID | CDR3 | ID | FR4 |
|---|---|---|---|---|---|---|---|---|---|---|---|---|---|---|---|
| 186 | T0263018B11 | 190 | EVQLVE | 11 | GFTFSS | 203 | WVRQAP | 12 | AISSGG | 204 | YADSVK | 189 | DTLYTS | 135 | WGQGTL |
| | (Y79S, | | SGGGVV | | YAMY | | GKGLEW | | GSTD | | GRFTIS | | LESYSY | | VTVSS |
| | T100bE, | | QPGGSL | | | | VS | | | | RDNSKN | | | | |
| | W100cS) | | RLSCAA | | | | | | | | TLSLQM | | | | |
| | | | S | | | | | | | | NSLRPE | | | | |
| | | | | | | | | | | | DTALYY | | | | |
| | | | | | | | | | | | CAA | | | | |

TABLE A-4
Sequences for CDRs according to Kabat numbering and frameworks
(“ID” refers to the given SEQ ID NO)

| ID | V_{HH} | ID | FR1 | ID | CDR1 | ID | FR2 | ID | CDR2 | ID | FR3 | ID | CDR3 | ID | FR4 |
|---|---|---|---|---|---|---|---|---|---|---|---|---|---|---|---|
| 186 | T0263018B11- | 202 | EVQLVE | 8 | SYAMY | 203 | WVRQAP | 9 | AISSGG | 205 | RFTISR | 189 | DTLYTS | 135 | WGQGTL |
| | (Y79S, | | SGGGVV | | | | GKGLEW | | GSTDYA | | DNSKNT | | LESYSY | | VTVSS |
| | T100bE, | | QPGGSL | | | | VS | | DSVKG | | LSLQMN | | | | |
| | W100cS) | | RLSCAA | | | | | | | | SLRPED | | | | |
| | | | SGFTFS | | | | | | | | TALYYC | | | | |
| | | | | | | | | | | | AA | | | | |

TABLE A-5
Sequences for CDRs according to Abm numbering and frameworks
(“ID” refers to the given SEQ ID NO)

| ID | V_{HH} | ID | FR1 | ID | CDR1 | ID | FR2 | ID | CDR2 | ID | FR3 | ID | CDR3 | ID | FR4 |
|---|---|---|---|---|---|---|---|---|---|---|---|---|---|---|---|
| 187 | T0263018B11- | 206 | DVQLVE | 11 | GFTFSS | 203 | WVRQAP | 12 | AISSGG | 204 | YADSVK | 189 | DTLYTS | 135 | WGQGTL |
| | (E1D, Y79S, | | SGGGVV | | YAMY | | GKGLEW | | GSTD | | GRFTIS | | LESYSY | | VTVSS |
| | T100bE, | | QPGGSL | | | | VS | | | | RDNSKN | | | | |
| | W100cS) | | RLSCAA | | | | | | | | TLSLQM | | | | |
| | | | S | | | | | | | | NSLRPE | | | | |
| | | | | | | | | | | | DTALYY | | | | |
| | | | | | | | | | | | CAA | | | | |

TABLE A-6
Sequences for CDRs according to Kabat numbering and frameworks
(“ID” refers to the given SEQ ID NO)

| ID | V_{HH} | ID | FR1 | ID | CDR1 | ID | FR2 | ID | CDR2 | ID | FR3 | ID | CDR3 | ID | FR4 |
|---|---|---|---|---|---|---|---|---|---|---|---|---|---|---|---|
| 187 | T0263018B11- | 207 | DVQLVE | 8 | SYAMY | 203 | WVRQAP | 19 | AISSGG | 205 | RFTISR | 189 | DTLYTS | 135 | WGQGTL |
| | (E1D, Y79S, | | SGGGVV | | | | GKGLEW | | GSTDYA | | DNSKNT | | LESYSY | | VTVSS |
| | T100bE, | | QPGGSL | | | | VS | | DSVKG | | LSLQMN | | | | |
| | W100cS) | | RLSCAA | | | | | | | | SLRPED | | | | |
| | | | SGFTFS | | | | | | | | TALYYC | | | | |
| | | | | | | | | | | | AA | | | | |

TABLE A-7
Sequences for CDRs according to AbM numbering and frameworks
(“ID” refers to the given SEQ ID NO)

| ID | V_{HH} | ID | FR1 | ID | CDR1 | ID | FR2 | ID | CDR2 | ID | FR3 | ID | CDR3 | ID | FR4 |
|---|---|---|---|---|---|---|---|---|---|---|---|---|---|---|---|
| 158 | T0263201B05 | 161 | DVQLVE | 155 | GFTFSD | 162 | WVRQAP | 156 | YISSDG | 163 | YADSVK | 157 | GGV | 164 | RGQGTL |
| | (E1D) | | SGGGLV | | YGMG | | GKGLEV | | GETS | | GRFTIS | | | | VTVSS |
| | | | QPGGSL | | | | VS | | | | RDNAKN | | | | |
| | | | RLSCAA | | | | | | | | MLYLQM | | | | |
| | | | S | | | | | | | | SSLKLE | | | | |
| | | | | | | | | | | | DTALYY | | | | |
| | | | | | | | | | | | CAK | | | | |

TABLE A-8
Sequences for CDRs according to Kabat numbering and frameworks
(“ID” refers to the given SEQ ID NO)

| ID | V_{HH} | ID | FR1 | ID | CDR1 | ID | FR2 | ID | CDR2 | ID | FR3 | ID | CDR3 | ID | FR4 |
|---|---|---|---|---|---|---|---|---|---|---|---|---|---|---|---|
| 158 | T0263201B05 | 165 | DVQLVE | 159 | DYGMG | 162 | WVRQAP | 160 | YISSDG | 167 | RFTISR | 157 | GGV | 164 | RGQGTL |
| | (E1D) | | SGGGLV | | | | GKGLEV | | GETSYA | | DNAKNM | | | | VTVSS |
| | | | QPGGSL | | | | VS | | DSVKG | | LYLQMS | | | | |
| | | | RLSCAA | | | | | | | | SLKLED | | | | |
| | | | SGFTFS | | | | | | | | TALYYC | | | | |
| | | | | | | | | | | | AK | | | | |

TABLE A-9
Sequences for CDRs according to Abm numbering and frameworks
(“ID” refers to the given SEQ ID NO)

| ID | V_{HH} | ID | FR1 | ID | CDR1 | ID | FR2 | ID | CDR2 | ID | FR3 | ID | CDR3 | ID | FR4 |
|---|---|---|---|---|---|---|---|---|---|---|---|---|---|---|---|
| 168 | T0263201B05 | 169 | EVQLVE | 155 | GFTFSD | 162 | WVRQAP | 156 | YISSDG | 163 | YADSVK | 157 | GGV | 164 | RGQGTL |
| | | | SGGGLV | | YGMG | | GKGLEV | | GETS | | GRFTIS | | | | VTVSS |
| | | | QPGGSL | | | | VS | | | | RDNAKN | | | | |
| | | | RLSCAA | | | | | | | | MLYLQM | | | | |
| | | | S | | | | | | | | SSLKLE | | | | |
| | | | | | | | | | | | DTALYY | | | | |
| | | | | | | | | | | | CAK | | | | |

TABLE A-10
Sequences for CDRs according to Kabat numbering and frameworks
(“ID” refers to the given SEQ ID NO)

| ID | V_{HH} | ID | FR1 | ID | CDR1 | ID | FR2 | ID | CDR2 | ID | FR3 | ID | CDR3 | ID | FR4 |
|---|---|---|---|---|---|---|---|---|---|---|---|---|---|---|---|
| 168 | T0263201B05 | 166 | EVQLVE | 159 | DYGMG | 162 | WVRQAP | 160 | YISSDG | 167 | RFTISR | 157 | GGV | 164 | RGQGTL |
| | | | SGGGLV | | | | GKGLEV | | GETSYA | | DNAKNM | | | | VTVSS |
| | | | QPGGSL | | | | VS | | DSVKG | | LYLQMS | | | | |
| | | | RLSCAA | | | | | | | | SLKLED | | | | |
| | | | SGFTFS | | | | | | | | TALYYC | | | | |
| | | | | | | | | | | | AK | | | | |

TABLE A-11
Sequences for CDRs according to AbM numbering and frameworks
(“ID” refers to the given SEQ ID NO)

| ID | V_{HH} | ID | FR1 | ID | CDR1 | ID | FR2 | ID | CDR2 | ID | FR3 | ID | CDR3 | ID | FR4 |
|---|---|---|---|---|---|---|---|---|---|---|---|---|---|---|---|
| 131 | T0263091D07 | 132 | EVQLVE | 128 | GFSFSD | 133 | WVRQAP | 129 | AISSGG | 134 | YADSVK | 130 | DYLSVP | 135 | WGQGTL |
| | | | SGGGVV | | YYMY | | GKGLEW | | SSTY | | GRFTIS | | DPSYEY | | VTVSS |
| | | | QPGGSL | | | | VS | | | | RDNSKN | | | | |
| | | | RLSCAA | | | | | | | | TVYLQM | | | | |
| | | | S | | | | | | | | NSLRPE | | | | |
| | | | | | | | | | | | DTALYY | | | | |
| | | | | | | | | | | | CAA | | | | |

TABLE A-12
Sequences for CDRs according to Kabat numbering and frameworks
(“ID” refers to the given SEQ ID NO)

| ID | V_{HH} | ID | FR1 | ID | CDR1 | ID | FR2 | ID | CDR2 | ID | FR3 | ID | CDR3 | ID | FR4 |
|---|---|---|---|---|---|---|---|---|---|---|---|---|---|---|---|
| 131 | T0263091D07 | 138 | EVQLVE | 136 | DYYMY | 133 | WVRQAP | 137 | AISSGG | 140 | RFTISR | 130 | DYLSVP | 135 | WGQGTL |
| | | | SGGGVV | | | | GKGLEW | | SSTYYA | | DNSKNT | | DPSYEY | | VTVSS |
| | | | QPGGSL | | | | VS | | DSVKG | | VYLQMN | | | | |
| | | | RLSCAA | | | | | | | | SLRPED | | | | |
| | | | SGFSFS | | | | | | | | TALYYC | | | | |
| | | | | | | | | | | | AA | | | | |

TABLE A-13
Sequences for CDRs according to AbM numbering and frameworks
(“ID” refers to the given SEQ ID NO)

| ID | V_{HH} | ID | FR1 | ID | CDR1 | ID | FR2 | ID | CDR2 | ID | FR3 | ID | CDR3 | ID | R4 |
|---|---|---|---|---|---|---|---|---|---|---|---|---|---|---|---|
| 144 | T0263204B12 | 147 | EVQLVE | 141 | GFTLDY | 148 | WFRQAP | 142 | CISSSG | 150 | YADSVK | 143 | DPPSYW | 135 | WGQGTL |
| | | | SGGGLV | | YAIG | | GKEREG | | DSTY | | GRFTIS | | TGTGCL | | VTVSS |
| | | | QPGGSL | | | | VL | | | | RDNAKN | | YGYRY | | |
| | | | RLSCAA | | | | | | | | TVYLQM | | | | |
| | | | S | | | | | | | | DSLNPE | | | | |
| | | | | | | | | | | | DTAVYY | | | | |
| | | | | | | | | | | | CAV | | | | |

TABLE A-14
Sequences for CDRs according to Kabat numbering and frameworks
(“ID” refers to the given SEQ ID NO)

| ID | V_{HH} | ID | FR1 | ID | CDR1 | ID | FR2 | ID | CDR2 | ID | FR3 | ID | CDR3 | ID | FR4 |
|---|---|---|---|---|---|---|---|---|---|---|---|---|---|---|---|
| 144 | T0263204B12 | 151 | EVQLVE | 145 | YYAIG | 148 | WFRQAP | 146 | CISSSG | 153 | RFTISR | 143 | DPPSYW | 135 | WGQGTL |
| | | | SGGGLV | | | | GKEREG | | DSTYYA | | DNAKNT | | TGTGCL | | VTVSS |
| | | | QPGGSL | | | | VL | | DSVKG | | VYLQMD | | YGYRY | | |
| | | | RLSCAA | | | | | | | | SLNPED | | | | |
| | | | SGFTLD | | | | | | | | TAVYYC | | | | |
| | | | | | | | | | | | AV | | | | |

### EXAMPLES

### Example 1

Generation and Expression of FcRn Antagonists Comprising an Fc Domain with or without the Addition of a FcRn Binding ISVD

Asymmetrical fusion polypeptide constructs of a FcRn binding Nanobody® VHH (ISVD) linked to an Fc domain of an IgG were generated using the Knob-in-Hole technology as commonly known in the art (and as described for instance in patent publication WO 1996/27011 by Genentech as well as scientific publications by Ridgway, J B et al., “‘Knobs-into-holes’ engineering of antibody CH3 domains for heavy chain heterodimerization”, Protein engineering 9,7 (1996): 617-21 and Merchant et al., “An efficient route to human bispecific IgG”, Nature Biotechnology 16, (1998): 677-681). The symmetrical control Fc construct without ISVD (Nanobody® VHH) was generated without the Knob-in-Hole technology. See, e.g., FIG. 1 and Table C-0.

DNA fragments of a FcRn binding Nanobody® VHH and/or an Fc domain of an IgG, obtained by PCR with specific combinations of forward and reverse primers each carrying a specific BpiI restriction site, were cloned in the appropriate expression vector via Golden Gate cloning (Engler C, Marillonnet S., “Golden Gate cloning”, Methods Mol Biol. 2014; 1116:119-31). After Sanger sequence confirmation, the plasmid DNA was then transfected into CHOEBNALT85 cells (QMCF Technology) for protein production. The Nanobody® VHH-Fc fusion polypeptide constructs or Fc chain only were purified from the cell supernatants using a protein A capture step followed by an ion exchange and/or size exclusion chromatography purification step.

### Example 2

### FcRn Binding Studies of FcRn Binding Nanobody® VHH-Fc Fusion Polypeptide Constructs

A set of Nanobody® VHH-Fc fusion polypeptide constructs (FcRn antagonists) was generated that consisted of an Fc domain with or without a Nanobody® VHH specifically binding to FcRn that does not compete with IgG for binding to FcRn. The Fc domain in all the constructs was IgG1 Fc YTE-KF (=M252Y, S254T, T256E, H433K, N434F) as described in WO 2015/100299 (comprising SEQ ID NO: 23 or 24, with the addition of knob in hole mutations in the Nanobody® VHH-Fc fusion polypeptide constructs to obtain asymmetrical molecules e.g., SEQ ID NO: 119 and 120, whereas the FcRn binding Nanobody® VHH used was 18B11(W100cS) (SEQ ID NO:15) or 18111 (E1D, W100cS) (SEQ ID NO: 185). The Nanobody® VHH sequence in these fusion polypeptide constructs was fused directly to the IgG1 hinge at the N-terminus of the Fc domain, see above sequence) or via a 35GS peptide linker, SEQ ID NO: 39, present in the C-terminus of the Fc domain, (as described in detail herein) (TP067 and TP069, respectively, see FIG. 1, and SEQ ID NOs: 124+125 and 126+127). A control Fc domain that does not contain a Nanobody® VHH (TP061, see FIG. 1) was generated.

The Nanobody® VHH-Fc protein constructs (FcRn antagonists) were characterized by affinity determination for human FcRn and cyno FcRn at pH 6.0 and pH 7.4 on the Biacore 8K+ instrument. For affinity measurements, ^{˜}200-400 of biotinylated human or cyno FcRn was captured on a Series S Sensor Chip SA. The Nanobody® VHH-Fc fusion proteins were injected at 9 different concentrations (between 0.5 and 1500 nM) and allowed to associate for 120 s at 30 μL/min and dissociate for 600 s.

The data could not be fitted with the 1:1 Langmuir dissociation model, and a Bivalent Analyte fitting model was used instead due to biphasic on-rate behaviour observed during analysis. The FcRn affinities obtained for TP061 were in line with the published data (see, e.g., Vaccaro, C., Zhou, J., Ober, R. et al., “Engineering the Fc region of immunoglobulin G to modulate in vivo antibody levels”, Nat. Biotechnol. 23, 1283-1288 (2005)). The Nanobody® VHH-Fc protein constructs containing the FcRn binding Nanobody® VHH 18B11(W100cS) showed an improved binding to FcRn due to an avidity effect (i.e., binding to FcRn via Fc and Nanobody® building block) (TP067 and TP069). A similar binding was seen for the N- or C-terminal fusion of 18B11(W100cS) to the Abdeg Fc (Fc domain) (TP067 and TP069).

TABLE 6
FcRn affinities at pH 6.0 or pH 7.4

| | Human FcRn | | Cynomolgus FcRn | |
|---|---|---|---|---|
| Construct | pH 6.0 | pH 7.4 | pH 6.0 | pH 7.4 |
| T-0263-00_TP061 | 1.72E−08M | No | 2.75E−08M | No |
| | | significant | | significant |
| | | binding | | binding |
| T-0263-00_TP067 | 6.92E−10M | 1.03E−08M | 5.41E−10M | 1.12E−08M |
| T-0263-00_TP069 | 8.73E−10M | 1.40E−08M | 6.83E−10M | 1.25E−08M |

### Example 3

### Development and Optimization of Serum PK Assay (Assay for Detection of FcRn Antagonist and Privigen® in Mouse Plasma

The concentration of each FcRn antagonist and a surrogate concentration of Privigen® (IgG) at each timepoint were determined by a bottom-up LC-MS2 assay. Plasma samples were spiked with two internal standards: SiluMAB MSQC7 to account for losses and interferences in the quantification of Privigen® and an in-house produced Fc-fusion Nanobody® construct to account for losses and interferences in the quantification of the FcRn antagonist. Subsequently, samples were diluted, reduced, carbamidomethylated and proteins were precipitated. The obtained pellets were digested with trypsin and the resulting surrogate peptides were analysed by LC-MS/MS. Calibration standards and QC samples were prepared by spiking FcRn antagonist, Privigen® and both internal standards in blank plasma, so that every single calibration standard aliquot contained all the four substances.

Analysis of the peptides was performed in an Agilent 1290 Infinity II UHPLC hyphenated to a Sciex 6500+ mass spectrometer. For separation, a column Aquity UPLC Peptide CSH C18 130Å 1.7 μm 50×2.1 mm (Waters) was flushed at 50° C. with a stepwise gradient of water/DMSO/formic acid (100/1/0.5; v/v/v) and acetonitrile/DMSO/formic acid (100/1/0.5; v/v/v) with a 0.50 mL min-1 flow. The mass spectrometer was operated in positive mode according manufacturer's instructions with the ion source at 5500 V and 450° C. Dwell times were 30 s. One multiple reaction transition corresponding to one unique peptide of the constant domain of the light chain of human antibodies was used as surrogate for the concentration of Privigen®. For the FcRn antagonists, one multiple reaction transition of one unique surrogate peptide in the Fc domain was used as surrogate for quantification. Both FcRn antagonist and Privigen were simultaneously quantified, in a single analytical run. Chromatographic peak areas were determined with the algorithm AutoPeak (Sciex OS). Concentrations were determined by using the ratio area of the analyte to the area of the corresponding internal standard in the same sample and comparing the results to the calibration curve obtained with the calibration standards.

### Example 4

In Vivo Study: Pharmacodynamics Study of hIgG Clearing in Transgenic Mice

A mouse study was performed in Tg32 mice (B6.Cg-Fcgrttm1Dcr Tg(FCGRT) 32Dcr/DcrJ) to determine IgG clearing by the FcRn antagonist constructs under investigation. To achieve relevant endogenous levels of hIgG in these mice, Tg32 mice were injected intravenously in the tail with 500 mg/kg Privigen®. 48 h after Privigen® administration, 9 mice/group were injected intravenously in the tail with either PBS (placebo group), 20 mg/kg of benchmark molecule TP061, or an equimolar concentration of test compounds TP067 or TP069, which contain either an N-terminal, or C-terminal fusion of the engineered Fc with a pH-dependent FcRn Nb, as described in detail above.

Blood was retrieved at different time points (3 mice per time point, staggered sampling scheme) and serum (plasma) was prepared. Serum (plasma) samples were analyzed by quantitative LC-MS/MS for the presence of Privigen® or for the presence of the TP061, TP067 and TP069 as described in Example 3. PK parameters for FcRn antagonists and Privigen® were obtained from non-compartmental analysis in Phoenix WinNonlin® (version 8.2.2.227. Certara) using the Plasma Data Module. Where applicable, sampling times with steep concentration decline of compound due to suspected ADA impact were excluded from analysis.

Mean (SD) Privigen® concentration vs time profiles are shown in FIG. 2 A and the clearance of IgG plotted as the percentage of the hIgG concentration is shown in FIG. 2 B.

For all treatment groups, Privigen® AUCs were determined and % reduction in Privigen® exposure calculated. Results are shown in Table 7.

TABLE 7
Privigen ® clearance

| | FcRn | PRIVIGEN | % Reduction |
|---|---|---|---|
| | antagonist | AUCₗₐₛₜ | In PRIVIGEN |
| Treatment | dose (mg/kg) | (h*μg/mL) | Exposure |
| PRIVIGEN | — | 528000 | — |
| PRIVIGEN + TP061 | 20 | 189000 | 64% |
| PRIVIGEN + TP067 | 25 | 103000 | 80% |
| PRIVIGEN + TP069 | 25 | 117000 | 78% |

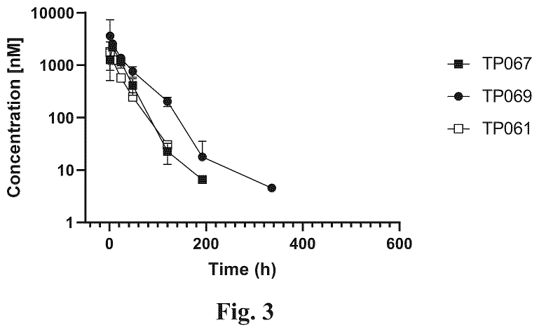

Mean (SD) TP061, TP067 and TP069 concentration vs time profiles are shown in FIG. 3.

PK parameters calculated via NCA analysis are listed in Table 8.

TABLE 8
PK parameters of FcRn antagonists, obtained by non-compartmental
analysis (NCA)

| | AUC_{inf} | t_{1/2} | Cl |
|---|---|---|---|
| Analyte | (ug*h/mL) | (h) | (mL/h/kg) |
| TP061 | 2380 | 22.9 | 8.39 |
| TP067 | 4900 | 18.5 | 5.10 |
| TP069 | 8600 | 31.1 | 2.91 |

We can conclude from the results that administration of TP067 and TP069 results in an increased IgG clearance, compared to TP061, while the clearance of TP067 and TP069 is reduced. In addition, the half-life of the FcRn antagonists of the present invention can be essentially maintained (see, e.g., TP067) or even improved (TP069) compared to constructs comprising a variant Fc domain only (TP061). See FIGS. 2 and 3 and Table 8.

### Example 5

Generation and Expression of FcRn Antagonists Comprising an IgG Competitor Domain with or without the Addition of a FcRn Binding Domain not Competing with IgG

### 1) The IgG Competitor Domain is an Fc Domain

Asymmetrical fusion polypeptide constructs of a FcRn binding Nanobody® VHH (ISVD) or a FcRn binding scFv linked to an Fc domain of an IgG were generated using the Knob-in-Hole technology as commonly known in the art (and as described for instance in patent publication WO 1996/27011 by Genentech as well as scientific publications by Ridgway, J B et al., “‘Knobs-into-holes’ engineering of antibody CH3 domains for heavy chain heterodimerization”, Protein engineering 9,7 (1996): 617-21 and Merchant et al., “An efficient route to human bispecific IgG”, Nature Biotechnology 16, (1998): 677-681). The Fc domain in the constructs was IgG1 Fc YDQY (=M252Y,T256D,T307QN434Y) (comprising SEQ ID NO: 121 or 122, containing the knob in hole mutations in the Nanobody® VHH-Fc fusion polypeptide constructs to obtain asymmetrical molecules; e.g. TPP-122905, TPP-122907, TPP-122908, TPP-122909, TPP-122910, TPP-122912 see Table C-1 below).

The symmetrical Fc constructs (e.g., TPP-122903 and TPP-122904) of a FcRn binding Nanobody® VHH (ISVD) linked to an Fc domain of an IgG were generated without the Knob-in-Hole technology (homodimeric Fc construct, comprising two times SEQ ID NO.: 123). The Fc domain in these symmetrical constructs was IgG1 Fc YDQY (=M252Y,T256D,T307Q,N434Y) (comprising SEQ ID NO: 123). The Nanobody® VHH's linked to each of the polypeptide chains or fragments of the Fc domain were T0263018B11(Y79S,T100bE,W100cS) (SEQ ID NO.: 186), for TPP-122903 and T0263091D07 (SEQ ID NO.: 131) for TPP-122904.

The symmetrical control Fc constructs (e.g., TPP-122902) without Nanobody® VHH were generated without the Knob-in-Hole technology (homodimeric Fc construct, comprising two times SEQ ID NO.: 123). The Fc domain in these symmetrical constructs was IgG1 Fc YDQY (=M252Y,T256D,T307Q,N434Y) (comprising SEQ ID NO: 123).

TPP-122902 comprises two polypeptides which comprise or consists of the human IgG1 Fc YDQY (SEQ ID NO.: 123). SEQ ID NO.: 123 is as follows: DKTHTCPPCPAPELLGGPSVFLFPPKPKDTLYISRDPEVTCVVVDVSHEDPEVKFNWYVDGVEVHNAKTK PREEQYNSTYRVVSVLQVLHQDWLNGKEYKCKVSNKALPAPIEKTISKAKGQPREPQVYTLPPSRDELTKN QVSLTCLVKGFYPSDIAVEWESNGQPENNYKTTPPVLDSDGSFFLYSKLTVDKSRWQQGNVFSCSVMHE ALHYHYTQKSLSLSPG

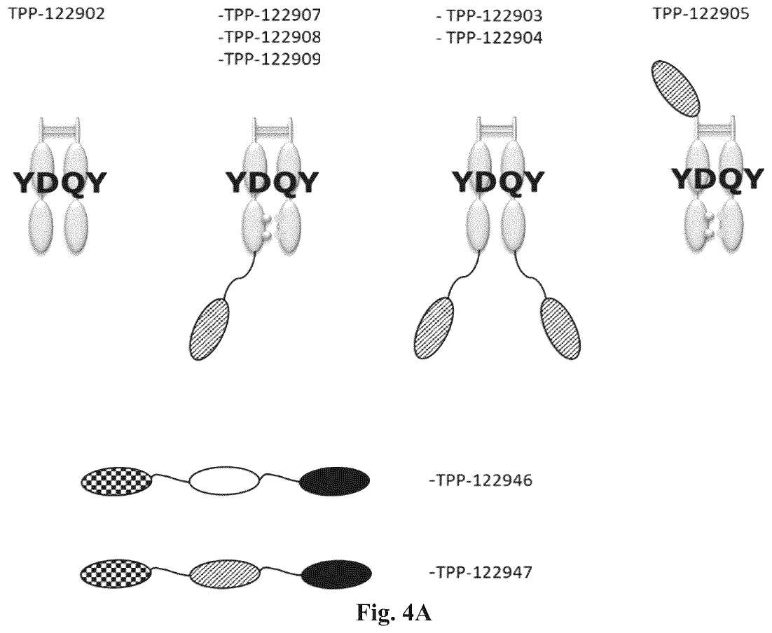

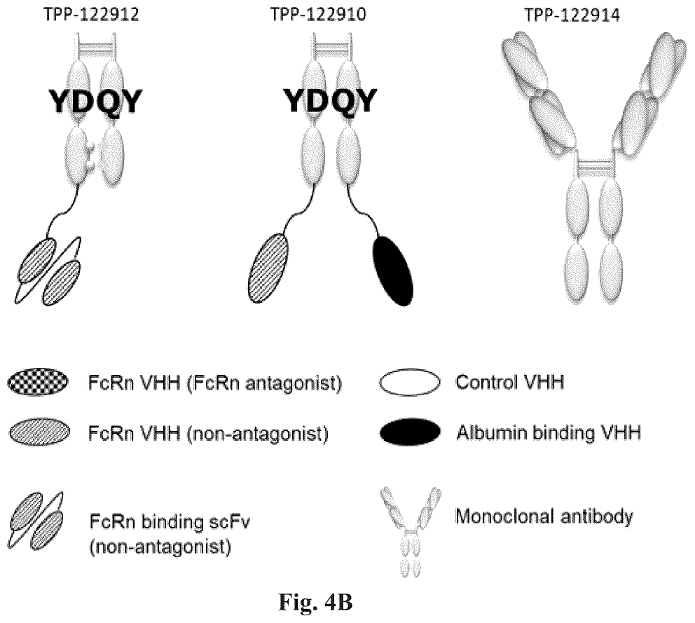

The FcRn binding domain that does not compete with IgG was a Nanobody® VHH (ISVD) in the above constructs. The FcRn binding ISVD used was a variant of T0263018B11 (SEQ ID NO. 15, 185, 186 or 187), T0263091D07 (SEQ ID NO.: 131) or T0263204B12 (SEQ ID NO.: 144) as described herein. The Nanobody® VHH (ISVD) sequences in these fusion proteins were fused via a linker (as described in detail herein) to the N- and/or C-terminus of the Fc chain, i.e., via an IgG1 hinge and/or a GS linker, respectively (see FIG. 4 and Table C-1).

In TPP-122912 the FcRn binding domain that does not compete with IgG was a FcRn binding scFv, SEQ ID NO.: 201: DIQLTQSPSSLSASVGDRVTLTCQATQDIDNNLNWYQQKPGKAPKLLIYDASNLETGVPSRFSGSGSGTDF TFTISDLQPEDVATYYCQQYYNLPLTFGGGTKVDIKRSRGGGGSGGGGSGGGGSLEMAEVQLVQSGAEV KKPGASVKVSCKASGYTFTSYDINWVRQATGQGLEWMGWMNPNSGNTGYAQKFQGRVTMTRNTSIS TAYMELSSLRSEDTAVYYCARGVDLGDGWGQGTLVTVSS

The Nanobody® VHH (ISVD)-Fc fusion polypeptide constructs, scFv-Fc fusion polypeptide constructs and Fc chain only polypeptides were expressed in CHO cells and purified from the cell supernatants using a protein A capture step followed by a size exclusion chromatography purification step.

### 2) The IgG Competitor Domain is an ISVD (e.g., Nanobody® VHH)

The FcRn antagonist formats without Fc consist of (1) an FcRn binding Nanobody® VHH (ISVD) that competes with IgG, (2) an FcRn binding Nanobody® VHH (ISVD) that does not compete with IgG or a control ISVD not binding to FcRn or to any other envisaged target and (3) an Nanobody® VHH (ISVD) binding to serum albumin for half-life extension. The FcRn binding ISVD that competes with IgG was T02632011B05(E1D) (SEQ ID NO.: 158) as described herein.

SEQ ID NO.: 158:
DVQLVESGGGLVQPGGSLRLSCAASGFTFSDYGMGWVRQAPGKGLEVVSY
ISSDGGETSYADSVKGRFTISRDNAKNMLYLQMSSLKLEDTALYYCAKGG
VRGQGTLVTVSS

The FcRn binding Nanobody® VHH (ISVD) that does not compete with IgG was T0263018B11(Y79S,T100bE,W100cS) (SEQ ID NO: 186) and the control ISVD was RSV001A04(L11V,V89L,Q108L) (SEQ ID NO.: 200). See constructs TPP-122946 (with control ISVD) and TPP-122947 (with T0263018B11(Y79S,T100bE,W100cS)), FIG. 4 and Table C-2.

SEQ ID NO.: 200:
EVQLVESGGGVVQAGGSLSISCAASGGSLSNYVLGWFRQAPGKERE0FVA
AINWRGDITIGPPNVEGRFTISRDNAKNTGYLQMNSLAPDDTALYYCGAG
TPLNPGAYIYDWSYDYWGRGTLVTVSS

The albumin binding ISVD was ALB23002 (SEQ ID NO: 58). The 3 domains were linked via 35GS linkers (SEQ ID NO: 39).

The Nanobody® VHH (ISVD) polypeptide construct without Fc was expressed in CHO cells and purified from the cell supernatants using a protein A capture step followed by a size exclusion chromatography purification step.

### 3) Control FcRn Antagonist

As a control, a monoclonal antibody FcRn antagonist was generated (TPP-122914 in FIG. 4, benchmark monoclonal antibody directed against FcRn for the treatment of IgG related diseases), produced in CHO cells and purified from the cell supernatants using a protein A capture step followed by a size exclusion chromatography purification step.

### Example 6

### Development and Optimization of an Assay for Detection of Privigen® in Mouse Plasma.

The concentration of Privigen® (human IgG) in each sample was determined by a bottom-up LC-MS2 assay. Plasma samples were spiked with SiluMAB MSQC7 to account for losses and interferences in the quantification of Privigen®

Subsequently, samples were diluted, reduced, carbamidomethylated and proteins were precipitated. The obtained pellets were digested with trypsin and the resulting surrogate peptides were analysed by LC-MS2 using reverse phase chromatography for separation and a triple quadrupole mass spectrometer for detection. Both instruments were operated according to manufacturer's instructions. One multiple reaction transition corresponding to one unique peptide of the constant domain of the light chain of human antibodies was used as surrogate for the concentration of Privigen®.

Chromatographic peak areas were automatically determined with a dedicated software (Analyst® or SciexOS®, both from Sciex™). The ratios between the area of the analyte (Privigen® or FcRn antagonist) and the area of the corresponding internal standard in the same sample were calculated and interpolated in the corresponding calibration curve.

### Example 7

In Vivo Pharmacodynamics (PD) Study: Evaluation of hIgG Clearing Potential of Different FcRn Antagonist Formats in FcRn Transgenic Mice

A mouse study was performed in Tg32 mice (B6.Cg-Fcgrttm1Dcr Tg(FCGRT) 32Dcr/DcrJ) to determine the potential of FcRn antagonists, listed under Example 5 and FIG. 4, to clear hIgG, under the form of Privigen®, from mouse plasma. To achieve relevant endogenous levels of hIgG in Tg32 mice, they were injected intravenously in the tail with 500 mg/kg Privigen®. 48 h after Privigen® administration, 3-6 mice/group were injected intravenously in the tail with either PBS (placebo group), 20 mg/kg of FcRn antagonist TPP-122902, or an equimolar amount of the other FcRn antagonists listed in detail above and in FIG. 4.

Blood was retrieved serially via microsampling at different time points (0.08h, 1 h, 6h, 24 h, 2d, 5d, 8d, 14d, 21d; n=3 mice per time point for constructs and n=6 for PBS group) and plasma was prepared. Plasma samples were analyzed by quantitative LC-MS/MS for the presence of Privigen®, as described in Example 6. AUC values for Privigen® were obtained from non-compartmental analysis in Phoenix WinNonlin® (version 8.2.2.227. Certara) using the Plasma Data Module.

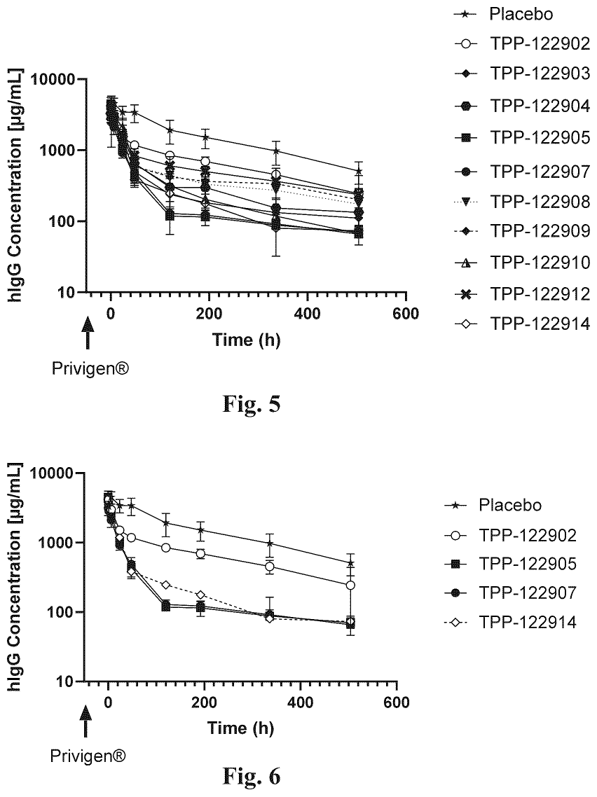

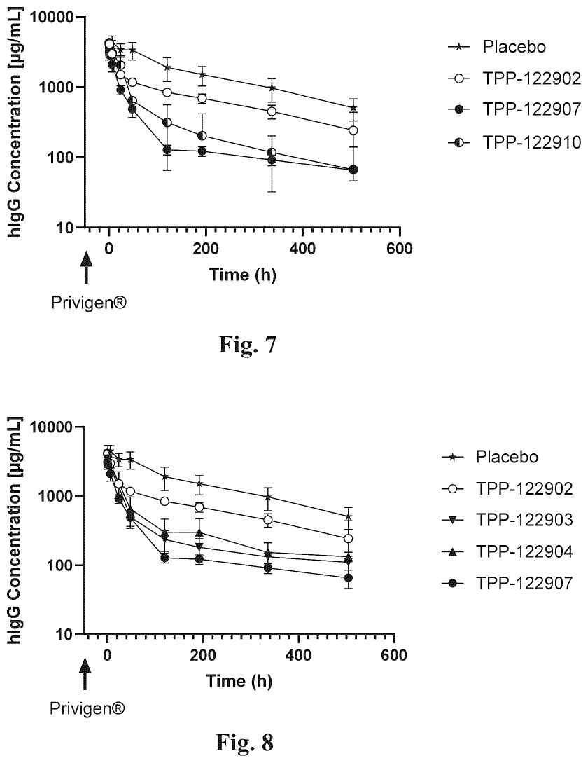

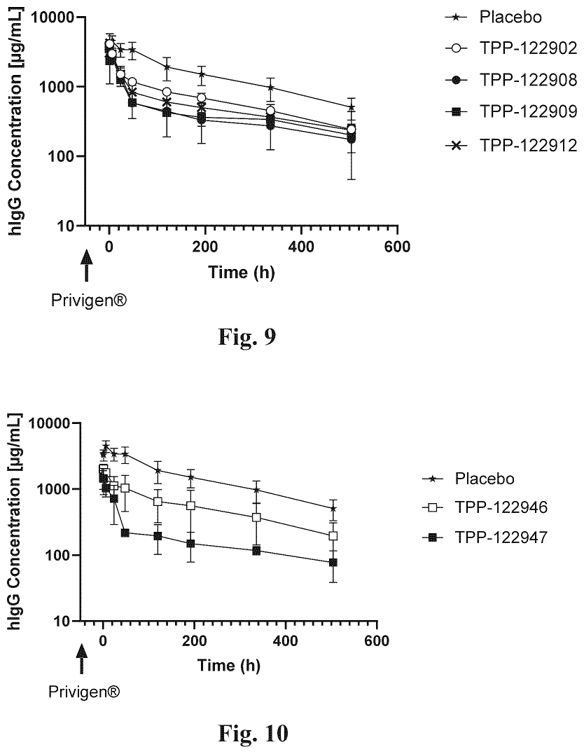

Mean (SD) Privigen® concentration vs time profiles of animals treated with PBS or FcRn antagonist constructs are shown in FIGS. 5-10. FIG. 5 provides the compiled Privigen® concentration vs time profiles for all constructs based on an antagonistic Fc domain while FIGS. 6-10 highlight subsets of FcRn antagonists of the current invention compared to placebo or reference constructs.

Mean Privigen® AUCs were determined and % reduction in Privigen® exposure calculated. Results are shown in Table 9.

TABLE 9
Privigen ® clearance

| | FcRn | PRIVIGEN | % Reduction |
|---|---|---|---|
| | antagonist | AUCₗₐₛₜ | In Privigen ® |
| Treatment | dose (mg/kg) | (h*ug/mL) | Exposure |
| PBS | — | 951000 | — |
| TPP-122902 | 20 | 453000 | 52 |
| TPP-122903 | 31 | 192000 | 80 |
| TPP-122904 | 31 | NA | NA |
| TPP-122905 | 25 | NA | NA |
| TPP-122907 | 25 | 135000 | 86 |
| TPP-122908 | 25 | 238000 | 75 |
| TPP-122909 | 25 | 316000 | 67 |
| TPP-122910 | 31 | 231000 | 76 |
| TPP-122912 | 31 | 396000 | 58 |
| TPP-122914 | 55 | 249000 | 74 |
| TPP-122946 | 15.5 | 347000 | 64 |
| TPP-122947 | 15.5 | 131000 | 86 |

NA: not analyzed

We can conclude from the results that fusion of at least one non-FcRn antagonistic (non-competing with IgG binding to FcRn) ISVD or alternative scaffold to an existing FcRn antagonistic Fc, results in improved FcRn antagonism compared to the antagonistic Fc alone, as evidenced by increased hIgG clearance in a relevant transgenic mouse model. In a similar manner, combination of a non-FcRn antagonistic ISVD to an FcRn antagonistic ISVD results in an increased hIgG clearance compared to the FcRn antagonistic ISVD alone.

### Items of the Present Invention

The present invention provides the following items:
1. A FcRn antagonist comprising:
   a) At least one first polypeptide specifically binding to an epitope on Fc receptor (FcRn); and
   b) At least a second polypeptide which competes with wild-type IgG1 Fc region for binding to FcRn.
2. The FcRn antagonist according to item 1, wherein the at least two polypeptides (a) and (b) comprised in the FcRn antagonist show a synergistic or cooperative effect, preferably wherein the synergistic or cooperative effect allows for an improved binding and/or a similar half-life of the FcRn antagonists, preferably an improved half-life of the FcRn antagonists, as compared with the binding and/or half-life of each of the at least two polypeptides (a) and (b) on their own.
3. The FcRn antagonist according to any one of items 1 or 2, wherein the at least one first polypeptide as defined in (a) competes with wild-type IgG1 Fc region for binding to FcRn.
4. The FcRn antagonist according to any one of items 1 to 3, wherein the at least one first polypeptide as defined in (a) does not compete with wild-type IgG1 Fc region for binding to FcRn, preferably wherein the at least one first polypeptide as defined in (a) comprises or consists of Affibody®, a scFv, a Fab, a Designed Ankyrin Repeat Protein (DARPin®), a Nanofitin® (aka affitin) and an immunoglobulin variable domain sequence (ISVD), preferably wherein, if the at least one first polypeptide as defined in (a) comprises or consists of a scFv, the scFv comprises or consists of SEQ ID NO.: 201.
5. The FcRn antagonist according to any one of items 1 to 4, wherein the at least one first polypeptide as defined in (a) comprises or consists of an immunoglobulin single variable domain (ISVD).
6. The FcRn antagonist according to any one of items 1 to 4, wherein the at least one first polypeptide as defined in (a) comprises or consists of a Fc domain or a fragment thereof.
7. The FcRn antagonist according to any one of items 1 to 5, wherein the FcRn antagonist comprises:
   a) At least one immunoglobulin single variable domain (ISVD) specifically binding to an epitope on Fc receptor (FcRn), wherein the ISVD does not compete with wild-type IgG1 Fc region for binding to FcRn; and
   b) At least one polypeptide which competes with wild-type IgG1 Fc region for binding to FcRn.
8. The FcRn antagonist according to any one of items 5 to 7, wherein the at least one ISVD does not compete with wild-type human serum albumin (HSA) for binding to FcRn.
9. The FcRn antagonist according to any one of items 5 to 8, wherein the FcRn epitope to which the at least one ISVD binds comprises at least one of the following amino acid residues 1A, 2E, 3S, 4H, 5L, 32P, 97E, 98L, 99G, 100P, 101D, 102N, 103T, 164R, 167L, 168E, 171R, 174L, 175E, 177K, 204Y, 205P, 206P, 207E, 208L, 209Q, 255Q, 256H, 257A, 259L, 260A, 261Q, and/or 262P, amino acid residues being numbered according to SEQ ID NO: 1.
10. The FcRn antagonist according to any one of items 1 to 9, wherein the at least one second polypeptide as defined in (b) is an antibody or a fragment thereof.
11. The FcRn antagonist according to item 10, wherein the antibody or a fragment thereof is a monoclonal antibody, a fusion antibody, a humanized antibody, a human antibody or a single chain antibody.
12. The FcRn antagonist according to any one of items 1 to 11, wherein the at least one polypeptide as defined in (b) comprises or consists of an immunoglobulin single variable domain (ISVD), preferably wherein the ISVD comprises a CDR1 (AbM numbering) that is the amino acid sequence of SEQ ID NO: 155, a CDR2 (AbM numbering) that is the amino acid sequence of SEQ ID NO: 156 and a CDR3 (AbM numbering) that is the amino acid sequence of SEQ ID NO: 157, more preferably wherein the ISVD comprises or consists of SEQ ID NO.: 158 (T0263201B05(E1D), DVQLVESGGGLVQPGGSLRLSCAASGFTFSDYGMGWVRQAPGKGLEVVSYISSDGGETSYADS VKGRFTISRDNAKNMLYLQMSSLKLEDTALYYCAKGGVRGQGTLVTVSS) or 168 (T0263201B05, EVQLVESGGGLVQPGGSLRLSCAASGFTFSDYGMGWVRQAPGKGLEVVSYISSDGGETSYADSV KGRFTISRDNAKNMLYLQMSSLKLEDTALYYCAKGGVRGQGTLVTVSS).
13. The FcRn antagonist according to any one of items 1 to 11, wherein the at least one polypeptide as defined in (b) comprises or consists of a Fc domain or a fragment thereof, preferably a dimeric Fc domain or fragment thereof, more preferably a heterodimeric Fc domain.
14. The FcRn antagonist according to any one of items 1 to 13, wherein the at least one polypeptide as defined in (b) specifically binds to FcRn with increased affinity relative to a wild-type IgG1 Fc region.
15. The FcRn antagonist according to any one of items 1 to 14, wherein the at least one polypeptide as defined in (b) specifically binds to FcRn with increased affinity at an acidic pH and at an extracellular physiological pH relative to a wild-type IgG Fc region, optionally wherein the acidic pH is about 6.0 and optionally wherein the non-acidic pH is about 7.4.
16. The FcRn antagonist according to any one of items 1 to 15, wherein the at least one polypeptide as defined in (b) specifically binds to FcRn with reduced pH dependence relative to a wild-type IgG1 Fc region.
17. The FcRn antagonist according to any one of items 1 to 16, wherein the at least one polypeptide as defined in (b) has altered affinity (increased or decreased) for CD16 as compared to a wild-type IgG1 Fc region.
18. The FcRn antagonist according to any one of items 13 to 17, wherein the Fc domain or fragment thereof comprises at least one, preferably all, of the following amino acids at the following positions:
   a) a tyrosine (Y) at amino acid position 252,
   b) a threonine (T) at amino acid position 254,
   c) a glutamic acid (E) at amino acid position 256,
   d) a lysine (K) at amino acid position 433,
   e) a phenylalanine (F) at amino acid position 434, and/or
   f) a tyrosine (Y) at amino acid position 436;
   according to EU numbering.
19. The FcRn antagonist according to any one of items 13 to 17, wherein the Fc domain or fragment thereof comprises a combination of the following four amino acid residues:
   a) a tyrosine (Y) at amino acid position 252,
   b) an aspartic acid (D) or a glutamic acid (E) at amino acid position 256,
   c) a tryptophan (W) or a glutamine (Q) at amino acid position 307, and
   d) a phenylalanine (F) or a tyrosine (Y) at amino acid position 434; according to EU numbering.
20. The FcRn antagonist according to item 19, wherein the Fc domain or fragment thereof comprises a combination of amino acid residues selected from the group consisting of:
   a) a tyrosine (Y) at amino acid position 252, an aspartic acid (D) at amino acid position 256, a glutamine (Q) at amino acid position 307, and a tyrosine (Y) at amino acid position 434;
   b) a tyrosine (Y) at amino acid position 252, a glutamic acid (E) at amino acid position 256, a tryptophan (W) at amino acid position 307, and a tyrosine (Y) at amino acid position 434;
   c) a tyrosine (Y) at amino acid position 252, a glutamic acid (E) at amino acid position 256, a glutamine (Q) at amino acid position 307, and a tyrosine (Y) at amino acid position 434;
   d) a tyrosine (Y) at amino acid position 252, an aspartic acid (D) at amino acid position 256, a glutamine (Q) at amino acid position 307, and a phenylalanine (F) at amino acid position 434;
   e) a tyrosine (Y) at amino acid position 252, an aspartic acid (D) at amino acid position 256, a tryptophan (W) at amino acid position 307, and a tyrosine (Y) at amino acid position 434; and
   f) a tyrosine (Y) at amino acid position 252, an aspartic acid (D) at amino acid position 256, a tryptophan (W) at amino acid position 307, and a phenylalanine (F) at amino acid position 434,
preferably wherein the Fc domain or fragment thereof comprises a combination of amino acid residues selected from the group consisting of:
   a) a tyrosine (Y) at amino acid position 252, an aspartic acid (D) at amino acid position 256, a glutamine (Q) at amino acid position 307, and a tyrosine (Y) at amino acid position 434.
21. The FcRn antagonist according to any one of items 18 to 20, wherein the at least one second polypeptide as defined in (b) comprises or consists of SEQ ID NO: 22-24 and/or 119-123, preferably SEQ ID NO: 23, 24 or 119-123.
22. The FcRn antagonist according to any one of items 5 to 21, wherein the FcRn epitope to which the at least one ISVD binds comprises at least one of the following combinations of amino acid residues:
   a) 4H and 5L,and/or
   b) 98L, 99G, 100P, 101D and 102N, and/or
   c) 167L, 171R, 174L, 175E and 177K, and/or
   d) 255Q, 256H, 257A, 259L, 260A and 262P,
   wherein the amino acid residues are numbered according to SEQ ID NO: 1.
23. The FcRn antagonist according to any one of items 5 to 22, wherein the FcRn epitope to which the at least one ISVD binds comprises at least one of the following combinations of amino acid residues:
   a) 2E, 3S, 4H and 5L, and/or
   b) 97E, 98L, 99G, 100P, 101D and 102N, and/or
   c) 98L, 99G, 100P, 101D, 102N and 103T, and/or
   d) 167L, 168E, 171R, 174L, 175E and 177K, and/or
   e) 205P, 206P and 207E, and/or
   f) 255Q, 256H, 257A, 259L, 260A, 261Q, and 262P,
   wherein the amino acid residues are numbered according to SEQ ID NO: 1.
24. The FcRn antagonist according to any one of items 5 to 23, wherein the FcRn epitope to which the at least one ISVD binds comprises at least one of the following combinations of amino acid residues:
   a) 1A, 2E, 3S, 4H and 5L, and/or
   b) 164R, 167L, 168E, 171R, 174L, 175E and 177K, and/or
   c) 204Y, 205P, 206P and 230E, and/or
   d) 205P, 206P, 207E and 208L,
   wherein the amino acid residues are numbered according to SEQ ID NO: 1.
25. The FcRn antagonist according to any one of items 5 to 24, wherein the FcRn epitope to which the at least one ISVD binds comprises at least the following amino acid residues 4H, 5L, 98L, 99G, 100P, 101D, 102N, 167L, 171R, 174L, 175E, 177K, 207E, 255Q, 256H, 257A, 259L, 260A, and 262P, wherein the amino acid residues are numbered according to SEQ ID NO: 1.
26. The FcRn antagonist according to any one of items 5 to 25, wherein the FcRn epitope to which the at least one ISVD binds comprises at least the following amino acid residues 2E, 3S, 4H, 5L, 32P, 97E, 98L, 99G, 100P, 101D, 102N, 103T, 167L, 168E, 171R, 174L, 175E, 177K, 205P, 206P, 207E, 255Q, 256H, 257A, 259L, 260A, 261Q, and 262P, wherein the amino acid residues are numbered according to SEQ ID NO: 1.
27. The FcRn antagonist according to any one of items 5 to 26, wherein the FcRn epitope to which the at least one ISVD binds comprises at least the following amino acid residues 1A, 2E, 3S, 4H, 5L, 32P, 97E, 98L, 99G, 100P, 101D, 102N, 103T, 164R, 167L, 168E, 171R, 174L, 175E, 177K, 204Y, 205P, 206P, 207E, 208L, 209Q, 255Q, 256H, 257A, 259L, 260A, 261Q, and 262P, wherein the amino acid residues are numbered according to SEQ ID NO: 1.
28. The FcRn antagonist according to any one of items 5 to 27, wherein the at least one ISVD specifically binds to the FcRn epitope in a pH-dependent manner, such that the binding affinity at an acidic pH (such as between 5.0 and 6.8) is at least three times higher than the binding affinity at an extracellular physiological pH (such as a pH of about 7.4).
29. The FcRn antagonist according to any one of items 5 to 28, wherein the at least one ISVD specifically binding to the FcRn epitope specifically binds to amino acid residues on FcRn that are not involved in binding of FcRn to serum albumin and/or that are not involved in binding of FcRn to IgG.
30. The FcRn antagonist according to any one of items 5 to 29, wherein the at least one ISVD specifically binding to the FcRn epitope consists of 4 framework regions (FR1 to FR4, respectively) and 3 complementarity determining regions (CDR1 to CDR3, respectively), and wherein:
   a) CDR1 (according to AbM) has the amino acid sequence of SEQ ID NO: 11 [=G F T F S S Y A M Y] and/or has an amino acid sequence having 4, 3, 2 or only 1 “amino acid difference(s)” (as defined herein) with the sequence of SEQ ID NO: 11; and
   b) CDR2 (according to AbM) has the amino acid sequence of SEQ ID NO: 12 [=A I S S G G G S T D] and/or has an amino acid sequence having 4, 3, 2 or only 1 “amino acid difference(s)” (as defined herein) with the sequence of SEQ ID NO: 12; and
   c) CDR3 (according to AbM) has the amino acid sequence of SEQ ID NO: 13 [=D T L Y T S L T S Y S Y], and/or has an amino acid sequence having 4, 3, 2 or only 1 “amino acid difference(s)” (as defined herein) with the sequence of SEQ ID NO: 13 or the CDR3 (according to AbM) has the amino acid sequence of SEQ ID NO: 189.
31. The FcRn antagonist according to any one of items 5 to 30, wherein the at least one ISVD comprises or consists of SEQ ID NO: 14 (T0263018B11-parent, EVQLVESGGGVVQPGGSLRLSCAASGFTFSSYAMYWVRQAPGKGLEWVSAISSGGGSTDYADSVKGRF TISRDNSKNTLYLQMNSLRPEDTALYYCAADTLYTSLTWYSYWGQGTLVTVSS), SEQ ID NO: 15 (T0263018B11-W100cS, EVQLVESGGGVVQPGGSLRLSCAASGFTFSSYAMYWVRQAPGKGLEWVSAISSGGGSTDYADSVKGRF TISRDNSKNTLYLQMNSLRPEDTALYYCAADTLYTSLTSYSYWGQGTLVTVSS), SEQ ID NO.: 185 (T0263018B11(E1D,W100cS), DVQLVESGGGVVQPGGSLRLSCAASGFTFSSYAMYWVRQAPGKGLEWVSAISSGGGSTDYADSVKGRF TISRDNSKNTLYLQMNSLRPEDTALYYCAADTLYTSLTSYSYWGQGTLVTVSS), SEQ ID NO.: 186 (T0263018B11(Y79S,T100bE,W100cS)),EVQLVESGGGVVQPGGSLRLSCAASGFTFSSYAMYWVRQ APGKGLEWVSAISSGGGSTDYADSVKGRFTISRDNSKNTLSLQMNSLRPEDTALYYCAADTLYTSLESYSY WGQGTLVTVSS), SEQ ID NO.: 187 (T0263018B11(D1E, Y79S,T100bE,W100cS), DVQLVESGGGVVQPGGSLRLSCAASGFTFSSYAMYWVRQAPGKGLEWVSAISSGGGSTDYADSVKGRF TISRDNSKNTLSLQMNSLRPEDTALYYCAADTLYTSLESYSYWGQGTLVTVSS), SEQ ID NO.: SEQ ID NO: 131 (T0263091D07, EVQLVESGGGVVQPGGSLRLSCAASGFSFSDYYMYWVRQAPGKGLEWVSAISSGGSSTYYADSVKGRFT ISRDNSKNTVYLQMNSLRPEDTALYYCAADYLSVPDPSYEYWGQGTLVTVSS) or SEQ ID NO: 144 (T0263204B12, EVQLVESGGGLVQPGGSLRLSCAASGFTLDYYAIGWFRQAPGKEREGVLCISSSGDSTYYADSVKGRFTIS RDNAKNTVYLQMDSLNPEDTAVYYCAVDPPSYWTGTGCLYGYRYWGQGTLVTVSS), or SEQ ID NO.: 158 (DVQLVESGGGLVQPGGSLRLSCAASGFTFSDYGMGWVRQAPGKGLEVVSYISSDGGETSYADSVKGRF TISRDNAKNMLYLQMSSLKLEDTALYYCAKGGVRGQGTLVTVSS), or SEQ ID NO.: 168 (EVQLVESGGGLVQPGGSLRLSCAASGFTFSDYGMGWVRQAPGKGLEVVSYISSDGGETSYADSVKGRFT ISRDNAKNMLYLQMSSLKLEDTALYYCAKGGVRGQGTLVTVSS).
32. The FcRn antagonist according to any one of items 1 to 31, wherein the antagonist further comprises at least one further moiety, preferably at least one further ISVD, such as an ISVD specifically binding to (human) serum albumin, and/or an ISVD specifically binding to a therapeutic target, preferably at least one ISVD specifically binding to (human) serum albumin.
33. The FcRn antagonist according to item 32, wherein the at least one further ISVD comprised therein consists of 4 framework regions (FR1 to FR4 respectively) and 3 complementarity determining regions (CDR1 to CDR3 respectively), and wherein:
   CDR1 is SFGMS (SEQ ID NO: 16),
   CDR2 is SISGSGSDTLYADSVKG (SEQ ID NO: 17) and
   CDR3 is GGSLSR (SEQ ID NO: 18),
   wherein the CDR sequences are determined according to Kabat; and/or
   wherein
   CDR1 is GFTFRSFGMS (SEQ ID NO: 19),
   CDR2 is SISGSGSDTL (SEQ ID NO: 20) and CDR3 is GGSLSR (SEQ ID NO: 21),
   wherein the CDR sequences are determined according to AbM numbering.
34. A composition comprising a FcRn antagonist as defined in any one of items 1 to 33.
35. The composition according to item 34, wherein the composition is a pharmaceutical composition.
36. Method for producing a FcRn antagonist as defined in any one of items 1 to 33, wherein the method comprises the steps of:
   a) expressing, in a suitable host cell or host organism or in another suitable expression system, a nucleic acid sequence encoding the FcRn antagonist as defined in any one of items 1 to 33; optionally followed by:
   b) isolating and/or purifying the FcRn antagonist as defined in any one of items 1 to 33.
37. Nucleic acid or nucleic acid sequence encoding a FcRn antagonist as defined in any one of items 1 to 33.
38. Vector comprising a nucleic acid or nucleic acid sequence as defined in item 37.
39. Non-human host or host cell comprising a vector comprising a nucleic acid sequence as defined in item 36 or expressing a nucleic acid sequence as defined in item 38.
40. The FcRn antagonist according to any one of items 1 to 33, or the composition according to any one of items 34 to 35 for use as a medicament.
41. The FcRn antagonist according to any one of items 1 to 33, or the composition according to any one of items 34 to 35 for use in the treatment of an IgG-mediated disorder.
42. The FcRn antagonist for use according to item 41, wherein the IgG-mediated disorder is an autoimmune disease.
43. The FcRn antagonist or composition for use according to item 42, wherein the autoimmune disease is selected from the group consisting of graft versus host disease (GVHD), systemic lupus erythematosus (SLE), myasthenia gravis, systemic sclerosis (SSc)/scleroderma, rheumatoid arthritis, psoriatic arthritis, osteoarthritis, diabetes, multiple sclerosis, pemphigus vulgaris, atopic dermatitis, psoriasis, asthma, allergy, idiopathic pulmonary fibrosis (IPF), idiopathic thrombocytopenia purpura (ITP), and hidradenitis suppurativa.
44. The FcRn antagonist or composition for use according to item 42, wherein the IgG-mediated disorder is hyperglobulinemia.
45. A FcRn antagonist according to any one of items 1 to 33 or the composition according to any one of items 34 to 35 for use in reducing the serum levels of an Fc-containing agent in a subject that has been administered the Fc-containing agent, wherein said polypeptide or composition is simultaneously or sequentially administered to said subject.
46. Kit comprising a FcRn antagonist according to any of items 1 to 33, a nucleic acid or nucleic acid sequence according to item 37, a vector according to item 38, or a host cell according to item 39.

1. A FcRn antagonist comprising:
a) at least one first polypeptide specifically binding to an epitope on Fc receptor (FcRn); and
b) at least a second polypeptide which competes with wild-type IgG1 Fc region for binding to FcRn.

2. The FcRn antagonist according to claim 1, wherein the at least two polypeptides (a) and (b) comprised in the FcRn antagonist show a synergistic or cooperative effect, preferably wherein the synergistic or cooperative effect allows for an improved binding and/or a similar half-life of the FcRn antagonists, preferably an improved half-life of the FcRn antagonists, as compared with the binding and/or half-life of each of the at least two polypeptides (a) and (b) on their own.

3. The FcRn antagonist according to claim 1, wherein the at least one first polypeptide as defined in (a) does not compete with wild-type IgG1 Fc region for binding to FcRn.

4. The FcRn antagonist according to claim 1, wherein the at least one first polypeptide as defined in (a) comprises or consists of an immunoglobulin single variable domain (ISVD).

5. The FcRn antagonist according to claim 4, wherein the FcRn epitope to which the at least one ISVD binds comprises at least one of the following amino acid residues 1A, 2E, 3S, 4H, 5L, 32P, 97E, 98L, 99G, 100P, 101D, 102N, 103T, 164R, 167L, 168E, 171R, 174L, 175E, 177K, 204Y, 205P, 206P, 207E, 208L, 209Q, 255Q, 256H, 257A, 259L, 260A, 261Q, and/or 262P, amino acid residues being numbered according to SEQ ID NO: 1.

6. The FcRn antagonist according to claim 5, wherein the FcRn epitope to which the at least one ISVD binds comprises at least one of the following combinations of amino acid residues:
a) 4H and 5L, and/or
b) 98L, 99G, 100P, 101D and 102N, and/or
c) 167L, 171R, 174L, 175E and 177K, and/or
d) 255Q, 256H, 257A, 259L, 260A and 262P,
amino acid residues being numbered according to SEQ ID NO: 1.

7. The FcRn antagonist according to claim 1, wherein the at least one polypeptide as defined in (b) comprises or consists of a Fc domain or a fragment thereof.

8. The FcRn antagonist according to claim 1, wherein the at least one polypeptide as defined in (b) specifically binds to FcRn with increased affinity relative to a wild-type IgG1 Fc region.

9. The FcRn antagonist according to claim 7, wherein the Fc domain or fragment thereof comprises at least one, preferably all, of the following amino acids at the following positions:
a) a tyrosine (Y) at amino acid position 252,
b) a threonine (T) at amino acid position 254,
c) a glutamic acid (E) at amino acid position 256,
d) a lysine (K) at amino acid position 433,
e) a phenylalanine (F) at amino acid position 434, and/or
f) a tyrosine (Y) at amino acid position 436;
according to EU numbering.

10. The FcRn antagonist according to claim 7, wherein the Fc domain or fragment thereof comprises a combination of the following four amino acid residues:
a) a tyrosine (Y) at amino acid position 252,
b) an aspartic acid (D) or a glutamic acid (E) at amino acid position 256,
c) a tryptophan (W) or a glutamine (Q) at amino acid position 307, and
d) a phenylalanine (F) or a tyrosine (Y) at amino acid position 434;
according to EU numbering.

11. The FcRn antagonist according to claim 1, wherein the at least one first polypeptide as defined in (a) comprises or consists of SEQ ID NO: 14, 15, 185, 186, 131, 144,187, 158 or 168, preferably SEQ ID NO: 15 or 186.

12. The FcRn antagonist according to claim 1, wherein the at least one second polypeptide as defined in (b) comprises or consists of SEQ ID NO: 22-24 and/or 119-123, preferably SEQ ID NO: 23, 24 or 119-123.

13. A fusion protein comprising the FcRn antagonists as defined in claim 1 and a further group, residue, moiety or binding unit.

14. A composition comprising the FcRn antagonist as defined in claim 1 and/or a fusion protein comprising the FcRn antagonist.

15. A method of treating an IgG-mediated disorder, the method comprising administering the FcRn antagonists as defined in claim 1 to a subject in need thereof, optionally wherein the IgG-mediated disorder is an autoimmune disease.

16. A nucleic acid or nucleic acid sequence encoding the FcRn antagonist as defined in claim 1.

17. A vector comprising a nucleic acid or nucleic acid sequence as defined in claim 16.

18. A non-human host or a host cell comprising a vector comprising the nucleic acid sequence as defined in claim 16.
